SEQUENCE LISTING (28 sequences)

SEQ ID NO: 1 - RNA, 18 nt, Artificial Sequence
  The top two siRNAs for viability assay
ccaguaccuagaacuuaa

SEQ ID NO: 2 - RNA, 19 nt, Artificial Sequence
  The top two siRNAs for vaibility assay - #4
agaaugagcuggcccacuu

SEQ ID NO: 3 - DNA, 21 bp, Artificial Sequence
  inserted LGP2 shRNA sequence
attcttgcggtcatcgaacag

SEQ ID NO: 4 - DNA, 24 bp, Artificial Sequence
  IFNbeta sense primer
aactttgacatccctgaggagatt

SEQ ID NO: 5 - DNA, 17 bp, Artificial Sequence
  IFNbeta antisense primer
gcggcgtcctccttctg

SEQ ID NO: 6 - DNA, 20 bp, Artificial Sequence
  GAPDH sense
ctctgctcctcctgttcgac

SEQ ID NO: 7 - DNA, 20 bp, Artificial Sequence
  GAPDH antisense
gttaaaagcagccctggtga

SEQ ID NO: 8 - DNA, 23 bp, Artificial Sequence
  mmcGAS
gaggaaauccgcugagucadtdt

SEQ ID NO: 9 - DNA, 21 bp, Artificial Sequence
  mIFN-beta forward
ggtggaatgagactattgttg

SEQ ID NO: 10 - DNA, 18 bp, Artificial Sequence
  mIFN-beta reverse
aagtggagagcagttgag

SEQ ID NO: 11 - DNA, 24 bp, Artificial Sequence
  m-cGAS forward
accggacaagctaaagaaggtgct

SEQ ID NO: 12 - DNA, 24 bp, Artificial Sequence
  m-cGAS reverse
gcagcaggcgttccacaactttat

SEQ ID NO: 13 - DNA, 21 bp, Artificial Sequence
  18S forward
cgtctgccctatcaactttcg

SEQ ID NO: 14 - DNA, 20 bp, Artificial Sequence
  18S reverse
tgccttccttggatgtggta

SEQ ID NO: 15 - RNA, 32 nt, Artificial Sequence
  U1 stem loop I sequence
gggagaaccaugaucacgaaggugguuuuccc

SEQ ID NO: 16 - RNA, 38 nt, Artificial Sequence
  U1 stem loop II sequence
gggcgaggcuuauccauugcacuccggaugugcucccc

SEQ ID NO: 17 - RNA, 26 nt, Artificial Sequence
  U1 stem loop III sequence
cgauuuccccaaaugugggaaacucg

SEQ ID NO: 18 - RNA, 31 nt, Artificial Sequence
  U1 stem loop IV sequence
uagugggggacugcguucgcgcuuuccccug

SEQ ID NO: 19 - DNA, 74 bp, Artificial Sequence
  U1 stem loops I and II sequence
gggagataccatgatcacgaaggtggttttcccagggcgaggcttatccattgcactccggatgtgctgacccc

SEQ ID NO: 20 - DNA, 73 bp, Artificial Sequence
  U1 stem loops II and III sequence
gggcgaggcttatccattgcactccggatgtgctgacccctgcgatttccccaaatgtgggaaactcgactgc

SEQ ID NO: 21 - RNA, 188 nt, Artificial Sequence
  U2 SEQUENCE
aucgcuucucggccuuuuggcuaagaucaaguguaguaucuguucuuaucaguuuaauaucugauacguccucuauccgaggacaauauauuaaauggauuuuuggagcagggagauggaauaggagcuugcuccguccacuccacgcaucgaccugguauugcaguaccuccaggaacggugcaccc

SEQ ID NO: 22 - DNA, 19 bp, Artificial Sequence
  Positive control (19-mer) -1
gcaugcgaccucuguuuga

SEQ ID NO: 23 - DNA, 19 bp, Artificial Sequence
  Positive control (19-mer) -2
cguacgcuggagacaaacu

SEQ ID NO: 24 - DNA, 19 bp, Artificial Sequence
  Negative control (19-mer) -1
gcaugcgaccucuguuuga

SEQ ID NO: 25 - DNA, 19 bp, Artificial Sequence
  Negative control (19-mer) -2
cguacgcuggagacaaacu

SEQ ID NO: 26 - DNA, 81 bp, Artificial Sequence
  m5
gacgaagaccacaaaaccagataaaaaattattttttatctggttttgtggtcttcgtctatagtgagtcgtattaatttc

SEQ ID NO: 27 - DNA, 101 bp, Artificial Sequence
  M8
gaaattaatacgactcactatagacgaagaccacaaaaccagataaaaaaaaaaaaaaaataattttttttttttttttatctggttttgtggtcttcgtc

SEQ ID NO: 28 - protein, 8 aa, Artificial Sequence
  SIY peptide
SIYRYYGL

(19) United States
(12) Patent Application Publication
(10) Pub. No.: US 2017/0268001 A1
(43) Pub. Date: Sep. 21, 2017

(54) RNAS WITH TUMOR RADIO/CHEMO-SENSITIZING AND IMMUNOMODULATORY PROPERTIES AND METHODS OF THEIR PREPARATION AND APPLICATION

(71) Applicant: THE UNIVERSITY OF CHICAGO, Chicago, IL (US)

(72) Inventors: Nikolai N. Khodarev, Villa Park, IL (US); Diana Rose E. Ranoa, Chicago, IL (US); Sean P. Pitroda, Chicago, IL (US); Ralph R. Weichselbaum, Chicago, IL (US)

(21) Appl. No.: 15/459,602

(22) Filed: Mar. 15, 2017

Related U.S. Application Data
(60) Provisional application No. 62/309,178, filed on Mar. 16, 2016.

(51) Int. Cl. C12N 15/113; A61K 45/06; A61N 5/10; A61K 31/7088

(52) U.S. Cl. CPC C12N 15/113; A61K 31/7088; A61K 45/06; A61N 5/1001; A61N 2005/1087; A61N 2005/1098

(57) ABSTRACT

Compositions, kits and methods for treating cancer in a subject in need thereof are disclosed involving one or more genes the suppression of which renders the cancer chemosensitive and/or radiosensitive.

### CROSS REFERENCE TO RELATED APPLICATIONS

This application claims the benefit of U.S. Provisional Patent Application Ser. No. 62/309,178, filed on Mar. 16, 2016, the contents of which are herein incorporated by reference in their entirety.

### BACKGROUND OF THE INVENTION

1. Field of the Invention

The present invention relates to the identification and control of gene targets for treatment of cancers, including chemoresistant and/or radioresistant cancers. Specifically, the present invention relates to compositions comprising at least one rbRNA (e.g., snRNA) or its functionally equivalent fragment for treating cancers, especially prior to an ionization radiation treatment.

2. Description of the Background of the Invention

Cancer is not fully understood on a molecular level and remains a leading cause of death worldwide. One of the deadliest forms of cancer is solid tumors. One such solid tumor is lung cancer, the most common cancer worldwide and the leading cause of cancer-related death in the United States. Approximately 219,000 new diagnoses and over 159,000 deaths from lung cancer occur annually in the United States. Approximately 85% of lung cancers are non-small cell histology (NSCLC), including lung adenocarcinomas, which are the most common lung cancer type in the U.S. Treatment of early and intermediate stage NSCLC usually involves surgery, stereotactic radiotherapy, or conventional radiotherapy with or without adjuvant chemotherapy. Chemotherapy regimens for lung cancer, either concurrent with radiotherapy (RT) or adjuvant to surgery, usually incorporate platinum-based drugs such as cisplatin or carboplatin, as this has been shown to confer a survival advantage when either combined with radiotherapy or in the adjuvant setting.

Standard fractionated radiotherapy as the primary treatment for NSCLC is reserved for patients with tumors too advanced to resect, who are medically unstable, whose disease has spread beyond the chest, or in the case of small or metastatic tumor hypofractionated stereotacktic body radiotherapy. The utility of postoperative radiotherapy is controversial and subsets of patients who are likely to benefit have been proposed. These include patients with advanced lymph node metastases (N2-N3 or extra-capsular extension) and close or positive surgical margins. However, clear clinical and/or molecular selection criteria for patients who may benefit from postoperative radiotherapy remains elusive. No prognostic or predictive signature to select patients with NSCLC who may benefit from radiotherapy or chemotherapy is consistently used in clinical practice at this time.

The activity of Jak/Stat dependent genes has been shown to predict the outcome of patients with lung cancer and their response to the adjuvant radiotherapy or chemotherapy. Stat1 (Signal Transducer and Activator of Transcription 1) is a member of the Stat family of proteins, which are mediators of Jak signaling. Stat1 is phosphorylated at the tyrosine 701 position by Jak kinases and translocates to the nucleus to activate the transcription of hundreds of Interferon-Stimulated Genes (ISGs).

Further, clinical trials of Jak/Stat pathway inhibitors in hematological malignancies are ongoing for the pharmacological suppression of the Stat-related pathways. Jak inhibitors currently available include either specific inhibitors of Jak2 or combined inhibitors of Jak1 and Jak2. The radiosensitizing effects of the Jak2 inhibitor TG101209 (TargeGen Inc., CAS 936091-14-4) were recently described in two lung cancer cell lines and were associated with suppression of the Stat3 pathway. TG101209 was developed to potentially inhibit myeloproliferative disorder-associated JAK2V617F and MPLW515L/K mutations. Activation of Jak2/Stat3 signaling was demonstrated in several other lung cancer cell lines and was associated with increased oncogenic potential, tumor angiogenesis, and EGFR signaling associated with progression of lung adenocarcinomas. Further, next-generation sequencing recently revealed constitutively active Jak2 mutation (V617F) in some lung cancer patients.

To date, few publications describe the application of these drugs in lung cancer models, and mechanisms of their action in lung cancer are still poorly understood. The majority of publications regarding the application of Jak inhibitors in solid tumors, including lung cancer, explain their action based on pathways activated by Stat3, Stat5 or not directly related to Stat signaling. Jak/Stat1 pathways in solid tumors are not described in the context of therapeutic effects of Jak inhibitors, though they are already described in some myelodysplastic diseases. It is believed that Jak1 kinase is activated by Jak2 kinase and both are necessary for activation of Stat1 and Stat3. It is also believed that Stat1 and Stat3 can form heterodimers with transcriptional activity. Additionally, genes induced by Jak2/Stat3 activation overlap with IFN/Stat1-dependent genes. Finally, constitutively active oncogenic Jak2 (Jak2V617F) induces genes overlapping with the Stat1-dependent genes.

While the importance of Jak/Stat signaling, in general, for cancers continues to be investigated, the role that downstream effector genes may play in tumors remains undefined. Consequently, there is an urgent and definite need to identify the downstream effector genes that may potentially have a role in tumor development associated with activation of the Jak/Stat pathway. Such genes may provide new targets for Jak-related therapy of cancers, including, for example, lung cancer, or for sensitization of cancers for chemotherapies and/or radiotherapies. Therefore, there is a need to determine the identities of downstream effector genes in the Jak/Stat pathway of cancer, including solid tumors, that may play a role in treating cancers, and to develop effective cancer therapies around these downstream effector genes. More effective and targeted cancer therapies with potentially fewer side effects are also needed. PCT application Ser. No. PCT/US2014/062228 describes compositions, kits and methods for treating cancer in a subject in need thereof are disclosed involving one or more genes the suppression of which renders the cancer chemosensitive and/or radiosensitive.

Accumulating data indicate a link between ionizing radiation (IR) and interferon (IFN) signaling. IFN signaling activates multiple interferon-stimulated genes (ISGs) and leads to growth arrest and cell death in exposed cell populations. It has been demonstrated that IR-induced tumor-derived type I IFN production is important for improved tumor responses. Interferons can sensitize tumor cells to radio/chemotherapy. At the same time, Type I interferons play critical role in regulation of immune response and regulation of targets of the current immune checkpoint therapy. However, molecular mechanisms governing tumor cell-intrinsic IR-mediated IFN activation are largely unknown. Applicants previously identified DEXH box RNA helicase LGP2 (DHX58) as a negative regulator of IR-induced cytotoxic IFN-beta production contributing to cell-autonomous radioprotective effects in cancer cells. LGP2 is a cytoplasmic RIG-I-like receptor (RLR) which suppresses IFN signaling in the response to viral double-stranded RNA. Therefore this finding implicated RNAs as potential inducers of IFN response and radiosensitizers. Currently different types of chemically synthesized RNA are used as adjuvant vaccines to improve response of tumors to anticancer therapy and stimulate host immune system. Labor and cost of optimization of chemical structure of such RNAs can be substantially reduced if natural prototypes with increased activity will be defined and appropriate test systems will be developed. Needed in the art are new approaches to identify and test different natural endogenous RNAs with ability to act as immunostimulators and tumor suppressors.

### SUMMARY OF THE INVENTION

In one aspect, the present invention relates to a composition for treating cancer in a subject in need thereof.

In one embodiment, the present invention relates to a composition for treating cancer in a subject in need thereof, and the composition comprises a therapeutically effective amount of at least one rbRNA (e.g., snRNA) or its functionally equivalent fragment, and a pharmaceutically acceptable carrier, wherein the at least one rbRNA (e.g., snRNA) or its functionally equivalent fragment activates primary RNA or DNA sensors and wherein the composition is administered to the subject before a dose of ionized radiation is administered on the subject.

In one embodiment, the present invention relates to a composition for treating cancer in a subject in need thereof, and the composition comprises a therapeutically effective amount of at least one rbRNA's (e.g., snRNA's) functionally equivalent fragment, and a pharmaceutically acceptable carrier, wherein the at least one rbRNA's (e.g., snRNA's) functionally equivalent fragment activates primary RNA or DNA sensors and wherein the composition is administered to the subject before a dose of ionized radiation is administered on the subject.

In one embodiment, the at least one rbRNA's (e.g., snRNA's) functionally equivalent fragment comprises a stem-loop region of the rbRNA (e.g., the snRNA),

In one embodiment, the present invention relates to a composition for treating cancer in a subject in need thereof, and the composition comprises a therapeutically effective amount of at least two rbRNA (e.g., snRNA) or their functionally equivalent fragments, and a pharmaceutically acceptable carrier, wherein the at least two rbRNA (e.g., snRNA) or their functionally equivalent fragment activates primary RNA or DNA sensors and wherein the composition is administered to the subject before a dose of ionized radiation is administered on the subject.

In one embodiment, the at least one rbRNA (e.g., snRNA) is selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3. In one embodiment, the at least one snRNA is U2 snRNA. In one embodiment, the at least one snRNA is U1 snRNA.

In one embodiment, the at least one rbRNA (e.g., snRNA) is selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

In one embodiment, the primary RNA or DNA sensor comprises at least one of RIG1, MDA5, DAI, IFI16, Aim2, and cGAS. In one preferred embodiment, the primary RNA or DNA sensor is RIG1.

In one embodiment, the at least two rbRNAs (e.g., snRNAs) are selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

In one embodiment, the at least two rbRNAs (e.g., snRNAs) comprise U2. In one embodiment, the at least two rbRNAs (e.g., snRNAs) comprise U1.

In one embodiment, the at least two rbRNAs (e.g., snRNAs) are selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

In one embodiment, the primary RNA or DNA sensors comprise at least one of RIG1, MDA5, DAI, IFI16, Aim2, and cGAS. In one preferred embodiment, the primary RNA or DNA sensors comprise at least RIG1.

In one embodiment, the composition further comprises another therapeutic agent.

In one embodiment, the other therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

In another aspect, the present invention relates to a method of treating cancer in a subject in need thereof. The method comprises the steps of (a) administering to the subject a pharmaceutical composition comprising: a therapeutically effective amount of at least one rbRNA (e.g., snRNA) or its functionally equivalent fragment, and a pharmaceutically acceptable carrier, wherein the at least one rbRNA (e.g., snRNA) or its functionally equivalent fragment activates a primary RNA or DNA sensor, and wherein the endogenous IFNbeta (IFNβ production of the subject is regulated, and (b) administering to the subject a therapeutic amount of ionizing radiation.

In one embodiment, the least one rbRNA (e.g., snRNA) or its functionally equivalent fragment is a double-stranded RNA.

In one embodiment, the at least one rbRNA (e.g., snRNA) is selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

In one embodiment, the at least one rbRNA (e.g., snRNA) is selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

In one embodiment, the at least one rbRNA (e.g., snRNA) is U2 snRNA. In one embodiment, the at least one rbRNA (e.g., snRNA) is U1 snRNA.

In one embodiment, the composition further comprises another therapeutic agent.

In one embodiment, the other therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

In one embodiment, the at least one rbRNA (e.g., snRNA) or its functionally equivalent fragment is further covalently attached to a reporter group.

In one embodiment, the pharmaceutically acceptable carrier comprises at least one of a nanocarrier, a conjugate, a nucleic-acid-lipid particle, a vesicle, an exosome, a protein capsid, a liposome, a dendrimer, a lipoplex, a micelle, a virosome, a virus like particle, and a nucleic acid complex.

In one embodiment, the primary RNA or DNA sensor comprises at least one of RIG1, MDA5, DAI, IFI16, Aim2, and cGAS. In one preferred embodiment, the primary RNA or DNA sensor is RIG1.

In one embodiment, the ionizing radiation comprises at least one of brachytherapy, external beam radiation therapy, and radiation from cesium, iridium, iodine, and cobalt.

In one embodiment, the subjection is a human being.

According to a first aspect, a method of treating cancer in a subject in need thereof in provided by regulation of endogenous IFNbeta (IFNβ production in the subject by, for example: 1) suppressing in a therapeutically effective amount at least one of a product or expression of an Interferon-Stimulated Gene (ISG) in the subject; 2) inducing a therapeutically effective amount of activation of Type I Interferon in the subject; 3) maintaining in a therapeutically effective amount activation of Type I Interferon in the subject; and/or 4) maintaining radio/chemoprotection of normal non-disease state tissue in the subject by suppressing in a therapeutically effective amount at least one of: i) a primary RNA or DNA sensor; ii) a major adaptor protein of a RNA/DNA-dependent pathway of IFN production; and/or iii) up-regulation or activation or gene transfer of two apical repressors of a RNA/DNA-dependent pathway of IFN production. The method may also include administering to the subject a therapeutic amount of ionizing radiation.

In one embodiment, the method includes suppressing the product or the expression of the Interferon-Stimulated Gene (ISG).

In yet another embodiment, the Interferon-Stimulated Gene (ISG) includes at least one RIG1-like receptor (RLR) family member.

In another embodiment, ionizing radiation induced cytotoxic IFNβ production is substantially maintained in the subject at levels substantially found prior to the administration of the ionizing radiation.

In yet another embodiment, Mitochondrial Antiviral Signaling Protein (MAVS)-dependent induction of endogenous IFNβ production is maintained in the subject at substantially the same level found in the subject prior to the administration of the ionizing radiation.

In other embodiments, the RIG1-like receptor (RLR) family member includes, for example, RIG1 (Retinoic Acid-inducible Gene 1), LGP2 (Laboratory of Genetics and Physiology 2), and/or MDA5 (Melanoma Differentiation-Associated Protein 5).

In further embodiments, suppressing of the Interferon-Stimulated Gene (ISG) results in suppression of growth or proliferation of the cancer, cell death of the cancer, and/or sensitization of the cancer to the ionizing radiation and/or chemotherapy.

In another embodiment, suppressing production of the Interferon-Stimulated Gene includes the suppression of expression of at least one Cytoplasmic Pattern-recognition Receptor (PRR) protein, including, for example, RIG1, LGP2, and/or MDA5.

In still other embodiments, the method of treating cancer includes maintaining activation of Type I Interferon in a subject to maintain ionizing radiation and chemotherapy sensitization in the subject.

In yet other embodiments, the method includes administering to a subject a therapeutic amount of an agent that maintains activation of Type I Interferon in the subject.

In one embodiment, the agent includes at least one of a shRNA, a siRNA, a micro-RNA mimic, an antisense oligonucleotide, a chemical, and a protein inhibitor.

In another embodiment, the agent down-regulates cytoplasmic DNA-sensoring pathway-exonuclease TREX1 (Three Prime Repair Exonuclease 1).

In yet another embodiment, the agent up-regulates at least one of DAI (DNA-dependent Activator of IFN regulatory factors), IFI16 (Gamma-interferon-inducible protein Ifi-16), and Aim2 (Interferon-inducible protein AIM2).

In another embodiment, the primary RNA or DNA sensor includes at least one of RIG1, MDA5, DAI, IFI16, Aim2, and cGAS.

In one embodiment, the major adaptor protein of the RNA/DNA-dependent pathway of IFN production includes MAVS and/or STING.

In yet another embodiment, the two apical repressors of the RNA/DNA-dependent pathway of IFN production include LGP2 and/or TREX1.

In another embodiment, ionizing radiation includes brachytherapy, external beam radiation therapy, or radiation from cesium, iridium, iodine, and/or cobalt.

In still another embodiment, the method of treating cancer includes inducing Type I Interferon production in a subject to maintain ionizing radiation and chemotherapy sensitization in the subject.

In one embodiment, the method includes administering to a subject a therapeutic amount of an agent that induces the Type 1 Interferon production in the subject.

In yet another embodiment, the agent enhances STING signaling.

In another embodiment, the agent increases cGAS levels in a subject, and in yet another embodiment, the agent enhances expression of a cGAS gene in a cancerous cell in the subject.

In another embodiment, the agent is cGAMP.

In still another embodiment, the agent activates at least one endosomal toll-like receptor (TRL) including, for example, TLR3, TLR7, TLR8 and TLR9.

In one embodiment, the agent interacts with at least one adaptor protein that includes at least one of myeloid differentiation primary-response protein 88 (MyD88) and TIR-domain-containing adaptor protein inducing IFN-β (TRIF).

In another embodiment, the agent is administered to a subject that increases levels of cGAS in a cancerous cell.

In yet another embodiment, the cGAS levels are greater than about 100% of a cancerous-state control cell.

In still another embodiment, the agent is delivered to a cancerous cell by a pharmaceutical carrier, including, for example, a nanocarrier, a conjugate, a nucleic-acid-lipid particle, a vesicle, a exosome, a protein capsid, a liposome, a dendrimer, a lipoplex, a micelle, a virosome, a virus like particle, a nucleic acid complexes, and combinations thereof.

In yet another embodiment, the agent is delivered into the cytosol of a dendritic cell.

In another aspect, a pharmaceutical composition for treating cancer in a subject in need thereof is provided that includes a therapeutically effective amount of an agent that regulates endogenous IFNbeta (IFNβ production in the subject.

In another aspect, a pharmaceutical composition for treating cancer in a subject in need thereof is provided that includes a therapeutically effective amount of an agent that induces a therapeutically effective amount of activation of Type I Interferon in the subject;

In one embodiment, the agent suppresses at least one of a product or the expression of an Interferon-Stimulated Gene (ISG) in the subject.

In yet another embodiment, the agent maintains activation of Type I Interferon in the subject.

In another embodiment, a pharmaceutical composition includes an agent that maintains radio/chemoprotection of normal non-disease state tissue in a subject by suppression of at least one of: i) a primary RNA or DNA sensor, ii) a major adaptor protein of a RNA/DNA-dependent pathway of IFN production, and iii) up-regulation or activation or gene transfer of two apical repressors of a RNA/DNA-dependent pathway of IFN production.

In still another embodiment, a pharmaceutical composition may contain one or more optional pharmaceutically acceptable carriers, diluents and excipients.

In yet another embodiment, a pharmaceutical composition includes an agent that suppresses at least one of the product or the expression of the Interferon-Stimulated Gene (ISG), which may include, for example, at least one RIG1-like receptor (RLR) family member.

In another embodiment, a pharmaceutical composition includes an agent maintains activation of Type I Interferon and includes at least one of a shRNA, a siRNA, a micro-RNA mimic, an antisense oligonucleotide, a chemical, and a protein inhibitor.

In yet another embodiment, a pharmaceutical composition includes an agent that down-regulates a cytoplasmic DNA-sensoring pathway-exonuclease TREX1 (Three Prime Repair Exonuclease 1).

In another embodiment, a pharmaceutical composition includes an agent that down-regulates a suppressor of cytoplasmic RNA-sensoring pathway-LGP2.

In yet another embodiment, a pharmaceutical composition includes an agent that up-regulates at least one of DAI (DNA-dependent Activator of IFN regulatory factors), IFI16 (Gamma-interferon-inducible protein Ifi-16), and Aim2 (Interferon-inducible protein AIM2).

In one embodiment, the pharmaceutical composition may also include a therapeutically effective amount of at least one antineoplastic agent and/or a radiotherapy agent.

In yet another embodiment, a pharmaceutical composition includes an agent that induces Type I Interferon production in the subject.

In another embodiment, a pharmaceutical composition includes an agent that enhances STING signaling.

In still another embodiment, a pharmaceutical composition includes an agent that increases cGAS levels in the subject.

In yet another embodiment, a pharmaceutical composition includes an agent that enhances expression of a cGAS gene in a cancerous cell in the subject.

In another embodiment, a pharmaceutical composition includes cGAMP.

In one embodiment, a pharmaceutical composition includes an agent that activates at least one endosomal toll-like receptor (TLR), including at least one of TLR3, TLR7, TLR8 and TLR9.

In yet another embodiment, a pharmaceutical composition includes an agent that increases level of cGAS in a cancerous cell, and in one embodiment cGAS levels are equal to or greater than about 100% of a cancerous state control cell.

In another embodiment, a pharmaceutical composition includes an agent that is delivered to the cancerous cell by a pharmaceutical carrier.

In still another embodiment, a pharmaceutical composition includes a pharmaceutical carrier that includes at least one of a nanocarrier, a conjugate, a nucleic-acid-lipid particle, a vesicle, an exosome, a protein capsid, a liposome, a dendrimer, a lipoplex, a micelle, a virosome, a virus like particle, and a nucleic acid complexes.

In yet another embodiment, a pharmaceutical composition includes an agent that is delivered into a cytosol of a dendritic cell.

In another aspect, a method of protecting normal non-disease state tissue from genotoxic stress is provided that includes suppressing in the tissue at least one of a product or the expression of an Interferon-Stimulated Gene in a therapeutically effective amount.

In one embodiment, suppressing production of the Interferon-Stimulated Gene includes administering to a tissue a neutralizing antibody to IFNβ or an antagonist of Type I IFN receptor (IFNAR1).

In yet another embodiment, administration of a neutralizing antibody or an antagonist substantially prevents cytotoxic effects of LGP2 depletion in the tissue.

In another embodiment, genotoxic stress includes exposure of a tissue to ionizing radiation, ultraviolet light, chemotherapy, and/or a ROS (Reactive Oxygen Species).

In one embodiment, a tissue is from a subject diagnosed with a cancer and the normal non-disease state tissue is substantially free of the cancer.

In yet another embodiment, a subject is a human.

In yet another aspect, a prognostic kit for use with a tissue having a high grade glioma is provided that includes at least one set of primers for QRT-PCR detection of LGP2 to determine expression levels of LGP2 in the tissue.

In one embodiment, high expression levels of LGP2 and low expression levels of LGP2 predicts improved prognosis in treating a high grade glioma.

In yet another embodiment, tissue is from brain tissue of a human subject.

In another embodiment, high expression levels of LGP2 are at least about 1.5 fold greater than an expression level of LGP2 in a normal non-disease state tissue of a human subject.

In yet another embodiment, low expression levels of LGP2 are at least about 1.5 fold less than an expression level of LGP2 in a normal non-disease state tissue of a human subject.

In still another embodiment, a prognostic kit may include at least one of a reagent for purification of total RNA from a tissue, a set of reagents for a qRT-PCR reaction, and a positive control for detection of LGP2 mRNA.

### BRIEF DESCRIPTION OF THE FIGURES

FIG. 1 shows the identification of LGP2 as pro-survival ISG. In each cell line tested 89 screened genes were ranked according to the ability of corresponding siRNAs to suppress cell viability as measured by CellTiter-Glo® luminescent assay (Promega, Madison, Wis.). FDR-corrected significance values for each gene across all tested cell lines were estimated by rank aggregation approach (see Methods). Data are presented as negative log-transformed false discovery ratios (FDR) for each gene on the basal level (closed triangles, right Y-axis) and 48 hours after irradiation at 3Gy (open diamonds, left Y axis);

FIGS. 2A, 2B, 2C and 2D show knockdown of LGP2 enhances radiation-induced killing. Cell death was quantified by flow cytometric analysis using Annexin-V and propidium iodide staining. Tumor cells were treated with IR (5Gy) 24 h post-transfection with indicated siRNA. FIG. 2A: Graphical representation of flow cytometric data in WiDr cells that were collected 48 h post-IR treatment. FIG. 2B: Quantification of flow cytometric experiments in D54, WiDr and Scc61 cells collected 48 h post-IR treatment. The data are represented as fold-change relative to siNT at 0Gy. FIG. 2C and FIG. 2D: Clonogenic survival curves in D54 (FIG. 2C) and Scc61 (FIG. 2D) cells transiently transfected with siNT or siLGP2 and irradiated at 0, 3, 5 or 7Gy. Data are represented in a semi-log scale. Western blots are representative of siRNA mediated knockdown of LGP2. In all experiments, data are presented as mean values of at least three independent measurements; error bars are standard deviations and significance was assessed using two-tailed t-test (* indicates p<0.05);

FIGS. 3A and 3B show overexpression of LGP2 inhibits radiation-induced killing. D54 cells were stably transfected by full-size p3×FLAG-CMV10-LGP2 (LGP2) or control p3×FLAG-CMB10 (Flag). Selected clones were propagated, plated in 6-well plates and irradiated at 0, 5 and 7Gy. FIG. 3A: Crystal violet staining of survived colonies 12 days after irradiation of cells, transfected with Flag (upper panel) or LGP2 (lower panel). FIG. 3B: Quantification of survival fraction of mock-transfected and LGP-transfected cells (see Methods). Representative Western blot of stable Flag and LGP2 clone is inserted into panel B;

FIG. 4 shows that LGP2 is radioinducible. D54, WiDr and Scc61 cells were irradiated at 6Gy; 72 hours post-IR cells lysates were analyzed by Western blotting;

FIGS. 5A, 5B, and 5C show that IR induces cytotoxic IFNβ response. FIG. 5A: Radiation-induced expression of IFNβ mRNA. IFNβ expression in D54, WiDr, SCC61 and HEK293 cells treated with or without 6 Gy IR was measured by qRT-PCR and normalized to GAPDH expression. Data are expressed as fold-change relative to non-irradiated cells. FIG. 5B: Radiation-induced activation of IFNβ promoter. HEK293 cells were transiently co-transfected with pGL3-Ifnβ and pRL-SV40. Firefly luciferase was normalized to Renilla luciferase and is expressed relative to non-irradiated cells at each collection time. FIG. 5C: Type I IFN receptor (IFNAR1) is needed for cytotoxicity induced by IR. Wild type (Wt) and IFNAR1^{-/-} MEFs were treated with the indicated doses of IR and collected 96 h post-IR. Viability was determined by methylene blue staining and extraction, followed by spectrophotometric quantification. Viability is shown relative to non-irradiated control cells. Data are represented as mean with standard deviation for assays performed in at least triplicates;

FIGS. 6A and 6B show that LGP2 inhibits IR-induced cytotoxic IFNβ. FIG. 6A: LGP2 suppresses IR-induced activation of IFNβ promoter. HEK293 cells were stably transduced with shRNA directed to LGP2 or non-targeting control (shNT). Cells were transfected with pGL3-Ifnβ and pRL-SV40, irradiated (indicated dose) and collected 72 h after IR. Firefly luciferase activity was normalized to Renilla luciferase activity and is expressed relative to non-irradiated cells. FIG. 6B: Neutralizing antibodies to IFNβ prevent cytotoxic effects of LGP2 depletion. D54 cells were depleted of LGP2 with siRNA (see FIG. 2C) and irradiated at 0, 3 or 6Gy in the presence or absence of neutralizing antibody to IFNβ (1 μg/mL). Cell viability was assessed 96 h post-IR using methylene blue assay. Data are normalized to non-targeting siRNA at 0 Gy and represented as mean with error bars showing standard deviation for assays performed at least in triplicate. Significance was measured using two-tailed t-test (*p<0.05);

FIGS. 7A, 7B, 7C, and 7D show that expression of LGP2 is associated with poor overall survival in patients with GBM. FIG. 7A: Expression of Interferon-Stimulated genes (ISGs) and LGP2 in the Phillips database (n=77). Yellow represents up-regulated and blue-down-regulated genes. Rows correspond to patients while columns correspond to individual genes in IRDS signature. FIG. 7B: Kaplan-Meier survival of LGP2-high (LGP2+) and LGP2-low (LGP2−) patients from Phillips database. FIG. 7C: Expression of ISGs and LGP2 in the TCGA database (n=382) and (FIG. 7D) Survival of LGP2+ and LGP2-patients in CGA database. p-values represent Cox proportional hazards test;

FIGS. 8A and 8B show activation of IFNβ by IR is suppressed by LGP2. Acute response to IR leads to activation of IFNβ and induction of ISGs with cytotoxic functions (Panel A). Chronic exposure to cytotoxic stress leads to constitutive expression of some ISGs with pro-survival functions and LGP2-dependent suppression of the autocrine IFNβ loop;

FIG. 9 shows schematics of cytoplasmic sensors for RNA and DNA. Two primary RNA sensors are RIG1 (DDX58) and MDA5 (IFIH1), while family of DNA sensors is redundant and includes, for example, cGAS (MB21D1), DAI (ZBP1, DLM1) AIM2, IFI16 and several other proteins. LGP2 (DHX58) represents apical suppressor of RNA-dependent pathway while exonuclease TREX1 (DNase III)-apical suppressor of DNA pathway. RNA pathway converges on adaptor protein MAVS (aka IPS1; VISA; CARDIFF) and DNA pathway converges on the adaptor protein STING (aka TMEM173; MPYS; MITA; ERIS). Both adaptor proteins activate NFkB-dependent, IRF3/IRF7-dependent transcription of Type I IFNs, which can further act through autocrine and paracrine loops as cytotoxins and/or signaling molecules. We found that for these pathways suppression of proteins with pro-IFN function (primary sensors, adaptor proteins) render cells radioresistant. On contrary, suppression of proteins with anti-IFN function (LGP2, TREX1) renders cells radiosensitive. These data are shown below in FIGS. 10-15;

FIG. 10 shows RT-PCR confirmation of stable shRNA-derived knock-downs (KDs) of STING, DAI and AIM2 genes in SCC61 cell line. In other experiments we used siRNAs or embryonic fibroblasts from transgenic (knock-out) mice;

FIG. 11 shows that suppression of STING in SCC61 cell line leads to the suppression of IR-induced IFN-beta and IFN-lambda, but not IL-1b;

FIG. 12 shows that KD of STING in SCC61 leads to radioprotection of cells;

FIG. 13 shows that KD of AIM2 in our experimental system leads to the suppression of IR-induced IFN-beta and IFN-lambda, which allows predict radioprotective effects of suppression of this protein;

FIG. 14 shows that suppression of TREX1 in SCC61 leads to radiosensitization of cells (see FIG. 1);

FIGS. 15A, 15B, 15C and 15D show that suppression of LGP2 in D54 and SCC61 leads to radiosensitization, while suppression of MAVS- to radioprotection_of cells. FIG. 15E shows that MAVS up-regulates transcription of IFN-beta, while LGP2 suppresses this MAVS-dependent effect and FIG. 15F shows schematics of interaction between LGP2 and MAVS in generation of IR-induced IFN-mediated cytotoxic response;

FIGS. 16A, 16B, 16C, 16D, 16E, and 16F show STING signaling providing an antitumor effect of radiation. MC38 tumors in WT mice and KO mice were treated locally one dose of 20Gy ionizing radiation (IR) or untreated. FIG. 16A: The antitumor effect of radiation was compromised by neutralization of type I IFNs. 500 μg anti-IFNAR was administered intratumorally on day 0 and 2 after radiation. FIG. 16B: MyD88 was non-essential for the antitumor effect of radiation. The tumor growth was shown in WT and MyD88^{-/-} mice after radiation. FIG. 16C: TRIF was dispensable for the antitumor effect of radiation. The tumor growth was shown in WT and TRIF^{-/-} mice after radiation. FIG. 16D: HMGB-1 was unnecessary for the antitumor effect of radiation. 200 μg anti-HMGB1 was administered i.p. on day 0 and 3 after radiation. FIG. 16E: CRAMP is dispensable for the antitumor effect of radiation. The tumor growth was shown in WT and CRAMP^{-/-} mice after radiation. FIG. 16F: STING was required for the antitumor effect of radiation. The tumor growth was shown in WT and STING^{-/-} mice after radiation. Representative data are shown from three (FIGS. 16A, 16B, 16C, 16D, 16E and 16F) experiments conducted with 5 (FIGS. 16A, 16B, 16C, and 16D) or 6 to 8 (FIGS. 16E and 16F) mice per group. Data are represented as mean±SEM. *P<0.05, **P<0.01 and ^{ns} No significant difference (Student's t test);

FIGS. 17A, 17B, and 17C show STING signaling in IFN-β induction by radiation. FIGS. 17A and 17B: STING signaling mediated the induction of IFN-β and CXCL10 by radiation. Tumors were excised on day 3 after radiation and homogenized in PBS with protease inhibitor. After homogenization, Triton X-100 was added to obtain lysates. ELISA assay was performed to detect IFN-β (FIG. 17A) and CXCL10 (FIG. 17B). FIG. 17C: STING signaling mediated the induction of type I IFN in dendritic cells after radiation. 72 hours after radiation, the single cell suspensions from tumors in WT mice and STING^{-/-} mice were sorted into CD11c⁺ and CD45⁻ populations. IFN-β mRNA level in different cell subsets were quantified by real-time PCR assay. Representative data are shown from three experiments conducted with 4 mice per group. Data are represented as mean±SEM. *P<0.05, **P<0.01 and ***P<0.001 (Student's t test);

FIGS. 18A, 18B, 18C and 18D show STING-IRF3 axis in dendritic cells is activated by irradiated-tumor cells. FIGS. 18A, 18B, and 18C: BMDCs were cultured with 40Gy-pretreated MC38-SIY^{hi} in the presence of fresh GM-CSF for 8 hours. Subsequently purified CD11c⁺ cells were co-cultured with isolated CD8⁺ T cells from naive 2C mice for three days and analyzed by ELISPOT assays. FIG. 18A: STING amplifying DCs function with the stimulation of irradiated-tumor cells. FIG. 18B: The deficiency of IRF3 impaired DC function with the stimulation of irradiated-tumor cells. FIG. 18C: IFN-β treatment rescued the function of STING^{-/-}DCs. long/ml IFN-β was added into the co-culture of BMDC and irradiated-tumor cells as described above. FIG. 18D: STING signaling mediated the induction of IFN-β in DCs by irradiated-tumor cells. Isolated CD11c⁺ cells as described above were incubated for additional 48 h and the supernatants were collected for ELISA assay. Representative data are shown from three (FIGS. 18A, 18B, 18C, and 18D) experiments. Data are represented as mean±SEM. *P<0.05, **P<0.01, ***P<0.001 and ^{ns} No significant difference (Student′s t test). See also FIG. 23;

FIGS. 19A, 19B, 19C, 19D, and 19E show cGAS role in dendritic cell sensing of irradiated-tumor cells. FIG. 19A: The mRNA level of cGAS in tumor-infiltrating CD11c⁺ was elevated after radiation. CD11c⁺ population was sorted from tumors at 72 hour after radiation. Real-time PCR assay was performed to quantify the mRNA level of cGAS. FIGS. 19B, 19C, and 19D: ELISPOT assays were performed as described in FIG. 18A. FIG. 19B: The function of BMDCs was compromised when cGAS was silenced. BMDCs were transfected with siRNA-non-targeting control and siRNA-cGAS. Two days later after transfection, the BMDCs were harvested for the co-culture assay. FIG. 19C: cGAS^{-/-} DCs stimulated with irradiated-tumor cells failed to cross-prime CD8⁺ T cells. FIG. 19D: DMXAA and IFN-β rescued the function of cGAS^{-/-} DCs. 10 ng/ml IFN-β was added into the co-culture of BMDC and irradiated-tumor cells as described above. The isolated CD11c⁺ cells were incubated with 100 μg/ml DMXAA for additional three hours. FIG. 19E: cGAS signaling mediated the induction of IFN-β in DCs by irradiated-tumor cells stimulation. Representative data are shown from three (FIGS. 19A, 19B, 19C, 19D and 19E) experiments. Data are represented as mean±SEM. **P<0.01 and ***P<0.001 (Student's t test). See also FIG. 24;

FIGS. 20A, 20B, 20C, 20D, and 20E show that STING signaling provides for effective adaptive immune responses mediated by type I IFN signaling on DCs after radiation. FIG. 20A: CD8⁺ T cells were required for the antitumor effects of radiation. 300 μg anti-CD8 mAb was administered i.p. every three days for a total of four times starting from the day of radiation. FIG. 20B: The function of tumor-specific CD8⁺ T cells was dependent on STING signaling following radiation. Eight days after radiation, tumor draining inguinal lymph nodes (DLNs) were removed from WT and STING^{-/-} mice. CD8⁺ T cells were purified and incubated with mIFN-γ pre-treated MC38 at the ratio of 10:1 for 48 hours and measured by ELISPOT assays. FIG. 20C: Exogenous IFN-β treatment rescued the function of CD8⁺ T cells in STING^{-/-} mice after radiation. 1×10¹⁰ viral particles of Ad-null or Ad-IFN-β was administered intratumorally on day 2 after radiation. Tumor DLNs were removed as described in (FIG. 20B). FIG. 20D: Anti-tumor effect of radiation was dependent on type I IFN signaling on dendritic cells. The tumor growth curve was analyzed in CD11c-Cre⁺IFNAR^{f/f} and IFNAR^{f/f} after radiation. FIG. 20E: The CD8⁺ T cell response was impaired in CD11c-Cre⁺IFNAR^{f/f} mice after radiation. Tumor DLNs were removed as described in (FIG. 20B). Representative data are shown from three (FIGS. 20A, 20B, 20C, 20D, and 20E) experiments conducted with 5-6 (FIGS. 20A and 20D) or 3-4 (FIGS. 20B and 20C and 20E) mice per group. Data are represented as mean±SEM. **P<0.01 and ***P<0.001 (Student's t test);

FIGS. 21A, 21B, 21C, and 21D show cGAMP treatment promotes the antitumor effect of radiation in a STING-dependent manner. FIGS. 21A and 21B: The administration of cGAMP enhanced the antitumor effect of radiation. MC38 tumors in WT and STING^{-/-} mice were treated by one dose of 20Gy. 10 μg 2′3′-cGAMP was administered intratumorally on day 2 and 6 after radiation. Tumor volume (FIG. 21A) and tumor-bearing mice frequency after IR (FIG. 21B) were monitored. FIG. 21C: cGAMP synergized with radiation to enhance tumor-specific CD8⁺ T cell response. 10 μg 2′3′-cGAMP was administered intratumorally on day 2 after radiation. Tumor DLNs were removed on day 8 after radiation for ELISPOT assays as described in FIG. 5B. FIG. 21D: The synergy of cGAMP and radiation is dependent on STING. ELISPOT assay was conducted as described in FIG. 5B. Representative data are shown from three experiments conducted with 5-7 (FIGS. 21A and 21B) or 3-4 (FIGS. 21C and 21D) mice per group. Data are represented as mean±SEM. **P<0.01 and ***P<0.001 (Student's t test in FIGS. 21A, 21C and 21D, and log rank (Mantel-Cox) test in FIG. 21B);

FIG. 22 shows schematic of proposed mechanism: cGAS-STING pathway is activated and orchestrates tumor immunity after radiation. Radiation results in the up-regulation of “find-me” and “eat-me” signals from tumor cells. During phagocytosis in dendritic cells, the DNA fragments hidden in irradiated-tumor cells are released from phagosomes to cytoplasm, acting as a danger signal. The cyclase cGAS binds tumor DNA, becomes catalytically active, and generate cGAMP as a second messenger. cGAMP binds to STING, which in turn activates IRF3 to induce type I IFN production. Type I IFN signaling on dendritic cells promotes the cross-priming of CD8⁺ T cells, leading to tumor control. Exogenous cGAMP treatment could optimize antitumor immune responses of radiation;

FIG. 23 shows the ability of WT, STING^{-/-} and IRF3^{-/-} BMDCs in the direct-priming of CD8⁺ T cells. BMDCs were stimulated with 20 ng/ml GM-CSF for 7 days. BMDCs were co-cultured with isolated CD8⁺ T cells from naive 2C mice at different ratios in the presence of 1 μg/ml SIY peptide for three days. The supernatants were harvested and subjected to CBA assay. Representative data are shown from three experiments. Data are represented as mean±SEM; and

FIGS. 24A and 24B show that irradiated-tumor cells are sensed by dendritic cells in a direct cell-to-cell contact manner. FIG. 24A: The floating DNA fragments were inessential for the ability of BMDCs to cross-priming of CD8⁺ T cells. 10 μg/ml DNase I was added in the incubation of BMDC and irradiated-MC38-SIY. The cross-priming of CD8⁺ T cells assay was performed. FIG. 24B: Cell-to-cell contact was responsible for the function of BMDCs with the stimulation of irradiated-tumor cells. Irradiated-MC38-SIY tumor cells were added into the insert and BMDCs were added into the well of Transwell-6 well Permeable plates with 0.4 μm pore size. Eight hours later, BMDCs were harvested and then incubated with CD8⁺ T cells for three days. Representative data are shown from three experiments. Data are represented as mean±SEM. ^{ns} No significant difference (Student's t test).

FIGS. 25A-25M show that MAVS is necessary for ionizing radiation-induced Type I interferon signalling. FIG. 25A shows the proposed mechanism of MAVS-dependent activation of Type I IFN signaling in the cellular response to IR. FIG. 25B shows transcriptional profiling of C57BL/6 wild-type (WT) and MAVS^{-/-} primary MEFs demonstrating MAVS-dependent expression of Type I IFN-stimulated genes (ISGs) 48 hours following exposure to IR (6 Gy). Heatmap displays differences in gene expression values between WT and MAVS^{-/-} MEFs; red indicates high expression and blue low expression. Inset shows qRT-PCR validation of Usp18, Ifit3, Stat1, Ddx58, and Cdkn1a gene expression values in WT and MAVS^{-/-} MEFs after IR treatment. FIG. 25C shows top-ranked cellular pathways (top) and functions (bottom) (Ingenuity Pathway Analysis) activated by IR in WT MEFs. Pie-chart displays the relative abundance of each functional category among all significant functions (P<0.05). IRF—interferon regulatory factor; PRR—pattern recognition receptor; JAK—Janus kinase; TYK—tyrosine kinase. FIGS. 25D and 25E show IFN-beta protein secretion (FIG. 25D) and caspase 3/7 activity (FIG. 25E) in WT and MAVS^{-/-} MEFs 48 hours following exposure to increasing doses of IR. FIG. 25F shows IFN-beta protein secretion and caspase 3/7 activity 48 hours following IR exposure of MAVS^{-/-} MEFs reconstituted by transient transfection of a full-length human MAVS construct (hMAVS) or an empty vector control (vector). FIGS. 25G, 25H and 25I show IR-induced IFN-beta (FIG. 25G), caspase 3/7 activity (FIG. 25H) and clonogenic survival (FIG. 25I) following siRNA-mediated suppression of MAVS (siMAVS) in human D54 glioblastoma cells. Scr—scrambled siRNA control. FIGS. 25J, 25K and 25L show IR-induced IFN-beta (FIG. 25J), caspase 3/7 activity (FIG. 25K) and clonogenic survival (FIG. 25L) following stable shRNA-mediated suppression of MAVS (shMAVS) in human HCT116 colorectal carcinoma cells. Depletion of MAVS increased Do values (dose required to reduce the fraction of surviving cells to 37%) from 1.01±0.02 Gy to 1.43±0.1 Gy (P=0.0025) in D54 and from 1.67±0.22 Gy to 2.36±0.09 Gy (P=0.0074) in HCT116 cells. Western blot analysis and representative scanned images of culture dishes after MAVS depletion and subsequent IR treatment are shown in the insets for (FIG. 25G), (FIG. 25I), (FIG. 25J), and (FIG. 25L). shM—shMAVS. Data are representative of three independent experiments. FIG. 25M shows relative tumor growth of shMAVS HCT116 tumor xenografts in athymic nude mice treated with IR (5 Gy×6 daily fractions). Data are representative of two experiments, each with n=5 mice per group. P values were determined using unpaired Student's t-test. Error bars are SEM. *P<0.05, **P<0.01, ***P<0.005.

FIGS. 26A, 26B, 26C and 26D show RLR pathway mediates radiation-induced gastrointestinal death following total body irradiation. FIG. 26A shows overall survival following total body irradiation (TBI, 5.5 Gy) of age-matched (9-12 weeks) wild-type (C57BL/6 or ICR background) and germline deleted LGP2^{-/-} (left), (middle), and MDA5^{-/-} (right) mice. Differences in survival were assessed using log-rank tests. *P<0.05, **P<0.01, n.s.—not significant. FIG. 26B shows IFN-beta quantification in mouse serum at specified time-points following exposure to TBI (5.5 Gy). Horizontal bar denotes mean value. Error bars are SEM. FIG. 26C shows small intestinal TUNEL staining of C57BL/6 wild-type (WT) and LGP2^{-/-} mice prior to and 7 days following total body irradiation at 5.5 Gy. Small intestinal cross-sections from LGP2^{-/-} mice exhibited greater intestinal crypt destruction (denoted by red arrows) as well as increased apoptosis (brown staining) in the crypt cells and the enterocytes lining the microvilli as compared to wild-type mice. FIG. 26D shows small intestinal TUNEL staining of C57BL/6 wild-type (WT), ICR RIG-I^{+/+} WT and ICR RIG-I^{-/-} mice prior to and 13 days following total body irradiation at 5.5 Gy. Small intestinal cross-sections from RIG-I^{-/-} mice showed minimal apoptotic staining in the enterocytes lining the microvilli as compared to wild-type mice. All images are representative of three replicates per condition. Magnification, 20×; scale bars, 0.11 μm.

FIGS. 27A, 27B, 27C and 27D show RIG-I orchestrates the MAVS-dependent Type I interferon response to ionizing radiation. FIG. 27A shows quantification of IR-induced IFN-beta secretion (left), caspase 3/7 activation (middle), and cell viability using XTT assay (right) in ICR RIG-I^{+/+} (WT) and RIG-I^{-/-} MEFs 48 hours after IR exposure. FIG. 27B shows IFN-beta protein secretion (left) and caspase 3/7 activation (right) 48 hours post-IR treatment following shRNA-mediated suppression of RIG-I (shRIG-I) in D54 cells. shScrambled—scrambled shRNA control. FIG. 27C shows relative tumor growth of shRIG-I D54 tumor xenografts in athymic nude mice treated with IR (5 Gy×6 daily fractions). shScr—scrambled shRNA control. Data are representative of three experiments, each with n=5 mice per group. FIG. 27D shows Caspase 3/7 activity of RIG-I^{-/-} and WT MEFs in response to increasing doses of cisplatin (left), doxorubicin (middle) and etoposide (right). Data are representative of three independent experiments. P values were determined using unpaired Student's t-test. Error bars are SEM. *P<0.05, **P<0.01, ***P<0.005.

FIGS. 28A, 28B, 28C, 28D, 28E and 28F show IR induces RIG-I binding to endogenous double-stranded RNAs. FIG. 28A shows that HEK293 reporter cells were irradiated after transfection with either an empty vector, a full length human RIG-I, a RIG-I lacking CARD domains (RIG-I helicase/CTD), or a RIG-I harboring K858A and K861A mutations in the C-terminal domain (RIG-I K858A-K861A), in addition to an IFN-beta promoter-driven luciferase construct. A Renilla reporter construct served as a transfection control. Data are presented as mean fold-change relative to the non-irradiated empty vector control. FIG. 28B shows that donor HEK293 cells were either unirradiated or treated with IR (3 or 6 Gy). Total RNA was purified and transferred to independent batches of HEK293 reporter cells transfected by RIG-I constructs as described in (FIG. 28A). A synthetic double-stranded RNA construct comprised of 5′-triphosphorylated dsRNA and an unphosphorylated counterpart served as positive and negative controls, respectively (inset). FIG. 28C shows experimental design for isolation and purification of RNA bound to RIG-I after exposure to IR. See methods for further details. FIG. 28D shows purified RNA from total cellular extracts (Lanes 2 and 3) and complexes with RIG-I (Lanes 4 and 5). Lane 1 is the marker. Data are representative of at least 3 independent experiments. FIG. 28E shows HEK293 cells over-expressing the HA-tagged full length RIG-I (Lanes 2 and 3), the RIG-I helicase-CTD mutant (Lanes 4 and 5) and the RIG-I K858A-K861A CTD mutant (Lanes 6 and 7) were either un-irradiated or exposed to IR (6 Gy), lysed and incubated with anti-HA monoclonal antibody to pulldown the respective WT and mutant RIG-I proteins. RIG-I diagrams illustrate the mechanism of RIG-I activation (adapted from Zheng and Wu et al., 2010). In the inactive/unbound conformation, the CARD domain of RIG-I is folded to block the helicase domain from RNA binding RNA, but allows the CTD to search for its ligand. Upon binding of the blunt end of a dsRNA molecule to the CTD, the CARD domain opens to allow the helicase domain to bind the remaining dsRNA molecule. Absence of the CARD domain in the helicase/CTD mutant enables higher affinity binding to dsRNA ligands as compared to the full length RIG-I. The lysine residues at amino acid positions 858 and 861 have previously demonstrated importance in latching onto the 5′-triphosphorylated end of viral dsRNA ligands. FIG. 28F shows RNA bound to RIG-I after exposure to IR (6 Gy) was treated with: RNase A (lane 3), dsRNA-specific RNase III (lane 4), single-strand specific nuclease S1 (lane 5) and DNase I (lane 7). Lane 2 shows the input and lanes 1 and 6 display markers.

FIGS. 29A, 29B, 29C, 29D, 29E, 29F, 29G and 29H shows that RIG-I binds U1 snRNA accumulated in the cytoplasm to mediate radiation-induced IFN-beta response. FIG. 29A shows that RIG-I binds diverse non-coding RNA molecules, majority of which are snRNAs. Graphic representation indicating the distribution of non-coding and repetitive RNA molecules bound to RIG-I following exposure to IR as compared to total irradiated cellular RNA. Transcripts were mapped to reference genomes using RepeatMasker. See Methods for further details. FIG. 29B shows qRT-PCR quantification of U1 RNA from purified RNA bound to ectopically expressing WT and K858A-K861A mutant RIG-I HEK293 cells exposed to IR (6 Gy) or left untreated. Cells were UV crosslinked at 150 mJ/cm² 48 hours post-IR treatment prior to cell lysis. U1 RNA levels were normalized to the geometric average of 3 housekeeping genes (18S rDNA, GAPDH, and (3-actin). Fold change was determined relative to un-irradiated controls. FIG. 29C shows that U1 RNA levels quantified by qRT-PCR from total cellular and RIG-I pulldowns in RIG-I overexpressing HEK293 and HCT116 cells. U1 RNA levels were normalized to the geometric average of 3 housekeeping genes (18S rDNA, GAPDH, and (3-actin). Fold change was determined relative to un-irradiated controls. Time course of cytosolic accumulation of U1 RNA measured by qRT-PCR from purified total cellular RNA following cellular fractionation of nuclear/mitochondrial and cytoplasmic fractions of HEK293 (FIG. 29D) and HCT116 cells (FIG. 29E) exposed to IR (6 Gy) or left untreated. FIG. 29F shows the structure of the U1 snRNA illustrating the four stem loop (SL) regions. FIG. 29G shows relative IFN-beta luciferase reporter activity of HEK293 cells following a 24 hour stimulation with synthetic oligonucleotides corresponding to U1 RNA stem loop (SL) regions I to IV or a combination of SL I+II and SL II+III. FIG. 29H show IFN-b levels in culture supernatant from ICR RIG-I^{+/+} and primary MEFs 24 hours post-stimulation with the same set of synthetic U1 oligonucleotides used in (FIG. 29G) The amount of U1 synthetic oligonucleotides used in all stimulation experiments was 1 μg. P values were determined using unpaired Student's t-test. Error bars are SEM. ***P<0.005.

FIGS. 30A, 30B, 30C, 30D and 30E show that radiation and chemotherapy activate Type I interferon-stimulated genes in cancer patients. FIG. 30A shows heatmap displaying the commonality of Type I ISG induction in human cervical, breast, and bladder cancers following genotoxic treatment. Black boxes denote treatment. Gene expression values were obtained from microarray analysis of matched pre- and post-treatment tumor biopsies. Overexpression defined as fold-change>1 in post-treatment biopsies as compared to matched pre-treatment biopsies. FIG. 30B shows type I ISG expression in pre- and post-chemoradiation specimens of human rectal cancer and matched normal tissue. FIG. 30C shows type I ISGs (n=81) distinguish breast cancer patients (GSE25055, n=310). ISG(+) defined by overexpression of type I ISGs (left). Black hash marks denote complete pathologic response (pCR) to pre-operative doxorubicin-based chemotherapy. FIG. 30D shows canonical pathways (top) and top-ranked gene network (bottom) from Ingenuity Pathway Analysis of Type I ISGs identified in (FIG. 30C). FIG. 30E (Left) shows frequency of pCR in ISG(+) and ISG(−) breast cancers treated with pre-operative doxorubicin-based chemotherapy. P value was determined by using Fisher's exact test. FIG. 30E (Middle) shows mean ISG expression (81 genes) in breast cancers which achieved a pCR to pre-operative chemotherapy vs. tumors with residual disease (non-pCR). P value was determined by using unpaired Student's t-test. Error bars are SEM. FIG. 30E (Right) shows Kaplan-Meier estimates of distant relapse-free survival (DRFS) in breast cancer patients with a pCR vs. non-pCR. Left: GSE25055 (n=310); right: GSE25065 (n=198). P values were determined by using log-rank tests.

FIGS. 31A, 31B, 31C and 31D show that MAVS is required for IR-induced cell killing. FIG. 31A shows western blot analyses of lysates from WT and MAVS^{-/-} primary MEFs 48 hours post-exposure to increasing doses of IR. The membranes were probed for MAVS, TBK1, phospho-TBK1, and IRF3. α-Tubulin antibody was used for loading control. FIG. 31B shows clonogenic survival of immortalized C57BL/6 wild-type (WT) and MAVS^{-/-} MEFs after exposure to increasing doses of IR (left). Representative scanned images of colonies are shown (right). FIG. 31C shows cell viability after siRNA-mediated suppression of MAVS (siMAVS) in the human D54 glioblastoma (left) and WiDr colon adenocarcinoma cell lines (right) in the response to IR as compared to a scrambled transfection controls. FIG. 31D shows wild-type primary MEFs were pre-incubated with neutralizing anti-IFNAR1 monoclonal antibody (1, 10, or 50 μg/ml) or an isotype control 90 minutes prior to IR treatment. Apoptotic induction was assessed by measurement of caspase 3/7 activation. *P<0.05, ***P<0.005.

FIGS. 32A, 32B and 32C show that LGP2 suppresses IFN-beta-dependent cytotoxicity. Wild-type (WT) and LGP2^{-/-} MEFs were assessed for IFN-beta secretion (FIG. 32A), caspase 3/7 activity (FIG. 32B), and clonogenic survival (FIG. 32C) following exposure to increasing doses of IR. Representative scanned images of colonies are shown (right). *P<0.05, ***P<0.005.

FIGS. 33A, 33B, 33C and 33D show that MAVS and RIG-I promote IFN-beta expression following IR treatment. Ectopic overexpression of MAVS (FIG. 33A), RIG-I (FIG. 33B), and MDA5 (FIG. 33C) in HEK293 cells co-transfected with the IFN-beta promoter-driven luciferase reporter and a Renilla reporter construct. Cells were subsequently irradiated 24 hours following transfection. Luminescence was measured at 48 hours and the relative IFN-beta luciferase activity was normalized to the non-irradiated cell control transfected with the empty vector. FIG. 33D shows that RIG-I mediates cell survival following exposure to IR. Cell viability of RIG-I^{-/-} MEFs reconstituted by full-length human RIG-I or transfected with an empty vector. *P<0.05, ***P<0.005.

FIGS. 34A, 34B, 34C and 34D show that RIG-I mediates apoptotic responses to IR and genotoxic chemotherapy drugs. FIG. 34A shows IFN-beta protein secretion and caspase 3/7 activation 48 hours post-IR in HCT116 cells treated with siRNA targeting RIG-I. FIG. 34B shows Caspase 3/7 activities after stable RIG-I knockdown (shRIG-I) of D54 and HCT116 tumor cells. FIG. 34C shows clonogenic survival of D54 and HCT116 shRIG-I. Depletion of RIG-I increased clonogenic Do values from 0.95±0.009 Gy to 1.68±0.15 Gy (p=0.001) in D54 and from 0.86±0.018 Gy to 1.23±0.119 Gy (p=0.006) in HCT116 cells. Anticancer treatment consisted of increasing doses of IR (FIG. 34B), cisplatin, doxorubicin or and etoposide (FIG. 34D). In all treatments, Caspase 3/7 activation 48 hours post-IR was used as read-out. Control cells were transfected with scrambled shRNA constructs. Scrambled—scrambled siRNA control; si-RIG-I#1—siRIG-I construct #1; si-RIG-I#2—siRIG-I construct #2; shScrambled—scrambled shRNA control; shRIG-I—shRIG-I plasmid construct. *P<0.05, **P<0.01, ***P<0.005.

FIGS. 35A, 35B, 35C and 35D show that U2 is enriched in RIG-I: RNA complexes and redistributes to the cytosol following irradiation. FIG. 35A shows quantification of U2 levels in RNA purified from RIG-I pulldown in HEK293 cells overexpressing either the full length RIG-I or the K858A-K861A RNA binding deficient mutant. FIG. 35B shows quantification of U2 levels in total cellular input RNA and pulldown RNA purified from RIG-I overexpressing HEK293 and HCT116 cells. For both (FIG. 35A) and (FIG. 35B), fold change in irradiated samples was normalized to the un-irradiated controls. The time courses of nuclear and cytoplasmic redistribution of U2 were quantified in both HEK293 (FIG. 35C) and HCT116 (FIG. 35D) post-IR. Fold change in the cytoplasmic fraction was normalized to the nuclear levels of U2 for each time point. *P<0.05, **P<0.01, ***P<0.005.

FIGS. 36A, 36B and 36C show that RIG-I protein expression is induced by ionizing radiation. Western blot analyses of cell lysates from C57BL/6 wild-type MEFs (FIG. 36A), as well as HCT116 (FIG. 36B) and WiDr tumor cell lines (FIG. 36C) harvested 48 hours post-IR treatment at increasing doses. For (FIG. 36B) and (FIG. 36C), targeted siRNA was used to knock-down RIG-I in human tumor cell lines. The band intensities were quantified using ImageJ software, and the reported values were normalized relative to the non-irradiated control per cell line. Scrambled—scrambled siRNA control, siRIG-I #1—siRIG-I construct #1, siRIG-I #2—siRIG-I construct #2.

FIGS. 37A and 37B show that full length in vitro transcribed U1 snRNA stimulates endogenous and ectopically expressed RIG-I in HEK293 IFN-beta luciferase reporter cells. FIG. 37A shows relative IFN-beta luciferase reporter activity in HEK293 cells stimulated for 24 hours with in vitro transcribed full length U1 snRNA. HEK293 cells were transfected with either an empty vector or the full length RIG-I. In addition, U1 was digested one hour before HEK293 stimulation by treatment with various nucleases: dsRNA-specific RNase III, RNase A, and single-strand specific nuclease S1. The positive and negative controls used in this experiment were the 5′-triphosphorylated 19-mer dsRNA and the corresponding unphosphorylated counterpart, respectively. FIG. 37B shows CIAP treatment of U1 reduced induction of IFN-beta promoter in HEK293 cells.

FIGS. 38A and 38B show that type I interferon-stimulated gene expression is associated with improved responses to pre-operative chemotherapy. FIG. 38A shows heatmap of 81 Type I ISGs distinguishing two molecular subgroups of breast cancer patients (GSE20194, n=278). ISG(+) defined by overexpression of type I ISGs (left). Black hash marks denote complete pathologic response (pCR) to pre-operative doxorubicin-based chemotherapy. FIG. 38B shows frequency of pCR in ISG(+) and ISG(−) breast cancer patients treated with pre-operative doxorubicin-based chemotherapy. P value was determined by using Fisher's exact test.

FIG. 39 shows that IR drastically increased stability of RNA in the tumor microenvironment (up to 52 hours) by using quantified fluorescent intensity. Pre-incubation of RNA with the jetPEI lipid further increased stability of RNA (see quantified fluorescent intensity table in FIG. 39).

FIG. 40 shows that injection of stem-loop structures of U1 in combination with jetPEI lipid and IR led to the 2-fold suppression of tumor growth as compared with IR only. MC38 tumors were irradiated at 20Gy and the irradiated tumors were injected with stem-loop regions of U1 at 1, 7 and 14 dayspost-IR. These data show that U1 endogenous RNA detected in complexes with RIG-I, demonstrated to induce IFN-beta promoter in vitro, is a potent radiosensitizer of tumor in preclinical animal model.

FIGS. 41A and 41B show that injections of RNA-lipid complexes in tumors led to upregulation of several ligands with pro-survival properties. To test what ligands can be activated by RNA delivery we used protein arrays with loaded probes for multiple mouse cytokines and chemokines. These experiments indicated that for improved suppressive effects of RNA ligands they may be combined with agents inhibiting pro-survival ligands induced by the given RNA. Overall this indicates that for further improvement of therapeutic potential of such RNA drug it is important to test pattern of cytokines induced by RNA injections.

### DESCRIPTION

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to, plus or minus 10% of the particular term.

The use of the terms “a,” “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The term “cancer,” as used herein, refers to a broad group of disease involving unregulated cell growth and division. Non-limiting examples of cancers include leukemias, lymphomas, carcinomas, and other malignant tumors, including solid tumors, of potentially unlimited growth that can expand locally by invasion and systemically by metastasis. Examples of cancers include any of those described herein, but are not limited to, cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

The term “cancer” may also include, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal; rectum, Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions.

The term “administering” or “administration of a composition” to a subject or patient, as used herein, refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

The term “treating,” “treatment of,” or “therapy of a condition or patient,” as used herein, refers to taking steps to obtain beneficial or desired results, including clinical results. Beneficial or desired clinical results include, but are not limited to, alleviation or amelioration of one or more symptoms of cancer; diminishment of extent of disease; delay or slowing of disease progression; amelioration, palliation, or stabilization of the disease state; or other beneficial results. Treatment of cancer may, in some cases, result in partial response or stable disease.

In one embodiment, the present invention relates to a composition comprising at least one RNA such as snRNA. Tables 1 and 2 show the lists of some exemplary RNAs such as snRNAs. In another embodiment, a composition of the present invention comprises at least two RNAs such as snRNAs.

In one embodiment, the present invention relates to a composition comprising at least one rbRNA. Table 5 shows the lists of some exemplary rbRNAs. In another embodiment, a composition of the present invention comprises at least two rbRNAs. In yet another embodiment, a composition of the present invention comprises a fragment of an rbRNA.

In one embodiment, the composition of the present invention further comprises one additional therapeutic agent.

The term “therapeutic agent,” as used herein, refers to a substance therapeutically effective for treating a disease condition. In one embodiment, the additional therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

In another embodiment, the additional therapeutic agent may be selected from the group consisting of taxanes; inhibitors of bcr-abl (such as Gleevec, dasatinib, and nilotinib); inhibitors of EGFR (such as Tarceva and Iressa); DNA damaging agents (such as cisplatin, oxaliplatin, carboplatin, topoisomerase inhibitors, and anthracyclines); and antimetabolites (such as AraC and 5-FU).

In yet other embodiments, the additional therapeutic agent may be selected from the group consisting of camptothecin, doxorubicin, idarubicin, Cisplatin, taxol, taxotere, vincristine, tarceva, the MEK inhibitor, UO 126, a KSP inhibitor, vorinostat, Gleevec, dasatinib, and nilotinib.

In another embodiment, the additional therapeutic agent is selected from the group consisting of Her-2 inhibitors (such as Herceptin); HDAC inhibitors (such as vorinostat), VEGFR inhibitors (such as Avastin), c-KIT and FLT-3 inhibitors (such as sunitinib), BRAF inhibitors (such as Bayer's BAY 43-9006) MEK inhibitors (such as Pfizer's PD0325901); and spindle poisons (such as Epothilones and paclitaxel protein-bound particles (such as Abraxane®).

In one embodiment, the present composition may be further combined with other therapies or anticancer agents. Other therapies or anticancer agents that may be used in combination with the inventive anticancer agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, dexamethasone, and cyclophosphamide. [00154] A compound of the present invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); atezolizumab; bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xelodag); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); nivolumab (Opdivo®); Nofetumomab (Verluma®); norharmane; Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pembrolizumab (Keytruda®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); rosmarinic acid; sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Val Star®); vinblastine (Velban®); vincristine (Oncovin®); vinorelbine (Navelbine®); zoledronate (Zometa®) and vorinostat (Zolinza®).

The term “ionizing radiation,” as used herein, refers to high-energy radiation and electromagnetic radiation and includes but is not limited to radiotherapy, x-ray therapy, irradiation, exposure to gamma rays, protons, alpha-particle or beta-particle irradiation, fast neutrons, and ultraviolet.

Treatment of a cancer in a subject in need thereof is provided herein, as are compositions, kits, and methods for treating cancer, and methods for identifying effector genes in the Jak/Stat pathway having a role in the treatment of cancer and therapies to treat cancer based on these effector genes. Such treatment of cancer may include maintaining ionizing radiation and/or chemotherapy sensitization of a tissue in the subject, maintaining radio/chemoprotection of normal non-disease state tissue in the subject, and/or protecting normal non-disease state tissue from genotoxic stress. A Jak/Stat dependent cancer may include any solid tumor, including lung, prostate, head and neck, breast and colorectal cancer, melanomas and gliomas, and the like. While the present disclosure may be embodied in different forms, several specific embodiments are discussed herein with the understanding that the present disclosure is to be considered only an exemplification and is not intended to limit the invention to the illustrated embodiments.

Radiotherapy used alone or in combination with surgery or chemotherapy is employed to treat primary and metastatic tumors in approximately 50-60% of all cancer patients. The biological responses of tumors to radiation have been demonstrated to involve DNA damage, modulation of signal transduction, and alteration of the inflammatory tumor microenvironment. Indeed, radiotherapy has been recently shown to induce antitumor adaptive immunity, leading to tumor control (Apetoh, L., Ghiringhelli, F., Tesniere, A., Obeid, M., Ortiz, C., Criollo, A., Mignot, G., Maiuri, M. C., Ullrich, E., Saulnier, P., et al. (2007). Toll-like receptor 4-dependent contribution of the immune system to anticancer chemotherapy and radiotherapy. Nat Med 13, 1050-1059; Lee, Y., Auh, S. L., Wang, Y., Burnette, B., Meng, Y., Beckett, M., Sharma, R., Chin, R., Tu, T., Weichselbaum, R. R., and Fu, Y. X. (2009). Therapeutic effects of ablative radiation on local tumor require CD8+ T cells: changing strategies for cancer treatment. Blood 114, 589-595). The blockade of immune checkpoints has been shown to improve the efficacy of radiotherapy on local and distant tumors in experimental systems and more recently in clinical observations (Deng, L., Liang, H., Burnette, B., Beckett, M., Darga, T., Weichselbaum, R. R., and Fu, Y. X. (2014). Irradiation and anti-PD-L1 treatment synergistically promote antitumor immunity in mice. J Clin Invest 124, 687-695; Postow, M. A., Callahan, M. K., Barker, C. A., Yamada, Y., Yuan, J., Kitano, S., Mu, Z., Rasalan, T., Adamow, M., Ritter, E., et al. (2012). Immunologic correlates of the abscopal effect in a patient with melanoma. N Engl J Med 366, 925-931). Furthermore, radiotherapy sculpts innate immune response in a type I IFNs-dependent manner to facilitate adaptive immune response (Burnette, B. C., Liang, H., Lee, Y., Chlewicki, L., Khodarev, N. N., Weichselbaum, R. R., Fu, Y. X., and Auh, S. L. (2011). The efficacy of radiotherapy relies upon induction of type i interferon-dependent innate and adaptive immunity. Cancer Res 71, 2488-2496). However, the molecular mechanism for host type I IFNs induction following local radiation had not yet been defined. We have also previously demonstrated that overexpression of Stat1-pathway plays an important role in the response of tumor cells to ionizing radiation (IR), though mechanisms were unclear.

Radiotherapy is the most common modality of the anti-tumor treatment and is used in the majority of known tumors as either the means to reduce initial tumor volume or adjuvant treatment to reduce chances of local or distant recurrence after primary surgical excision of the tumor. Often in the post-surgery treatment chemotherapy is prescribed but the outcome of the chemotherapy-treated patients does not exceed 5% success over not-treated patients. It is now believed that downstream effector genes in the Jak/Stat pathway have a causal role in treatment-resistant cancers, including solid tumors, and if downstream effector genes can be identified having a direct relationship to treatment resistance, new therapies could be developed for treatment resistant cancers.

We have now discovered that the Rig-I-like receptor (RLR) LGP2 is a potent regulator of tumor cell survival. It is believed that LGP2 suppresses the RNA-activated cytoplasmic RLR pathway and inhibits the mitochondrial antiviral signaling protein (MAVS)-dependent induction of endogenous IFNbeta (IFNβ) production. It is further believed that suppression of LGP2 leads to enhanced IFNbeta expression resulting in increased tumor cell killing, while suppression of MAVS leads to protection of tumor cells from ionizing radiation-induced killing. Neutralizing antibodies to IFNbeta protect tumor cells from the cytotoxic effects of IR.

Consistent with this observation, mouse embryonic fibroblasts (MEFs) from IFNalpha Receptor I knock-out mice (IFNAR1−/−) are radioresistant compared to wild-type MEFs. In high grade gliomas, where survival rates correlate with response to radiotherapy, elevated levels of LGP2 expression are associated with poor clinical outcomes. It is contemplated that these results demonstrate that the cellular response to radiation occurs through RLR-dependent pathways of the innate immune response to pathogens converging on the induction of IFNbeta.

We also demonstrate that another cytoplasmic DNA sensing pathway responsible for activation of Type I Interferons also contain members, which suppression can lead to radioprotection or radiosensitization. Apical suppressor of cytoplasmic DNA-sensoring pathway-exonuclease TREX1 protect cells from IR and its down-regulation by shRNA (small hairpin RNA) renders SCC61 cells radiosensitive. Contrary to this suppression of adapter protein STING, responsible for DNA-dependent activation of Type I IFNs, render cells radioresistant. This connection we have discovered reveals novel pathways by which IR causes cellular cytotoxicity and identifies previously unrecognized targets to enhance tumor cell killing by radio/chemotherapy or protect normal tissues from genotoxic stress.

Maintaining Type I IFN production can be achieved, for example, by suppression of negative regulators of RNA and DNA dependent pathways as LGP2 and TREX1. Activation of Type I IFN production can be measured by means known in the art, including, for example, QRT-PCR, or hybridization of mRNA with specific probes on custom arrays or commercial arrays available from, for example, Affymetrix Inc., Agilent Technologies, Inc., Nanostring Technologies, Inc., GeneQuant (GE Healthcare, Little Chalfont, United Kingdom) or Luminex Corp., or using protein detection by ELISA.

While the bane of radiotherapy (IR) of cancer is the emergence of radioresistant cells, we have also discovered that radioresistance is induced by LGP2, a resident RIG-I like receptor protein also known as RNA helicase DHX58. IR induces interferon and stimulates accumulation of LGP2. In turn LGP2 shuts off the synthesis of interferon and blocks its cytotoxic effects. Ectopic expression of LGP2 enhances resistance to IR whereas depletion enhances cytotoxic effects of IR. Herein we show that LGP2 is associated with radioresistance in numerous diverse cancer cell lines. Examination of available databases links expression of LGP2 with poor prognosis in cancer patients.

From our observations, we contemplate that cytoplasmic pattern-recognition receptors (PRRs) are also potent targets for radio/chemosensitization of tumor cells or protection of normal cells from genotoxic stress, including, for example, exposure to IR, ultraviolet light (UV), chemotherapy, and/or ROS (Reactive Oxygen Species). We further contemplate from our observations that the pathway of Type I IFN production is a target for radio/chemosensitization or protection. Further, it is believed that RIG1-like receptors (RLRs), including RIG1 (Retinoic Acid-inducible Gene 1), LGP2, MDA5 and other molecules of this type, are responsible for activation of IFN response through interaction with cytoplasmic RNA, and are targets for radio/chemosensitization or protection. It is further contemplated that MAVS (also known as IPS1 (Interferon-beta Promoter Stimulator 1)) are an effector protein of RNA-dependent pathway of IFN production and are a target for normal tissues radioprotection or (through activation) tumor radio/chemosensitization. We further contemplate that cytoplasmic DNA sensors and regulatory molecules like TREX1, DAI, IFI16, Aim2 and other molecules of this type as targets for radio/chemosensitization or protection; and STING or TMEM173 or MPYS (plasma membrane tetraspanner) (a.k.a. MITA or EMS) as target for normal tissues radio/chemoprotection or through activation-tumor radio/chemosensitization. Further, a method where tumor radio/chemosensitization may be achieved by suppression of the apical repressors of the RNA/DNA-dependent pathways of IFN production are further contemplated herein as is a method where normal tissue radio/chemoprotection may be achieved by suppression of the major effector proteins of the RNA/DNA-dependent pathways of IFN production. A further method where protection of normal tissues from toxic effects of IR and chemotherapy may be achieved by depletion of IFNs (e.g., with neutralizing Abs) or agonists of IFNAR1 (interferon-alpha receptor 1) (e.g., such as with an antagonist of IFNAR1), is also contemplated as are prognostic markers for patients with high grade gliomas where high expression of LGP2 predicts poor prognosis while low expression of LGP2 predicts improved prognosis.

In another aspect of the present disclosure, we now demonstrate that STING, but not MyD88, provides for type I IFN-dependent antitumor effects of radiation. As shown herein, STING in dendritic cells (DCs) controlled radiation-mediated IFN-β induction and were activated by irradiated-tumor cells. The cytosolic DNA sensor cyclic GMP-AMP synthase (cGAS) mediated DCs sensing of irradiated-tumor cells. Moreover, STING provided for radiation-induced adaptive immune responses, which relied on type I IFN signaling on DCs. Exogenous IFN-β treatment rescued cGAS/STING-deficient immune responses. Accordingly, enhancing STING signaling by cGAMP administration promoted antitumor efficacy of radiation. Our results reveal that the molecular mechanism of radiation-mediated antitumor immunity depends on a proper cytosolic DNA-sensing pathway, pointing towards a new understanding of radiation and host interactions. Furthermore, we uncover herein a new strategy to improve radiotherapy by cGAMP treatment. For example, it is contemplated that administration of a therapeutic amount of 2′3′-Cgamp (InvivoGen; cyclic [G(2′,5′)pA(3′,5′)p]); CAS 1441190-66-4), and/or one or more therapeutically active derivatives or mimics thereof, to a subject in need thereof promotes antitumor efficacy of radiation therapy as compared to an untreated control subject. For example, cGAMP can be formulated for injection via intravenous, intramuscular, sub-cutaneous, intratumoral, and/or intraperitoneal routes. Typically, for a human adult (weighing approximately 70 kilograms), an effective amount or therapeutically effective amount can be administered by those skilled in the art. For example, a subject is administered from about 0.01 mg to about 3000 mg (including all values and ranges there between), or from about 5 mg to about 1000 mg (including all values and ranges there between), or from about 10 mg to about 100 mg (including all values and ranges there between). A dose may be administered on an as needed basis or every 1, 2, 3, 4, 5, 6, 7, 8, 9, 10, 11, 12, 18, or 24 hours (or any range drivable therein) or 1, 2, 3, 4, 5, 6, 7, 8, 9, or 10 times per day (or any range derivable therein). The subject may be treated for 1, 2, 3, 4, 5, 6, 7, 8, 9, 10 or more days (or any range derivable therein) or until tumor has disappeared or been reduced. cGAMP can be administered 1, 2, 3, 4, 5, 6, 7, 8, 9, or more times. It is also contemplated that other agents that enhance STING signaling may also be utilized in the therapeutic methods described herein to promote antitumor efficacy of radiation in a subject, including, for example other STING activators such as members of the combretastatin (CAS 82855-09-2) family of phenols, including combretastatin A-1 (combretastatin A1 diphosphate (OXi4503 or CA1P); CAS 109971-63-3), combretastatin B-1 (CAS 109971-64-4), combretastatin A-4 (CAS 117048-59-6), and derivatives and analogs thereof such as Ombrabulin™ (Sanofi-Aventis, (CAS 181816-48-8, 253426-24-3(HCL)); or DMXAA (also known as Vadimezan™ or ASA404)(Novartis, CAS 117570-53-3).

In yet another aspect of the present disclosure, it is contemplated that radiation causes tumor cell nucleic acids and/or stress proteins to trigger the activation of TLRs-MyD88/TRIF signaling. Although not wishing to be bound by theory, it is believed based on published research that the innate immune system is the major contributor to host-defense in response to pathogens invasion or tissue damage. The initial sensing of infection and injury is mediated by pattern recognition receptors (PRRs), which recognize pathogen-associated molecular patterns (PAMPs) and damage-associated molecular patterns (DAMPs). The first-identified and well-characterized of class of PRRs I are the toll-like receptors (TLRs), which are responsible for detecting PAMPs and DAMPs outside the cell and in endosomes and lysosomes. Under the stress of chemotherapy and targeted therapies, the secretion of HMGB-1, which binds to TLR4, has been reported to be essential to antitumor effects. However, whether the same mechanism dominates radiotherapy has yet to be determined. Four endosomal TLRs (TLR3, TLR7, TLR8 and TLR9) that respond to microbial and host-mislocalized nucleic acids in cytoplasm have more recently been revealed. Through interaction of the adaptor proteins, myeloid differentiation primary-response protein 88 (MyD88) and TIR-domain-containing adaptor protein inducing IFN-β (TRIF), the activation of these four endosomal TLRs leads to significant induction of type I IFN production. Given that radiation induces production of type I IFNs, it is contemplated herein that the trigger for activation of TLRs-MyD88/TRIF signaling is by tumor cell nucleic acid and/or stress proteins generated by radiotherapy.

Although not wishing to be bound by theory, it is believed for activation of TLR3 in a subject, the subject can be administered polyinosine-polycytidylic acid poly(I:C) (0.4 mg/kg); a double-stranded DNA; a double-stranded RNA; or stathmin (Entrez Gene ID: 3925 (human), 16765 (mouse)) or a stathmin-like protein (0.4 m/kg), which is generally understood to be a protein with an α-helix structure having an amino acid homology of at least about 85%, or at least about 90%, or at least about 92% to that of amino acid residues 44-138 of human stathmin (Entrez Gene ID: 3925), including, for example, SCGIO ((Superior Cervical Ganglion 10; stathmin-2; STMN2, SCG10, SCHN10; Entrez Gene ID: 11075 (human), 20257 (mouse)), SCLIP (SCGlO-like protein; stathmin-3; STMN3; Entrez Gene ID: 50861 (human), 20262 (mouse)), and RB3 (stathmin-4; WO2007089151), and analogs and derivatives thereof such as, for example, natural or synthetic amino acid analogs thereof. A contemplated effective dose administered daily can be determined by those skilled in the art and can range, for example, from about 0.01 μg/kg to 1 g/kg or from about 0.5 μg/kg to about 400 mg/kg body weight as described in U.S. patent application Ser. No. 12/162,916. Contemplated compounds for the activation of TLR7 or TLR8 are described in U.S. Pat. No. 7,560,436. For example, TLR7 can be activated by administering to a subject imidazoquinoline compounds (for example, R-848 (InvivoGen, CAS 144875-48-9), 3M-13 and 3M-019 (both by 3M Pharmaceuticals, St. Paul, Minn.)) and those described in U.S. Pat. Nos. 4,689,338, 4,929,624, 5,238,944, 5,266,575, 5,268,376, 5,346,905, 5,352,784, 5,389,640, 5,395,937, 5,494,916, 5,482,936, 5,525,612, 6,039,969 and 6,110,929. Other contemplated TLR7 activators include guanosine analogs, pyrimidinone compounds such as bropirimine and bropirimine analogs and the like. Imidazoquinoline compounds include, but are not limited to imiquimod (also referred to as Aldara, R-837, S-26308; InvivoGen, CAS 99011-02-6). TLR8 can be activated by, for example, administering to a subject an imidazoquinoline compound (for example, 3M-2 and 3M-3 (both by 3M Pharmaceuticals, St. Paul, Minn.); or R-848 (InvivoGen, CAS 144875-48-9)). It is further contemplated for activation of TLR9, a subject can be administered one or more CpG oligodeoxynucleotides (or CpG ODN), which are short single-stranded synthetic DNA molecules. Each CpG contains a cytosine triphosphate deoxynucleotide and a guanine triphosphate deoxynuclerotide, with a phosphodiester link between consecutive nucleotides. It is believed that the CpG motifs classified as pathogen-associated molecular patterns (PAMPs) are recognized by TLR9, which is expressed in B cells and in plasmacytoid dendritic cells in humans and some primates. CpG useful in the present disclosure may be from microbial DNA or synthetically produced, and are generally categorized into five classes: 1) Class A (Type D), 2) Class B (Type K), 3) Class C, 4) Class P, and 5) Class S. Class A ODN includes ODN 2216, which stimulates large amounts of Type I interferon production, including IFNα, induces the maturation of plasmacytoid dendritic cells, and is a strong activator of NK cells through indirect cytokine signaling. Class A ODN is generally characterized by the presences of a poly G sequence at the 5′ end, the 3′ end, or both, a partially phosphorothioated-modified backbone, an internal palindrome sequence and GC dinucleotides contained within the internal palindrome. Class B ODN includes ODN 2006 (InvivoGen, ODN 7909, PF_3512676) and ODN 2007 (InvivoGen), which is a strong stimulator of human B cell and monocyte maturation and to a lesser extent a stimulator of IFNα and the maturation of pDC. Structural characteristics of Class B ODN include an about a 18 to 28 nucleotide length, a fully phosphorothioated (PS-modified) backbone and one or more 6mer CpG motif 5′-Pu Py C G Py Pu-3′.

Although there are no direct activators of MyD88 or TRIF known at this time, it is contemplated that as agents are discovered or developed that interact with these proteins, these agents can be used and incorporated into the therapeutic methods and disclosure described herein.

A newly defined endoplasmic reticulum associated protein STING (stimulator of interferon genes) has also been demonstrated to be a mediator for type I IFN induction by intracellular exogenous DNA in a TLR-independent manner. Cytosolic detection of DNA activates STING in the cytoplasm, which binds to TBK1 (TANK-binding kinase 1) and IKK (IκB kinase), that in turn activates the transcription factors IRF3 (interferon regulatory factor 3)/STATE, and NF-κB (nuclear factor κB), respectively. Subsequently, nuclear translocation of these transcription factors leads to the induction of type I IFNs and other cytokines that participate in host defense. In the past six years, STING has been demonstrated to be essential for the host protection against DNA pathogens through various mechanisms. STING is also a mediator for autoimmune diseases which are initiated by the aberrant cytoplasmic DNA. Following the recognition of cytosolic DNA, cGAMP synthase (cGAS) catalyzes the generation of 2′ to 5′ cyclic GMP-AMP (cGAMP), which binds to and activates STING signaling. More recently, cGAS has been considered as a universal cytosol DNA sensor for STING activation, such as in the setting of viral infection and lupus erythematosus. Now we elucidate the role of host cGAS-STING in the sensing of irradiated-tumor cells. Here, we demonstrate that radiotherapy is dominated by a distinct mechanism different from chemotherapy and targeted therapies with antibodies, which rely on HMGB-1-TLR4-MyD88 interaction. Antitumor effects of radiation are controlled by newly defined cGAS-STING-dependent cytosolic DNA sensing pathway, which drives a rigorous innate immune response and a robust adaptive immune response to radiation.

In another aspect of the present disclosure, it is contemplated that an agent administered to a subject undergoing radiotherapy that increases cGAS levels in a cancerous cell as compared to an untreated cancerous state control cell, promotes antitumor efficacy of the radiation as compared to an untreated (that is, no agent is administered to the subject undergoing radiotherapy) control subject. While not wishing to be bound by theory, is it believed that cGAS mediates type I IFN production to enhance the function of dendritic cells in response to irradiated-tumor cells. We therefore contemplate that DNA from irradiated-tumor cells delivered into the cytosol of dendritic cells binds to cGAS to trigger STING-dependent type I IFN induction. Although cancer type, tissue and/or subject dependent, it is contemplated that elevated cGAS levels generally greater than about 10%, 25%, 50%, 75%, 100% or greater in a treated cancerous cells as compared to an untreated control cell provides the desired antitumor efficacy in a subject undergoing radiotherapy for a particular cancer. Such agents that increase cGAS levels in a cell include, for example DNA damaging agents used in the clinic at clinical doses. In one embodiment, the agent is delivered to a cancerous cell by a pharmaceutical carrier such as a nanocarrier, a conjugate, a nucleic-acid-lipid particle, a vesicle, a exosome, a protein capsid, a liposome, a dendrimer, a lipoplex, a micelle, a virosome, a virus like particle, a nucleic acid complexes, and mixtures and derivatives thereof. In yet another embodiment, the agent is delivered into the cytosol of the subject's dendritic cell by, for example, the pharmaceutical carrier via intratumoral (IT), intraveinous (IV), and/or intraperitoneal (IP) administration.

Therefore, this disclosure provides insight into understanding the mechanism of radiation-mediated tumor regression and forms new strategies for improvements in radiotherapy efficacy in cancer patients.

High and low expression of LGP2 refers to expression levels of about +/−1.5 fold, respectively, as related to average level of expression of this gene in investigated and published databases.

Reactive Oxygen Species (ROS) are molecules containing oxygen and generally very chemically reactive. Examples include oxygen ions and peroxides. ROS also is created as a natural by-product of the normal metabolism of oxygen, but when a cell is exposed to environmental stress such as UV or heat exposure, ROS levels can increase dramatically resulting in significant cell damage known as oxidative stress. Such damage includes damage to cellular proteins, lipids and DNA, that may lead to fatal lesions in a cell that contributes to carcinogenesis. Ionizing radiation may also generate ROS in a cell and may result in considerable damage to the cell.

As used herein, the term “patient” refers to a human or non-human mammalian patient suffering from a condition in need of treatment. In one embodiment of the present invention, the condition may be a cancer.

The term “RIG-1 binding RNAs” or “rbRNAs,” as used herein refers to any RNA capable of binding to Retinoic acid inducible gene-1 (RIG-1) and capable of stimulating interferon production. US Patent Application publication of US 2016/0046943 discloses some exemplary rbRNAs.

A shRNA (small hairpin RNA or short hairpin RNA) is a sequence of RNA getting its name from a tight hairpin turn that can be used to silence target gene expression via RNA interference (RNAi). Expression of shRNA in cells is generally known in the art and is typically accomplished by the delivery of plasmids or through viral or bacterial vectors.

A siRNA (small interfering RNA (siRNA) (also known as short interfering RNA or silencing RNA) is a class of double-stranded RNA molecules, 20-25 base pairs in length. siRNA plays a role in several important pathways including the RNA interference (RNAi) pathway and the RNAi-related pathways. siRNA may, for example, interfere with the expression of specific genes with complementary nucleotide sequence.

The term “double-stranded RNA” or “dsRNA,” as used herein, refers to a RNA with two complementary strands, similar to the DNA found in all cells. dsRNA forms the genetic material of some viruses (double-stranded RNA viruses). Double-stranded RNA such as viral RNA or siRNA can trigger RNA interference in eukaryotes, as well as interferon response in vertebrates.

The term “small nuclear ribonucleic acid” or “snRNA,” also commonly referred to as U-RNA, as used herein refers to a class of small RNA molecules that may be found within the splicing speckles and Cajal bodies of the cell nucleus in eukaryotic cells. The length of an average snRNA may be approximately 150 nucleotides. For example, U1 may include 127 nucleotides. Among them, U1 stem loop I includes 32 nucleotides, U1 stem loop II includes 38 nucleotides, U1 stem loop III includes 26 nucleotides, and U1 stem loop IV includes 31 nucleotides. U2 may include 188 nucleotides. M5 may include 81 nucleotides. M8 may include 101 nucleotides. snRNAs may be transcribed by either RNA polymerase II or RNA polymerase III, and studies have shown that their primary function is in the processing of pre-messenger RNA (hnRNA) in the nucleus. snRNAs have also been shown to aid in the regulation of transcription factors (7SK RNA) or RNA polymerase II (B2 RNA), and maintaining the telomeres.

snRNAs may always be associated with a set of specific proteins, and the complexes are referred to as small nuclear ribonucleoproteins (snRNP, often pronounced “snurps”). Each snRNP particle is composed of several Sm proteins, the snRNA component, and snRNP-specific proteins. The most common snRNA components of these complexes are known, respectively, as: U1 spliceosomal RNA, U2 spliceosomal RNA, U4 spliceosomal RNA, U5 spliceosomal RNA, and U6 spliceosomal RNA. Their nomenclature derives from their high uridine content.

A large group of snRNAs are known as small nucleolar RNAs (snoRNAs). These are small RNA molecules that play an essential role in RNA biogenesis and guide chemical modifications of ribosomal RNAs (rRNAs) and other RNA genes (tRNA and snRNAs). They may be located in the nucleolus and the Cajal bodies of eukaryotic cells (the major sites of RNA synthesis), where they are called scaRNAs (small Cajal body-specific RNAs).

In one embodiment of the present invention, snRNAs may be dsRNAs.

snRNA may often be divided into two classes based upon both common sequence features as well as associated protein factors such as the RNA-binding LSm proteins. The first class, known as Sm-class snRNA, consists of U1, U2, U4, U4atac, U5, U7, Ulf, and U12. Sm-class snRNA may be transcribed by RNA polymerase II. The pre-snRNA may be transcribed and receive the usual 7-methylguanosine five-prime cap in the nucleus. They are then exported to the cytoplasm through nuclear pores for further processing. In the cytoplasm, the snRNA receive 3′ trimming to form a 3′ stem-loop structure, as well as hypermethylation of the 5′ cap to form trimethylguanosine. The 3′ stem structure is necessary for recognition by the survival of motor neuron (SMN) protein. This complex assembles the snRNA into stable ribonucleoproteins (RNPs). The modified 5′ cap is then required to import the snRNP back into the nucleus. All of these uridine-rich snRNA, with the exception of U7, form the core of the spliceosome. Splicing, or the removal of introns, is a major aspect of post-transcriptional modification, and takes place only in the nucleus of eukaryotes. U7 snRNA has been found to function in histone pre-mRNA processing.

The second class, known as Lsm-class snRNA, consists of U6 and U6atac. Lsm-class snRNAs may be transcribed by RNA polymerase III and never leave the nucleus, in contrast to Sm-class snRNA. Lsm-class snRNAs contain a 5′-γ-monomethylphosphate cap and a 3′ stem-loop, terminating in a stretch of uridines that form the binding site for a distinct heteroheptameric ring of Lsm proteins.

The term “U1” or “U1 snRNP,” as used herein, refers to the initiator of spliceosomal activity in the cell by base pairing with the hnRNA. In the major spliceosome, experimental data has shown that the U1 snRNP may be present in equal stoichiometry with U2, U4, U5, and U6 snRNP. However, U1 snRNP's abundance in human cells may be far greater than that of the other snRNPs.

The term “functionally equivalent fragment(s),” as used herein, refers to any fragments of the rbRNAs (e.g., snRNAs) that exhibit binding specificity and activity that is substantially equivalent to the rbRNAs (e.g., snRNAs) from which it/they is/are derived. The term “substantially equivalent,” as used herein, refers to any fragment having at least 80%, preferably 85%, or more preferably 90% binding specificity and activity of the rbRNAs (e.g., snRNAs) from which it/they is/are derived. In one preferred embodiment, a functionally equivalent fragment may at least comprise the double-stranded regions of the rbRNAs (e.g., snRNAs) from which it/they is/are derived. In one embodiment of the present invention, a functionally equivalent fragment may be a chemically synthesized RNA comprising at least the double-stranded regions of the rbRNAs (e.g., snRNAs) from which it/they is/are derived. In one embodiment, a functionally equivalent fragment may be chemically synthesized RNA comprising a stem-loop region. A functionally equivalent fragment may be chemically synthesized RNA comprising two stem-loops regions. The fragments may be modified at the 5′-end to comprise a phosphorylation or cap-0.

In one aspect, the present invention discloses a composition for treating cancer in a subject in need thereof. In one embodiment, the composition for treating cancer in a subject in need thereof, comprising a therapeutically effective amount of at least one rbRNA (e.g., snRNA) or its functionally equivalent fragment, and a pharmaceutically acceptable carrier, wherein the at least one rbRNA (e.g., snRNA) or its functionally equivalent fragment activates primary RNA or DNA sensors and wherein the composition is administered to the subject before a dose of ionized radiation is administered on the subject.

Applicants identify a list of polynucleotides which can be used as tumor radio/chemosensitizers and immune stimulators. In one embodiment, the polynucleotides are double-stranded. In one preferred embodiment, the polynucleotides are rbRNAs (e.g., snRNAs).

Examples 3-5 describe some exemplary rbRNAs (e.g., snRNAs) with tumor radio/chemo-sensitizing and immunomodulatory properties and methods of their preparation and application. Specifically, Table 3 shows a list of rbRNAs (e.g., snRNAs) according to one embodiment of the present invention. Table 4 shows a list of rbRNAs (e.g., snRNAs) according to another embodiment of the present invention.

Examples 3-5 demonstrate that U1, U2 and other rbRNAs (e.g., snRNAs) in Tables 3, 4 and 5 were produced as enriched expression products of primary RNA sensors such as RIG-I under IR. Examples 3-5 further demonstrate that these rbRNAs (e.g., snRNAs) are natural endogenous RNAs which are capable of binding to RIG-I and other RNA sensor proteins and induce Type I IFN, thereby affecting tumor response to radio/chemotherapy and immune system.

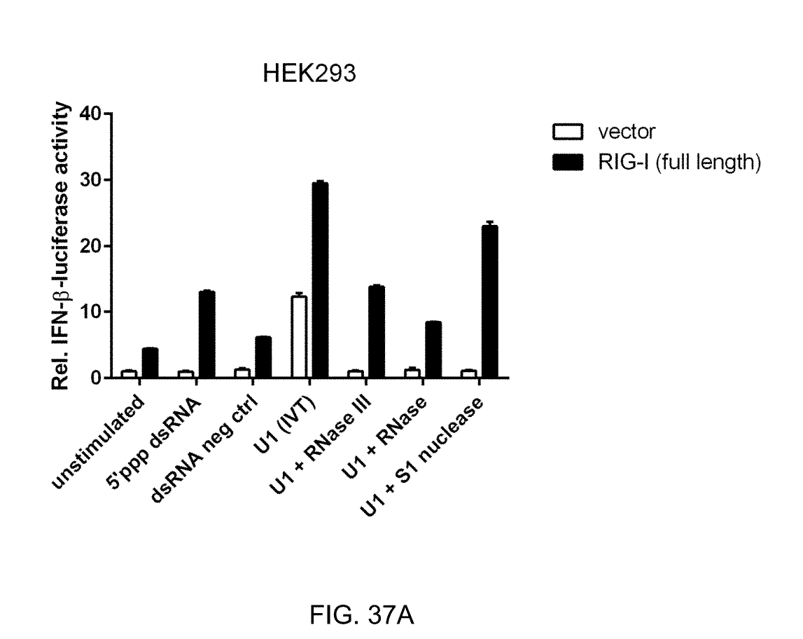

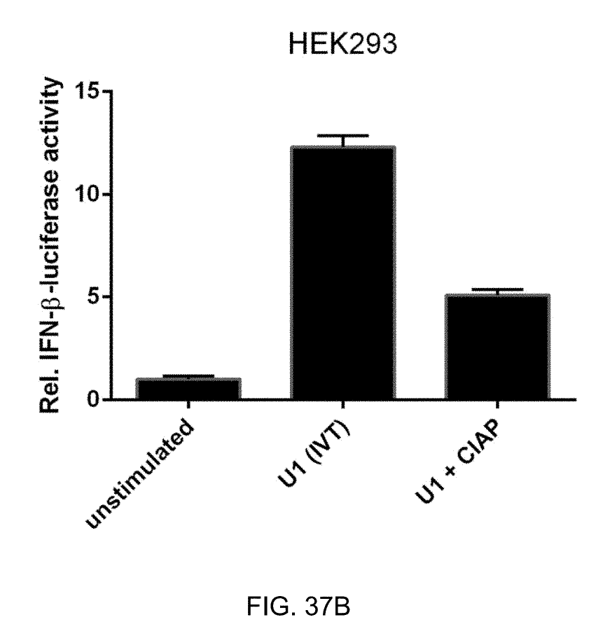

For example, FIG. 37A shows that U1 snRNA has potent IFN-beta stimulatory activity in RIG-1 overexpressing cells and is capable of activating endogenous RIG-1 in HEK293 cells.

Further, Examples 3-5 show that these small endogenous rbRNAs (e.g., snRNAs) such as U1 snRNA can be successfully delivered into a tumor microenvironment and show positive effects of tumor treatment along with IR on their persistence in the tumor bed. These data demonstrate that U1 or U2 endogenous snRNAs and other rbRNAs (e.g., snRNAs), may induce IFN-beta promoter in vitro, and may be used as a potent radiosensitizer of tumor in preclinical animal model.

In one embodiment, a composition for treating cancer comprises at least one of such rbRNA (e.g., snRNA) such as U1 or U2 endogenous snRNAs or their functionally equivalent fragments.

In one embodiment, the functionally equivalent fragments of the rbRNAs (e.g., snRNAs) may be naturally existing RNAs or chemically synthesized RNAs.

In one specific embodiment, the functionally equivalent fragments may at least comprise the double-stranded regions of corresponding endogenous rbRNAs (e.g., snRNAs).

In another specific embodiment, the rbRNA comprises a modification of the 5′ end. In one embodiment the modification is a tri-phosphorylation or a 5′ cap (cap-0).

In one specific embodiment, the rbRNA (e.g., snRNA) is U1 snRNA. In another embodiment, the snRNA is U2 snRNA. Applicants envision that either U1 or U2 snRNA may be used in combination with at least another snRNA from Table 3, Table 4 or Table 5.

In one embodiment, the rbRNA (e.g., snRNA) is M5. In one embodiment, M5 has the sequence of 5′ gacgaagaccacaaaaccagataaaaaattattttttatctggttttgtggtcttcgtctatagtgagtcgtattaatttc 3′ (SEQ ID NO:26).

In one embodiment, the rbRNA (e.g., snRNA) is M8. In one embodiment, M8 has the sequence of 5′ gaaattaatacgactcactatagacgaagaccacaaaaccagataaaaaaaaaaaaaaaataattttttttttttttttatctggttttgtggtcttcg tc 3′ (SEQ ID NO:27).

Previous literatures such as J. Virol. doi:10.1128/JVI.00845-15 (Chiang et al., “Sequence-specific modifications enhance the broad spectrum antiviral response activated by RIG-I agonists”) include sequences of M5, M8 and other RNAs. Applicants envision that other RNAs may also be used in the present invention.

In one embodiment, the rbRNA (e.g., snRNA) of the present invention is selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

In another embodiment, the rbRNA (e.g., snRNA) of the present invention is selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

In one embodiment, Applicants envision that one might use at least two rbRNAs (e.g., snRNAs) selected from the group consisting of U1, U2, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

In one embodiment, Applicants envision that one might use at least two rbRNAs (e.g., snRNAs) selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

In one specific embodiment, the at least two rbRNAs (e.g., snRNAs) comprise U1 snRNA.

In one specific embodiment, the at least two rbRNAs (e.g., snRNAs) comprise U2 snRNA.

In one embodiment, the composition for treating cancer may comprise a pharmaceutically acceptable carrier. In one embodiment, the pharmaceutically acceptable carrier comprises at least one of a nanocarrier, a conjugate, a nucleic-acid-lipid particle, a vesicle, an exosome, a protein capsid, a liposome, a dendrimer, a lipoplex, a micelle, a virosome, a virus like particle, and a nucleic acid complexes.

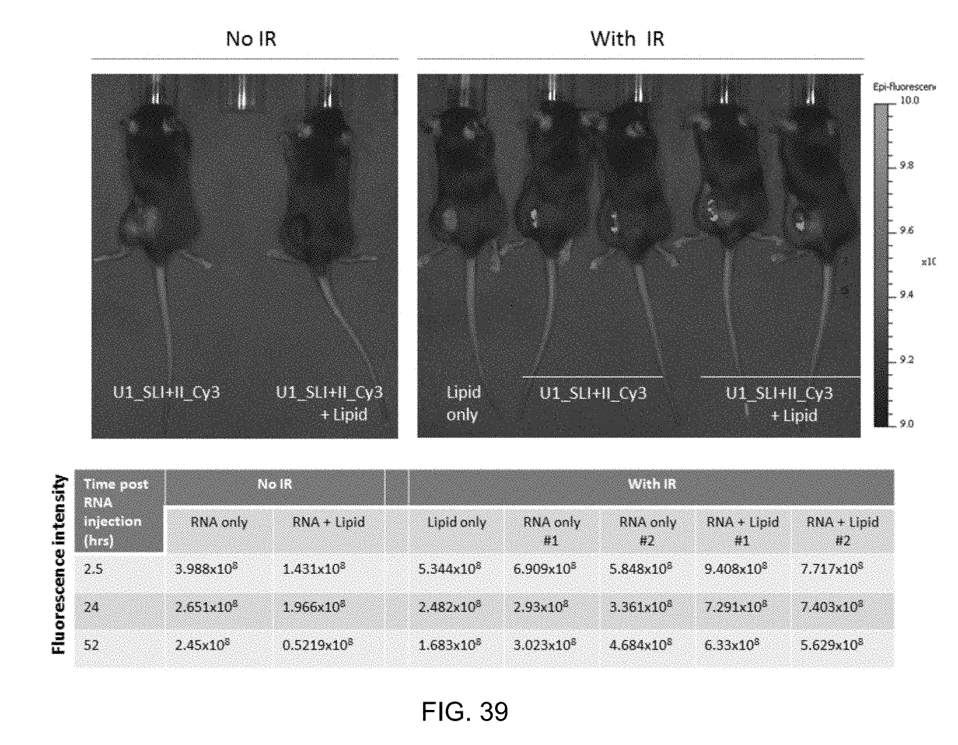

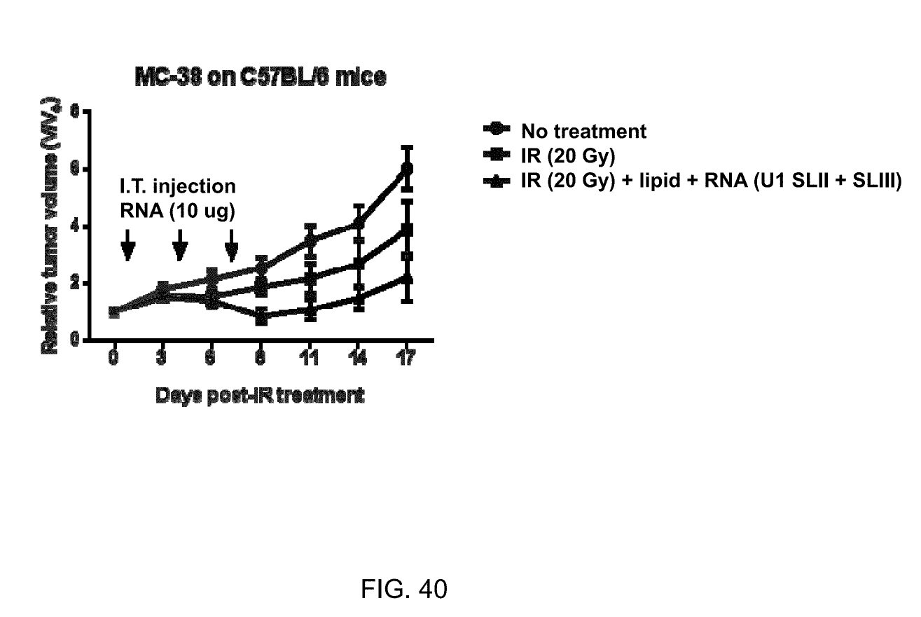

In one specific embodiment, the pharmaceutically acceptable carrier is a lipid. For example, FIGS. 39 and 40 show that jetPEI lipid may be used to stabilize rbRNAs (e.g., snRNAs) of the present invention.

In one embodiment, the rbRNAs (e.g., snRNAs) of the present invention are activators for primary RNA or DNA sensors. In one specific embodiment, the primary RNA or DNA sensor comprises at least one of RIG1, MDA5, DAI, IFI16, Aim2, and cGAS. In one preferred embodiment, the primary RNA or DNA sensor is RIG1. For example, Examples 3-5 use RIG1 as the exemplary primary RNA sensor. Applicants envision that the present composition is applicable to any other primary RNA or DNA sensor as discussed above or as appreciated by one skilled in the art.

In one embodiment, the composition of the present invention further comprises another therapeutic agent. In one embodiment, the other therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

In another embodiment, the other therapeutic agent is selected from the group consisting of abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); zoledronate (Zometa®) and vorinostat (Zolinza®).

In another embodiment, the present composition may also be combined with standard and SBRT radiotherapy and chemotherapy in oncology. One may also consider individual applications of such rbRNA (e.g., snRNA) drugs in conditions, associated with viral infections, wound healing, fibrosis, chronical inflammation and others as appreciated by one skilled in the art.

In one embodiment, the present composition may be administered to the subject before a dose of ionized radiation is administered on the subject. In one preferred embodiment, the dose of ionized radiation administered on the subject is in the range of 3-50 Gy, preferably 5-30 Gy, and more preferably 6-20Gy.

In another aspect, the present invention is a method of treating cancer in a subject in need thereof.

In one embodiment, the method of treating cancer in a subject in need thereof. The method comprises the steps of (a) administering to the subject a pharmaceutical composition comprising a therapeutically effective amount of at least one rbRNA (e.g., snRNA) or its functionally equivalent fragment, and a pharmaceutically acceptable carrier, wherein the at least one rbRNA (e.g., snRNA) or its functionally equivalent fragment activates a primary RNA or DNA sensor, and wherein the endogenous IFNbeta (IFNβ production of the subject is regulated, and (b) administering to the subject a therapeutic amount of ionizing radiation.

In one embodiment, the at least one rbRNA (e.g., snRNA) or its functionally equivalent fragment is a double-stranded RNA.

In one embodiment, the at least one rbRNA (e.g., snRNA) is selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

In one embodiment, the at least one rbRNA (e.g., snRNA) is selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

In one embodiment, the at least one rbRNA (e.g., snRNA) is U1 snRNA.

In one embodiment, the at least one rbRNA (e.g., snRNA) is U2 snRNA.

In one embodiment, the at least one rbRNA (e.g., snRNA) is M5.

In one embodiment, the at least one rbRNA (e.g., snRNA) is M8.

In one embodiment, the composition used in the present method further comprises another therapeutic agent. In one embodiment, the other therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

In another embodiment, the other therapeutic agent may be any therapeutic agent as discussed in this application.

In one embodiment, the at least one rbRNA (e.g., snRNA) or its functionally equivalent fragment may be further covalently attached to a reporter group. This would allow one to monitor stability of injected RNAs using in vivo or ex vivo microscopy or any non-invasive imaging approach to trace labelled molecules in the tumor microenvironment.

In one embodiment, the pharmaceutically acceptable carrier used in the present method comprises at least one of a nanocarrier, a conjugate, a nucleic-acid-lipid particle, a vesicle, a exosome, a protein capsid, a liposome, a dendrimer, a lipoplex, a micelle, a virosome, a virus like particle, and a nucleic acid complexes.

In one specific embodiment, the pharmaceutically acceptable carrier is a lipid.

As discussed above, the at least one rbRNA (e.g., snRNA) or its functionally equivalent fragment activates a primary RNA or DNA sensor.

In one embodiment, the primary RNA or DNA sensor comprises at least one of RIG1, MDA5, DAI, IFI16, Aim2, and cGAS.

In one specific embodiment, the primary RNA or DNA sensor is RIG1.

In one embodiment, the ionizing radiation comprises at least one of brachytherapy, external beam radiation therapy, and radiation from cesium, iridium, iodine, and cobalt.

In one embodiment, the subjection is a human being.

LPG2, MDA5, and RIG-1 are members of the RIG-1-like receptor dsRNA helicase enzyme family. In humans, LGP2 (Laboratory of Genetics and Physiology 2) is encoded by the DHX58 gene; RIG-1 (retinoic acid-inducible gene 1) is encoded by the DDX58 gene; and MDA5 (Melanoma Differentiation-Associated protein 5) is encoded by the IFIH1 gene. LGP2 (Human Entrez GeneID: 79132; Mouse Entrez GeneID: 80861) may also be identified by the symbols LGP-2, DHX58, D11LGP2, D11lgp2e, and RLR-3; RIG-1 (Human Entrez GeneID: 23586; Mouse Entrez GeneID: 230073) may also be identified by the symbols RIGI, DDX58, and RLR-1; and MDA5 (Human Entrez GeneID: 64135; Mouse Entrez GeneID: 71586) may also be identified as MDA-5, IFIHI, Hlcd, IDDM19, and RLR-2.

MAVS (Mitochondrial antiviral-signaling protein) is a protein that in humans is encoded by the MAVS gene. The MAVS protein (Human Entrez GeneID: 57506; Mouse Entrez GeneID: 228607) may also be identified by the symbols CARDIF; IPS-1, IPS1, and VISA.

In humans, TREX1 (Three prime repair Exonuclease 1) is an enzyme that is encoded by the TREX1gene. TREX1 (Human Entrez GeneID: 11277; Mouse Entrez GeneID: 22040) may also be identified by the symbols AGS1, CRV, DRN3, and HERNS.

DAI (DNA-dependent Activator of IFN regulatory factors), also identified as DLM-1/ZBP1, functions as a DNA sensor in humans and is generally thought to activate the innate immune system.

IFI16 (Gamma-interferon-inducible protein Ifi-16) in humans is a protein that is encoded by the IFI16 gene. IFI16 (Human Entrez GeneID: 3428; Mouse Entrez GeneID: 15951) may also be identified by the symbols IFI-16, IFNGIP1 and PYHIN2, and be known as interferon-inducible myeloid differentiation transcriptional activator.

AIM2 (Interferon-inducible protein AIM2) is a protein that in humans is encoded by the AIM2 gene and a member of the IFI16 family. AIM2 (Human Entrez GeneID: 9447; Mouse Entrez GeneID: 383619) may also be known as Absent In Melanoma 2 and by the symbol PYHIN4.

STING (Stimulator of Interferon (IFN) Genes) in humans is encoded by the TMEM173 gene and may also be identified by the symbols TMEM173, ERIS, MITA, MPYS, and NET23.

cGAS (cyclic-GMP-AMP synthase) in humans is encoded by the MB21D1/C6orf150 gene and may also be identified by the symbols cGAS, MB21D1, and C6orf150. cGAS may also be known as cGAMP synthase.

It is further contemplated that a treatment regimen may include administering an antineoplastic agent (e.g., chemotherapy) along with IR (or radiotherapy) to treat a resistant cancer cell. An illustrative antineoplastic agent or chemotherapeutic agent include, for example, a standard taxane. Taxanes are produced by the plants of the genus Taxus and are classified as diterpenes and widely uses as chemotherpy agents including, for example, paclitaxel, (Taxol®, Bristol-Meyers Squibb, CAS 33069-62-4) and docetaxel (Taxotere®, Sanofi-Aventis, CAS 114977-28-5). Other chemotherapeutic agent include semi-synthetic derivatives of a natural taxoid such as cabazitaxel (Jevtana®, Sanofi-Aventis, CAS 183133-96-2). Other chemotherapeutic agent also include an androgen receptor inhibitor or mediator. Illustrative androgen receptor inhibitors include, a steroidal antiandrogen (for example, cyperterone, CAS 2098-66-0); a non-steroidal antiandrogen (for example, flutamide, Eulexin®, Schering-Plough, CAS 13311-84-7); nilutamide (Nilandron®, CAS 63612-50-0); enzalutamide (Xtandi®, Medivation®, CAS 915087-33-1); bicalutamide (Casodex, AstraZeneca, CAS 90357-06-5); a peptide antiandrogen; a small molecule antiandrogen (for example, RU58642 (Roussel-Uclaf SA, CAS 143782-63-2); LG120907 and LG105 (Ligand Pharmaceuticals); RD162 (Medivation, CAS 915087-27-3); BMS-641988 (Bristol-Meyers Squibb, CAS 573738-99-5); and CH5137291(Chugai Pharmaceutical Co. Ltd., CAS 104344603904)); a natural antiandrogen (for example, ataric acid (CAS 4707-47-5) and N-butylbensensulfonamide (CAS 3622-84-2); a selective androgen receptor modulator (for example, enobosarm (Ostarine®, Merck & Company, CAS 841205-47-8); BMS-564,929 (Bristol-Meyer Squibb, CAS 627530-84-1); LGD-4033 (CAS 115910-22-4); AC-262,356 (Acadia Pharmaceuticals); LGD-3303 (Ganolix Lifescience Co., Ltd., 9-chloro-2-ethyl-1-methyl-3-(2,2,2-trifluoroethyl)-3H-pyrrolo[3,2-f]quinolin-7(6H)-one; 5-40503, Kaken Pharmaceuticals, 2-[4-(dimethylamino)-6-nitro-1,2,3,4-tetrahydroquinolin-2-yl]-2-methylpropan-1-ol); andarine (GTx-007, S-4, GTX, Inc., CAS 401900-40-1); and S-23 (GTX, Inc., (2S)—N-(4-cyano-3-trifluoromethylphenyl)-3-(3-fluoro-4-chlorophenoxy)-2-hydroxy-2-methyl-propanamide)); or those described in U.S. Patent Appln. No. 2009/0304663. Other neoplastic agents or chemotherapeutic agents that may be used include, for example: alkylating agents such as nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; biological response modifiers such as interferon alphenomes; other agents such as platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MTH); adrenocortical suppressant such as mitotane (o,p′-DDD) and aminoglutethimide; taxol analogues/derivatives; hormone agonists/antagonists such as flutamide and tamoxifen; and GnRH and analogues thereof. Examples of other chemotherapeutic can be found in Cancer Principles and Practice of Oncology by V. T. Devita and S. Hellman (editors), 6.sup.th edition (Feb. 15, 2001), Lippincott Williams & Wilkins Publishers.

Radiotherapy is based on ionizing radiation delivered to a target area that results in death of reproductive tumor cells. Some examples of radiotherapy include the radiation of cesium, palladium, iridium, iodine, or cobalt and is usually delivered as ionizing radiation delivered from a linear accelerator or an isotopic source such as a cobalt source. Also variations on linear accelerators are Cyberkine and Tomotherapy. Particle radiotherapy from cyclotrons such as Protons or Carbon nuclei may be employed. Also radioisotopes delivered systemically such as p32 or radiou 223 may be used. The external radiotherapy may be systemic radiation in the form of stereotacktic radiotherapy total nodal radiotherapy or whole body radiotherapy but is more likely focused to a particular site, such as the location of the tumor or the solid cancer tissues (for example, abdomen, lung, liver, lymph nodes, head, etc.). The radiation dosage regimen is generally defined in terms of Gray or Sieverts time and fractionation, and must be carefully defined by the radiation oncologist. The amount of radiation a subject receives will depend on various consideration but the two important considerations are the location of the tumor in relation to other critical structures or organs of the body, and the extent to which the tumor has spread. One illustrative course of treatment for a subject undergoing radiation therapy is a treatment schedule over a 5 to 8 week period, with a total dose of 50 to 80 Gray (Gy) administered to the subject in a single daily fraction of 1.8 to 2.0 Gy, 5 days a week. A Gy is an abbreviation for Gray and refers to 100 rad of dose.

Radiotherapy can also include implanting radioactive seeds inside or next to a site designated for radiotherapy and is termed brachytherapy (or internal radiotherapy, endocurietherapy or sealed source therapy). For prostate cancer, there are currently two types of brachytherapy: permanent and temporary. In permanent brachytherapy, radioactive (iodine-125 or palladium-103) seeds are implanted into the prostate gland using an ultrasound for guidance. Illustratively, about 40 to 100 seeds are implanted and the number and placement are generally determined by a computer-generated treatment plan known in the art specific for each subject. Temporary brachytherapy uses a hollow source placed into the prostate gland that is filled with radioactive material (iridium-192) for about 5 to about 15 minutes, for example. Following treatment, the needle and radioactive material are removed. This procedure is repeated two to three times over a course of several days.

Radiotherapy can also include radiation delivered by external beam radiation therapy (EBRT), including, for example, a linear accelerator (a type of high-powered X-ray machine that produces very powerful photons that penetrate deep into the body); proton beam therapy where photons are derived from a radioactive source such as iridium-192, caesium-137, radium-226 (no longer used clinically), or colbalt-60; Hadron therapy; multi-leaf collimator (MLC); and intensity modulated radiation therapy (IMRT). During this type of therapy, a brief exposure to the radiation is given for a duration of several minutes, and treatment is typically given once per day, 5 days per week, for about 5 to 8 weeks. No radiation remain in the subject after treatment. There are several ways to deliver EBRT, including, for example, three-dimensional conformal radiation therapy where the beam intensity of each beam is determined by the shape of the tumor. Illustrative dosages used for photon based radiation is measured in Gy, and in an otherwise healthy subject (that is, little or no other disease states present such as high blood pressure, infection, diabetes, etc.) for a solid epithelial tumor ranges from about 60 to about 80 Gy, and for a lymphoma ranges from about 20 to about 40 Gy. Illustrative preventative (adjuvant) doses are typically given at about 45 to about 60 Gy in about 1.8 to about 2 Gy fractions for breast, head, and neck cancers.

When radiation therapy is a local modality, radiation therapy as a single line of therapy is unlikely to provide a cure for those tumors that have metastasized distantly outside the zone of treatment. Thus, the use of radiation therapy with other modality regimens, including chemotherapy, have important beneficial effects for the treatment of metastasized cancers.

Radiation therapy has also been combined temporally with chemotherapy to improve the outcome of treatment. There are various terms to describe the temporal relationship of administering radiation therapy and chemotherapy, and the following examples are illustrative treatment regimens and are generally known by those skilled in the art and are provided for illustration only and are not intended to limit the use of other combinations. “Sequential” radiation therapy and chemotherapy refers to the administration of chemotherapy and radiation therapy separately in time in order to allow the separate administration of either chemotherapy or radiation therapy. “Concomitant” radiation therapy and chemotherapy refers to the administration of chemotherapy and radiation therapy on the same day. Finally, “alternating” radiation therapy and chemotherapy refers to the administration of radiation therapy on the days in which chemotherapy would not have been administered if it was given alone.

It should be noted that other therapeutically effective doses of radiotherapy can be determined by a radiation oncologist skilled in the art and can be based on, for example, whether the subject is receiving chemotherapy, if the radiation is given before or after surgery, the type and/or stage of cancer, the location of the tumor, and the age, weight and general health of the subject.

It is further contemplated that subsets of gene targets, including those identified or described herein, could be used as a therapeutic tool for diagnosing and/or treating a tumor or cancer. For example, siRNA pools (or other sets of molecules individually specific for one or more predetermined targets including, for example, shRNA pools, small molecules, and/or peptide inhibitors, collectively “expression inhibitors” or “active ingredients” or “active pharmaceutical ingredients”) may be generated based on one or more (e.g., 2 or 4 or 8 or 12, or any number) targets and used to treat a subject in need thereof (e.g., a mammal having a chemoresistant or radioresistant cancer). Upon rendering of the subject's cancer chemosensitive and/or radiosensitive, therapeutic intervention in the form of antineoplastic agents and/or ionizing radiation as known in the art (see for example, U.S. Pat. No. 6,689,787, incorporated by reference) may be administered to reduce and/or eliminate the cancer. It is contemplated that therapeutic intervention may occur before, concurrent, or subsequent the treatment to render the subject chemosensitive or radiosensitive. It is further envisioned that particular subsets of targets may be advantageous over others based on the particular type of cancer and/or tissue of origin for providing a therapeutic effect. Administration of such therapies may be accomplished by any means known in the art.

In one embodiment, a kit may include a panel of siRNA pools directed at one or more targets as identified by or in the present disclosure. It is envisioned that a particular kit may be designed for a particular type of cancer and/or a specific tissue. The kit may further include means for administering the panel to a subject in need thereof. In addition, the kit may also include one or more antineoplastic agents directed at the specific type of cancer against which the kit is directed and one or more compounds that inhibit that Jak/Stat pathway.

Kits may further be a packaged collection of related materials, including, for example, a single and/or a plurality of dosage forms each approximating an therapeutically effective amount of an active ingredient, such as, for example, an expression inhibitor and/or a pharmaceutical compound as described herein that slows, stops, or reverses the growth or proliferation of a tumor or cancer or kills tumor or cancer cells, and/or an additional drug. The included dosage forms may be taken at one time, or at a prescribed interval. Contemplated kits may include any combination of dosage forms.

A kit may also be a prognostic kit for use with a tissue suffering from or having a cancer, including, for example, a tissue taken from a subject suffering from a high grade glioma. The prognostic kit may contain at least one set of primers for QRT-PCR detection of LGP2 to determine expression levels of LGP2 in the tissue. The prognostic kit may also include at least one of: a reagent for purification of total RNA from the tissue, a set of reagents for a QRT-PCR reaction, and/or a positive control for detection of LGP2 mRNA. Generally, high expression levels of LGP2 and low expression levels of LGP2 predict improved prognosis in treating the cancer in the tissue or the subject from which the tissue was derived. The tissue may also be from any part of the subject in which the cancer is present including, for example, tissue from the brain. As for thresholds of prognosis for LGP2 levels, the use of high and low+/−1.5 fold as related to average level of expression of this gene in investigated and published databases can be used. For example, “high expression” levels of LGP2 may be, for example, at least about 1.5 fold greater than an expression level of LGP2 in a normal non-disease state tissue; while “low expression” levels of LGP2 may be, for example, at least about 1.5 fold less than an expression level of LGP2 in a normal non-disease state tissue.

In some embodiments the rbRNAs are attached to a “reporter group.” The reporter group, for example, can be a Renilla luciferase reporter, a radioactive isotope, a fluorophore, or a fluorescent protein. In a specific embodiment, the radioactive isotope is gadallinium, thallium, technetium, iodine, yttrium, metaiodobenzylguanidine, samarium, strontium, caesium, cobalt, iridium, palladium, or ruthenium.

In another embodiment, a method of treating a subject in need thereof includes administering to the subject one or more molecules that target one or more genes such as siRNA and/or shRNA pools. The method may further include, for example, treatment of the subject with one or more antineoplastic agents, ionizing radiation, and/or one or more compounds that inhibit that Jak/Stat pathway.

Suppression of a gene refers to the absence of expression of a gene or a decrease in expression of a gene or suppression of a product of a gene such as the protein encoded by the given gene as compared to the activity of an untreated gene. Suppression of a gene may be determined by detecting the presence or absence of expression of a gene or by measuring a decrease of expression of a gene by any means known in the art including, for example, detecting a decrease in the level of the final gene product, such as a protein, or detecting a decreased level of a precursor, such as mRNA, from which gene expression levels may be inferred when compared to normal gene activity, such as a negative (untreated) control. Any molecular biological assay to detect mRNA or an immunoassay to detect a protein known in the art can be used. A molecular biological assay includes, for example, polymerase chain reaction (PCR), Northern blot, Dot blot, or an analysis method with microarrays. An immunological assay includes, for example, ELISA (enzyme-linked immunosorbent assay) with a microtiter plate, radioimmunoassay (MA), a fluorescence antibody technique, Western blotting, or an immune structure dyeing method. Suppression of a gene may also be inferred biologically in vivo, in situ, and/or in vitro, by the suppression of growth or proliferation of a tumor or cancer cell, cell death of a tumor or cancer cell, and/or the sensitization of a tumor or cancer cell to chemotherapy and/or radiotherapy. Illustratively, a therapeutically effective amount or a therapeutically effective amount of gene suppression in a subject results in the suppression of growth or proliferation of a tumor or cancer cell, cell death of the tumor or cancer cell, sensitization of the tumor or cancer cell to chemotherapy and/or radiotherapy, and/or protecting normal non-disease state tissue from genotoxic stress. As each subject is different and each cancer is different, the quantitative amount to achieve a therapeutically effective amount in a subject may be determined by a trained professional skilled in the area on a case by case basis. Illustratively, a therapeutically effective amount of gene suppression may include, for example, less than or equal to about 95% of normal gene activity, or less than or equal to about 90% of normal gene activity, or less than or equal to about 85% of normal gene activity, or less than or equal to about 80% of normal gene activity, or less than or equal to about 75% of normal gene activity, or less than or equal to about 65% of normal gene activity, or less than or equal to about 50% of normal gene activity, or less than or equal to about 35% of normal gene activity, or less than or equal to about 25% of normal gene activity, or less than or equal to about 15% of normal gene activity, or less than or equal to about 10% of normal gene activity, or less than or equal to about 7.5% of normal gene activity, or less than or equal to about 5% of normal gene activity, or less than or equal to about 2.5% of normal gene activity, or less than or equal to about 1% of normal gene activity, or less than or equal to about 0% of normal gene activity.

Suppression of identified genes individually or in combination combined with ionizing radiation and/or any chemotherapeutic agents may improve the outcome of patients treated with the ionizing radiation or any chemotherapy agent or any treatment designed to improve outcome of the cancer patients if such treatment is combined with the suppression of any of these genes or their combination.

Based on the functional groups, we also contemplate that suppression of the chemokine signaling, or suppression of negative regulators of interferon response, or suppression of protein degradation or mitochondria-related anti-apoptotic molecules or anti-viral proteins or extracellular matrix proteins (ECM) alone or in combination with ionizing radiation or any chemotherapy drug or any treatment designed to improve outcome of the cancer patients will improve cancer treatment. This is based on the functional associations between detected targets. DHX58 (also known as LGP2) is known as an apical suppressor of RNA dependent activation of the Type I interferons alpha and beta. IFITM1 and OASL are known anti-viral proteins. USP18 and HERC5 are enzymes involved in protein ISGylation/de-ISGylation, known to protect proteins from ubiquitin-dependent degradation in proteosome complex, while PSMB9 and PSMB10 are proteasome subunits. EPSTL1, LGALS3P and TAGLN are involved in the structure and functional regulation of ECM. CXCL9 and CCL2 are chemokines with multiple functions including growth-promoting functions for tumor cells.

Jak (Janus kinase) refers to a family of intracellular, nonreceptor tyrosine kinases and includes four family members, Janus 1(Jak-1), Janus 2 (Jak-2), Janus 3 (Jak-3), and Tyrosine kinase 2 (Tyk2).

Stat (Signal Transducer and Activator of Transcription) plays a role in regulating cell growth, survival and differentiation and the family includes Stat1, Stat2, Stat3, Stat4, Stat5 (Stat5a and Stat5b), and Stat6.

The term “subject” refers to any organism classified as a mammal, including mice, rats, guinea pigs, rabbits, dogs, cats, cows, horses, monkeys, and humans.

As used herein, the term “cancer” refers to a class of diseases of mammals characterized by uncontrolled cellular growth. The term “cancer” is used interchangeably with the terms “tumor,” “solid tumor,” “malignancy,” “hyperproliferation” and “neoplasm.” Cancer includes all types of hyperproliferative growth, hyperplasic growth, neoplastic growth, cancerous growth or oncogenic processes, metastatic tissues or malignantly transformed cells, tissues, or organs, irrespective of histopathologic type or stage of invasiveness. Illustrative examples include, lung, prostate, head and neck, breast and colorectal cancer, melanomas and gliomas (such as a high grade glioma, including glioblastoma multiforme (GBM), the most common and deadliest of malignant primary brain tumors in adult humans).

As used herein, the phrase “solid tumor” includes, for example, lung cancer, head and neck cancer, brain cancer, oral cancer, colorectal cancer, breast cancer, prostate cancer, pancreatic cancer, and liver cancer. Other types of solid tumors are named for the particular cells that form them, for example, sarcomas formed from connective tissue cells (for example, bone cartilage, fat), carcinomas formed from epithelial tissue cells (for example, breast, colon, pancreas) and lymphomas formed from lymphatic tissue cells (for example, lymph nodes, spleen, thymus). Treatment of all types of solid tumors regardless of naming convention is within the scope of this invention.

As used herein, the term “chemoresistant” refers to a tumor or cancer cell that shows little or no significant detectable therapeutic response to an agent used in chemotherapy.

As used herein, the term “radioresistant” refers to a tumor or cancer cell that shows little or no significant detectable therapeutic response to an agent used in radiotherapy such as ionizing radiation.

As used herein, the term “chemosensitive” refers to a tumor or cancer cell that shows a detectable therapeutic response to an agent used in chemotherapy.

As used herein, the term “radiosensitive” refers to a tumor or cancer cell that shows a detectable therapeutic response to an agent used in radiotherapy.

As used herein, the phrases “chemotherapeutic agent,” “cytotoxic agent,” “anticancer agent,” “antineoplastic agent” and “antitumor agent” are used interchangeably and refer to an agent that has the effect of inhibiting the growth or proliferation, or inducing the killing, of a tumor or cancer cell. The chemotherapeutic agent may inhibit or reverse the development or progression of a tumor or cancer, such as for example, a solid tumor.

As used herein, the term “chemotherapy” refers to administration of at least one chemotherapeutic agent to a subject having a tumor or cancer.

As used herein, the term “radiotherapy” refers to administration of at least one “radiotherapeutic agent” to a subject having a tumor or cancer and refers to any manner of treatment of a tumor or cancer with a radiotherapeutic agent. A radiotherapeutic agent includes, for example, ionizing radiation including, for example, external beam radiotherapy, stereotatic radiotherapy, virtual simulation, 3-dimensional conformal radiotherapy, intensity-modulated radiotherapy, ionizing particle therapy and radioisotope therapy.

Compositions herein may be formulated for oral, rectal, nasal, topical (including buccal and sublingual), transdermal, vaginal, injection/injectable, and/or parenteral (including subcutaneous, intramuscular, intravenous, intratumoral, and intradermal) administration. Other suitable administration routes are incorporated herein. The compositions may be presented conveniently in unit dosage forms and may be prepared by any methods known in the pharmaceutical arts. Examples of suitable drug formulations and/or forms are discussed in, for example, Hoover, John E. Remington's Pharmaceutical Sciences, Mack Publishing Co., Eston, Pa.; 18.sup.th edition (1995); and Liberman, H. A. and Lachman, L. Eds., Pharmaceutical Dosage Forms, Marcel Decker, New York, N.Y., 1980. Illustrative methods include the step of bringing one or more active ingredients into association with a carrier that constitutes one or more accessory ingredients. In general, the compositions may be prepared by bringing into association uniformly and intimately one or more active ingredients with liquid carriers or finely divided solid carriers or both, and then, if necessary, shaping the product.

Pharmaceutical formulations may include those suitable for oral, intramuscular, rectal, nasal, topical (including buccal and sub-lingual), vaginal or parenteral (including intramuscular, subcutaneous, intratumoral, and intravenous) administration or in a form suitable for administration by inhalation or insufflation. One or more of the compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

A salt may be a pharmaceutically suitable (i.e., pharmaceutically acceptable) salt including, but not limited to, acid addition salts formed by mixing a solution of the instant compound with a solution of a pharmaceutically acceptable acid. A pharmaceutically acceptable acid may be, for example, hydrochloric acid, methanesulphonic acid, fumaric acid, maleic acid, succinic acid, acetic acid, benzoic acid, oxalic acid, citric acid, tartaric acid, carbonic acid or phosphoric acid.

Suitable pharmaceutically-acceptable salts may further include, but are not limited to salts of pharmaceutically-acceptable inorganic acids, including, for example, sulfuric, phosphoric, nitric, carbonic, boric, sulfamic, and hydrobromic acids, or salts of pharmaceutically-acceptable organic acids such propionic, butyric, maleic, hydroxymaleic, lactic, mucic, gluconic, benzoic, succinic, phenylacetic, toluenesulfonic, benezenesulfonic, salicyclic sulfanilic, aspartic, glutamic, edetic, stearic, palmitic, oleic, lauric, pantothenic, tannic, ascorbic, and valeric acids.

Various pharmaceutically acceptable salts include, for example, the list of FDA-approved commercially marketed salts including acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, mitrate, pamoate, pantothenate, phosphate, diphosphate, polygalacturonate, salicylate, stearate, subacetate, succinate, sulfate, tannate, tartrate, teoclate, and triethiodide.

A hydrate may be a pharmaceutically suitable (i.e., pharmaceutically acceptable) hydrate that is a compound formed by the addition of water or its elements to a host molecule (for example, the free form version of the compound) including, but not limited to, monohydrates, dihydrates, etc. A solvate may be a pharmaceutically suitable (i.e., pharmaceutically acceptable) solvate, whereby solvation is an interaction of a solute with a solvent which leads to stabilization of the solute species in a solution, and whereby the solvated state is an ion in a solution complexed by solvent molecules. Solvates and hydrates may also be referred to as “analogues” or “analogs.”

A prodrug may be a compound that is pharmacologically inert but is converted by enzyme or chemical action to an active form of the drug (i.e., an active pharmaceutical ingredient) at or near the predetermined target site. In other words, prodrugs are inactive compounds or partially active compounds that yield an active compound upon metabolism in the body, which may or may not be enzymatically controlled. Prodrugs may also be broadly classified into two groups: bioprecursor and carrier prodrugs. Prodrugs may also be subclassified according to the nature of their action. Bioprecursor prodrugs are compounds that already contain the embryo of the active species within their structure, whereby the active species are produced upon metabolism.

Carrier prodrugs are formed by combining the active drug (e.g., active ingredient) with a carrier species forming a compound having desirable chemical and biological characteristics, whereby the link is an ester or amide so that the carrier prodrug is easily metabolized upon absorption or delivery to the target site. For example, lipophilic moieties may be incorporated to improve transport through membranes. Carrier prodrugs linked by a functional group to carrier are referred to as bipartite prodrugs. Prodrugs where the carrier is linked to the drug by a separate structure are referred to as tripartite prodrugs, whereby the carrier is removed by an enzyme-controlled metabolic process, and whereby the linking structure is removed by an enzyme system or by a chemical reaction. A hydroxy-protecting group includes, for example, a tert-butyloxy-carbonyl (t-BOC) and t-butyl-dimethyl-silyl (TBS). Other hydroxy protecting groups contemplated are known in the art.

In another embodiment, a dosage form and/or composition may include one or more active metabolites of the active ingredients in place of or in addition to the active ingredients disclosed herein.

Dosage form compositions containing the active ingredients may also contain one or more inactive pharmaceutical ingredients such as diluents, solubilizers, alcohols, binders, controlled release polymers, enteric polymers, disintegrants, excipients, colorants, flavorants, sweeteners, antioxidants, preservatives, pigments, additives, fillers, suspension agents, surfactants (for example, anionic, cationic, amphoteric and nonionic), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

As used herein, an oral dosage form may include capsules (a solid oral dosage form consisting of a shell and a filling, whereby the shell is composed of a single sealed enclosure, or two halves that fit together and which are sometimes sealed with a band and whereby capsule shells may be made from gelatin, starch, or cellulose, or other suitable materials, may be soft or hard, and are filled with solid or liquid ingredients that can be poured or squeezed), capsule or coated pellets (solid dosage form in which the drug is enclosed within either a hard or soft soluble container or “shell” made from a suitable form of gelatin; the drug itself is in the form of granules to which varying amounts of coating have been applied), capsule coated extended release (a solid dosage form in which the drug is enclosed within either a hard or soft soluble container or “shell” made from a suitable form of gelatin; additionally, the capsule is covered in a designated coating, and which releases a drug or drugs in such a manner to allow at least a reduction in dosing frequency as compared to that drug or drugs presented as a conventional dosage form), capsule delayed release (a solid dosage form in which the drug is enclosed within either a hard or soft soluble container made from a suitable form of gelatin, and which releases a drug (or drugs) at a time other than promptly after administration, whereby enteric-coated articles are delayed release dosage forms), capsule delayed release pellets (solid dosage form in which the drug is enclosed within either a hard or soft soluble container or “shell” made from a suitable form of gelatin); the drug itself is in the form of granules to which enteric coating has been applied, thus delaying release of the drug until its passage into the intestines), capsule extended release (a solid dosage form in which the drug is enclosed within either a hard or soft soluble container made from a suitable form of gelatin, and which releases a drug or drugs in such a manner to allow a reduction in dosing frequency as compared to that drug or drugs presented as a conventional dosage form), capsule film-coated extended release (a solid dosage form in which the drug is enclosed within either a hard or soft soluble container or “shell” made from a suitable form of gelatin; additionally, the capsule is covered in a designated film coating, and which releases a drug or drugs in such a manner to allow at least a reduction in dosing frequency as compared to that drug or drugs presented as a conventional dosage form), capsule gelatin coated (a solid dosage form in which the drug is enclosed within either a hard or soft soluble container made from a suitable form of gelatin; through a banding process, the capsule is coated with additional layers of gelatin so as to form a complete seal), and capsule liquid filled (a solid dosage form in which the drug is enclosed within a soluble, gelatin shell which is plasticized by the addition of a polyol, such as sorbitol or glycerin, and is therefore of a somewhat thicker consistency than that of a hard shell capsule; typically, the active ingredients are dissolved or suspended in a liquid vehicle).

Oral dosage forms contemplated herein also include granules (a small particle or grain), pellet (a small sterile solid mass consisting of a highly purified drug, with or without excipients, made by the formation of granules, or by compression and molding), pellets coated extended release (a solid dosage form in which the drug itself is in the form of granules to which varying amounts of coating have been applied, and which releases a drug or drugs in such a manner to allow a reduction in dosing frequency as compared to that drug or drugs presented as a conventional dosage form), pill (a small, round solid dosage form containing a medicinal agent intended for oral administration), powder (an intimate mixture of dry, finely divided drugs and/or chemicals that may be intended for internal or external use), elixir (a clear, pleasantly flavored, sweetened hydroalcoholic liquid containing dissolved medicinal agents; it is intended for oral use), chewing gum (a sweetened and flavored insoluble plastic material of various shapes which when chewed, releases a drug substance into the oral cavity), or syrup (an oral solution containing high concentrations of sucrose or other sugars; the term has also been used to include any other liquid dosage form prepared in a sweet and viscid vehicle, including oral suspensions).

Oral dosage forms contemplated herein may further include a tablet (a solid dosage form containing medicinal substances with or without suitable diluents), tablet chewable (a solid dosage form containing medicinal substances with or without suitable diluents that is intended to be chewed, producing a pleasant tasting residue in the oral cavity that is easily swallowed and does not leave a bitter or unpleasant after-taste), tablet coated (a solid dosage form that contains medicinal substances with or without suitable diluents and is covered with a designated coating), tablet coated particles (a solid dosage form containing a conglomerate of medicinal particles that have each been covered with a coating), tablet delayed release (a solid dosage form which releases a drug or drugs at a time other than promptly after administration, whereby enteric-coated articles are delayed release dosage forms), tablet delayed release particles (a solid dosage form containing a conglomerate of medicinal particles that have been covered with a coating which releases a drug or drugs at a time other than promptly after administration, whereby enteric-coated articles are delayed release dosage forms), tablet dispersible (a tablet that, prior to administration, is intended to be placed in liquid, where its contents will be distributed evenly throughout that liquid, whereby term ‘tablet, dispersible’ is no longer used for approved drug products, and it has been replaced by the term ‘tablet, for suspension’), tablet effervescent (a solid dosage form containing mixtures of acids, for example, citric acid, tartaric acid, and sodium bicarbonate, which release carbon dioxide when dissolved in water, whereby it is intended to be dissolved or dispersed in water before administration), tablet extended release (a solid dosage form containing a drug which allows at least a reduction in dosing frequency as compared to that drug presented in conventional dosage form), tablet film coated (a solid dosage form that contains medicinal substances with or without suitable diluents and is coated with a thin layer of a water-insoluble or water-soluble polymer), tablet film coated extended release (a solid dosage form that contains medicinal substances with or without suitable diluents and is coated with a thin layer of a water-insoluble or water-soluble polymer; the tablet is formulated in such manner as to make the contained medicament available over an extended period of time following ingestion), tablet for solution (a tablet that forms a solution when placed in a liquid), tablet for suspension (a tablet that forms a suspension when placed in a liquid, which is formerly referred to as a ‘dispersible tablet’), tablet multilayer (a solid dosage form containing medicinal substances that have been compressed to form a multiple-layered tablet or a tablet-within-a-tablet, the inner tablet being the core and the outer portion being the shell), tablet multilayer extended release (a solid dosage form containing medicinal substances that have been compressed to form a multiple-layered tablet or a tablet-within-a-tablet, the inner tablet being the core and the outer portion being the shell, which, additionally, is covered in a designated coating; the tablet is formulated in such manner as to allow at least a reduction in dosing frequency as compared to that drug presented as a conventional dosage form), tablet orally disintegrating (a solid dosage form containing medicinal substances which disintegrates rapidly, usually within a matter of seconds, when placed upon the tongue), tablet orally disintegrating delayed release (a solid dosage form containing medicinal substances which disintegrates rapidly, usually within a matter of seconds, when placed upon the tongue, but which releases a drug or drugs at a time other than promptly after administration), tablet soluble (a solid dosage form that contains medicinal substances with or without suitable diluents and possesses the ability to dissolve in fluids), tablet sugar coated (a solid dosage form that contains medicinal substances with or without suitable diluents and is coated with a colored or an uncolored water-soluble sugar), and the like.

Injection and infusion dosage forms (i.e., parenteral dosage forms) include, but are not limited to, the following. Liposomal injection includes or forms liposomes or a lipid bilayer vesicle having phospholipids that encapsulate an active drug substance. Injection includes a sterile preparation intended for parenteral use. Five distinct classes of injections exist as defined by the USP. Emulsion injection includes an emulsion comprising a sterile, pyrogen-free preparation intended to be administered parenterally. Lipid complex and powder for solution injection are sterile preparations intended for reconstitution to form a solution for parenteral use.

Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization.

Solution injection involves a liquid preparation containing one or more drug substances dissolved in a suitable solvent or mixture of mutually miscible solvents that is suitable for injection. Solution concentrate injection involves a sterile preparation for parenteral use that, upon addition of suitable solvents, yields a solution suitable for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of microspheres by the solid particles.

A parenteral carrier system may include one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives.

Inhalation dosage forms include, but are not limited to, aerosol being a product that is packaged under pressure and contains therapeutically active ingredients that are released upon activation of an appropriate valve system intended for topical application to the skin as well as local application into the nose (nasal aerosols), mouth (lingual and sublingual aerosols), or lungs (inhalation aerosols). Inhalation dosage forms further include foam aerosol being a dosage form containing one or more active ingredients, surfactants, aqueous or nonaqueous liquids, and the propellants, whereby if the propellant is in the internal (discontinuous) phase (i.e., of the oil-in-water type), a stable foam is discharged, and if the propellant is in the external (continuous) phase (i.e., of the water-in-oil type), a spray or a quick-breaking foam is discharged. Inhalation dosage forms also include metered aerosol being a pressurized dosage form consisting of metered dose valves which allow for the delivery of a uniform quantity of spray upon each activation; powder aerosol being a product that is packaged under pressure and contains therapeutically active ingredients, in the form of a powder, that are released upon activation of an appropriate valve system; and aerosol spray being an aerosol product which utilizes a compressed gas as the propellant to provide the force necessary to expel the product as a wet spray and being applicable to solutions of medicinal agents in aqueous solvents.

Pharmaceutically suitable inhalation carrier systems may include pharmaceutically suitable inactive ingredients known in the art for use in various inhalation dosage forms, such as (but not limited to) aerosol propellants (for example, hydrofluoroalkane propellants), surfactants, additives, suspension agents, solvents, stabilizers and the like.

As used herein, the term “delivery-enhancing agents” refers to any agents which enhance the release or solubility (e.g., from a formulation delivery vehicle), diffusion rate, penetration capacity and timing, uptake, residence time, stability, effective half-life, peak or sustained concentration levels, clearance and other desired intranasal delivery characteristics (e.g., as measured at the site of delivery, or at a selected target site of activity such as the bloodstream or central nervous system) of a snRNA or its functionally equivalent fragment or other biologically active compound(s).

A transdermal dosage form may include, but is not limited to, a patch being a drug delivery system that often contains an adhesive backing that is usually applied to an external site on the body, whereby the ingredients either passively diffuse from, or are actively transported from some portion of the patch, and whereby depending upon the patch, the ingredients are either delivered to the outer surface of the body or into the body; and other various types of transdermal patches such as matrix, reservoir and others known in the art. The “pharmaceutically suitable transdermal carrier system” includes pharmaceutically suitable inactive ingredients known in the art for use in various transdermal dosage forms, such as (but not limited to) solvents, adhesives, diluents, additives, permeation enhancing agents, surfactants, emulsifiers, liposomes, and the like.

Commonly used techniques for the introduction of the nucleic acid molecules into cells (for example, the cytosol of a dendritic cell), tissues, and organisms that can also be used in the present disclosure include the use of various carrier systems, reagents and vectors, including, for example, pharmaceutically-acceptable carriers such as nanocarriers, conjugates, nucleic-acid-lipid particles, vesicles, exosomes, protein capsids, liposomes, dendrimers, lipoplexes, micelles, virosomes, virus like particles, nucleic acid complexes, and mixtures thereof. Nanocarriers generally range in the size from about 1 nm to about 100 nm or about 200 nm in diameter, and can be made from, for example, micelles, polymers, carbon-based materials, liposomes, and other substances known to those skilled in the art.

The dosing of an agent of the present disclosure to a human subject may be determined by those skilled in the art based upon known methods such as animal studies and clinical trials involving human subjects. For example, Budman D R, Calvert, A H, and Rowinsky E K, Handbook of Anticancer Drug Development, describes dose-escalation studies to find the maximum tolerable dosage (MTD) along with dose-limiting toxicity (DLT). Generally, the starting dose can be derived by allometric scaling from dosing studies in mice. The lethal dose (LD₁₀) is also determined in mice. Following mice studies, 1/10 of the mouse LD₁₀ is administered to a cohort of healthy subjects. Escalating dose administers a dose 100%, 67%, 50%, 40%, and 33% thereafter of the previously described dose ( 1/10 mouse LD₁₀) (in other words, the second dose level is 100% greater than the first, the third is 67% greater than the second and so forth) to determine the pharmacokinetics of the agent in the subjects, which is then used to determine proper dosing regimens, including dosage amounts, routes of administration, timing of administration, etc. This is followed by more dosing studies in diseased subjects to determine a therapeutically effective dosage parameters in treating the disease in a broader population of subjects. Suitable dosage amounts and dosing regimens may also be in consideration of a variety of factors, including one or more particular conditions being treated, the severity of the one or more conditions, the genetic profile, age, health, sex, diet, and weight of the subject, the route of administration alone or in combination with pharmacological considerations including the activity, efficacy, bioavailability, pharmacokinetic, and toxicological profiles of the particular compound employed, whether a drug delivery system is utilized and/or whether the drug is administered as part of a drug combination. Therefore, the dosage regimen to be employed may vary widely and may necessarily deviate from the dosage regimens set forth herein.

In regard to an expression inhibitor of the present disclosure, it is contemplated that dosage forms may include an amount of one or more expression inhibitors (or inhibitors of expression) ranging from about 1 to about 1400 mg, or about 5 to about 100 mg, or about 25 to about 800 mg, or about 100 to about 500 mg, or 0.1 to 50 milligrams (±10%), or about 10 to about 100 milligrams (±10%), or about 5 to about 500 milligrams (±10%), or about 0.1 to about 200 milligrams (±10%), or about 1 to about 100 milligrams (±10%), or about 5 to about 50 milligrams (±10%), or about 30 milligrams (±10%), or about 20 milligrams (±10%), or about 10 milligrams (±10%), or about 5 milligrams (±10%), per dosage form, such as, for example, a tablet, a pill, a bolus, and the like.

A dosage form of the present disclosure may be administered to a subject in need thereof, for example, once per day, twice per day, once every 6 hours, once every 4 hours, once every 2 hours, hourly, twice an hour, twice a day, twice a week, or monthly.

The phrase “therapeutically effective” is intended to qualify the amount that will achieve the goal of improvement in disease severity and/or the frequency of incidence over non-treatment, while limiting, reducing, or avoiding adverse side effects typically associated with disease therapies. A “therapeutic effect” relieves to some extent one or more of the symptoms of a cancer disease or disorder. In reference to the treatment of a cancer, a therapeutic effect refers to one or more of the following: 1) reduction in the number of cancer cells by, for example, killing the cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disease or disorder; and/or 7) relieving or reducing the side effects associated with the administration of an anticancer agent. “Therapeutic effective amount” is intended to qualify the amount required to achieve a therapeutic effect. For example, a therapeutically effective amount of an expression inhibitor (or inhibitors of expression) may be any amount that begins to improve cancer treatment in a subject. In one embodiment, an effective amount of an expression inhibitor used in the therapeutic regime described herein may be, for example, about 1 mg, or about 5 mg, or about 10 mg, or about 25 mg, or about 50 mg, or about 100 mg, or about 200 mg, or about 400 mg, or about 500 mg, or about 600 mg, or about 1000 mg, or about 1200 mg, or about 1400 mg, or from about 10 to about 60 mg, or about 50 mg to about 200 mg, or about 150 mg to about 600 mg per day. Further, another effective amount of an expression inhibitor used herein may be that which results in a detectable blood level of above about 1 ng/dL, 5, ng/dL, 10 ng/dL, 20, ng/dL, 35 ng/dL, or about 70 ng/dL, or about 140 ng/dL, or about 280 ng/dL, or about 350 ng/dL, or lower or higher.

The term “pharmaceutically acceptable” is used herein to mean that the modified ion is appropriate for use in a pharmaceutical product. Pharmaceutically acceptable cations include metallic ions and organic ions. Other metallic ions include, but are not limited to appropriate alkali metal salts, alkaline earth metal salts and other physiological acceptable metal ions. Exemplary ions include aluminium, calcium, lithium, magnesium, potassium, sodium and zinc in their usual valences. Organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

It is further contemplated that one active ingredient may be in an extended release form, while an optional second, third, or fourth other active ingredient, for example, may or may not be, so the recipient experiences, for example, a spike in the second, third, or fourth active ingredient that dissipates rapidly, while the first active ingredient is maintained in a higher concentration in the blood stream over a longer period of time. Similarly, one of the active ingredients may be an active metabolite, while another may be in an unmetabolized state, such that the active metabolite has an immediate effect upon administration to a subject whereas the unmetabolized active ingredient administered in a single dosage form may need to be metabolized before taking effect in the subject.

Also contemplated are solid form preparations that include at least one active ingredient which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like. Solutions or suspensions may be applied topically and/or directly to the nasal cavity, respiratory tract, eye, or ear by conventional means, for example with a dropper, pipette or spray.

Alternatively, one or more of the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier may form a gel in the nasal cavity. The powder composition may be presented in unit dose form, for example, in capsules or cartridges of, for example, gelatin, or blister packs from which the powder may be administered by means of an inhaler.

The pharmaceutical preparations may be in unit dosage forms. In such form, the preparation may be subdivided into unit doses containing appropriate quantities of the active component. The unit dosage form can be a packaged preparation, such as a kit or other form, the package containing discrete quantities of preparation, such as packeted tablets, capsules, liquids or powders in vials or ampoules. Also, the unit dosage form can be a capsule, tablet, cachet, or lozenge, or it can be the appropriate number of any of these in packaged form.

The present disclosure is further illustrated by the following examples, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

### EXAMPLES

### Example 1. The RIG-I Like Receptor LGP2 Protects Tumor Cells from Ionizing Radiation

Methods

Gene Selection

We compiled 14 gene expression datasets containing interferon-stimulated genes in cancer cells as shown below in Table No. 1.

TABLE NO. 1
Fourteen Gene Expression Datasets

| PMID | Citation |
|---|---|
| 14755057 | Khodarev N N, et al. STAT1 is overexpressed in tumors selected for |
| | radioresistance and confers protection from radiation in transduced sensitive |
| | cells. Proc Natl Acad Sci USA (2004) 101(6): 1714-1719 |
| 15657362 | Becker M, et al. Distinct gene expression patterns in a tamoxifen-sensitive human |
| | mammary carcinoma xenograft and its tamoxifen-resistant subline MaCa |
| | 3366/TAM. Mol Cancer Ther (2005) January; 4(1): 151-68 |
| 16075456 | Pedersen M W, et al. Analysis of the epidermal growth factor receptor specific |
| | transcriptome: effect of receptor expression level and an activating mutation. J |
| | Cell Biochem 2005 Oct. 1; 96(2): 412-27 |
| 16652143 | Patterson S G, et al. Novel role of Stat1 in the development of docetaxel |
| | resistance in prostate tumor cells. Oncogene 2006 Oct. 5; 25(45): 6113-22 |
| 17072862 | Fryknas M, et al. STAT1 signaling is associated with acquired crossresistance to |
| | doxorubicin and radiation in myeloma cell lines. Int J Cancer 2007 Jan. 1; |
| | 120(1): 189-95 |
| 17440099 | Tsai M H, et al. Gene expression profiling of breast, prostate, and glioma cells |
| | following single versus fractionated doses of radiation. Cancer Res 2007 Apr. |
| | 15; 67(8): 3845-52 |
| 17868458 | Buess M, et al. Characterization of heterotypic interaction effects in vitro to |
| | deconvolute global gene expression profiles in cancer. Genome Biol |
| | 2007; 8(9): R191 |
| 20197756 | Meng Y, et al. Ad. Egr-TNF and local ionizing radiation suppress metastases by |
| | interferon-beta-dependent activation of antigen-specific CD8+ T cells. Mol Ther |
| | 2010 May; 18(5): 912-20 |
| 20682643 | Luszczek W, et al. Combinations of DNA methyltransferase and histone |
| | deacetylase inhibitors induce DNA damage in small cell lung cancer cells: |
| | correlation of resistance with IFN-stimulated gene expression. Mol Cancer Ther |
| | 2010 August; 9(8): 2309-21 |
| 20875954 | Dobbin E, et al. Proteomic analysis reveals a novel mechanism induced by the |
| | leukemic oncogene Tel/PDGFRβ in stem cells: activation of the interferon |
| | response pathways. Stem Cell Res 2010 November; 5(3): 226-43 |
| 21074499 | Chen E, et al. Distinct clinical phenotypes associated with JAK2V617F reflect |
| | differential STAT1 signaling. Cancer Cell 2010 Nov. 16; 18(5): 524-35 |
| 21185374 | Englert N A, et al. Persistent and non-persistent changes in gene expression result |
| | from long-term estrogen exposure of MCF-7 breast cancer cells. J Steroid |
| | Biochem Mol Biol 2011 February; 123(3-5): 140-50 |
| 23056240 | Pitroda S P, et al. Tumor endothelial inflammation predicts clinical outcome in |
| | diverse human cancers. PLoS One 2012; 7(10): e46104 |
| NA | Khodarev N N, et al. (unpublished) |

Probe set IDs for each dataset were annotated using Ingenuity Pathway Analysis (IPA-http://www.ingenuity.com/). Genes were included in the final screening set if they were in the IRDS or if they were reported in ≧2 other studies. After initial inclusion, all selected genes were screened in the Interferome database (http://www.interferome.org/) to select genes activated by IFNs. In total, 89 candidate ISGs (Interferon Stimulated Genes) downstream from IFN/Stat were identified below in Table No. 2.

TABLE NO. 2
Identified Candidate ISGs

| | | Entrez |
|---|---|---|
| Gene Symbol | Gene Name | Gene ID |
| ABCC3 | ATP-binding cassette, sub-family C (CFTR/MRP), member 3 | 8714 |
| B2M | beta-2-microglobulin | 567 |
| BST2 | bone marrow stromal cell antigen 2 | 684 |
| CCL2 | chemokine (C-C motif) ligand 2 | 6347 |
| CCL5 | chemokine (C-C motif) ligand 5 | 6352 |
| CCNA1 | cyclin A1 | 8900 |
| CD74 | CD74 molecule, major histocompatibility complex, class II | 972 |
| | invariant chain | |
| CMPK2 | cytidine monophosphate (UMP-CMP) kinase 2, mitochondrial | 129607 |
| CTSS | cathepsin S | 1520 |
| CXCL1 | chemokine (C-X-C motif) ligand 1 (melanoma growth | 2919 |
| | stimulating activity, alpha) | |
| CXCL10 | chemokine (C-X-C motif) ligand 10 | 3627 |
| CXCL3 | chemokine (C-X-C motif) ligand 3 | 2921 |
| CXCL9 | chemokine (C-X-C motif) ligand 9 | 4283 |
| DAZ1 | deleted in azoospermia 1 | 1617 |
| DDX58 | DEAD (Asp-Glu-Ala-Asp) box polypeptide 58 | 23586 |
| DDX60 | DEAD (Asp-Glu-Ala-Asp) box polypeptide 60 | 55601 |
| DDX60L | DEAD (Asp-Glu-Ala-Asp) box polypeptide 60-like | 91351 |
| DHX58 | DEXH (Asp-Glu-X-His) box polypeptide 58 | 79132 |
| (LGP2) | | |
| DTX3L | deltex 3-like (Drosophila) | 151636 |
| EIF2AK2 | eukaryotic translation initiation factor 2-alpha kinase 2 | 5610 |
| EPSTI1 | epithelial stromal interaction 1 (breast) | 94240 |
| GBP1 | guanylate binding protein 1, interferon-inducible, 67 kDa | 2633 |
| GBP2 | guanylate binding protein 2, interferon-inducible | 2634 |
| HERC5 | hect domain and RLD 5 | 51191 |
| HERC6 | hect domain and RLD 6 | 55008 |
| HNMT | histamine N-methyltransferase | 3176 |
| IFI16 | interferon, gamma-inducible protein 16 | 3428 |
| IFI27 | interferon, alpha-inducible protein 27 | 3429 |
| IFI35 | interferon-induced protein 35 | 3430 |
| IFI44 | interferon-induced protein 44 | 10561 |
| IFI44L | interferon-induced protein 44-like | 10964 |
| IFI6 | interferon, alpha-inducible protein 6 | 2537 |
| IFIH1 | interferon induced with helicase C domain 1 | 64135 |
| IFIT1 | interferon-induced protein with tetratricopeptide repeats 1 | 3434 |
| IFIT2 | interferon-induced protein with tetratricopeptide repeats 2 | 3433 |
| IFIT3 | interferon-induced protein with tetratricopeptide repeats 3 | 3437 |
| IFITM1 | interferon induced transmembrane protein 1 (9-27) | 8519 |
| IFITM2 | interferon induced transmembrane protein 2 (1-8D) | 10581 |
| IFITM3 | interferon induced transmembrane protein 3 (1-8U) | 10410 |
| IGFBP3 | insulin-like growth factor binding protein 3 | 3486 |
| IL7R | interleukin 7 receptor | 3575 |
| IRF1 | interferon regulatory factor 1 | 3659 |
| IRF7 | interferon regulatory factor 7 | 3665 |
| IRF9 | interferon regulatory factor 9 | 10379 |
| ISG15 | ISG15 ubiquitin-like modifier | 9636 |
| LAMP3 | lysosomal-associated membrane protein 3 | 27074 |
| LGALS3BP | lectin, galactoside-binding, soluble, 3 binding protein | 3959 |
| LY6E | lymphocyte antigen 6 complex, locus E | 4061 |
| LY96 | lymphocyte antigen 96 | 23643 |
| MARCKS | myristoylated alanine-rich protein kinase C substrate | 4082 |
| MCL1 | myeloid cell leukemia sequence 1 (BCL2-related) | 4170 |
| MGP | matrix Gla protein | 4256 |
| MX1 | myxovirus (influenza virus) resistance 1, interferon-inducible | 4599 |
| | protein p78 (mouse) | |
| MX2 | myxovirus (influenza virus) resistance 2 (mouse) | 4600 |
| NLRC5 | NLR family, CARD domain containing 5 | 84166 |
| NMI | N-myc (and STAT) interactor | 9111 |
| OAS1 | 2′,5′-oligoadenylate synthetase 1, 40/46 kDa | 4938 |
| OAS2 | 2′,5′-oligoadenylate synthetase 2, 69/71 kDa | 4939 |
| OAS3 | 2′,5′-oligoadenylate synthetase 3, 100 kDa | 4940 |
| OASL | 2′,5′-oligoadenylate synthetase-like | 8638 |
| PARP12 | poly (ADP-ribose) polymerase family, member 12 | 64761 |
| PLSCR1 | phospholipid scramblase 1 | 5359 |
| PRIC285 | peroxisomal proliferator-activated receptor A interacting | 85441 |
| | complex 285 | |
| PSMB10 | proteasome (prosome, macropain) subunit, beta type, 10 | 5699 |
| PSMB8 | proteasome (prosome, macropain) subunit, beta type, 8 (large | 5696 |
| | multifunctional peptidase 7) | |
| PSMB9 | proteasome (prosome, macropain) subunit, beta type, 9 (large | 5698 |
| | multifunctional peptidase 2) | |
| RNF213 | ring finger protein 213 | 57674 |
| RSAD2 | radical S-adenosyl methionine domain containing 2 | 91543 |
| RTP4 | receptor (chemosensory) transporter protein 4 | 64108 |
| SAMD9 | sterile alpha motif domain containing 9 | 54809 |
| SAMD9L | sterile alpha motif domain containing 9-like | 219285 |
| SAMHD1 | SAM domain and HD domain 1 | 25939 |
| SP110 | SP110 nuclear body protein | 3431 |
| SRGN | serglycin | 5552 |
| STAT1 | signal transducer and activator of transcription 1, 91 kDa | 6772 |
| TAGLN | transgelin | 6876 |
| TAP1 | transporter 1, ATP-binding cassette, sub-family B (MDR/TAP) | 6890 |
| THBS1 | thrombospondin 1 | 7057 |
| TIMP3 | TIMP metallopeptidase inhibitor 3 | 7078 |
| TNFSF10 | tumor necrosis factor (ligand) superfamily, member 10 | 8743 |
| TPD52L1 | tumor protein D52-like 1 | 7164 |
| TRIM14 | tripartite motif-containing 14 | 9830 |
| TRIM21 | tripartite motif-containing 21 | 6737 |
| UBA7 | ubiquitin-like modifier activating enzyme 7 | 7318 |
| UBE2L6 | ubiquitin-conjugating enzyme E2L 6 | 9246 |
| USP18 | ubiquitin specific peptidase 18 | 11274 |
| VAMP5 | vesicle-associated membrane protein 5 (myobrevin) | 10791 |
| WARS | tryptophanyl-tRNA synthetase | 7453 |
| XAF1 | XIAP associated factor 1 | 54739 |

siRNA Screen

siRNA screening of the selected ISGs was performed as follows. On day 1, Lipofectamine RNAiMAX diluted in Opti-MEM (Life Technologies) was added to 0.075 μL/well using a Tecan Freedom EVO 200 robotic liquid handling station to the previously prepared 384-well microplates (Corning/3712) containing immobilized individual siRNAs (Dharmacon siGENOME) plated in triplicate for each target ISG. Cells were added using a Thermo Electron MultiDrop Combi dispenser at 500 cells/well in 50 μL of RPMI 1640 media supplemented withl0% FCS. The final siRNA concentration in each well was 50 nM. Plates were incubated overnight at 37° C., and on day 2 were treated with IR at a dose of 3 Gy or untreated. Plates were further incubated at 37° C. and then assayed for viability at 48 hours post-IR using the highly sensitive luciferase-based CellTiterGlo® assay (Promega, Madison, Wis.). Luminescent reagent was added using a Thermo Electron MultiDrop Combi, and luminescent measurements were taken 90 minutes later using Molecular Devices Analyst GT. This platform was provided by the Cellular Screening Core (CSC), Institute for Genomics & Systems Biology, University of Chicago.

Individual siRNAs against LGP2 were validated in HCT116 and MCF10A cell lines by viability assay. Viability was assayed at 120 hours post-transfection (72 hours post-IR) using the CellTiter-Glo® Luminescent Cell Viability Assay (Promega, Madison, Wis.). This experiment was repeated to confirm reproducibility of the data. The top two siRNA's were selected for subsequent qRT-PCR experiments to confirm suppression of LGP2 mRNA on the basal level and after IFNβ treatment. Based on these data, two individual siRNA were selected and used in all subsequent experiments: #3: (SEQ ID NO:1, 5′-CCAGUACCUAGAACUUAA-3′) and #4 (SEQ ID NO:2, 5′-AGAAUGAGCUGGCCCACUU-3′)

Cell Cultures

B6 Wt and B6/IFNAR1^{-/-} mice were generously provided by Yang-Xin Fu at the University of Chicago and used in accordance with the animal experimental guidelines set by the Institute of Animal Care and Use Committee. Primary murine embryonic fibroblasts (MEFs) were obtained from 13.5d postcoitus embryos and cultivated in DMEM supplemented with 10% FBS, non-essential amino acids and penicillin/streptomycin for no more than 7 passages as previously described. MEFs were immortalized with a retrovirus expressing SV40-large T antigen (Addgene plasmid 13970. Tumor cell lines used for siRNA screen and subsequent experiments were: Scc61 and Nu61 (head and neck squamous cell carcinoma); D54, T98G and U251 (glioblastoma multiforme); WiDr and HCT116 (colorectal carcinoma); MDA-MB-231 and MCF7 (breast adenocarcinoma); MCF10a (immortalized human mammary epithelial cells); DU154 (prostate cancer); A549 and NCI-H460 (lung adenocarcinoma); and T24 (bladder cancer). Cell lines were cultivated as follows: Scc61 and Nu61 in DMEM/F12 with 20% FBS, 1% P/S, and 1% HC; D54, T98G and WiDr in MEM with 10% FBS and 1% P/S; U251, HCT116, MDA-MB-231, MCF7, in DMEM high glucose with 10% FBS and 1% P/S; MCF10A MEBM with MEGM kit (ATCC), cholera toxin (100 ng/mL), and 1% P/S; DU145 in DMEM F12 with 10% FBS and 1% P/S; A549 and NCI-H460 in RPMI with 10% FBS and 1% P/S; T24 in McCoy's 5A Medium with 10% FBS and 1% P/S.

Retro- and Lentiviral Production and Transduction

Retrovirus was produced using complete packaging ecotropic Plat-E cells (Cell Biolabs) by FUGENE mediated transfection of pBABE-puro SV40 LT (Zhao J J, et al. (2003) Human mammary epithelial cell transformation through the activation of phosphatidylinositol 3-kinase. Cancer cell 3(5):483-495). Lentivirus was produced by co-transfection of VSVG, VPR and pLKO.1 lentiviral vector with inserted LGP2 shRNA sequence (SEQ ID NO:3, ATTCTTGCGGTCATCGAACAG, Thermo Scientific) or non-targeting control (Thermo Scientific) into HEK293X cells. Supernatants containing infectious viral particles were harvested 48 h post-transfection and passed through a 0.45 μm filter. Infections of exponentially growing cells were performed with virus-containing supernatant supplemented with 8 μg/mL polybrene. In lentiviral shRNA experiments, transduced cells were continually selected in the presence of puromycin (1-2 μg/ml).

Western Blotting

Western blotting was performed as described previously (Khodarev N N, et al. (2007) Signal transducer and activator of transcription 1 regulates both cytotoxic and prosurvival functions in tumor cells. Cancer Res 67(19):9214-9220). The following antibodies were utilized: anti-LGP2 (sc134667; Santa Cruz) (1:1,000) and anti-Actin-HRP (Sc47778, Santa Cruz) (1:5000). Secondary antibodies conjugated to horseradish peroxidase (HRP) (Santa Cruz) were used at 1:10,000. Experimental findings were confirmed in at least three independent experiments.

qRT-PCR

Total RNA was extracted using TRIzol reagent (Invitrogen), treated with DNase I (Invitrogen) and reverse transcribed using SuperScript III (Invitrogen), and the cDNA products were resuspended in 20 μl of H₂O and used for PCR with Fast SYBR green master mix and a StepOnePlus real-time PCR system (both from Applied Biosystems). The following human gene-specific primers were used: IFNβ sense primer 5′-AACTTTGACATCCCTGAGGAGATT-3′(SEQ ID NO:4) and antisense primer 5′-GCGGCGTCCTCCTTCTG-3′(SEQ ID NO:5); GAPDH sense 5′-CTCTGCTCCTCCTGTTCGAC-3′(SEQ ID NO:6) and antisense 5′-GTTAAAAGCAGCCCTGGTGA-3′ (SEQ ID NO:7). All samples were amplified in duplicate and every experiment was repeated independently at least two times. Relative gene expression was determined using the 2^{-ΔΔCT} method, with GAPDH as the internal control.

Luciferase Assay

To measure IFNβ promoter activity, HEK293 cells were transiently co-transfected using Fugene (Roche) with pGL3-Ifnβ-Luc (Lin R, Genin P, Mamane Y, & Hiscott J (2000) Selective DNA binding and association with the CREB binding protein coactivator contribute to differential activation of alpha/beta interferon genes by interferon regulatory factors 3 and 7. Molecular and cellular biology 20(17):6342-6353) and an expression plasmid carrying the Renilla luciferase gene driven by the SV40 promoter (Promega). In some experiments, co-transfection mixes also included p3×FLAG-CMV10-LGP2 (Bamming D & Horvath C M (2009) Regulation of signal transduction by enzymatically inactive antiviral RNA helicase proteins MDA5, RIG-I, and LGP2. J Biol Chem 284(15):9700-9712) expression plasmid (or p3×FLAG-CMV10control). The following day, cells were irradiated at indicated dose and collected at indicated time in passive lysis buffer (Promega). Firefly and Renilla luciferase activities were measured using a dual-luciferase assay system (Promega). For siRNAs experiments, siRNA against LGP2 (see above) or non-targeting (Dharmacon,) were transfected with RNAimax 24 h prior to transfection of luciferase/Renilla plasmids. Mean luciferase values were normalized and quantified from duplicate runs for each of at least three separate experiments.

Viability Assay

To determine cell viability, cells were plated in triplicate in 96-well plates at a density of 3,000 cells per well and treated with increasing amounts of ionizing radiation. At the indicated time, cells were stained using 0.4% methylene blue in 50% methanol (Leonova K I, et al. (2013) p53 cooperates with DNA methylation and a suicidal interferon response to maintain epigenetic silencing of repeats and noncoding RNAs. Proc Natl Acad Sci USA 110(1):E89-98). Dye was extracted from stained cells using 3% HCl solution for spectrophotometric quantitation at 660 nm. In some experiments, neutralizing antibodies to IFNβ (PBL Interferon Source, 1 μg/mL) or isotype control IgG₁ (RD Systems) were incubated with cells 1 h prior irradiation.

Clonogenic Assay

Cells were seeded to form colonies in p60 plates and treated the next day with 1, 3, 5, or 7Gy IR. When sufficiently large colonies with at least 50 cells were visible (approximately 12-15 days), the plates were fixed with methanol and stained with crystal violet as previously described. Colonies with more than 50 cells were counted and the surviving fraction was calculated (Mauceri H J, et al. (1998) Combined effects of angiostatin and ionizing radiation in antitumour therapy. Nature 394(6690):287-291). For siRNAs experiments, the indicated siRNA was transfected 24 h prior to plating for the clonogenic assay. In overexpression experiments, D54 cells were transfected with p3×FLAG-CMV10 or p3×FLAG-CMV10-LGP2, selected in G418 for two weeks (200 μg/mL) and individual clones were verified for stable LGP2 expression and assessed in clonogenic assays.

Flow cytometric analysis. Single-cell suspensions of cells were isolated and incubated with anti-annexin V and propidium iodide according to the manufacturer's instructions (Annexin V Apoptosis Detection Kit, eBioscience). Samples were analyzed on a FACSCanto flow cytometer (BD Biosciences), and data were analyzed with FlowJo software (TreeStar, Inc.).

Statistical Analysis

A. siRNA Screen Analysis.

For each of the basal level and IR screens, the intensities of the plate were first log 2 transformed and then normalized with normalized percent inhibition (NPI) method to correct for plate effect. The normalized intensities were further divided by the per-plate median absolute deviations (MAD) in order to adjust the variance. The procedures were performed using Bioconductor package cellHTS2 (Boutros M, Bras L P, & Huber W (2006) Analysis of cell-based RNAi screens. Genome biology 7(7):R66). To identify the genes that lead to the most consistent decrement in cell viability when suppressed across 14 cell lines, we conducted a rank aggregation on the gene rank lists obtained from basal level and IR screens, separately. The Robust Rank Aggregation (RAA) algorithm implemented in R package RobustRankAggreg was applied (Kolde R, Laur S, Adler P, & Vilo J (2012) Robust rank aggregation for gene list integration and meta-analysis. Bioinformatics 28(4):573-580). Briefly, the RRA method assumes a null model where the ranks of each gene are uniformly distributed over the rank lists. For each plate, the 89 genes were sorted in descending order of their median normalized intensity of the three replicates. Then for each position in the sorted list, the probability that a randomly sampled rank from the null model has a lower rank value than the value at that position in the sorted list can be calculated. The minimum of the resulting probabilities over all positions in the sorted list is defined as the rank score of the gene, which can then be converted into an estimated P-value of the gene through Bonferroni correction (Dunn O J (1961) Multiple Comparisons Among Means. Journal of the American Statistical Association 56(293):52-64). The derived P-values are subject to multiple testing correction to control the false discovery rate (FDR) by Benjamini-Hochberg procedure (Benjamini Y & Hochberg Y (1995) Controlling the False Discovery Rate—a Practical and Powerful Approach to Multiple Testing. J Roy Stat Soc B Met 57(1):289-300). To further evaluate the stability of Bonferroni corrected P-values, we applied leave-one-out permutation test on the robust rank aggregation algorithm (Vosa U, et al. (2013) Meta-analysis of microRNA expression in lung cancer. International Journal of Cancer 132(12):2884-2893.). The analysis was conducted by performing RRA on a subset of 14 gene lists with one randomly selected list excluded. The procedure was repeated 100,000 times and the P-values from each permutation for each gene were then averaged.

B. Database Analysis.

Glioblastoma datasets were collected from the Cancer Genome Atlas (CGA) (n=382) and Phillips et al. study (n=77) (Phillips H S, et al. (2006) Molecular subclasses of high-grade glioma predict prognosis, delineate a pattern of disease progression, and resemble stages in neurogenesis. Cancer cell 9(3):157-173). Only patients with a history of prior radiation therapy were included in the analysis. mRNA expression values were normalized to the median value across all patient samples within each respective dataset. Gene expression data were visualized using hierarchical clustering. ISG expression was based on the mRNA expression of interferon-inducible genes as reviewed in (Khodarev N R, B, Weichselbaum, R (2012) Molecular Pathways: Interferon/Stat1 pathway: role in the tumor resistance to genotoxic stress and aggressive growth Clinical Cancer Research 18(11):1-7). Kaplan-Meier survival analysis with a log-rank test was used to compare overall survival for LGP2-positive patients, defined as 1.5-fold increased expression above the group median, versus LGP2-negative patients. Cox proportional hazard analysis of overall survival was performed to determine the hazard ratio for overall survival of LGP2-positiveversus LGP2-negative patients. All analyses were performed using JMP 9.0 (SAS Institute Inc.; Cary, N.C.). A p-value ≦0.05 was considered statistically significant.

C. Quantitative Data Analysis.

Data are presented as means±standard deviations (SD) for three or more representative experiments. Statistical significance was calculated using Student's t test.

Discussion

Several studies have shown that the response of tumor cells to ionizing radiation (IR) is associated with Interferon (IFN)-mediated signaling (Khodarev N N, et al. (2004) STAT1 is overexpressed in tumors selected for radioresistance and confers protection from radiation in transduced sensitive cells. Proc Natl Acad Sci USA 101(6):1714-1719; Khodarev N N, et al. (2007) Signal transducer and activator of transcription 1 regulates both cytotoxic and prosurvival functions in tumor cells. Cancer Res 67(19):9214-9220; Tsai M H, et al. (2007) Gene expression profiling of breast, prostate, and glioma cells following single versus fractionated doses of radiation. Cancer Res 67(8):3845-3852; John-Aryankalayil M, et al. (2010) Fractionated radiation therapy can induce a molecular profile for therapeutic targeting. Radiat Res 174(4):446-458; Cheon H, Yang J, & Stark G R (2011) The functions of signal transducers and activators of transcriptions 1 and 3 as cytokine-inducible proteins. J Interferon Cytokine Res 31(1):33-40; Amundson S A, et al. (2004) Human in vivo radiation-induced biomarkers: gene expression changes in radiotherapy patients. Cancer Res 64(18):6368-6371). IFN signaling leads to the induction of multiple Interferon-Stimulated Genes (ISGs) (Borden E C, et al. (2007) Interferons at age 50: past, current and future impact on biomedicine. Nat Rev Drug Discov 6(12):975-990; Samuel C E (2001) Antiviral actions of interferons. Clin Microbiol Rev 14(4):778-809, table of contents), and activates growth arrest and cell death in exposed cell populations (Kotredes K P & Gamero A M (Interferons as inducers of apoptosis in malignant cells. J Interferon Cytokine Res 33(4):162-170). However, the precise mechanism of IR-mediated induction of IFN signaling is unknown. Tumor cell clones that survive an initial cytotoxic insult are subsequently resistant to exposure to both IR and pro-death components of IFN signaling (Khodarev N R, B, Weichselbaum, R (2012) Molecular Pathways: Interferon/Stat1 pathway: role in the tumor resistance to genotoxic stress and aggressive growth Clinical Cancer Research 18(11):1-7). These clones express IFN dependent enhanced levels of constitutively expressed ISGs, which overlap in part with ISGs initially induced by cytotoxic stress. Many of these constitutively expressed ISGs have been characterized as anti-viral genes (Perou C M, et al. (1999) Distinctive gene expression patterns in human mammary epithelial cells and breast cancers. Proc Natl Acad Sci USA 96(16):9212-9217). Recently, enhanced levels of constitutively expressed ISGs have been reported in advanced cancers and were often associated with a poor prognosis related to aggressive tumor growth, metastatic spread, resistance to a IR/chemotherapy, or combinations of these factors (Perou C M, et al. (1999) Distinctive gene expression patterns in human mammary epithelial cells and breast cancers. Proc Natl Acad Sci USA 96(16):9212-9217; Weichselbaum R R, et al. (2008) An interferon-related gene signature for DNA damage resistance is a predictive marker for chemotherapy and radiation for breast cancer. Proc Natl Acad Sci USA 105(47):18490-18495; Martin D N, Starks A M, & Ambs S (Biological determinants of health disparities in prostate cancer. Curr Opin Oncol 25(3):235-241; Duarte C W, et al. (Expression signature of IFN/STAT1 signaling genes predicts poor survival outcome in glioblastoma multiforme in a subtype-specific manner. PLoS One 7(1):e29653; Hix L M, et al. (Tumor STAT1 transcription factor activity enhances breast tumor growth and immune suppression mediated by myeloid-derived suppressor cells. J Blot Chem 288(17):11676-11688; Haricharan S & Li Y (STAT signaling in mammary gland differentiation, cell survival and tumorigenesis. Mol Cell Endocrinol; Camicia R, et al. (BAL1/ARTD9 represses the anti-proliferative and pro-apoptotic IFNgamma-STAT1-IRF1-p53 axis in diffuse large B-cell lymphoma. J Cell Sci 126(Pt 9):1969-1980). The studies presented herein are based on the hypothesis that a specific set of constitutively expressed ISGs, whose enhanced expression by cytotoxic stress, confers a selective advantage to individual tumor clones (Cheon H, Yang J, & Stark G R (2011) The functions of signal transducers and activators of transcriptions 1 and 3 as cytokine-inducible proteins. J Interferon Cytokine Res 31(1):33-40.; Kotredes K P & Gamero A M (Interferons as inducers of apoptosis in malignant cells. J Interferon Cytokine Res 33(4):162-170; Khodarev N R, B, Weichselbaum, R (2012) Molecular Pathways: Interferon/Stat1 pathway: role in the tumor resistance to genotoxic stress and aggressive growth Clinical Cancer Research 18(11):1-7; Weichselbaum R R, et al. (2008) An interferon-related gene signature for DNA damage resistance is a predictive marker for chemotherapy and radiation for breast cancer. Proc Natl Acad Sci USA 105(47):18490-18495; Cheon H, et al. (2013) IFNbeta-dependent increases in STAT1, STAT2, and IRF9 mediate resistance to viruses and DNA damage. The EMBO journal 32(20):2751-2763).

To test this hypothesis, we designed a targeted siRNA screen against 89 ISGs selected from 2 sources. The first included ISGs identified in our earlier screen and designated the Interferon-Related DNA Damage Signature (IRDS) (Khodarev N N, et al. (2004) STAT1 is overexpressed in tumors selected for radioresistance and confers protection from radiation in transduced sensitive cells. Proc Natl Acad Sci USA 101(6):1714-1719; Weichselbaum R R, et al. (2008) An interferon-related gene signature for DNA damage resistance is a predictive marker for chemotherapy and radiation for breast cancer. Proc Natl Acad Sci USA 105(47):18490-18495). The second set included related ISG signatures that have been reported in the literature (as described above in Methods and in Table No. 1). The 89 genes were individually targeted in 14 tumor cell lines derived from malignant gliomas, lung, breast, colon, head and neck, prostate and bladder cancers.

One of our most significant finding from this screen was that the RNA helicase LGP2 (DHX58) confers survival and mediates the response to IR of multiple tumor cell lines. LGP2, an abbreviation of Laboratory of Genetics and Physiology 2, acts as a suppressor of the RNA-activated cytoplasmic RIG-1-like receptors pathway (Malur M, Gale M, Jr., & Krug R M (2013) LGP2 downregulates interferon production during infection with seasonal human influenza A viruses that activate interferon regulatory factor 3. J Virol 86(19):10733-10738; Komuro A & Horvath C M (2006) RNA- and virus-independent inhibition of antiviral signaling by RNA helicase LGP2. J Virol 80(24):12332-12342). This pathway is a subtype of pattern recognition receptors responsible for primary recognition of pathogen and host-associated molecular patterns and the subsequent activation of Type I interferon production that orchestrates an innate immune response (Akira S, Uematsu S, & Takeuchi O (2006) Pathogen recognition and innate immunity. Cell 124(4):783-801; Kawasaki T, Kawai T, & Akira S (2011) Recognition of nucleic acids by pattern-recognition receptors and its relevance in autoimmunity. Immunol Rev 243(1):61-73; Multhoff G & Radons J (2012) Radiation, inflammation, and immune responses in cancer. Front Oncol 2:58). In addition to its role in inhibiting IFNβ expression, Suthar et al. recently demonstrated that LGP2 governs CD8+ T cell fitness and survival by inhibiting death-receptor signaling (Suthar M S, et al. (2012) The RIG-I-like receptor LGP2 controls CD8(+) T cell survival and fitness. Immunity 37(2):235-248). Here we demonstrate that suppression of LGP2 leads to an enhanced IFNβ expression and increased killing of tumor cells. Our results thereby provide the first mechanistic connection between IR-induced cytotoxic response in tumor cells and the LGP2-IFNβ pathway.

An siRNA screen targeting 89 Interferon Stimulated Genes (ISGs) in 14 different cancer cell lines pointed to the RIG-I-like receptor LGP2 (Laboratory of Genetics and Physiology 2, also RNA helicase DHX58) as playing a key role in conferring tumor cell survival following cytotoxic stress induced by ionizing irradiation (IR). Studies on the role of LGP2 revealed the following; (i) Depletion of LGP2 in 3 cancer cells lines resulted in significant increase in cell death following IR, (ii) Ectopic expression of LGP2 in cells increased resistance to IR, and (iii) IR induced enhanced LGP2 expression in 3 cell lines tested.

Our studies designed to define the mechanism by which LGP2 acts point to its role in regulation of IFNβ. Specifically, (i) Suppression of LGP2 leads to enhanced IFNβ (ii) Cytotoxic effects following IR correlated with expression of IFNβ inasmuch as inhibition of IFNβ by neutralizing antibody conferred resistance to cell death, and (iii) Mouse embryonic fibroblasts (MEFs) from IFN Receptor 1 knock-out mice (IFNAR1^{-/-}) are radioresistant compared to wild-type MEFs. The role of LGP2 in cancer may be inferred from cumulative data showing elevated levels of LGP2 in cancer cells are associated with more adverse clinical outcomes. Our results below indicate that cytotoxic stress exemplified by IR induces IFNβ and enhances the expression of LGP2. Enhanced expression of LGP2 suppresses the ISGs associated with cytotoxic stress by turning off the expression of IFNβ.

Results

Expression of LGP2 is Associated with Tumor Cell Survival.

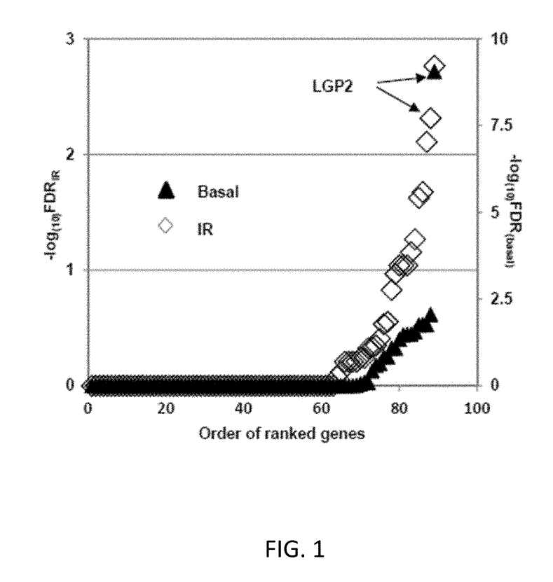

On the basis of our earlier studies (Khodarev N N, et al. (2004) STAT1 is overexpressed in tumors selected for radioresistance and confers protection from radiation in transduced sensitive cells. Proc Natl Acad Sci USA 101(6):1714-1719; Khodarev N N, et al. (2007) Signal transducer and activator of transcription 1 regulates both cytotoxic and prosurvival functions in tumor cells. Cancer Res 67(19):9214-9220; Weichselbaum R R, et al. (2008) An interferon-related gene signature for DNA damage resistance is a predictive marker for chemotherapy and radiation for breast cancer. Proc Natl Acad Sci USA 105(47):18490-18495; Khodarev N N, et al. (2009) STAT1 pathway mediates amplification of metastatic potential and resistance to therapy. PLoS One 4(6):e5821), we hypothesized the existence of ISGs that are constitutively expressed in aggressive cancers and confer pro-survival functions following cytotoxic stress caused by DNA damaging agents. To identify the key members of this group, we compiled a list of ISGs associated with aggressive tumors from multiple published studies (see Table No. 1). In total, 89 genes identified in Table No. 2 were selected for further evaluation based on either inclusion in the IRDS (Weichselbaum R R, et al. (2008) An interferon-related gene signature for DNA damage resistance is a predictive marker for chemotherapy and radiation for breast cancer. Proc Natl Acad Sci USA 105(47):18490-18495) or inclusion in at least two reported ISG-related signatures. To test whether expression of these genes conferred a survival advantage to tumor cells we performed a targeted siRNA screen in a panel of 14 cell lines consisting of 2 lung cancer, 3 high grade glioma, 3 breast cancer and normal breast epithelium, 2 colon cancer, 2 head and neck cancer, 1 bladder cancer, and 1 prostate cancer cell lines. Each tumor cell line, both untreated and after exposure to 3 Gy, was targeted with pooled siRNAs against each of the selected 89 genes and scored on the basis of cell viability. To identify genes with pro-survival functions common across multiple cell lines tested we used a rank aggregation approach assuming each cell line was an independent dataset (Adler P, et al. (2009) Mining for coexpression across hundreds of datasets using novel rank aggregation and visualization methods. Genome biology 10(12):R139; Boulesteix A L & Slawski M (2009) Stability and aggregation of ranked gene lists. Briefings in bioinformatics 10(5):556-568). With different modes of normalizations and perturbations LGP2 was invariably the top ranked gene in unirradiated cells (See FIG. 1). In addition, LGP2 was among the top ranked genes conferring survival to multiple cancer cell lines after irradiation at 3Gy. The focus of this report is on the role of LGP2 in the regulation of cell survival.

LGP2 Blocks Apoptosis Induced by IR.

The desirable endpoint of radiotherapy is induction of apoptosis in irradiated cells. To define the role of LGP2 in determination of the outcome of IR treatment we tested the effects of depletion of LGP2 on induction of apoptosis by IR in WiDr, D54, and Scc61 cancer cell lines. As detailed in Methods and in the figure legends the cell lines were transfected with non-targeted (scrambled) siRNA (siNT) or targeted (siLGP2) siRNA and either mock-irradiated or irradiated (5 Gy) 24 hrs after transfection. The cells were stained with Annexin V and propidium iodide and scored for both markers by flow cytometry 48 hours after IR or mock treatment. The results were as follows:

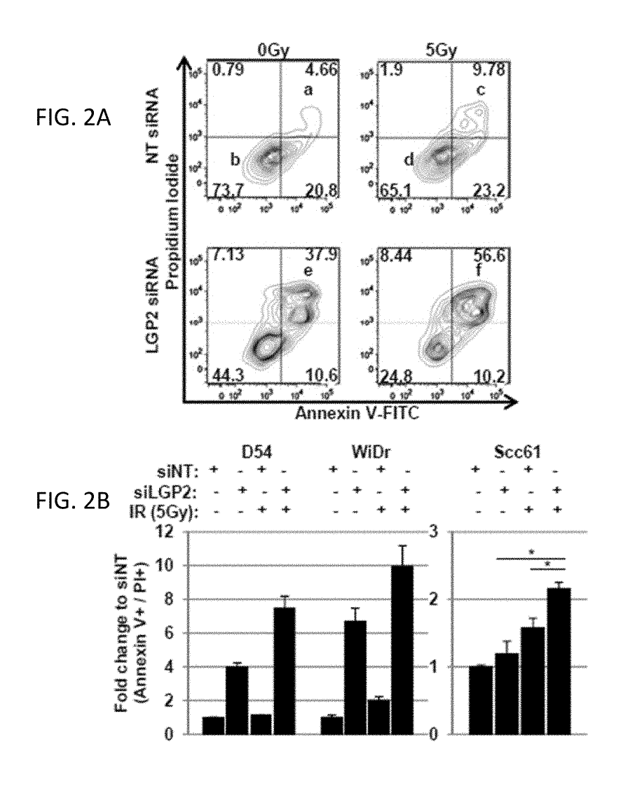

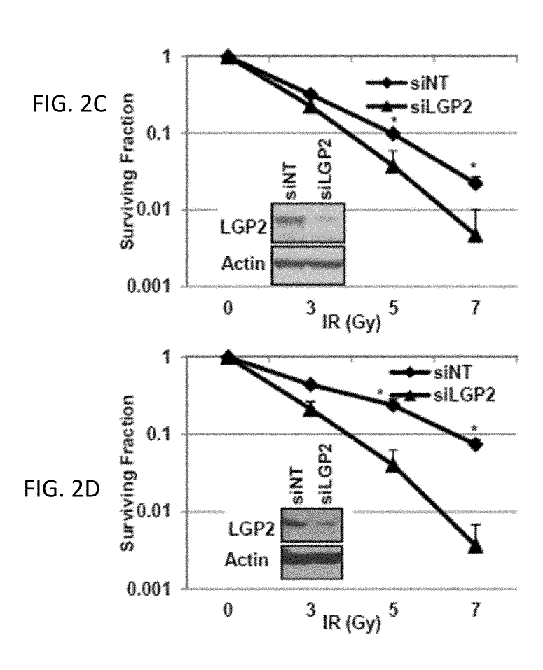

As shown in FIG. 2A and in FIG. 2B, transfection of WiDr cells with a non-targeting (scrambled) siRNA (siNT) led to a small (4.66%) increase in double-positive cells (FIG. 2A, panel a), while 73.7% of the cell population remained viable under these conditions (FIG. 2A, panel b). Irradiation of siNT-transfected cells led to an approximately 2-fold increase in cell death (9.8%) with an 8.6% reduction in viable cells (65.1%) (FIG. 2A, panels c and d, respectively). Suppression of LGP2 alone led to an increase in double-positive cells to 37.9% (8.1-fold increase) (FIG. 2A panel e). The combination of LGP2 suppression followed by irradiation led to further accumulation of double-positive cells to 56.6%; a 12.1-fold increase relative to the non-irradiated siNT control (FIG. 2A, panel f).

Similar data were obtained with D54 and Scc61 cells (FIG. 2B). As shown in FIG. 2B (left panel), siRNA knockdown of LGP2 in the D54 cells led to a 4-fold increase in cell death at baseline and a 7.5-fold increase following irradiation. The same conditions led to 6.4-fold cell death at baseline and 10-fold induction following IR in the WiDr cell line (FIG. 2B, left panel). A similar pattern was found in the Scc61 cell line (FIG. 2B, right panel, p<0.05). Clonogenic survival analyses revealed that siRNA-mediated depletion of LGP2 reduced radioresistance in both D54 and Scc61 cell lines. Compared to siNT control, irradiation of LGP2 depleted cells lead to 4.7 fold decrease in the survival fraction in D54 cells (p=0.014) and a 20.3-fold decrease in the survival fraction of Scc61 cells (p=0.00056) at 7Gy (FIGS. 2C and D, respectively). We conclude that suppression of LGP2 results in apoptosis and radiosensitization.

Overexpression of LGP2 Protects Cells from IR.

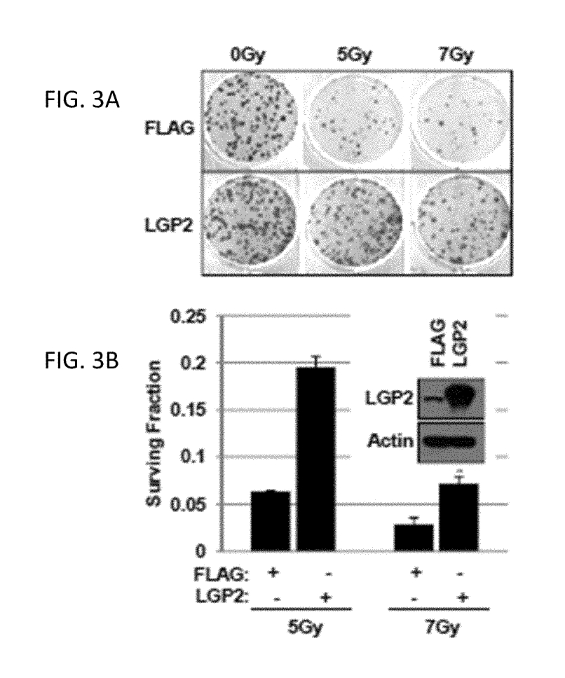

To verify the conclusion that LGP2 protects tumor cells cytotoxic effects of radiotherapy, we investigated the clonogenic survival of tumor cells expressing the full-length cDNA of LGP2. In this experiment, D54 cells were stably transfected with the plasmid p3×FLAG-CMV10-LGP2 encoding LGP2 or control p3×FLAG-CMV10 (Flag). Positive clones were plated in 6-well plates and exposed to 0, 5 or 7Gy. The amounts of LGP2 protein in mock (Flag) transfected and LGP2 transfected cells are shown in the insert in FIG. 3B. FIG. 3A shows the surviving cell colonies stained with crystal violet 12 days after irradiation. Panel B shows the fraction of mock-transfected and LGP2-transfected cells that survived exposure to IR quantified as described in materials and methods. We conclude that ectopic expression of LGP2 confers increased resistance to IR.

IR Induces Expression of LGP2.

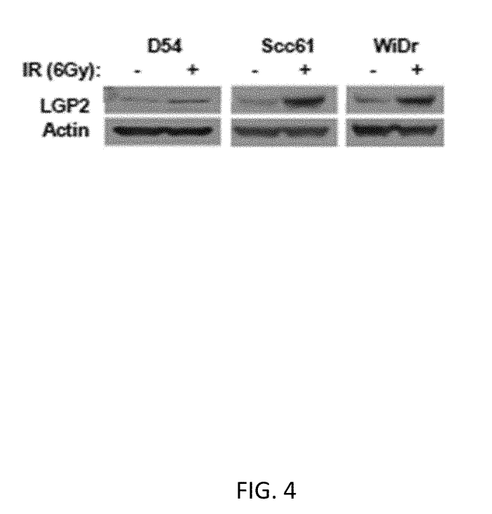

We next asked if exposure to IR would up-regulate LGP2 expression in tumor cells. In this experiment D54, Scc61 and WiDr cells were mock-treated or exposed to 6 Gy. The cells were harvested 72 hrs after IR, solubilized, and tested for the presence of LGP2 by immunoblotting with anti-LGP2 antibody; Actin served as loading control. As shown in FIG. 4, a significant increase in LGP2 expression was observed in IR treated cells. We conclude that IR induces the expression of LGP2.

IR Induces Cytotoxic Type I IFN.

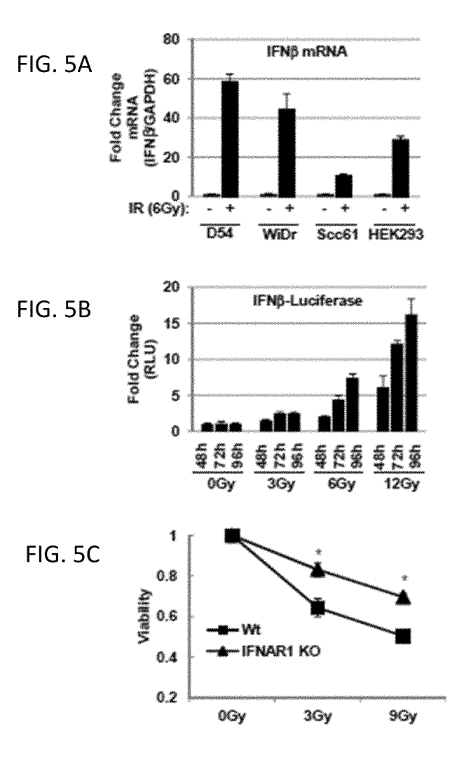

LGP2 functions to suppress Type I IFN production in response to viral infection or transfection of double-stranded RNA mimetics (Komuro A & Horvath C M (2006) RNA- and virus-independent inhibition of antiviral signaling by RNA helicase LGP2. J Virol 80(24):12332-12342; Saito T, et al. (2007) Regulation of innate antiviral defenses through a shared repressor domain in RIG-I and LGP2. Proc Natl Acad Sci USA 104(2):582-587; Yoneyama M, et al. (2005) Shared and unique functions of the DExD/H-box helicases RIG-I, MDA5, and LGP2 in antiviral innate immunity. Journal of immunology 175(5):2851-2858; Komuro A, Bamming D, & Horvath CM (2008) Negative regulation of cytoplasmic RNA-mediated antiviral signaling. Cytokine 43(3):350-358; Rothenfusser S, et al. (2005) The RNA helicase Lgp2 inhibits TLR-independent sensing of viral replication by retinoic acid-inducible gene-I. Journal of immunology 175(8):5260-5268). The objective of the studies described in this section was to determine whether IR induces a Type 1 IFN response. In these studies D54, WiDr, Scc61 or HEK293 cells were mock-treated or exposed to 6 Gy. The cells were harvested 72 hrs after IR, and IFNβ expression relative to GAPDH was determined by real time-PCR. As shown in FIG. 5A, exposure to IR increased the relative expression of IFNβ mRNA in D54, WiDR, SCC61 and HEK293 cell lines by 58, 42, 12 and 28-fold respectively. In a complementary approach, we investigated the ability of IR to activate a plasmid reporter under the control of IFNβ promoter (IFNβ-Luc) (Lin R, Genin P, Mamane Y, & Hiscott J (2000) Selective DNA binding and association with the CREB binding protein coactivator contribute to differential activation of alpha/beta interferon genes by interferon regulatory factors 3 and 7. Molecular and cellular biology 20(17):6342-6353). In these experiments HEK293 cells were co-transfected with IFNβ-Luc and pRL-SV40. At 24 hrs after transfection, cells were mock-treated or exposed to 3, 6, or 12 Gy. Cells were harvested 48, 72 or 96 hrs and analyzed for dual luciferase activity. As shown in FIG. 5B, IR activated IFNβ expression in a dose- and time-dependent manner.

To determine if induction of IFNβ by IR was cytotoxic, we determined the relative radiosensistivity of immortalized murine embryo fibroblasts lacking the Type I IFN receptor 1 (IFNAR1^{-/-}) as compare to wild type MEFs (Wt). In this experiments, IFNAR1^{-/-} and Wt MEFs were mock-treated or exposed to 3 or 9 Gy. Cells were assessed for viability 96 hrs after IR as described in Material and Methods. FIG. 5C shows that IFNAR1^{-/-} MEFs are radioresistant as compared to Wt MEFs. We conclude that IR induces the production of cytotoxic Type I Interferon.

Depletion of LGP2 Enhances IFNβ Dependent Cytotoxicity.

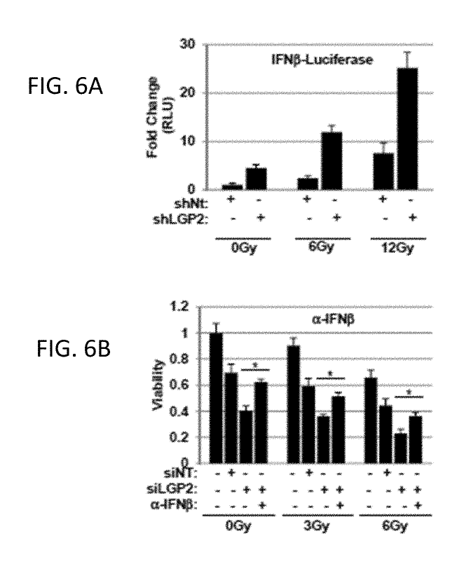

We next assessed the role of LGP2 in regulating the IR-induced IFNβ response. HEK293 cells were transduced with lentiviral shRNA to stably reduce the levels of LGP2 or control non-targeting (shNT). Stably transduced cells were co-transfected with IFNβ-Luc and pRL-SV40, mock-treated or exposed to 6 or 12 Gy and collected 72 hrs after IR. Suppression of LGP2 led to a significant increase in IFNβ reporter activity at mock-treated and greatly increased IR-induced IFNβ (FIG. 6A).

We next examined whether the radiosensitizing effects of LGP2 depletion were associated with a release of cytotoxic IFNβ. In this experiment, D54 cells were incubated with neutralizing antibodies against IFNβ and mock treated or exposed to 3 or 6 Gy; viability was assessed 96 hrs after IR. As shown in FIG. 6B, neutralizing antibodies against IFNβ partially restored viability of D54 cells with LGP2 knockdown to the level of control cells (siNT). These data are consistent with earlier studies from our laboratory demonstrating that neutralizing antibodies to IFNs partially protected human tumor xenografts from IR-mediated cytotoxicity (Khodarev N N, et al. (2007) Signal transducer and activator of transcription 1 regulates both cytotoxic and prosurvival functions in tumor cells. Cancer Res 67(19):9214-9220). These data also indicate that IR-induced tumor cell killing is mediated, in part, by the production of autocrine IFNβ (Khodarev N N, et al. (2007) Signal transducer and activator of transcription 1 regulates both cytotoxic and prosurvival functions in tumor cells. Cancer Res 67(19):9214-9220; Khodarev N R, B, Weichselbaum, R (2012) Molecular Pathways: Interferon/Stat1 pathway: role in the tumor resistance to genotoxic stress and aggressive growth Clinical Cancer Research 18(11):1-7). We conclude that LGP2 suppresses IR induced cytotoxic IFNβ production in tumor cells.

LGP2 Expression Predicts Poor Clinical Outcome in High Grade Gliomas.

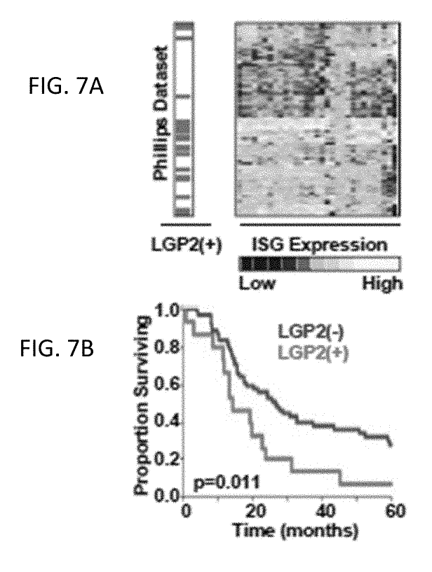

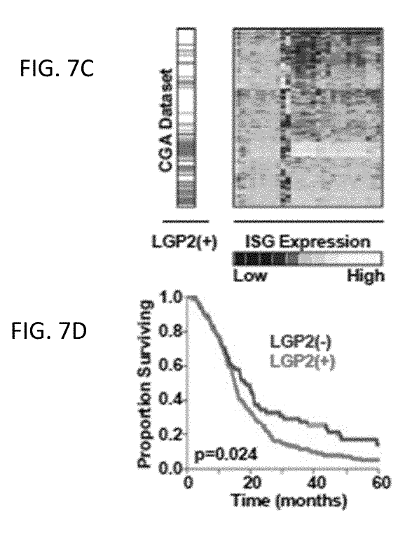

The studies described above suggest that depletion of LGP2 increases radiosensitivity whereas overexpression of LGP2 increases radioresistance of tumor cells. A key question is whether the results presented here are consistent with clinical experience and in particular the clinical outcomes in patients undergoing radiotherapy. Multiple studies have demonstrated an overall survival benefit for post-operative radiation therapy after surgical resection compared to surgery alone in the management of newly diagnosed glioblastoma multiforme (GBM) (Walker M D, et al. (1978) Evaluation of BCNU and/or radiotherapy in the treatment of anaplastic gliomas. A cooperative clinical trial. Journal of neurosurgery 49(3):333-343; Kristiansen K, et al. (1981) Combined modality therapy of operated astrocytomas grade III and IV. Confirmation of the value of postoperative irradiation and lack of potentiation of bleomycin on survival time: a prospective multicenter trial of the Scandinavian Glioblastoma Study Group. Cancer 47(4):649-652; Laperriere N, Zuraw L, Cairncross G, & Cancer Care Ontario Practice Guidelines Initiative Neuro-Oncology Disease Site G (2002) Radiotherapy for newly diagnosed malignant glioma in adults: a systematic review. Radiotherapy and oncology: journal of the European Society for Therapeutic Radiology and Oncology 64(3):259-273). In addition, the response of GBM tumors to radiation predicts the patient lifespan after treatment. In this regard, we described elsewhere that ISG expression correlated with poor overall survival in patients with GBM (Duarte C W, et al. (Expression signature of IFN/STAT1 signaling genes predicts poor survival outcome in glioblastoma multiforme in a subtype-specific manner. PLoS One 7(1):e29653). To investigate whether LGP2 gene expression is also related to clinical outcomes in patients with GBM, we analysed two independent GBM datasets from the Cancer Genome Atlas (CGA, see http://cancergenome.nih.gov/) (n=382) and the Phillips et al. study (n=77) (Phillips H S, et al. (2006) Molecular subclasses of high-grade glioma predict prognosis, delineate a pattern of disease progression, and resemble stages in neurogenesis. Cancer cell 9(3):157-173). In FIGS. 7A and 7C the relative expression of ISGs separates each dataset into ISG-positive and ISG-negative groups. FIGS. 7A and 7C further demonstrate that expression of LGP2 is highly associated with expression of ISGs. To examine the association of LGP2 expression with patient survival, we compared overall survival in the patient cohorts with relatively high and relatively low expression of LGP2. As is shown in FIGS. 7B and 7D, high expression of LGP2 was significantly associated with a 2.3-fold increased risk for death in the Phillips dataset (p=0.011, Cox proportional hazards test) and a 1.4-fold increased risk for death in the TCGA dataset (p=0.024). These data demonstrate that LGP2 gene expression is associated with poor clinical outcome in patients with GBM and support our hypothesis that this protein may serve as a potential biomarker and target for the radiosensitization of high grade gliomas.

Conclusions

The salient features of the results are as follows:

(i) We demonstrated a correlation between expression of LPG2 and resistance to IR in most of the 14 human cancers cell lines of diverse origins. In follow up studies we demonstrated that depletion of LGP2 enhanced cytotoxic sequelae of IR whereas overexpression of LGP2 increased the fraction of cells resistant to cytotoxicity induced by IR.

(ii) LGP2 is a constitutive cytoplasmic protein whose accumulation is enhanced by IFN and hence it is defined as an ISG. Several studies have identified a link between ISGs and aggressive tumor phenotypes with poor outcomes or radio/chemoresistance (Cheon H, Yang J, & Stark G R (2011) The functions of signal transducers and activators of transcriptions 1 and 3 as cytokine-inducible proteins. J Interferon Cytokine Res 31(1):33-40; Khodarev N R, B, Weichselbaum, R (2012) Molecular Pathways: Interferon/Stat1 pathway: role in the tumor resistance to genotoxic stress and aggressive growth Clinical Cancer Research 18(11):1-7). In studies designed to explore in more detail the interaction between LGP2, IFN and IR we showed that IR induces both IFNβ and enhances the accumulation of LPG2, that overexpression of LGP2 causes a significant reduction of IFNβ gene expression and lastly, that inhibition of IFNβ by neutralizing antibody results in increased resistance to cytotoxic effects induced by IR.

(iii) A survey of available databases suggests a correlation between the expression of LGP2 and poor outcomes in patients with malignant glioblastoma.

The significance of the studies presented here are as follows:

(i) Expression of LGP2 emerged as necessary and on the basis of the effects of ectopic expression as sufficient for enabling enhanced survival of cancer cells exposed to cytotoxic doses of IR. Since chemotherapeutic drugs may mimic the effects of IR, LGP2 may indeed be the primary but perhaps not unique ISG to block cytotoxic manifestations associated with IFN production in cells subjected to DNA damaging agents. Therefore it is contemplated that identification of the mechanism by which LGP2 acts to block IFN production may be a key to development of adjunct therapies to block its function and enhance therapeutic outcomes.

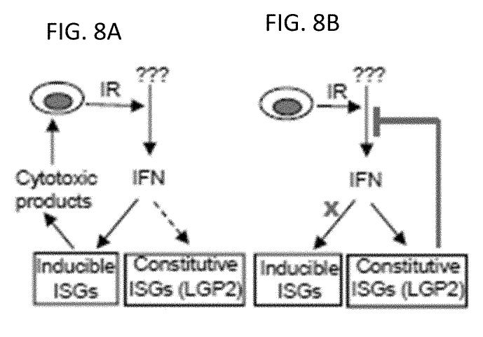

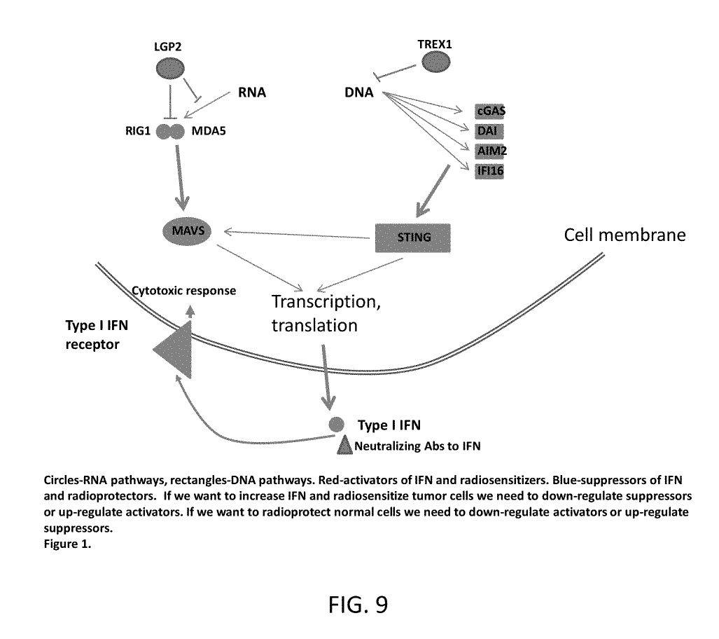

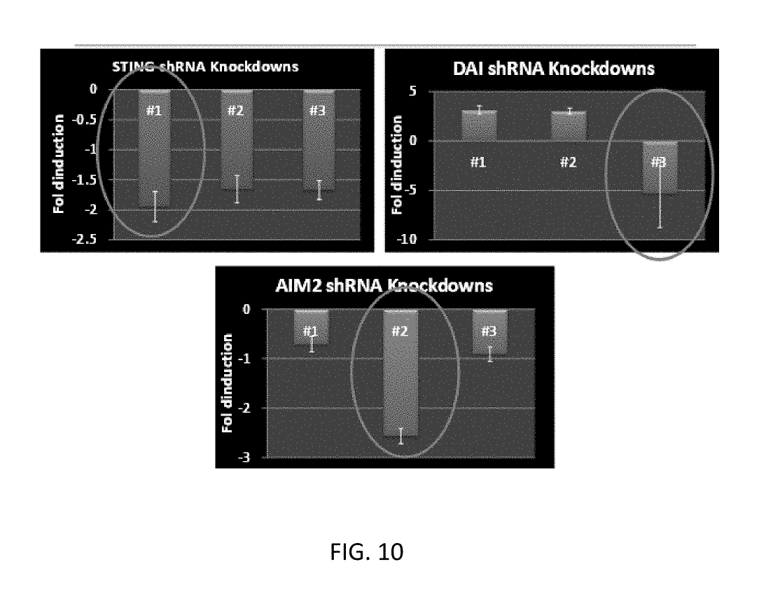

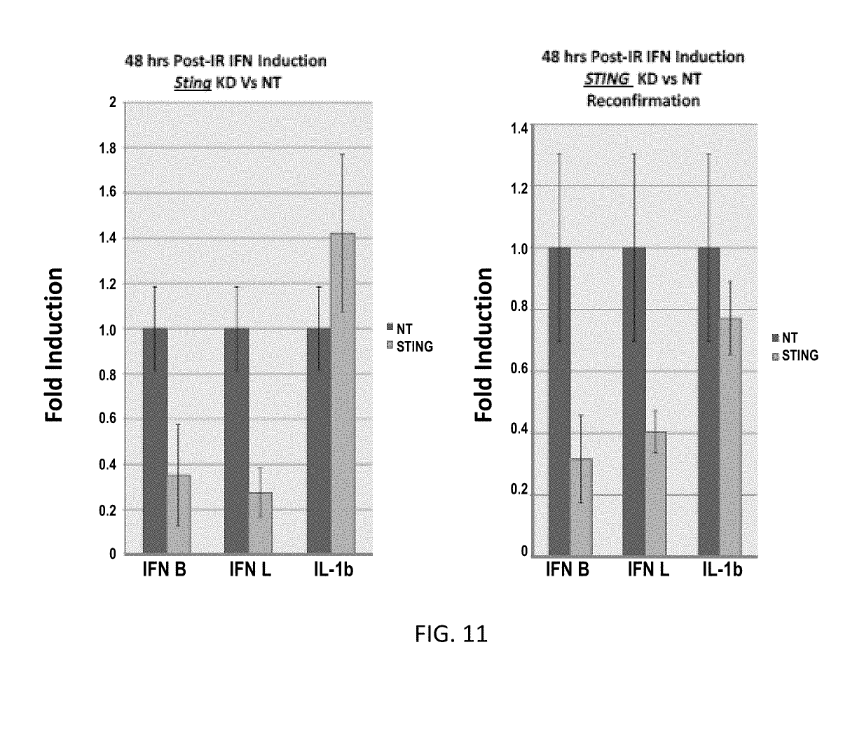

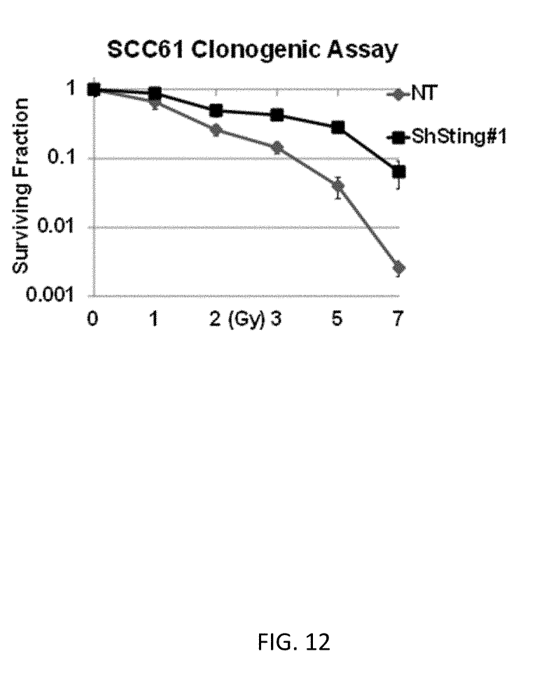

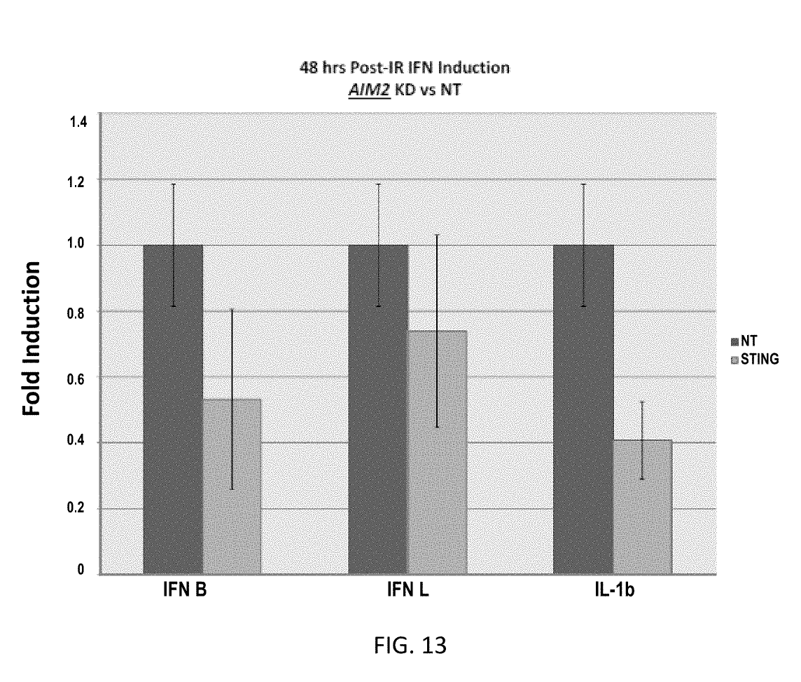

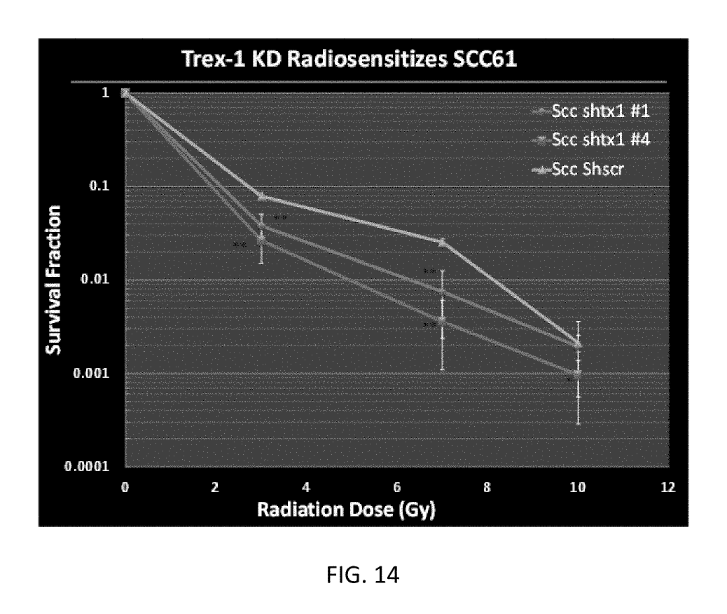

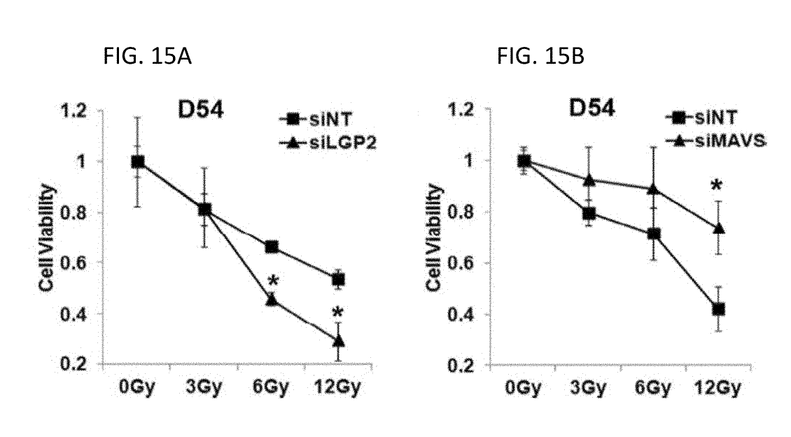

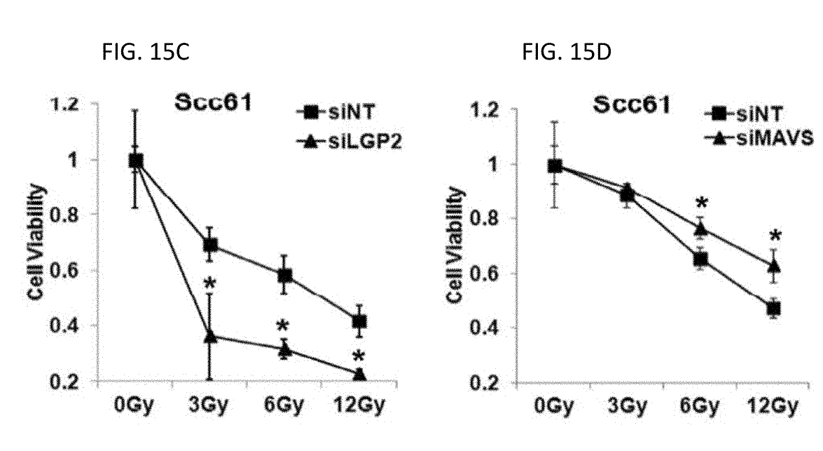

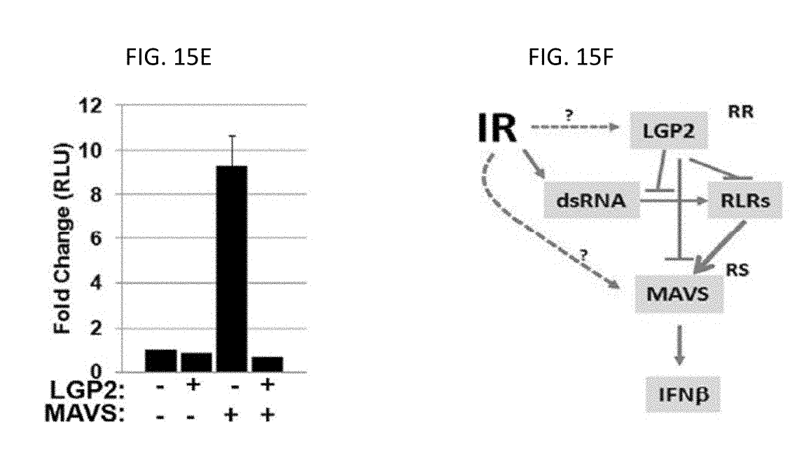

(ii) In light of the overwhelming evidence that LGP2 is a constitutive cellular protein whose accumulation is enhanced by IFN the obvious question is under what conditions is LGP2 inoperative and what activates its anti-IFN functions. In principle, LGP2 acts as a classic feedback inhibitor (FIG. 8) that is activated by an unknown mechanism. The solution to this puzzle is likely to greatly accelerate the mean by which its function could be blocked.

### Example 2. STING Signaling Mediates Antitumor Effects of Radiation

Methods

Mice

Six- to eight-week old C57BL/6J mice were purchased from Harlan. MyD88^{-/-}, TRIF^{-/-}, CRAMP^{-/-}, 2C CD8⁺ T cell receptor (TCR)-Tg, CD11c-Cre-Tg mice were purchased from The Jackson Laboratory. IFNAR1^{flox/flox} mice were kindly provided by Dr. Ulrich Kalinke of the Institute for Experimental Infection Research, Hanover, Germany. STING^{-/-} mice were kindly provided by Dr. Glen N. Barber of University of Miami School of Medicine, Miami. IRF3^{-/-} mice were kindly provided by T. Taniguchi of University of Tokyo, Tokyo, Japan. All the mice were maintained under specific pathogen free conditions and used in accordance to the animal experimental guidelines set by the Institute of Animal Care and Use Committee. This study has been approved by the Institutional Animal Care and Use Committee of the University of Chicago.

Tumor Growth and Treatments

1×10⁶ MC38 tumor cells were subcutaneously injected into the flank of mice.

Tumor volumes were measured along three orthogonal axes (a, b, and c) and calculated as tumor volume=abc/2. Tumors were allowed to grow for 9-10 days and treated by local radiation (Deng et al., 2014). Briefly, the body was protected with a lead cover and the tumor was exposed, allowing local radiation. Tumors were irradiated using RS-2000 Biological Irradiator (RAD SOURCE) at the dose of 20Gy with 160 kV and 25 mA. For type I IFN blockade experiments, 200 μg anti-IFAR1 mAb was intratumorally injected on day 0 and 2 after radiation. For HMGB-1 blockade experiments, 200 μg anti-HMGB-1 mAb (clone 3B1, generated by inventors) was administered i.p. on day 0 and 3 after radiation. For CD8⁺ T cell depletion experiments, 300 μg anti-CD8 mAb (Clone 2.43, BioXCell) was delivered 5 times by i.p. injection every three days starting one day before radiation. For exogenous IFN-β treatment experiments, 1×10¹⁰ viral particles of Ad-IFN-β (Burnette, B., et al., The Efficacy of Radiotherapy Relies upon Induction of Type I Interferon-Dependent Innate and Adaptive Immunity, Cancer Res Apr. 1, 2011 71; 2488; (doi: 10.1158/0008-5472.CAN-10-2820)) were intratumorally administered on day 2 after radiation. Ad-null was used as negative control. For cGAMP treatment experiments, 10 μg 2′3′-Cgamp (InvivoGen; cyclic [G(2′,5′)pA(3′,5′)p]); CAS 1441190-66-4) in PBS was intratumorally administered on day 2 and 6 after radiation at a dose of 0.45 μg/mg.

In Vitro Culture and Function Assay of BMDCs

Single-cell suspensions of bone marrow cells were obtained from C57BL/6J, STING^{-/-} and IRF3^{-/-} mice. Bone marrow from cGAS^{-/-} mice was kindly provided by Dr. Zhijian J. Chen of University of Texas Southwestern Medical Center, Dallas. The cells were placed in 10 cm petri dish and cultured in RPMI-1640 medium containing 10% fetal bovine serum (DENVILLE), supplemented with 20 ng/ml GM-CSF. Fresh media with GM-CSF was added into culture on day 3. BMDCs (bone marrow-derived dendritic cells) were harvest for stimulation assay on day 7. 8×10⁶ MC38-SIY^{hi} cells were plated into 10 cm cell culture dishes overnight, and then pretreated with 40Gy and incubated for 5 hours. BMDCs were added and co-cultured with MC38-SIY″ cells at the ratio of 1:1 in the presence of fresh GM-CSF for additional 8 hours. Subsequently purified CD11c⁺ cells with EasySep™ Mouse CD11c Positive Selection Kit II (STEMCELL) were incubated with isolated CD8⁺ T cells from naive 2C mice for three days. For the bypassing assay, 10 ng/ml murine IFN-β was added in the co-culture of BMDCs and tumor cells, or 100 μg/ml DMXAA was added into isolated CD11c⁺ cells with additional 3 h incubation. For IFN-β detection, BMDCs were co-cultured with tumor cells at the ratio of 1:1 for additional 8 hours, and 1×10⁶ cells/ml purified CD11c⁺ cells were seed into 96-well plates for 48 hours.

RNA Interference

siRNAs (Mission siRNA) against murine cGAS and control siRNA were purchased from Sigma as described. BMDCs were transfected with siRNA by Lipofectamine RNAiMAX Reagent (Invitrogen) at a final concentration of 50 nM: mmcGAS 5′-GAGGAAAUCCGCUGAGUCAdTdT-3′ (SEQ ID NO:8); MissionsiRNA Universal Negative control 1. Forty-eight hours after transfection, cells were used for further experiments.

RNA Extraction and Quantitative Real-Time RT-PCR

Total RNA from sorted cells was extracted with the RNeasy Micro Kit (QIAGEN) and reversed-transcribed with Seniscript Reverse Transcription Kit (QIAGEN). Real-time RT-PCR was performed with SSoFast EvaGreen supermix (Bio-Rad) according to the manufacturer's instructions and different primer sets on StepOne Plus (Applied Biosystems). Data were normalized by the level of 18S expression in each individual sample. 2^{-ΔΔCt} method was used to calculate relative expression changes.

ELISA

Tumor tissues were excised on day 3 after radiation and homogenized in PBS with protease inhibitor. After homogenization, Triton X-100 was added to obtain lysates. Cell culture supernatants were obtained from isolated CD11c⁺ cells after 48 h-incubation with fresh GM-CSF. The concentration of IFN-β and CXCL10 was measured with VeriKine-HS™ Mouse Interferon Beta Serum ELISA Kit (PBL Assay Science) and mouse CXCL10 Quantikine ELISA kit (R&D) in accordance with the manufacturer's instructions, respectively.

Measurement of IFNγ-Secreting CD8⁺ T Cells by ELISPOT Assay

For bone-marrow CD11c⁺ cells functional assay, 2×10⁴ purified CD11c⁺ cells with were incubated with isolated CD8⁺ T cells from naive 2C mice with EasySep™ Mouse CD8a Positive Selection Kit (STEMCELL) for three days at the ratio of 1:10. For tumor-specific CD8⁺ T cells functional assay, eight days after radiation, tumor DLNs were removed and CD8⁺ T cells were purified. MC38 tumor cells were exposed to 20 ng/ml murine IFN-γ for 24 hr prior to plating with purified CD8⁺ T. 2×10⁵CD8⁺ T cells were incubated with MC38 at the ratio of 10:1 for 48 hours. 96-well HTS-IP plate (Millipore) was pre-coated with 2.5 m/ml anti-IFN-γ antibody (clone R4-6A2, BD Pharmingen) overnight at 4° C. After co-culture, cells were removed, 2 μg/ml biotinylated anti-IFN-γ antibody (clone XMG1.2, BD Pharmingen) was added, and the plate was incubated for 2 h at room temperature or overnight at 4° C. Avidin-horseradish peroxidase (BD Pharmingen) with a 1:1000 dilution was then added and the plate was incubated for 1 h at room temperature. The cytokine spots of IFN-γ were developed according to product protocol (Millipore).

Cell Lines and Reagents

MC38 is a murine colon adenocarcinoma cell line. MC38-SIY was selected for a single clone after being transduced by lentivirus expressing human EGFR (L858R)-SIY. Anti-mIFNAR1 neutralizing mAb (clone MAR1-5A3) and anti-CD8 depleting mAb (clone 2.43) were purchased from BioXcell (West Lebanon, N.H.). Anti-HMGB-1 neutralizing mAb (clone 3B1) was produced in house. Anti-HMGB-1 mAb is capable of neutralizing HMGB-1 in vivo. Conjugated antibodies against CD11b, CD11c and CD45, and 7-AAD were purchased from BioLegend. 2′3′-cGAMP was purchased from InvivoGen. DMXAA was purchased from Selleck Chemicals. Murine IFN-β, murine IFN-γ and murine GM-CSF was purchased from PEPROTECH.

Direct Priming Assay

Bone-marrow CD11c⁺ cells were co-cultured with purified CD8⁺ T cells from 2C mice in the presence of 1 μg/ml SIY peptide (SIYRYYGL (SEQ ID NO:28)) for three days. The supernatants were harvested for IFN-γ detection.

Flow Cytometric Sorting and Analysis

To obtain single cell suspensions, tumor tissues were cut into small pieces and mechanical dissociated with the gentleMACS™ Dissociators (Miltenyi Biotech). Then tumor tissues were digested by 1 mg/ml collagenase IV (Sigma) and 0.2 mg/ml DNase I (Sigma) for 30 min at 37° C. For the staining, single cell suspensions were blocked with anti-FcR (clone 2.4G2, BioXcell) and then stained with antibodies against CD11c, CD11b and CD45, and 7-AAD. Cells were performed on FACSAria II Cell Sorter (BD). For Mouse IFN-γ Flex Set CBA assay, IFN-γ detection in the supernatants was performed on FACSCalibur Flow Cytometer (BD). Data were analyzed with FlowJo Software (ThreeStar).

Primer Sequences for Real-Time PCR

Primer sequences for quantitative real-time PCR were as follows:

mIFN-β forward
(SEQ ID NO: 9)
5′-GGTGGAATGAGACTATTGTTG-3′,
mIFN-β reverse
(SEQ ID NO: 10)
5′-AAGTGGAGAGCAGTTGAG-3′;
m-cGAS forward
(SEQ ID NO: 11)
5′-ACCGGACAAGCTAAAGAAGGTGCT-3′,
m-cGAS reverse
(SEQ ID NO: 12)
5′-GCAGCAGGCGTTCCACAACTTTAT-3′;
and
18S forward
(SEQ ID NO: 13)
5′-CGTCTGCCCTATCAACTTTCG-3′,
18S reverse
(SEQ ID NO: 14)
5′-TGCCTTCCTTGGATGTGGTA-3′.

Statistical Analysis

Experiments were repeated three times. Data were analyzed using Prism 5.0 Software (GraphPad) and presented as mean values ±SEM. The P values were assessed using two-tailed unpaired Student t tests and p<0.05 was considered significant. For tumor-bearing mice frequency, statistics were done with the log rank (Mantel-Cox) test.

Discussion

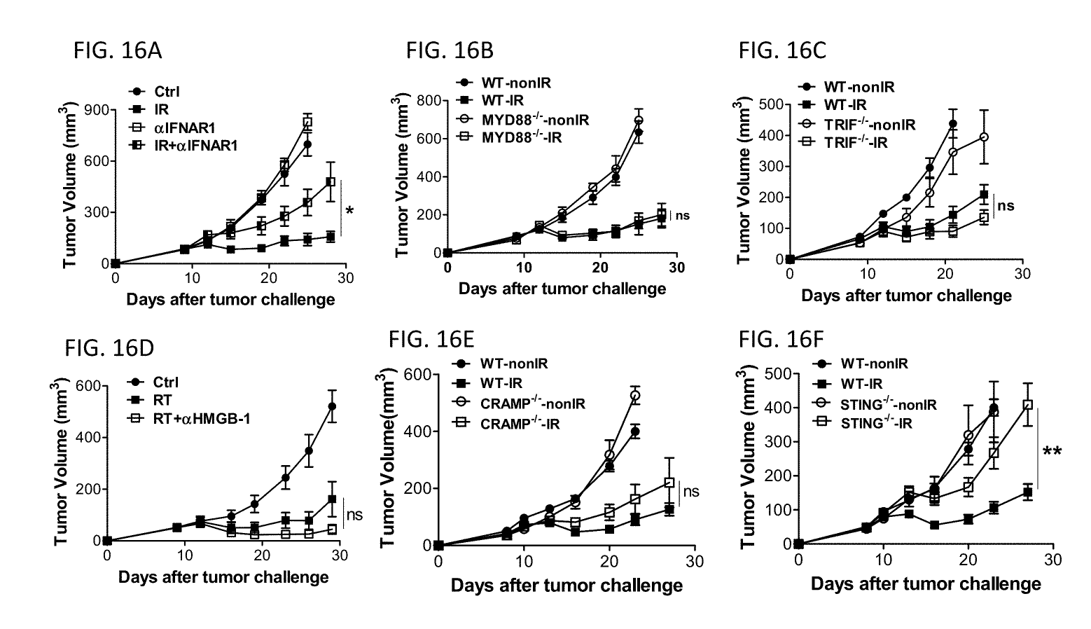

We previously demonstrated that antitumor effects of radiation were dependent on type I IFN signaling by utilizing IFNAR1^{-/-} mice (Burnette et al., 2011). To rule out the possibility that failure of tumors to respond to radiation was due to the intrinsic or developmental deficiency of IFNAR^{-/-} mice, we administered blocking antibody against IFNAR1 in wild type (WT) mice following radiation. The results were similar to the effects observed in the knockout (KO) mice in that the antitumor effect of radiation was greatly attenuated by the neutralization of type I IFNs signaling with antibodies (FIG. 16A). The prevailing understanding of type I induction by the detection of DAMPs is dominated by the activation of TLRs (Chen and Nunez, 2010; Kono and Rock, 2008). The adaptor proteins MyD88 and TRIF mediate the induction of type I IFNs by TLRs activation with DAMPs recognition (Desmet and Ishii, 2012). In addition, it has been demonstrated that MyD88 is essential for antitumor immunity of chemotherapy and targeted therapies with anti-HER2 (Apetoh et al., 2007; Park et al., 2010; Stagg et al., 2011). To test the role of MyD88 upon radiation, we implanted tumor cells on flanks of WT and MyD88^{-/-} mice. The inhibition of tumor growth post radiation was comparable between WT and MyD88^{-/-} mice (FIG. 16B). This surprising result demonstrates that MyD88 in the host is dispensable for antitumor effect of radiation. To examine whether TRIF is important for the antitumor effect of radiation, we injected tumor cells into WT and TRIF^{-/-} mice. The deficiency of TRIF in the host failed to reverse tumor inhibition by radiation (FIG. 16C). This result is consistent with our previous observation, confirming that TRIF is redundant for antitumor effect of radiation (Burnette et al., 2011). HMGB-1 secretion has been shown to be essential for antitumor immunity of chemotherapy and targeted therapies with anti-HER2 (Apetoh et al., 2007; Park et al., 2010). Similar to chemotherapy and targeted therapies, radiotherapy induces cell stress and result in the secretion of DAMPs. To examine whether HMGB-1 secretion is critical for the antitumor effect of radiation, we blocked HMGB-1 with antibodies following radiation. Tumor control of radiation was unaffected by anti-HMGB-1 treatment (FIG. 16D), suggesting that HMGB-1 secretion is also not required for the antitumor effect of radiation. The cathelicidin-related antimicrobial peptide (CRAMP in mice and LL37 in human) has been identified as a mediator of type I IFN induction by binding self-DNA to trigger TLR9-MyD88 pathway (Diana et al., 2013; Lande et al., 2007). To validate the possibility that CRAMP is responsible for the radiation response, we inoculated tumor cells into WT and CRAMP^{-/-} mice. The deficiency of CRAMP was unable to dampen the antitumor effect of radiation (FIG. 16E), indicating that CRAMP is unnecessary for radiation response. Taken together, these data indicate that well-characterized TLRs-dependent molecular mechanisms involved in chemotherapy and targeted therapies using antibodies are not responsible for antitumor efficacy of radiation. Also, these results raise the possibility that a unique molecular mechanism which is TLRs-independent for type I IFN induction mediates the antitumor effect of radiation.

Recently, STING-mediated cytosolic DNA sensing cascade has been demonstrated to be one major mechanism of TLR-independent type I IFN induction. This process requires TBK1 and its downstream transcription factor, IRF3 (Desmet and Ishii, 2012; Wu and Chen, 2014). To determine the role of STING in radiation response, we implanted tumor cells on flanks of WT and STING^{-/-} mice to monitor tumor growth curve. Without radiation treatment, the tumor growth was identical in WT mice and in STING^{-/-} mice. In contrast, the tumor burden was significantly reduced by radiation in WT mice, whereas the deficiency of STING in the host significantly impaired the antitumor effect of radiation (FIG. 16F), demonstrating that STING signaling is important for the antitumor effect of radiation. Taken together, these results suggest that newly-defined STING-dependent cytosolic DNA sensing pathway, not well-characterized TLRs-dependent nucleic acids sensing pathways, mediates the antitumor effect of radiation.

Results

STING Signaling Controls Type I IFN Induction and Innate Immune Responses Upon Radiation

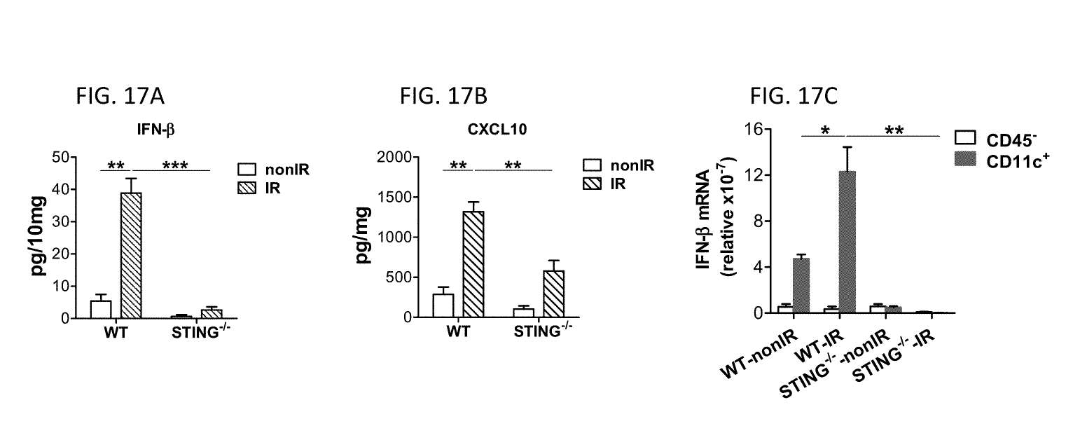

To test whether STING was responsible for type I induction following radiation, we measured the protein level of IFN-β in tumors. The induction of IFN-β in tumors was significantly abrogated in the absence of STING in the host after radiation (FIG. 17A). To validate whether STING mediates type I IFN induction, we determined the protein level of CCL10, a type I IFN-stimulated gene (Ablasser et al., 2013; Holm et al., 2012). The induction of CXCL10 in tumors was markedly diminished after radiation in the STING-deficient host (FIG. 17B), confirming that radiation-mediated type I IFN induction is determined by the presence of STING. These results indicate that STING in the host, not in tumor cells, mediates type I induction by radiation. Next, to determine in which cell population STING mediates type I IFN induction, we performed quantitative real-time PCR assay of IFN-β in different sorted cell populations from tumors after radiation. We observed that DCs (CD11c⁺) were the major producer of IFN-β after radiation, compared to CD45⁻ population and the rest of myeloid cells (data not shown), whereas radiation-mediated the induction of IFN-β mRNA by DCs was abolished in the host with STING deficiency (FIG. 17C). Together, these data suggest that host STING controls radiation-mediated type I IFN induction in tumors and that the presence of STING in tumor-infiltrating DCs plays a major role in type I IFN induction after radiation.

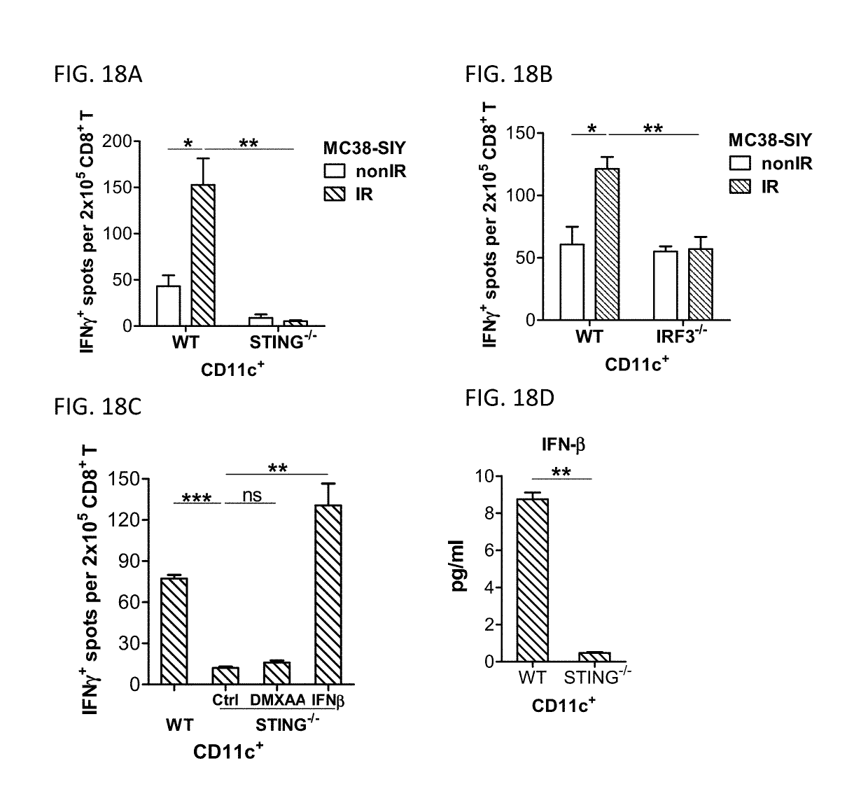

To determine whether STING signaling is activated by irradiated-tumor cells and whether it is essential to cross-priming of DCs for CD8⁺ T cells, a cross-priming assay was conducted with BMDCs from WT and STING^{-/-} mice. The function of DCs was significantly elevated by the stimulation of irradiated-tumor cells compared to non-irradiated-tumor cells, whereas the deficiency of STING in DC resulted in failed responses of DCs to cross-prime T cells (FIG. 18A). It has been demonstrated that STING-dependent type I IFN production is mediated by IRF3 phosphorylation (Wu and Chen, 2014). To confirm that STING-associated downstream for radiation-mediated type I IFN production is essential to the function of DCs, we performed cross-priming assay with WT-BMDCs and IRF3^{-/-}BMDCs. Similar to STING^{-/-} BMDC, IRF3^{-/-} BMDCs failed to cross-prime CD8⁺ T cells with the stimulation of irradiated-tumor cells (FIG. 18B). These results indicate that STING-IRF3 axis in DCs is activated by irradiated-tumor cells, in turn, the activation of the STING-IRF3 axis predominates the cross-priming ability of DCs.

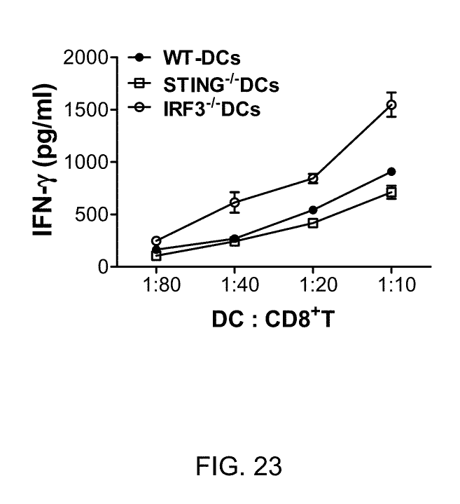

To determine whether exogenous IFN-β treatment rescues the functions of STING^{-/-}BMDCs, we added IFN-β into the co-culture system of BMDCs and tumor cells. The functions of STING^{-/-}BMDCs were restored in the presence of exogenous IFN-β treatment (FIG. 18C). Recently, it has been demonstrated that DMXAA binds to murine STING and activates STING signaling to induce type I IFN production (Gao et al., 2013b). DMXAA fails to rescue the function of STING^{-/-} BMDCs, confirming activation of STING is required to increase cross-priming through IFN pathway (FIG. 18C). Next, to rule out the possibility that the discrepancy in priming ability of STING^{-/-} DCs and IRF3^{-/-} DCs are due to intrinsic defects of these cells, a direct priming assay was performed with peptide stimulation. Remarkably, no significant difference was observed between WT-BMDCs and STING^{-/-} BMDCs function in priming 2C cells with the stimulation of SIY peptide (FIG. 23). It suggests that DC has not intrinsic defect in cross priming. IRF3^{-/-} DCs were even more efficient than WT DCs in priming 2C cells with SIY peptide stimulation (FIG. 23), probably due to pro-apoptotic function of IRF3. To validate STING signaling is activated by irradiated-tumor cells, we determined the production of IFN-β by WT-BMDCs and STING^{-/-} BMDCs stimulated by irradiated-tumor cells. The protein level of IFN-β was remarkably reduced in STING^{-/-} BMDCs compared to WT-BMDCs (FIG. 18D). These results indicate that activation of STING by irradiated-tumor cells controls type I IFN induction in DCs and this process is a pivotal contributor to the ability of DCs to cross-prime CD8⁺ T cells. On the other hand, these results raise the possibility that STING molecules in DCs are activated by a certain stimulator, presumably DNA, provided by irradiated-tumor cells.

cGAS Mediates Dendritic Cell Sensing of Irradiated-Tumor Cells

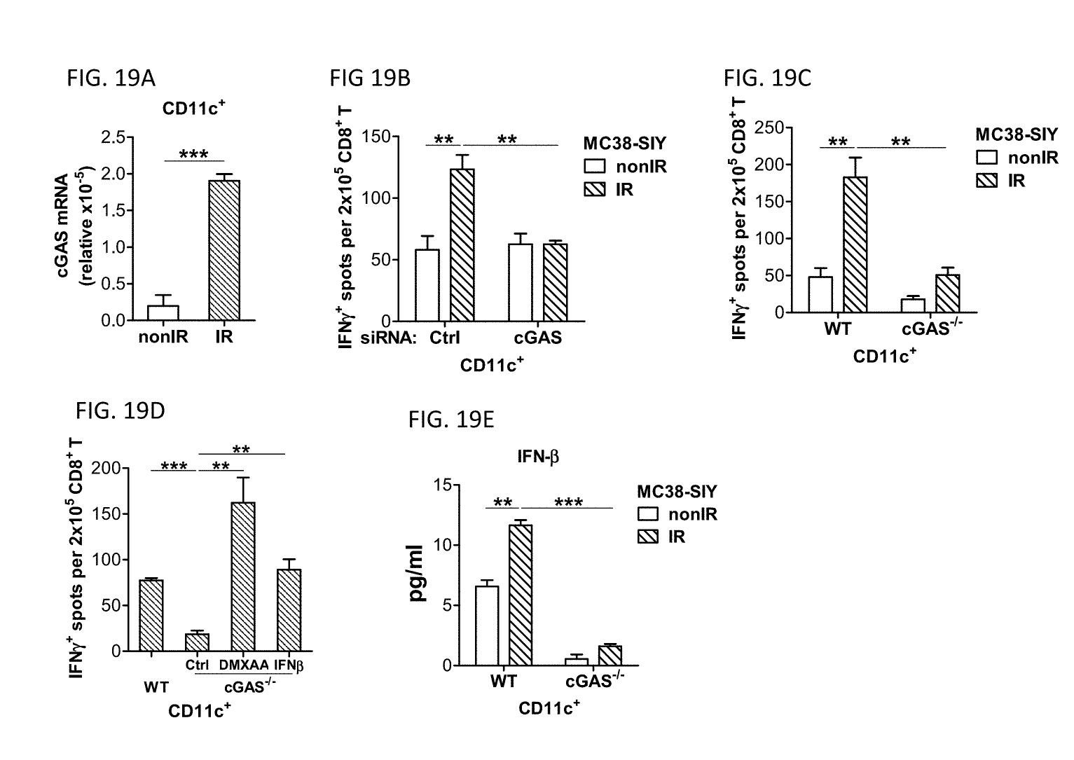

Recent studies have shown that cGAS is a cytosolic DNA-sensing enzyme that catalyses the production of cyclic GMP-AMP (cGAMP), a second-messenger activator of STING-dependent type I IFN production (Wu and Chen, 2014). Furthermore, elevation of cGAS mRNA level in CD11c⁺ cells from tumors is observed after radiation (FIG. 19A), indicating that cGAS in DC is likely induced by its substrate, cytosol DNA, following radiation. To interrogate whether cGAS is required for DCs sensing of irradiated-tumor cells to stimulate adaptive immunity, we silenced cGAS in BMDCs using siRNA. The silencing of cGAS in BMDCs greatly diminished the function of DCs compared to the silencing of non-target controls, when stimulated with irradiated-tumor cells (FIG. 19B). To validate the role of cGAS in DCs sensing of irradiated-tumor cells, we compared the function of BMDCs from WT and cGAS^{-/-} mice. In contrast to WT BMDCs, cGAS^{-/-} BMDCs failed to cross-prime 2C cells in response to stimulation by irradiated-tumor cells (FIG. 19C), confirming that cGAS is important for DCs sensing of irradiated-tumor cells. To map whether cGAS-STING-type I IFN axis determines the function of BMDCs, we performed bypass experiments with the treatment of exogenous IFN-β and DMXAA. The functions of cGAS^{-/-} BMDCs were restored with IFN-β and DMXAA treatment, respectively (FIG. 19D). To further confirm that cGAS is required for the BMDCs sensing of irradiated-tumor cells, we determined the production of IFN-β in WT-BMDCs and cGAS^{-/-} BMDCs after stimulation of irradiated-tumor cells. The protein level of IFN-β was greatly decreased in cGAS^{-/-} BMDCs compared to WT-BMDCs (FIG. 19E). Therefore, these results indicate that cGAS mediates type I IFN production to enhance the function of DCs in response to irradiated-tumor cells. Also, these results suggest that DNA from irradiated-tumor cells is delivered into the cytosol of DCs and then binds to cGAS to trigger STING-dependent type I IFN induction.

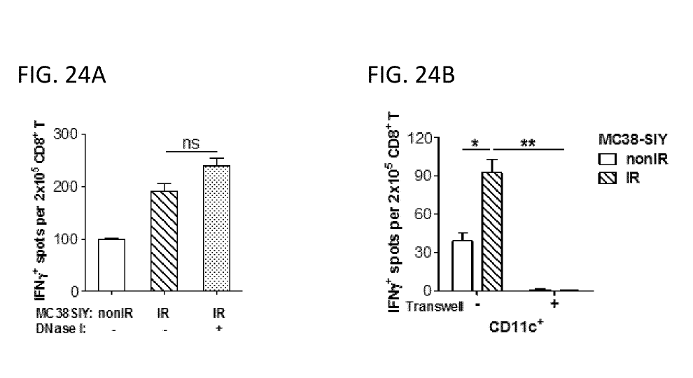

We next determine how DNA from irradiated-tumor cells is delivered into the cytosol of DCs. With the damaging effects of radiation, the cells might either lose membrane integrity and release endogenous DNA fragments which are engulfed by DCs, or maintain membrane integrity and DNA fragments are transferred by phagocytosis. In the presence of DNase I, the priming ability of DCs response was not impaired when stimulated by irradiated-tumor cells (FIG. 24A), suggesting that DCs unlikely engulf floating naked DNA fragments. To test whether DNA is delivered by exosome vesicles, BMDCs were stimulated with irradiated-tumor cells in a contact or a non-contact system. Separating BMDCs and irradiated-tumor cells via a trans-well screen which only allows media to travel freely, completely abolished the functions of DCs (FIG. 24B), indicating DNA delivery is mediated by direct cell-to-cell contact, not exosome vesicles. Taken together, these results suggest that DNA from irradiated-tumor cells is sensed by host cGAS during cell-cell contact engulfing process, such as phagocytosis.

STING Signaling Promotes Adaptive Immune Responses Upon Radiation

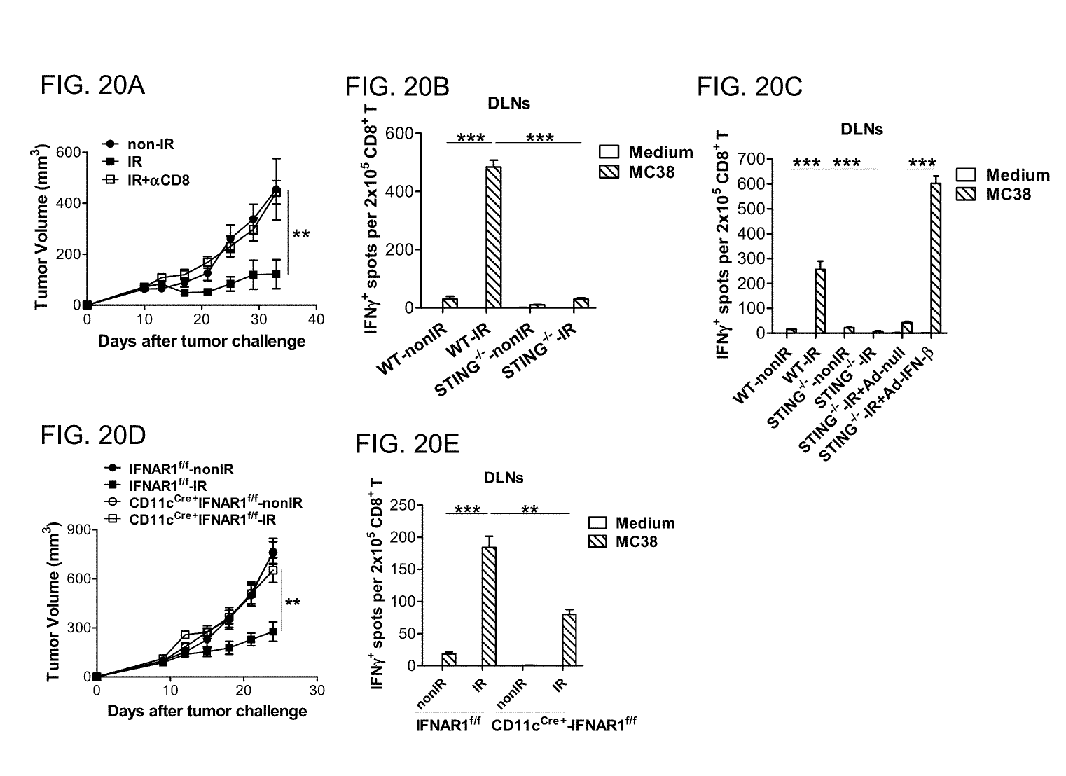

Our previous studies have shown that adaptive immune responses play an important role for the anti-tumor effect with either radiation alone or combined immunotherapy (Deng et al., 2014; Lee et al., 2009; Liang et al., 2013). To validate the role of CD8⁺ T cells after radiation in the current tumor model, MC38, depleting antibodies against CD8⁺ T cells were administrated following radiation. In agreement with our previous reports, the anti-tumor effect of radiation was greatly reduced with the depletion of CD8⁺ T cells after radiation (FIG. 20A), mimicking the tumor growth curve in STING^{-/-} mice post radiation. We sought to examine whether the failure of response to radiation in STING^{-/-} mice is due to impairment in the function of CD8⁺ T cells. To test whether STING signaling impacts a tumor antigen-specific CD8⁺ T cell response, we performed ELISPOT assay with purified CD8⁺ T cells from tumor draining lymph nodes (DLNs). Radiation induced a robust tumor antigen-specific CD8⁺ T cell responses in WT mice, whereas the antigen-specific CD8⁺ T cell responses in STING^{-/-} mice after radiation were significantly diminished (FIG. 20B). To confirm that the impairment of CD8⁺ T cell responses in STING^{-/-} mice post radiation is due to the insufficient induction of type I IFNs, STING^{-/-} mice received intratumorally treatment with Ad-IFN-β following radiation. Exogenous IFN-β treatment was able to restore the CD8⁺ T cell functions in STING^{-/-} mice after radiation (FIG. 20C). In addition, the intrinsic defect of CD8⁺ T cell responses has previously been examined through the vaccination of ovalbumin and incomplete Freunds adjuvant. The CD8⁺ T cell response in STING^{-/-} mice and WT mice was demonstrated to be equivalent (Ishikawa et al., 2009). As a result, these data together show that the reduction of type I IFNs, not intrinsic defect of T cells, accounts for inadequate adaptive immune responses in STING^{-/-} mice after radiation. Together, these results suggest that STING signaling is important for radiation-induced antitumor adaptive immune response.

To further determine whether DCs are responsible for the type I IFN signaling after radiation, we implanted tumor cells into CD11c^{Cre+}-IFNAR1^{f/f} mice and IFNAR1^{f/f} mice. Conditional deletion of IFNAR1 on DCs hampered the antitumor effect of radiation (FIG. 20D), demonstrating that type I IFN signaling on DCs are responsible for antitumor effects of radiation. Next, we determined the CD8⁺ T cell response in DLNs of CD11c^{Cre+}-IFNAR1^{f/f} mice and IFNAR1^{f/f} mice following radiation. The CD8⁺ T cell function was remarkably compromised in DLNs of CD11c^{Cre+}-IFNAR1^{f/f} mice versus IFNAR1^{f/f} mice following radiation (FIG. 20E). These results indicate that type I IFN signaling on DCs is required for antitumor efficacy of radiation by boosting adaptive immune responses.

cGAMP Treatment and Radiation Synergistically Amplify the Antitumor Immune Responses

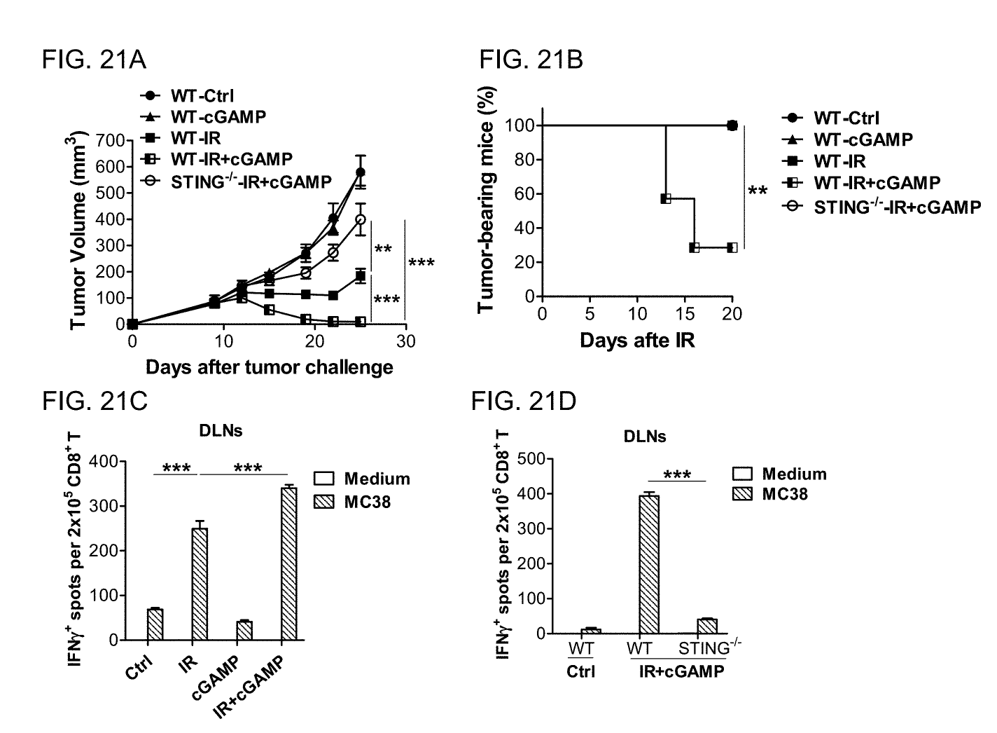

It has been demonstrated that 2′3′-cGAMP (cyclic [G(2′,5′)pA(3′,5′)p]) is generated in mammalian cells by cGAS in response of double-stranded DNA in the cytoplasm. 2′3′-cGAMP is potent to activate innate immune responses by binding STING and subsequently inducing TBK1-IRF3-dependent IFN-β production (Gao et al., 2013a; Wu et al., 2013; Zhang et al., 2013). We hypothesized that exogenous 2′3′-cGAMP treatment improves the antitumor effect of radiation by enhancing STING activation. To test this hypothesis, 2′3′-cGAMP was intratumorally administrated after radiation at a dose of 10 μg administered to mice 6-8 weeks of age of approximately 25-35 g each. Treatment with a combination of 2′3′-cGAMP and radiation effectively reduce tumor burden compared to 2′3′-cGAMP or radiation alone in WT mice, suggesting cGAMP treatment can reduce tumor radiation resistance, a common cause of tumor relapse (FIGS. 21A and 21B). In contrast, the synergy of 2′3′-cGAMP and radiation was abrogated in STING^{-/-} mice (FIGS. 21A and 21B). Together, these data indicate boosting the activation of STING signaling is able to remarkably inhibit tumor growth. To address whether the combination of 2′3′-cGAMP and radiation enhances tumor-specific T cell responses, ELISPOT assay were performed with isolated CD8⁺ T cells from DLNs, co-cultured with IFN-γ-treated MC38. The number of tumor-specific IFN-γ-producing CD8⁺ T cells was significantly increased in DLNs of mice that received combination treatment compared with those that received radiation or 2′3′-cGAMP alone (FIG. 21C). However, the robust antitumor CD8⁺ T cell response induced by the combination of 2′3′-cGAMP and radiation was dampened by the deficiency of STING in the host (FIG. 21D). Together, these results indicate that 2′3′-cGAMP treatment reduces radiation resistance by further enhancing tumor-specific CD8⁺ T cell functions and that the synergy is dependent on the presence of STING in the host, not in tumor cells.

Conclusions

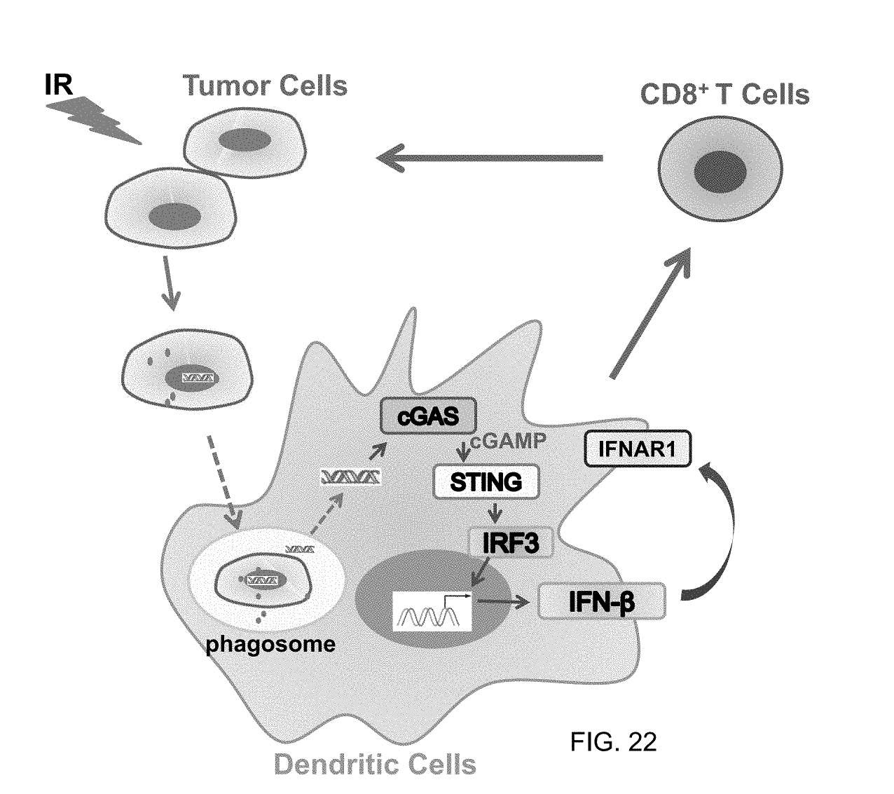

Radiation has been demonstrated to induce adaptive immune responses to mediate tumor regression (Apetoh et al., 2007; Lee et al., 2009). The induction of type I IFNs by radiation is essential for the function of CD8⁺ T cells (Burnette et al., 2011). Although the importance of type I IFNs has been elucidated by utilizing the mice with whole body depletion of IFNAR1, which immune cells are responsible for type I IFN responses after radiation remained unsolved. More importantly, because the stimuli of type I IFN induction are diverse, discerning the mechanism responsible for type I IFN induction by radiation has been elusive. Various nucleic acid-sensing pathways from different subcellular compartments have been reported to play a critical role in inducing type I IFNs in response to pathogen infection and tissue injury (Desmet and Ishii, 2012; Wu and Chen, 2014). Indeed, radiation induces cell stress and causes excess DNA breaks, indicating that nucleic acid-sensing pathway likely account for the induction of type I IFNs upon radiation. We identify that cGAS-STING dependent-cytosolic DNA sensing pathway in DCs is required for type I IFN induction after radiation, and then the type I IFN signaling on DCs determines radiation-mediated adaptive immune responses. In addition, enhancing STING signaling by exogenous cGAMP treatment facilitates the antitumor effect of radiation. Therefore, our current study reveals that cGAS-STING-dependent cytosolic DNA sensing pathway is a key mediator of tumor immune responses to therapeutic radiation (See FIG. 22).

This study shows that type I IFN responses in DCs dictate the efficacy of antitumor radiation and proposed that HMGB-1 release by dying tumor cells and MyD88 signaling in the host are dispensable for radiation treatment. In contrast, chemotherapeutic agents and anti-HER2 antibody treatment have been demonstrated to depend on a distinct immune mechanism to trigger adaptive immune responses (Apetoh et al., 2007; Park et al., 2010). Anti-HER2 treatment and chemotherapy require HMGB-1 release from dying tumor cells, and TLR4 and its adaptor MyD88 on DCs. The interaction of HMGB-1 and TLR4 potentiates the processing of dying tumor cells by DCs, leading to efficient cross-priming of CD8⁺ T cells. However, antitumor effects of chemotherapy have been shown to depend on MyD88 signaling but not TLR4 (Iida et al., 2013). The inconsistencies are likely due to the treatment schedule including the tumor size of starting treatment and the dose of chemotherapeutic agent. Although MyD88 signaling has been shown to be necessary for the vaccination with irradiated-tumor cells, it is unanticipated that this signaling is dispensable in radiation treatment of established tumors. Nevertheless, our study demonstrates that the induction of type I IFNs by radiation depends on STING signaling, validating that a particular molecular mechanism mediates antitumor immune responses to radiation. Therefore, it is evident that therapeutic radiation-mediated antitumor immunity depends on a proper cytosolic DNA sensing pathway.

It has been shown that cGAS-STING sensing pathway is a key component in activating innate immune response to various DNA from pathogens, including virus, bacteria and parasites (Gao et al., 2013b; Lahaye et al., 2013; Li et al., 2013; Lippmann et al., 2011; Sharma et al., 2011). Also, cGAS-STING signaling pathway might play a dominant role in response to transfected DNA. Two groups have linked this signaling with DNA vaccines performed by intramuscular electroporation. One report found that TBK1 mediates antigen-specific B cell and T cell immune response after DNA vaccination through type I IFN induction (Ishii et al., 2008). Another report pointed out that STING is essential for DNA vaccine-induced adaptive immune responses (Ishikawa et al., 2009). However, whether DNA from dying cells acts as DAMPs to provoke immune responses remains unclear. The release of DNA from dying host cells has been shown to stimulate adaptive immune responses in the TBK1-IRF3-type I IFN-dependent manner, leading to alum adjuvant activity (Marichal et al., 2011). Specifically, oxidized self-DNA released from dying cells has been demonstrated to activate cGAS-STING-dependent cytosolic DNA sensing pathway as a mechanistic interpretation of UV-exposed skin lesions (Bernard et al., 2012). Our results uncover that cGAS-STING-dependent cytosolic DNA sensing pathway mediates the efficacy of therapeutic radiation. Moreover, cGAS-STING signaling is important for direct DCs sensing of irradiated-tumor cells as tested by an in vitro assay. It is likely that cytosol DNA from irradiated-tumor cells is a mediator to activate cGAS-STING signaling in DCs. Although DNA can be sensed by T cells and induce costimulatory responses, this process is independent on known DNA sensing pathways, including STING signaling (Imanishi et al., 2014). In addition, our result shows that DCs are major producer of type I IFNs following radiation. We propose that cGAS-STING signaling in DCs plays a key role in the sensing of irradiated-tumor cell DNA to induce subsequent tumor-specific CD8⁺ T cell responses.

How DNA from irradiated-tumor cells is delivered into the cytosol of DCs remains unknown. DNA binding proteins such as LL37 are prevalent in neutrophil extracellular traps (NETs) and enhance cytoplasmic delivery of DNA (Diana et al., 2013; Lande et al., 2007). Indeed, several reports have shown that STING signaling is activated by DNA-LL37 complex (Chamilos et al., 2012; Gehrke et al., 2013). However, our results ruled out the possibility that DNA is delivered either by free floating form or by complex forms. Our data show that the direct cell-to-cell contact is required for the delivery of DNA from irradiated tumor cells, suggesting that phagocytosis mediates DNA delivery. Indeed, several groups have observed that phagosomal instability allows the content of this compartment to access to the cytosol, such as bacterial RNA (Sander et al., 2011). It is therefore possible that DNA from irradiated-tumor cells is delivered into the cytosol of DCs during membrane fusing process. Moreover, radiation is able to induce tumor cells and phagocytes to generate ROS, and then oxidated DNA modified by ROS is resistant to cytosolic exonuclease TREX-1-mediated degradation (Gehrke et al., 2013; Moeller et al., 2004). It is contemplated that radiation-induced ROS maintains the stability of tumor cell DNA during delivery into the cytosol of DCs. Therefore, we conclude that mapping out how tumor cell DNA traverses into the cytosol of DC will lead to further therapeutic targets using the present disclosure.

In summary, we demonstrate that the adaptor protein STING instead of MyD88 and TRIF provides for the antitumor effect of radiation and the induction of type I IFNs. The DNA sensor cGAS is important for DCs sensing of nucleic acids from irradiated-tumor cells. Moreover, cGAS-STING-IRF3-Type I IFNs cascade through autocrine action in DCs mediates robust adaptive immune responses to radiation. In addition, exogenous cGAMP treatment synergizes with radiation to control tumors. Therefore, our findings reveal a novel molecular mechanism of radiation-mediated antitumor immunity and highlight the potential to improve radiotherapy by cGAMP administration and/or by increasing the levels of cGAS in a cancerous cell.

### Example 3. RNAs with Tumor Radio/Chemo-Sensitizing and Immunomodulatory Properties and Methods of their Preparation and Application

Examples 3-5 include examples for RNAs with tumor radio/chemo-sensitizing and immunomodulatory properties and methods of their preparation and application.

Table 3 shows tope 50 snRNAs according to one embodiment of the present invention.

TABLE 3
Top 50 RIG-I binding RNAs (RbRNAs) according
to RepeatMasker annotations

| | | log2FC | log2FC | Mean |
|---|---|---|---|---|
| RNA Species | RNA Class | (set 1) | (set 2) | log2FC |
| U1 | snRNA | 5.988 | 0.312 | 3.150 |
| U2 | snRNA | 5.983 | 1.914 | 3.948 |
| LTR25-int | LTR | 4.172 | 1.586 | 2.879 |
| tRNA-Leu-TTA | tRNA | 3.556 | 1.251 | 2.403 |
| LTR6A | LTR | 2.688 | 1.274 | 1.981 |
| MamGypsy2-LTR | LTR | 2.271 | 1.935 | 2.103 |
| L1MA2 | LINE | 2.240 | 2.073 | 2.156 |
| SSU-rRNA_Hsa | rRNA | 2.206 | 5.834 | 4.020 |
| tRNA-Ile-ATT | tRNA | 1.806 | 0.831 | 1.319 |
| tRNA-Ser-TCG | tRNA | 1.794 | 0.162 | 0.978 |
| G-rich | Other | 1.759 | 0.224 | 0.991 |
| tRNA-Ser-TCA_{—} | tRNA | 1.618 | 0.615 | 1.116 |
| LTR103_Mam | LTR | 1.608 | 2.770 | 2.189 |
| MER76 | LTR | 1.556 | 1.197 | 1.376 |
| tRNA-Ala-GCG | tRNA | 1.536 | 0.854 | 1.195 |
| MER21A | LTR | 1.515 | 1.494 | 1.505 |
| tRNA-Pro-CCG | tRNA | 1.448 | 0.411 | 0.929 |
| tRNA-Leu-CTG | tRNA | 1.445 | 1.025 | 1.235 |
| tRNA-Val-GTG | tRNA | 1.393 | 0.170 | 0.782 |
| LTR21A | LTR | 1.334 | 1.879 | 1.606 |
| GA-rich | Other | 1.331 | 0.757 | 1.044 |
| tRNA-Pro-CCA | tRNA | 1.250 | 0.266 | 0.758 |
| tRNA-Pro-CCY | tRNA | 1.244 | 0.107 | 0.676 |
| tRNA-Gln-CAG | tRNA | 1.234 | 1.136 | 1.185 |
| tRNA-Gly-GGA | tRNA | 1.225 | 0.716 | 0.970 |
| LTR06 | LTR | 1.151 | 3.182 | 2.166 |
| tRNA-Val-GTA | tRNA | 1.143 | 0.868 | 1.005 |
| LTR78 | LTR | 1.120 | 1.622 | 1.371 |
| AmnSINE2 | SINE | 1.114 | 1.073 | 1.094 |
| Charlie17 | Other | 1.100 | 2.147 | 1.623 |
| | Transposable Element | | | |
| tRNA-Gly-GGY | tRNA | 1.085 | 0.232 | 0.659 |
| LTR16E1 | LTR | 1.068 | 0.994 | 1.031 |
| AluYk2 | SINE | 1.044 | 0.006 | 0.525 |
| LTR46-int | LTR | 1.038 | 2.871 | 1.954 |
| Eulor2B | Other | 0.996 | 1.634 | 1.315 |
| | Transposable Element | | | |
| MER70B | LTR | 0.991 | 0.916 | 0.953 |
| MARE6 | LINE | 0.933 | 2.532 | 1.733 |
| tRNA-Thr-ACA | tRNA | 0.889 | 0.100 | 0.494 |
| Charlie9 | Other | 0.871 | 2.422 | 1.647 |
| | Transposable Element | | | |
| LTR2B | LTR | 0.865 | 0.702 | 0.783 |
| X9_LINE | LINE | 0.861 | 1.444 | 1.152 |
| tRNA-Arg-CGA | tRNA | 0.861 | 1.073 | 0.967 |
| LTR30 | LTR | 0.824 | 2.076 | 1.450 |
| LTR58 | LTR | 0.814 | 3.443 | 2.128 |
| MSR1 | Other | 0.811 | 0.627 | 0.719 |
| AluJo | SINE | 0.801 | 0.126 | 0.463 |
| FRAM | SINE | 0.782 | 0.137 | 0.460 |
| MamGyp-int | LTR | 0.774 | 1.592 | 1.183 |
| tRNA-Arg-AGA | tRNA | 0.750 | 0.168 | 0.459 |
| HY3 | scRNA | 0.736 | 0.704 | 0.720 |

Table 4 shows tope 50 snRNAs according to another embodiment of the present invention.

TABLE 4
Top 50 RIG-I binding RNAs (RbRNAs)
according to Gencode annotations

| | | log2FC | log2FC | Mean |
|---|---|---|---|---|
| RNA Species | RNA Class | (set 1) | (set 2) | log2FC |
| EEF1A1P12 | Pseudogene | 8.991 | 6.816 | 7.904 |
| EEF1A1P22 | Pseudogene | 8.772 | 6.618 | 7.695 |
| RPL31P63 | Pseudogene | 7.723 | 5.628 | 6.676 |
| RP11-472I20.1 | Pseudogene | 7.464 | 5.304 | 6.384 |
| RNA28S5 | Pseudogene | 7.276 | 5.196 | 6.236 |
| RP11-506M13.3 | lincRNA | 7.201 | 5.112 | 6.156 |
| MTND4P12 | Pseudogene | 7.169 | 4.979 | 6.074 |
| RPL7P19 | Pseudogene | 7.100 | 5.033 | 6.067 |
| MCTS2P | Pseudogene | 7.089 | 4.924 | 6.006 |
| RP11-386I14.4 | antisense | 5.412 | 6.379 | 5.895 |
| RP11-506B6.3 | Pseudogene | 6.947 | 4.781 | 5.864 |
| RPS4XP13 | Pseudogene | 6.988 | 4.622 | 5.805 |
| RP11-332M2.1 | sense_intronic | 6.896 | 4.567 | 5.731 |
| RP11-380B4.3 | lincRNA | 6.710 | 4.655 | 5.682 |
| EEF1A1P25 | Pseudogene | 6.710 | 4.655 | 5.682 |
| RPS4XP2 | Pseudogene | 6.625 | 4.493 | 5.559 |
| RBBP4P1 | Pseudogene | 6.489 | 4.472 | 5.481 |
| RP11-304F15.3 | antisense | 6.340 | 4.366 | 5.353 |
| RP4-604A21.1 | Pseudogene | 6.340 | 4.366 | 5.353 |
| RPL7P16 | Pseudogene | 6.340 | 4.366 | 5.353 |
| RP11-165H4.2 | Pseudogene | 6.321 | 4.211 | 5.266 |
| CTB-36O1.7 | Pseudogene | 6.667 | 3.782 | 5.224 |
| CTD-2006C1.6 | Pseudogene | 6.209 | 4.133 | 5.171 |
| RP11-563H6.1 | Pseudogene | 6.171 | 4.116 | 5.144 |
| RP5-890O3.9 | sense_intronic | 6.171 | 4.116 | 5.144 |
| RPL23P8 | Pseudogene | 4.698 | 5.437 | 5.067 |
| CTA-392E5.1 | lincRNA | 4.328 | 5.576 | 4.952 |
| RP5-857K21.11 | Pseudogene | 4.447 | 5.307 | 4.877 |
| AC139452.2 | Pseudogene | 5.839 | 3.900 | 4.869 |
| RP11-393N4.2 | Pseudogene | 5.815 | 3.882 | 4.849 |
| RP11-133K1.1 | Pseudogene | 4.478 | 5.172 | 4.825 |
| RP11-378J18.8 | antisense | 5.623 | 3.650 | 4.636 |
| RPL5P34 | Pseudogene | 4.306 | 4.867 | 4.586 |
| RPS4XP3 | Pseudogene | 4.089 | 5.003 | 4.546 |
| RAD21-AS1 | antisense | 6.082 | 2.874 | 4.478 |
| EEF1A1P4 | Pseudogene | 3.853 | 5.014 | 4.433 |
| MT-TL1 | Mt_tRNA | 4.010 | 4.851 | 4.431 |
| HNRNPA3P3 | Pseudogene | 3.999 | 4.802 | 4.400 |
| RP13-216E22.4 | lincRNA | 5.557 | 3.229 | 4.393 |
| RPL5P23 | Pseudogene | 5.557 | 3.229 | 4.393 |
| SLIT2-IT1 | sense_intronic | 3.593 | 5.121 | 4.357 |
| RP11-785H5.1 | Pseudogene | 3.714 | 4.946 | 4.330 |
| RP11-627K11.1 | Pseudogene | 3.508 | 5.115 | 4.311 |
| RP11-750B16.1 | Pseudogene | 3.814 | 4.744 | 4.279 |
| EEF1B2P3 | Pseudogene | 4.156 | 4.330 | 4.243 |
| RP11-17A4.1 | Pseudogene | 3.753 | 4.681 | 4.217 |
| CTD-2161E19.1 | Pseudogene | 5.294 | 3.103 | 4.199 |
| AC022210.2 | Pseudogene | 3.690 | 4.613 | 4.152 |
| HNRNPA1P35 | Pseudogene | 3.042 | 5.189 | 4.116 |

TABLE 5
All mapped reads identified using RepeatMasker

| RNA Species | RNA Class | log2FC |
|---|---|---|
| U1 | snRNA | 5.988 |
| U2 | snRNA | 5.983 |
| LTR25-int | LTR | 4.172 |
| tRNA-Leu-TTA | tRNA | 3.556 |
| LTR6A | LTR | 2.688 |
| MamGypsy2-LTR | LTR | 2.271 |
| L1MA2 | LINE | 2.240 |
| SSU-rRNA_Hsa | rRNA | 2.206 |
| tRNA-Ile-ATT | tRNA | 1.806 |
| tRNA-Ser-TCG | tRNA | 1.794 |
| G-rich | Other | 1.759 |
| tRNA-Ser-TCA_{—} | tRNA | 1.618 |
| LTR103_Mam | LTR | 1.608 |
| MER76 | LTR | 1.556 |
| tRNA-Ala-GCG | tRNA | 1.536 |
| MER21A | LTR | 1.515 |
| tRNA-Pro-CCG | tRNA | 1.448 |
| tRNA-Leu-CTG | tRNA | 1.445 |
| tRNA-Val-GTG | tRNA | 1.393 |
| LTR21A | LTR | 1.334 |
| GA-rich | Other | 1.331 |
| tRNA-Pro-CCA | tRNA | 1.250 |
| tRNA-Pro-CCY | tRNA | 1.244 |
| tRNA-Gln-CAG | tRNA | 1.234 |
| tRNA-Gly-GGA | tRNA | 1.225 |
| LTR06 | LTR | 1.151 |
| tRNA-Val-GTA | tRNA | 1.143 |
| LTR78 | LTR | 1.120 |
| AmnSINE2 | SINE | 1.114 |
| Charlie17 | Other | 1.100 |
| | Transposable Element | |
| tRNA-Gly-GGY | tRNA | 1.085 |
| LTR16E1 | LTR | 1.068 |
| AluYk2 | SINE | 1.044 |
| LTR46-int | LTR | 1.038 |
| Eulor2B | Other | 0.996 |
| | Transposable Element | |
| MER70B | LTR | 0.991 |
| MARE6 | LINE | 0.933 |
| tRNA-Thr-ACA | tRNA | 0.889 |
| Charlie9 | Other | 0.871 |
| | Transposable Element | |
| LTR2B | LTR | 0.865 |
| X9_LINE | LINE | 0.861 |
| tRNA-Arg-CGA | tRNA | 0.861 |
| LTR30 | LTR | 0.824 |
| LTR58 | LTR | 0.814 |
| MSR1 | Other | 0.811 |
| AluJo | SINE | 0.801 |
| FRAM | SINE | 0.782 |
| MamGyp-int | LTR | 0.774 |
| tRNA-Arg-AGA | tRNA | 0.750 |
| HY3 | scRNA | 0.736 |
| MER92C | LTR | 0.715 |
| tRNA-Met_{—} | tRNA | 0.709 |
| UCON85 | Other | 0.695 |
| AluSc8 | SINE | 0.693 |
| Penelope1_Vert | LINE | 0.692 |
| Helitron2Na_Mam | Other | 0.689 |
| | Transposable Element | |
| Zaphod2 | Other | 0.681 |
| | Transposable Element | |
| OldhAT1 | Other | 0.664 |
| | Transposable Element | |
| tRNA-Thr-ACY | tRNA | 0.661 |
| AluSg4 | SINE | 0.655 |
| LTR45B | LTR | 0.643 |
| L1PB1 | LINE | 0.633 |
| UCON23 | Other | 0.628 |
| | Transposable Element | |
| tRNA-Phe-TTY | tRNA | 0.620 |
| UCON80_AMi | Other | 0.611 |
| HSMAR1 | Other | 0.611 |
| | Transposable Element | |
| LTR22B1 | LTR | 0.608 |
| AluSg7 | SINE | 0.598 |
| MER9a3 | LTR | 0.598 |
| FLAM_A | SINE | 0.597 |
| AmnSINE1 | SINE | 0.596 |
| HERVS71-int | LTR | 0.596 |
| A-rich | Other | 0.579 |
| X6A_LINE | LINE | 0.573 |
| UCON70 | Other | 0.569 |
| Tigger3d | Other | 0.543 |
| | Transposable Element | |
| MIR1_Amn | SINE | 0.538 |
| LTR5A | LTR | 0.533 |
| AluSc | SINE | 0.533 |
| AluSx3 | SINE | 0.527 |
| MER97b | Other | 0.517 |
| | Transposable Element | |
| LTR13A | LTR | 0.516 |
| SVA_F | Other | 0.514 |
| | Transposable Element | |
| MER61A | LTR | 0.508 |
| tRNA-Lys-AAG | tRNA | 0.491 |
| AluY | SINE | 0.489 |
| L1MB1 | LINE | 0.489 |
| AluSq2 | SINE | 0.463 |
| U7 | snRNA | 0.456 |
| LTR13 | LTR | 0.443 |
| L1PB4 | LINE | 0.407 |
| AluJr | SINE | 0.389 |
| LTR75_1 | LTR | 0.385 |
| HERVFH21-int | LTR | 0.383 |
| Charlie12 | Other | 0.378 |
| | Transposable Element | |
| LTR48B | LTR | 0.363 |
| AluSx1 | SINE | 0.363 |
| LTR1B | LTR | 0.343 |
| LTR16D1 | LTR | 0.342 |
| tRNA-Leu-CTA_{—} | tRNA | 0.332 |
| X8_LINE | LINE | 0.330 |
| LTR12F | LTR | 0.326 |
| SVA_D | Other | 0.321 |
| | Transposable Element | |
| MER51C | LTR | 0.319 |
| LTR41 | LTR | 0.319 |
| MER49 | LTR | 0.316 |
| MER52C | LTR | 0.314 |
| MamGypLTR3 | LTR | 0.305 |
| tRNA-Met | tRNA | 0.305 |
| Tigger23a | Other | 0.296 |
| | Transposable Element | |
| MER51D | LTR | 0.290 |
| UCON8 | Other | 0.288 |
| | Transposable Element | |
| LTR10B2 | LTR | 0.276 |
| Eutr2 | Other | 0.269 |
| | Transposable Element | |
| UCON73 | Other | 0.239 |
| | Transposable Element | |
| LTR61 | LTR | 0.233 |
| LTR12_{—} | LTR | 0.222 |
| LTR35 | LTR | 0.219 |
| Tigger19b | Other | 0.212 |
| | Transposable Element | |
| FLAM_C | SINE | 0.206 |
| MST-int | LTR | 0.203 |
| Alu | SINE | 0.202 |
| MER131 | Other | 0.196 |
| | Transposable Element | |
| MamRep38 | Other | 0.190 |
| | Transposable Element | |
| EuthAT-N1a | Other | 0.180 |
| | Transposable Element | |
| MER91B | Other | 0.178 |
| | Transposable Element | |
| Tigger17 | Other | 0.177 |
| | Transposable Element | |
| LTR26E | LTR | 0.175 |
| tRNA-Ser-TCY | tRNA | 0.170 |
| MLT1O | LTR | 0.167 |
| LTR19C | LTR | 0.165 |
| tRNA-Glu-GAG_{—} | tRNA | 0.164 |
| MamRep564 | Other | 0.150 |
| MER54B | LTR | 0.150 |
| MER102a | Other | 0.148 |
| | Transposable Element | |
| tRNA-Thr-ACG | tRNA | 0.145 |
| LTR108a_Mam | LTR | 0.138 |
| LTR41B | LTR | 0.131 |
| AluSz | SINE | 0.125 |
| LTR33C | LTR | 0.105 |
| LTR3B_{—} | LTR | 0.095 |
| LTR33B | LTR | 0.085 |
| MER68-int | LTR | 0.083 |
| AluJb | SINE | 0.075 |
| MamRTE1 | LINE | 0.066 |
| U8 | snRNA | 0.065 |
| MER65D | LTR | 0.063 |
| LTR35A | LTR | 0.063 |
| LTR13_{—} | LTR | 0.060 |
| MER77 | LTR | 0.058 |
| MARNA | Other | 0.055 |
| | Transposable Element | |
| LTR10F | LTR | 0.053 |
| LTR22E | LTR | 0.048 |
| LTR40b | LTR | 0.037 |
| LFSINE_Vert | SINE | 0.030 |
| LTR89B | LTR | 0.027 |
| LTR10A | LTR | 0.025 |
| tRNA-Leu-TTA_m_{—} | tRNA | 0.024 |
| LSU-rRNA_Hsa | rRNA | 0.020 |
| X10b_DNA | Other | 0.015 |
| | Transposable Element | |
| LTR12D | LTR | 0.014 |
| HERV1_LTRa | LTR | 0.013 |
| MER9a2 | LTR | 0.011 |
| LTR5B | LTR | 0.009 |
| MamTip2 | Other | 0.009 |
| | Transposable Element | |
| EUTREP16 | LTR | 0.003 |

### Example 4. Cancer Therapies Activate RIG-I-Like Receptor Pathway Through Endogenous Non-Coding RNAs

Emerging evidence indicates that ionizing radiation (IR) and chemotherapy activate Type I interferon (IFN) signaling in tumor and host cells. However, the mechanism of induction is poorly understood. We identified a novel radioprotective role for the DEXH box RNA helicase LGP2 (DHX58) through its suppression of IR-induced cytotoxic IFN-beta (Widau et al., 2014). LGP2 inhibits activation of the RIG-I-like receptor (RLR) pathway upon binding of viral RNA to the cytoplasmic sensors RIG-I (DDX58) and MDA5 (IFIH1) and subsequent IFN signaling via the mitochondrial adaptor protein MAVS (IPS1). Here we show that MAVS is necessary for IFN-beta induction and interferon-stimulated gene expression in the response to IR. Suppression of MAVS conferred radioresistance in normal and cancer cells. Germline deletion of RIG-I, but not MDA5, protected mice from death following total body irradiation, while deletion of LGP2 accelerated the death of irradiated animals. In human tumors depletion of RIG-I conferred resistance to IR and different classes of chemotherapy drugs. Mechanistically, IR stimulated the binding of cytoplasmic RIG-I with small endogenous non-coding RNAs (sncRNAs), which triggered IFN-beta activity. We demonstrate that the small nuclear RNAs U1 and U2 translocate to the cytoplasm after IR treatment, thus stimulating the formation of RIG-I: RNA complexes and initiating downstream signaling events. Taken together, these findings suggest that the physiologic responses to radio-/chemo-therapy converge on an antiviral program in recruitment of the RLR pathway by a sncRNA-dependent activation of RIG-I which commences cytotoxic IFN signaling. Importantly, activation of interferon genes by radiation or chemotherapy is associated with a favorable outcome in patients undergoing treatment for cancer. To our knowledge, this is the first demonstration of a cell-intrinsic response to clinically relevant genotoxic treatments mediated by an RNA-dependent mechanism.

Introduction

Accumulating data indicate a link between ionizing radiation (IR) and interferon (IFN) signaling. IFN signaling activates multiple interferon-stimulated genes (ISGs) and leads to growth arrest and cell death in exposed cell populations (Amundson et al., 2004; Khodarev et al., 2007; Tsai et al., 2007; Khodarev et al., 2005). It has been demonstrated that IR-induced tumor-derived type I IFN production is important for improved tumor responses (Burnette et al., 2011; Lim et al., 2014), suggesting that Type I IFN is an essential part of IR-delivered tumor cytotoxicity and/or activation of the immune system (Khodarev et al., 2007; Burnette et al., 2012; Khodarev et al., 2012). However, molecular mechanisms governing tumor cell-intrinsic IR-mediated IFN activation are largely unknown.

Recently we identified DEXH box RNA helicase LGP2 (DHX58) as a negative regulator of IR-induced cytotoxic IFN-beta production contributing to cell-autonomous radioprotective effects in cancer cells (Widau et al., 2014). LGP2 is a cytoplasmic RIG-I-like receptor (RLR) which suppresses IFN signaling in the response to viral double-stranded RNA (Bruns and Horvath, 2014). RLRs are members of pattern recognition receptors (PRRs) which mediate the induction of IFN signaling in the response to pathogens due to abnormal accumulation of ribonucleic acids in the cytoplasm or extracellular space (Akira et al., 2006). RLRs are the part of innate immunity, evolved in the eukaryotic cells for protection from pathogenes based on the molecular recognition of macromolecules, specific for these foreign organisms (see Akira et al., 2006; Loo and Gale, 2011; Iwasaki and Medzhitov, 2015; Medzhitov and Janeway, 2000 for reviews). Identification of LGP2 as the regulatory protein in the response to IR posed an intriguing question about implication of pathogen RNA recognition systems in the response to IR damage, traditionally associated with DNA damage recognition systems.

RLRs are presented by 3 major primary RNA sensors (RIG-I, MDA5 and LGP2) and one common adapter protein MAVS (Mitochondrial anti-viral signaling protein-see FIG. 1a). RIG-I and MDA5 are activated through binding with RNA molecules, which release their CARD domains and activates their interactions with MAVS (see Goubau et al., 2014; Cai and Chen, 2014; Reikine et al., 2014). Activated MAVS recruits IRF3 and NFkB and eventually leads to the activation of IFN-beta, through multiple intermediate steps which are still under investigation. LGP2 has context-specific functions, but often acts as the suppressor of RNA-dependent IFN-beta production (see Bruns and Horvath, 2014; Bruns et al., 2014 for reviews), consistent with our observations of the LGP2 functions in the response of various types of tumor cells to IR (Widau et al., 2014).

RIG-I and MDA5 are able to recognize foreign viral RNAs based on their primary and secondary structure, size, structure of 5′ends of RNAs and/or recognition of methylated patterns in the 5′ capping structures of RNAs (Goubau et al., 2014; Hagmann et al., 2013; Devarkar et al., 2016). As well, concentration of RNAs in the cytoplasmic fraction may be important in activation of these primary RNA sensors (Boelens et al., 2014).

In the current paper we used combination of genetic, biochemical and bioinformatics approaches to systematically investigate effects of the each component of RLR pathway on the ability of IR and chemotherapy to kill normal and tumor cells and produce IFN-beta. Our data indicate that RLR pathway is necessary and sufficient in the ability of IR and chemotherapy to induce cytotoxic response and IFN-beta production. RLR pathway is activated by endogenous small non-coding RNAs which are accumulated in the cytoplasm in the response to genotoxic stress, binds to RIG-I and activate down-stream IFN-beta. RLR pathway confers tumors response in in vivo xenograft models and is responsible for the lethal gastrointestinal injury after total body irradiation (TBI). Finally, using analysis of the currently available databases we demonstrated that RLR pathway is involved in the response to radio/chemotherapy in the cervical, breast, bladder and rectal cancer, which warrants design of the appropriate biomarkers for clinical applications and search for druggable targets responsible for regulation of this pathway (Khodarev et al., 2012; Weichselbaum et al., 2008; Duarte et al., 2012).

Results

1. MAVS is Necessary and Sufficient for the Ability of IR to Induce IFN Signaling and Cell Killing

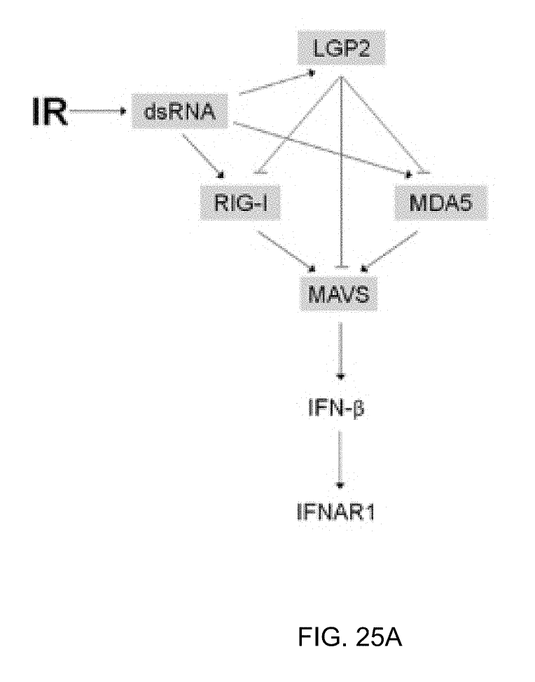

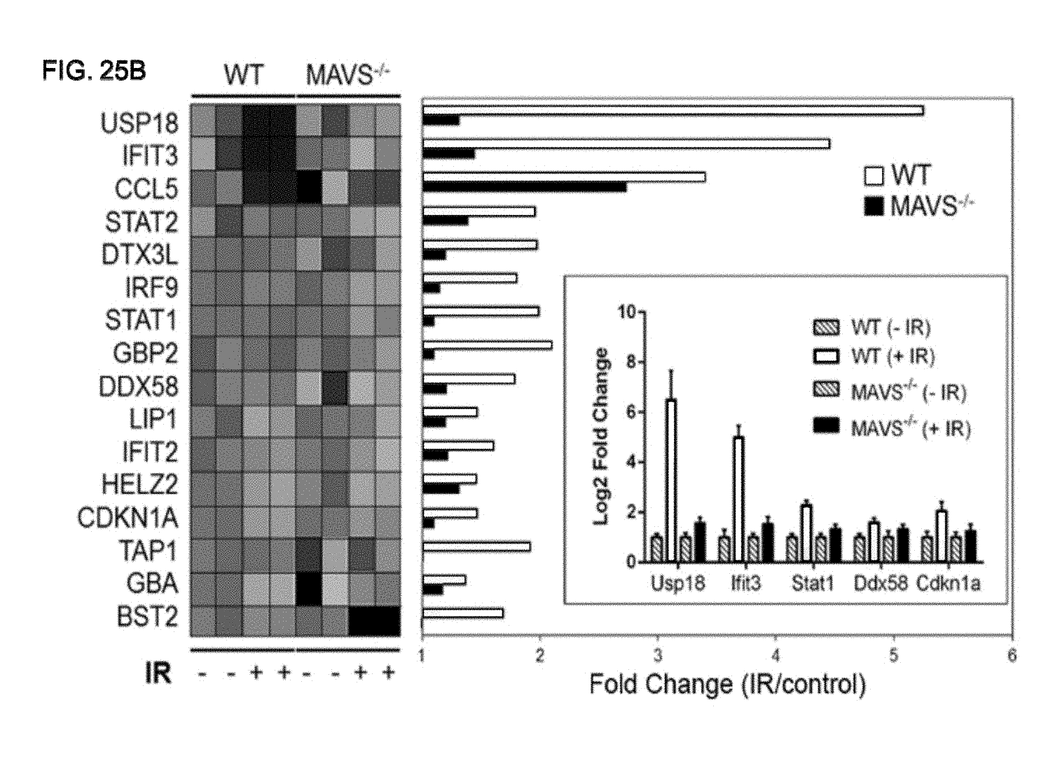

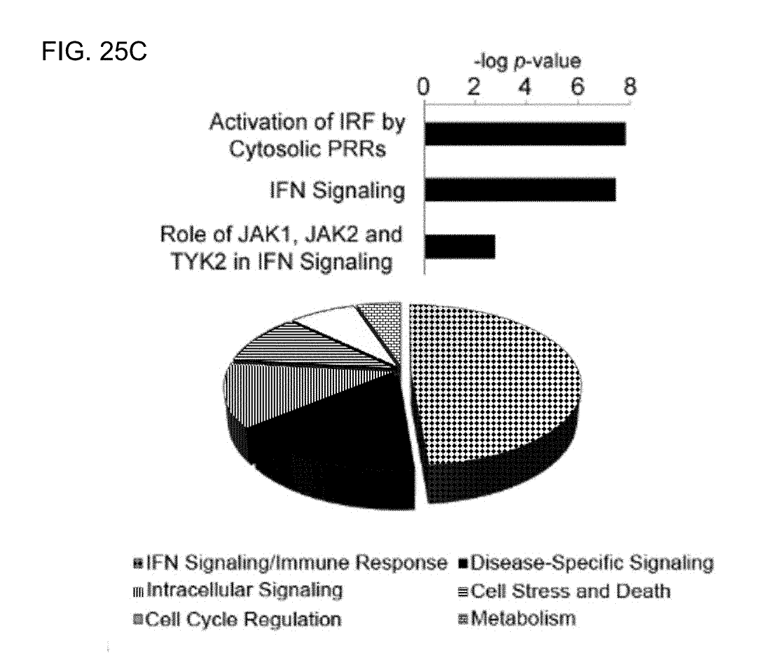

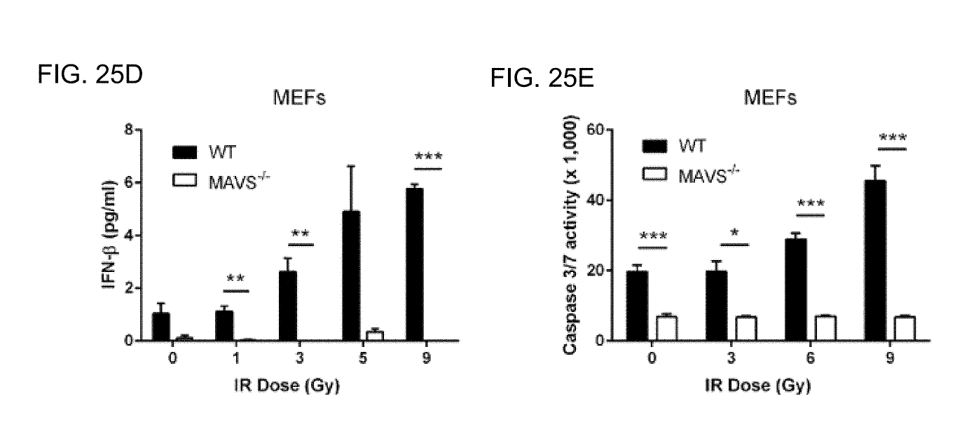

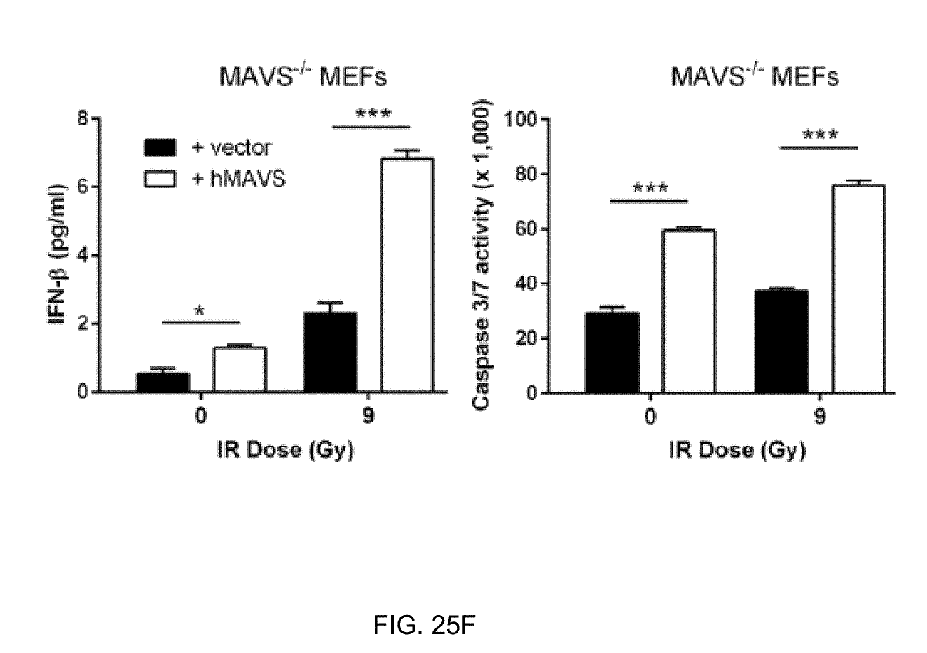

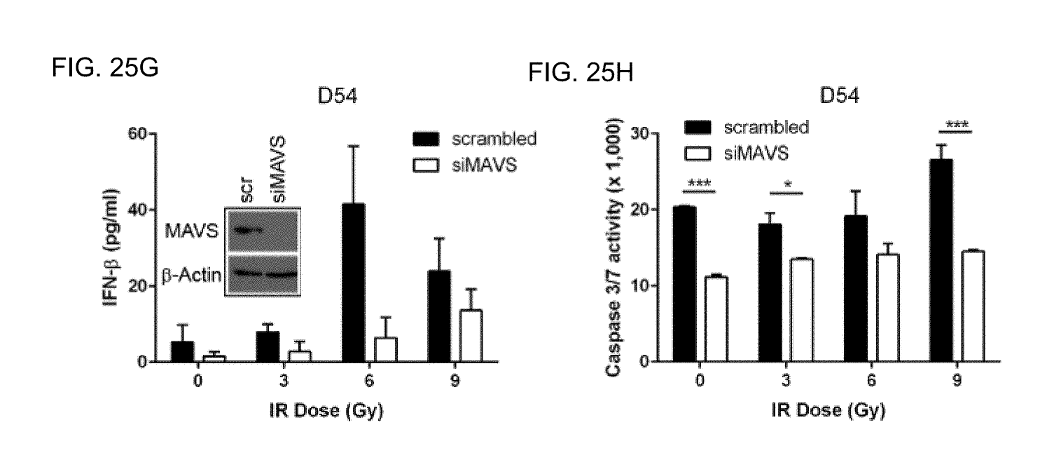

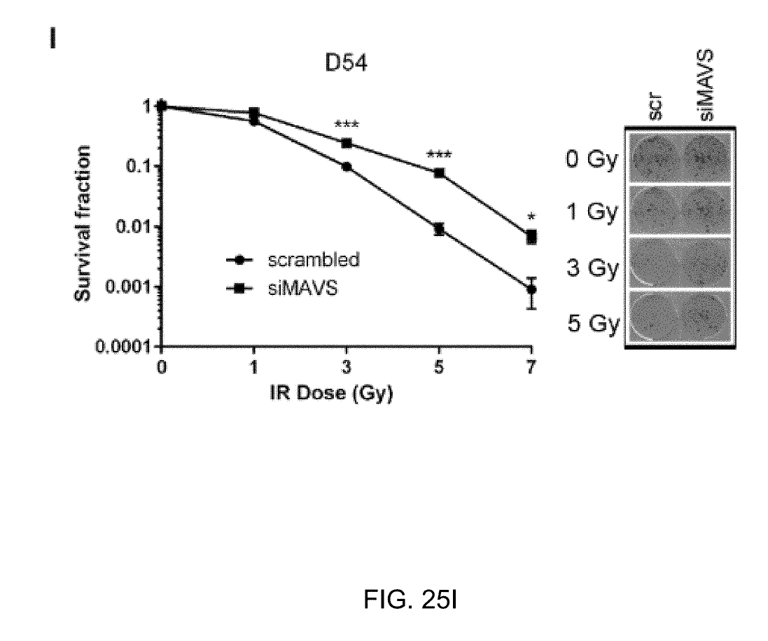

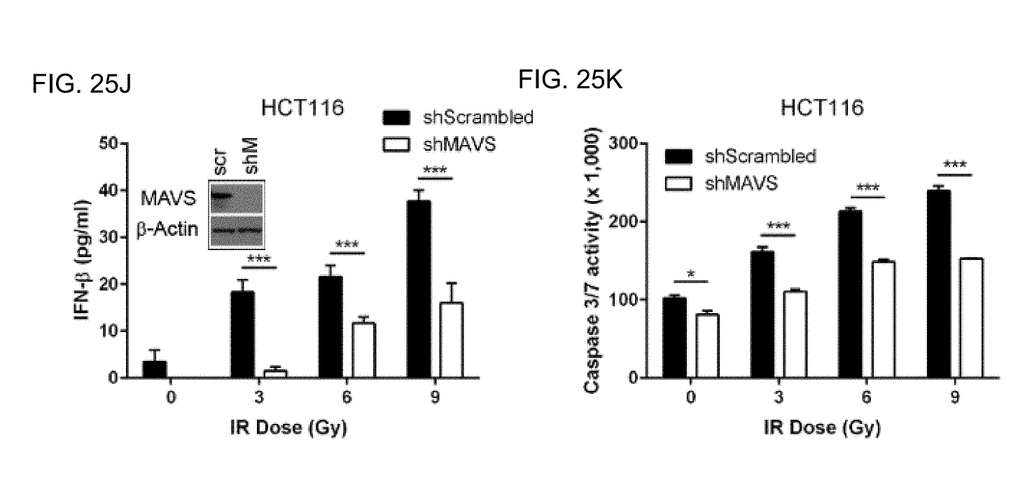

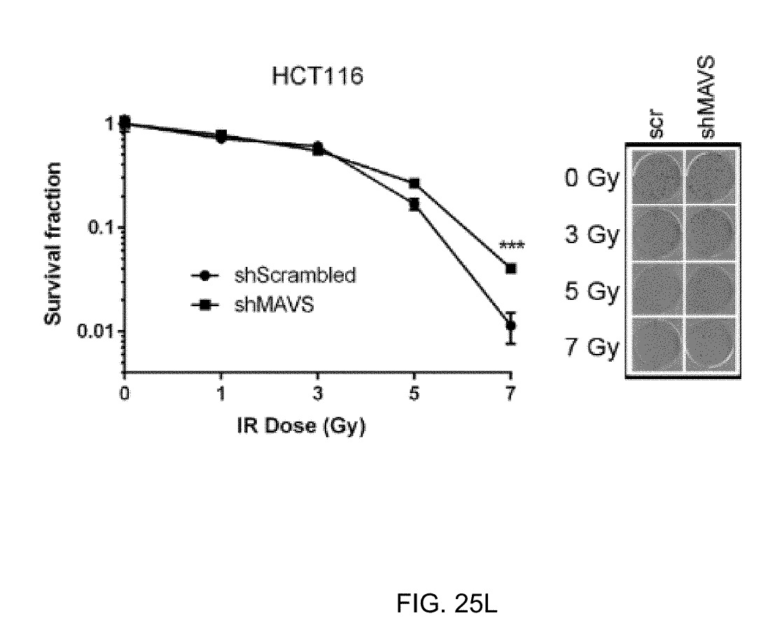

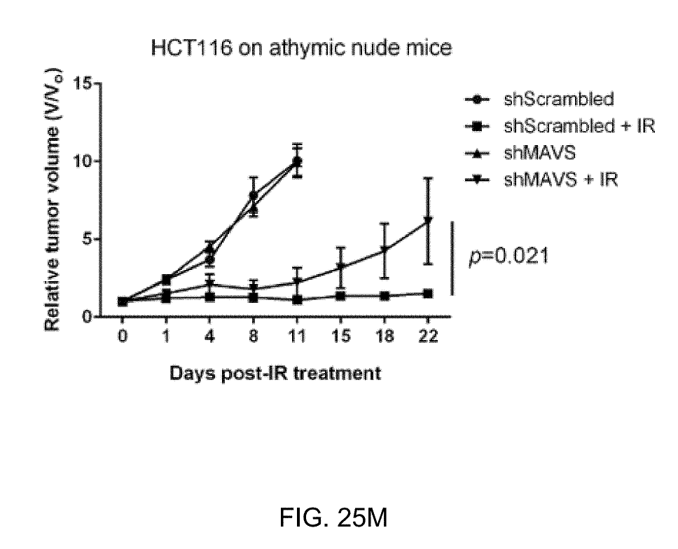

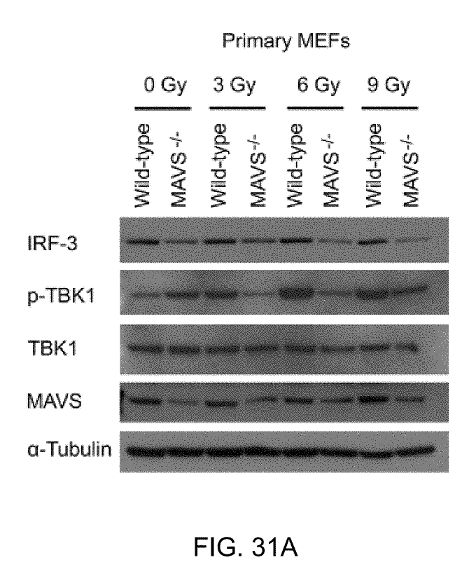

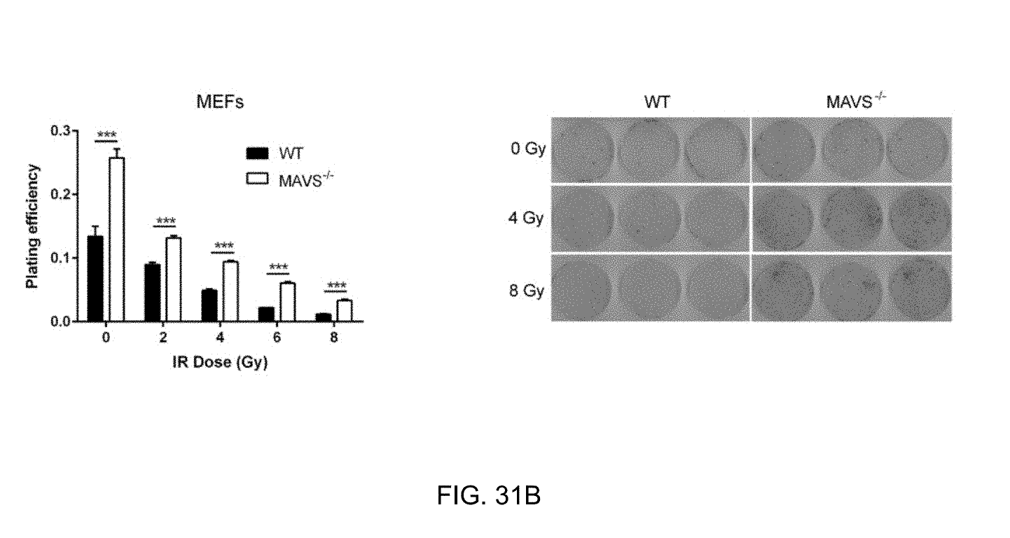

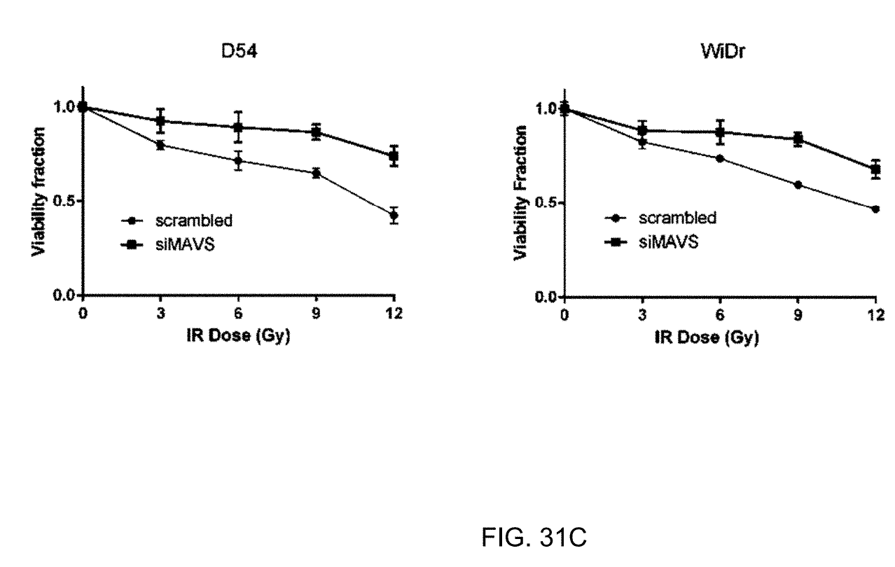

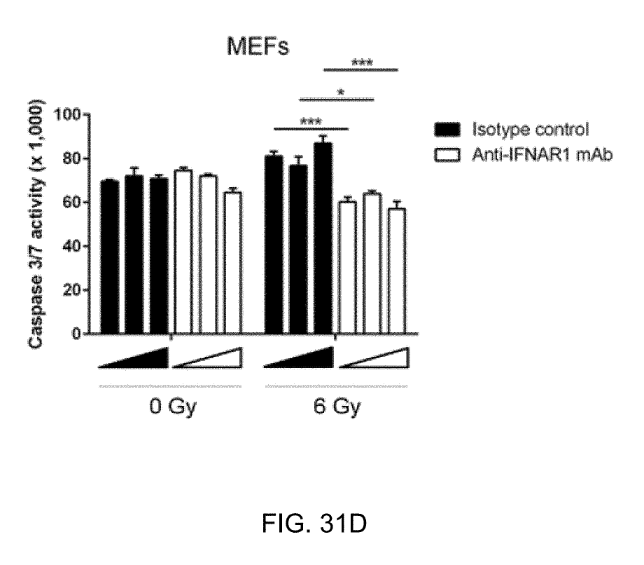

We identified the role for RLR signaling in the response to IR. Following irradiation, endogenous RNA moieties are upregulated in the cytoplasm and thereby recognized by cytoplasmic RNA sensors (FIG. 25A). Irradiation (6 Gy) induced the overexpression of 82 genes in C57BL/6 wild-type (WT) primary mouse embryonic fibroblasts (MEFs) at 48 hours following treatment. Sixteen of these genes were identified as type I ISGs (FIGS. 25B and 25C). Notably, expression of RIG-I (DDX58), but not MDA5 (IFIH1), was induced by IR. In contrast, MAVS^{-/-} MEFs failed to induce type I ISG expression in irradiated cells (FIG. 25B). IR led to a dose-dependent accumulation of IFN-beta in WT MEFs which was absent in MAVS^{-/-} MEFs (FIG. 25D). Consistently, Western blot analyses reveal that MAVS^{-/-} MEFs have lower phosphorylated TBK1 and basal IRF3 levels compared to the WT controls in response to increasing dose of IR (FIG. 31A). WT MEFs also demonstrated an IR dose-dependent activation of caspases 3/7 which was blunted in MAVS^{-/-} MEFs (FIG. 25E). The differences in caspase activation paralleled differences in clonogenic survival of WT and MAVS^{-/-} SV40-transformed MEFs (FIG. 31B). Reconstitution of MAVS restored IFN-beta production and IR-induced caspase activation in MAVS^{-/-} MEFs (FIG. 25F). Consistent with these findings, IR induced a cytotoxic IFN-beta response in human D54 glioblastoma (FIGS. 25G, 25H and 25I) and HCT116 colorectal carcinoma cell lines (FIGS. 25J, 25K and 25L) which was suppressed by MAVS depletion. Interestingly, basal production and IR-induced levels of secreted IFN-beta were higher in tumor cells as compared with primary fibroblasts. MAVS knockdown in WiDr human colon adenocarcinoma cells also conferred radioresistance (FIG. 31C). We then investigated the response to IR of the corresponding tumors established as hind limb xenografts in athymic nude mice. As shown in FIG. 25M, depletion of MAVS led to a significant tumor regrowth following IR with no apparent effect on untreated tumors.

Type I IFN receptor signaling was necessary for the cell death following IR exposure as evidenced by suppression of IR-induced apoptosis after administration of neutralizing anti-IFNAR1 monoclonal antibody (FIG. 31D). Taken together, these data demonstrated that MAVS-dependent signaling confers IR-mediated cytotoxicity through IFN-beta production.

2. RIG-1 is the Critical RNA Sensor, Responsible for IR-Induced and Chemotherapy Induced Cell Killing.

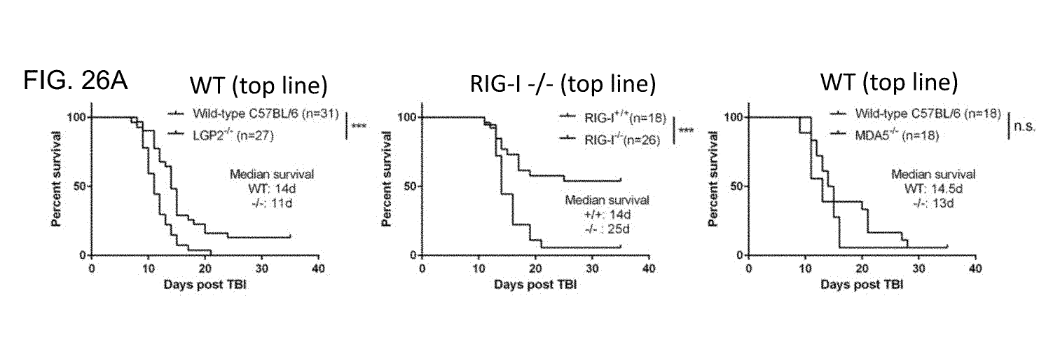

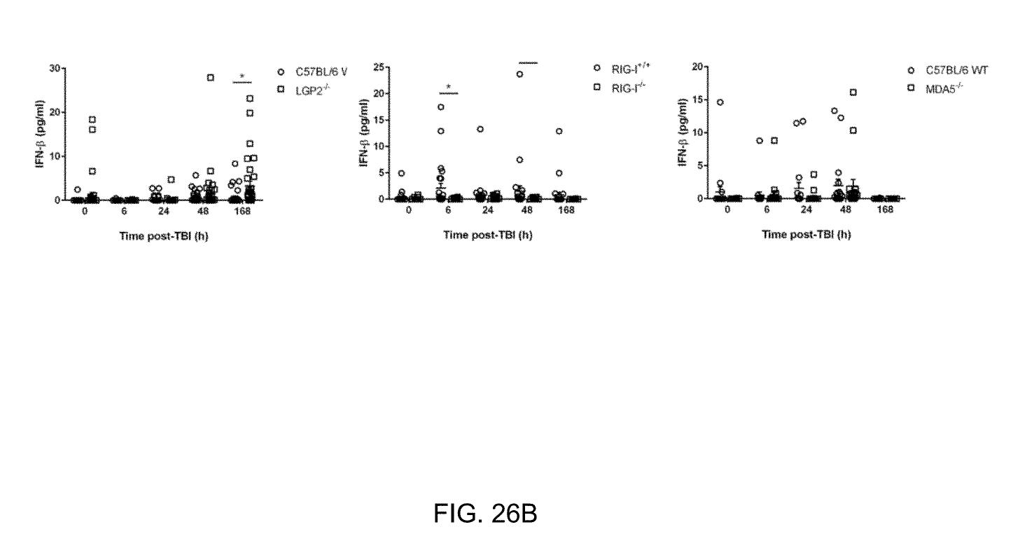

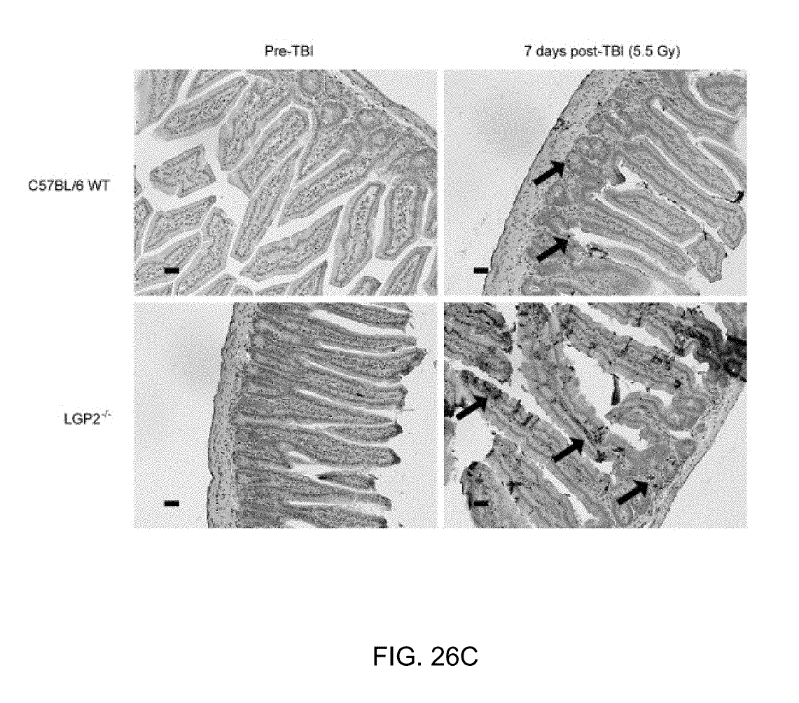

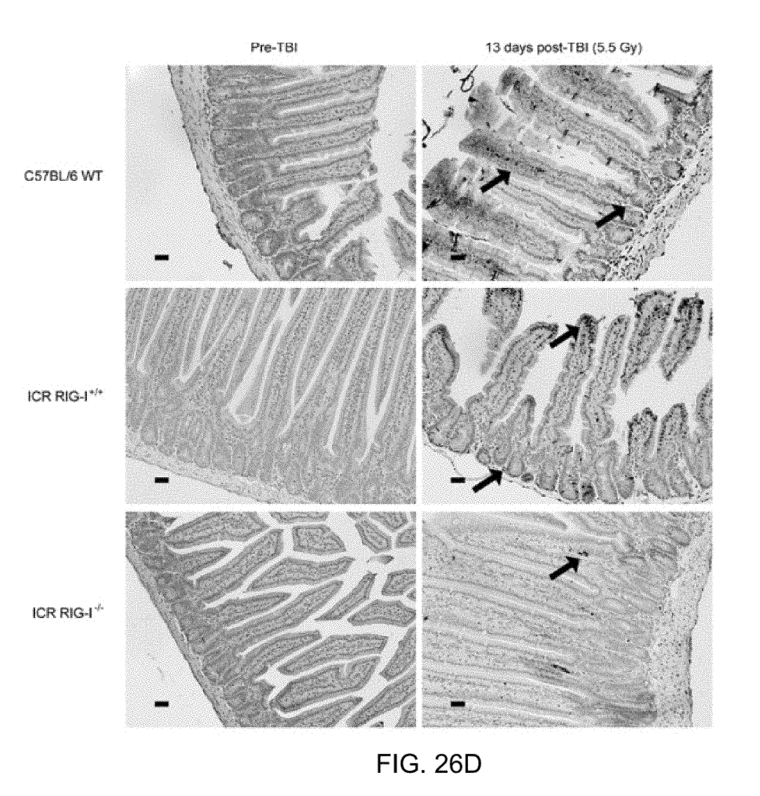

RNA sensing via MAVS-dependent signaling is mediated by three RNA sensors—LGP2, RIG-I and MDA5. RIG-I and MDA5 promote MAVS activation, while LGP2 is thought to regulate RIG-I and MDA5 in cell- and viral-specific context (Yoneyama et al., 2005; Rothenfusser et al., 2005; Komuro and Horvath, 2006). We tested whether LGP2, RIG-I, and MDA5 contribute to the total body irradiation (TBI; 5.5 Gy) response. We found that LGP2 conferred radioprotection, while RIG-I mediated radiosensitivity (FIG. 26A). LGP2 expression inversely correlated with IFN-beta secretion, whereas RIG-I promoted IFN-beta production in the response to TBI (FIG. 26B). LGP2−/− mice demonstrated elevated levels of apoptosis in intestinal crypt cells and epithelial cells comprising the microvilli and lamina propria as compared to wild-type animals (FIG. 26C), which is consistent with death due to radiation-induced gastrointestinal injury (Anno et al., 2003; Hall and Giaccia, 2006). In contrast, RIG-I−/− mice showed minimal IR-induced intestinal apoptosis and exhibited higher survival rates compared to the RIG-I+/+ controls (FIG. 26D). On the other hand, MDA5 exerted no measurable effect on radiosensitivity or IFN-beta production (FIG. 26A).

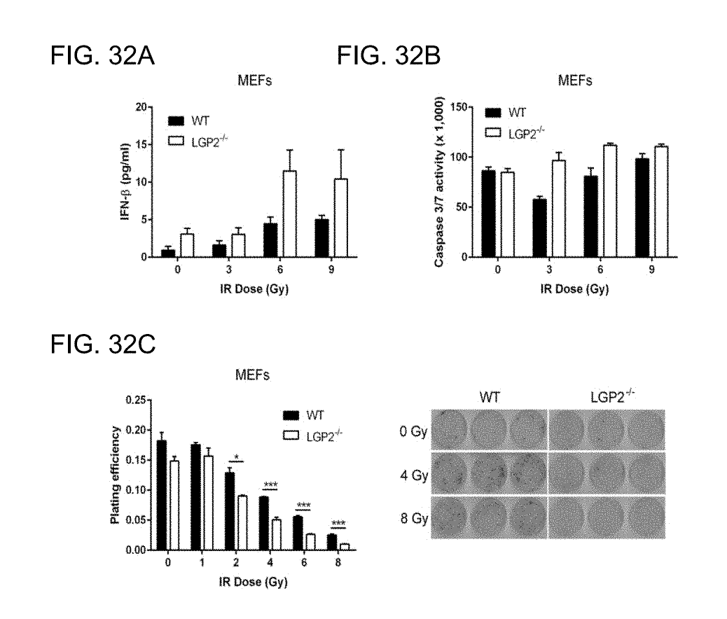

At the cellular level, LGP2−/− MEFs exhibited increased IFN-beta production, caspase 3/7 activation, and decreased clonogenic survival after IR exposure (FIGS. 32A, 32B and 32C). These data supported the notion that LGP2 suppresses IR-induced RIG-I-dependent IFN-beta signaling (Widau et al., 2014). The data indicated that RLR-dependent Type I IFN production is an important component of the lethal effects of IR, which may contribute to the GI death, induced by TBI.

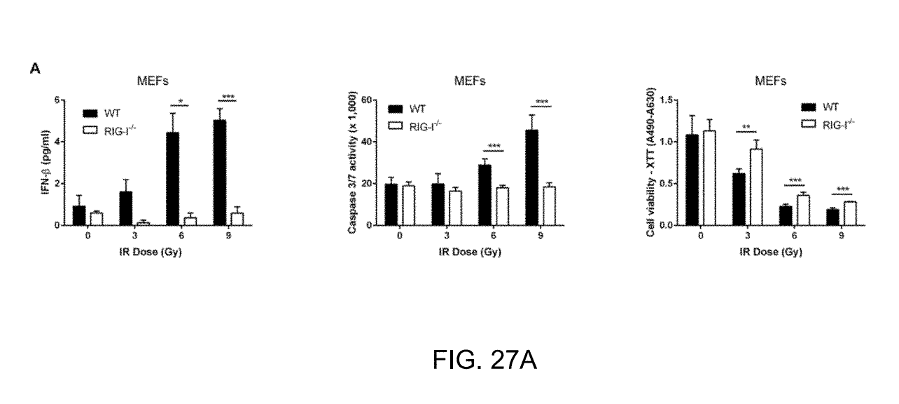

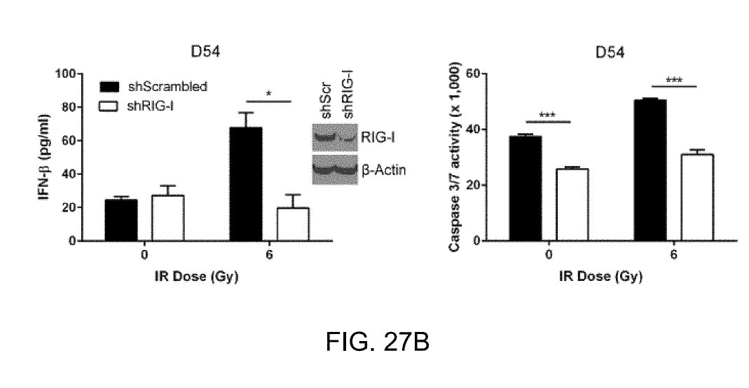

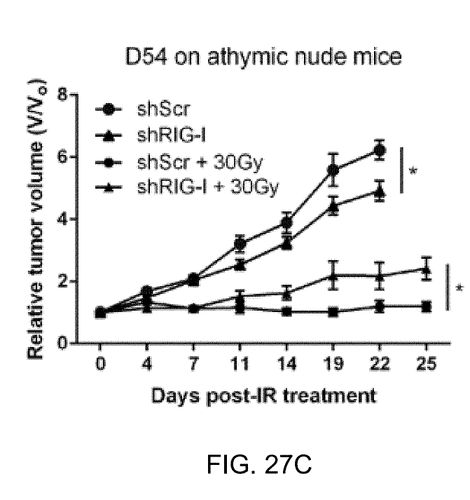

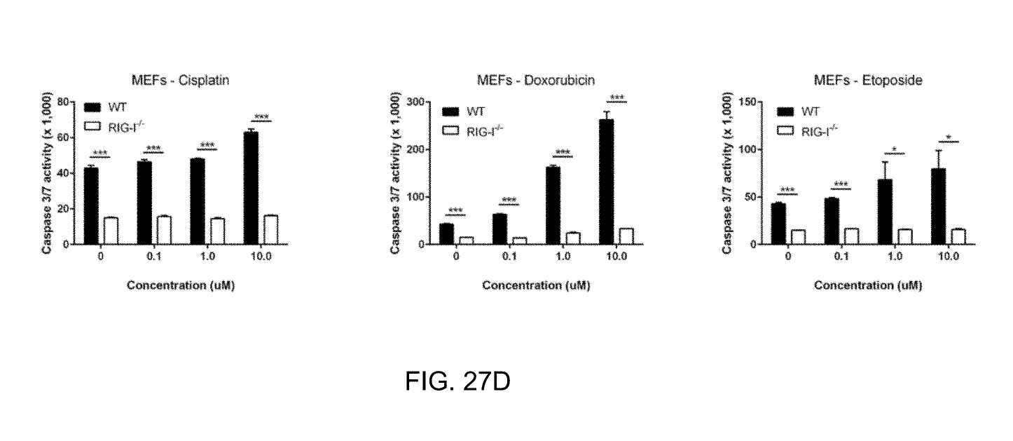

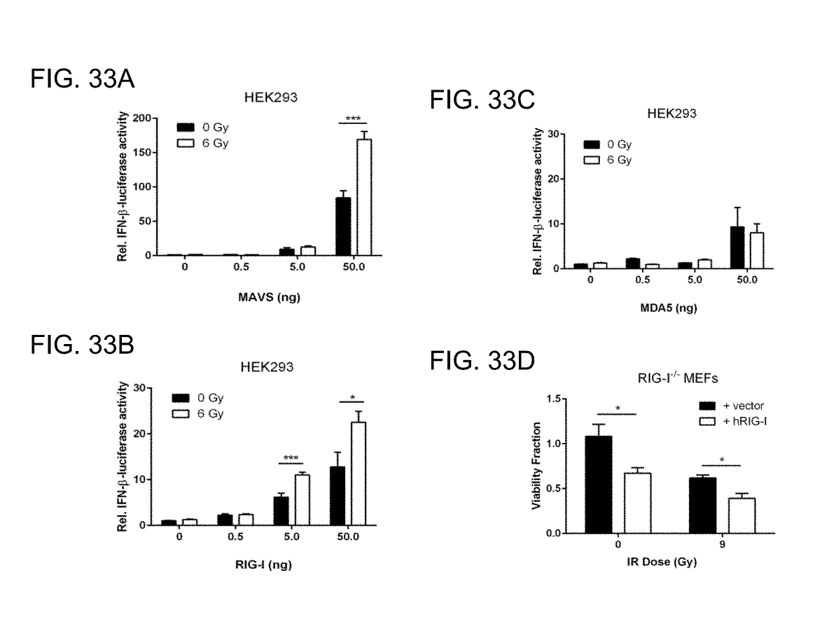

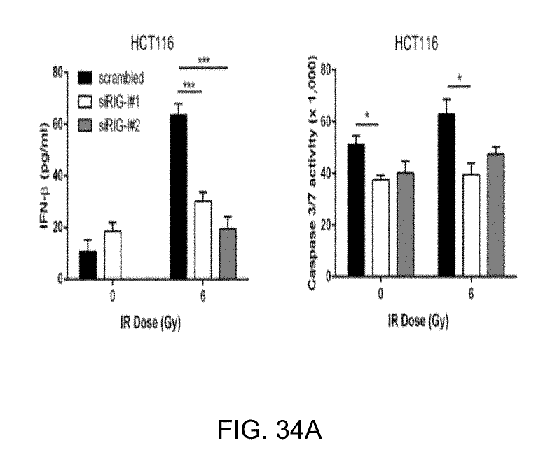

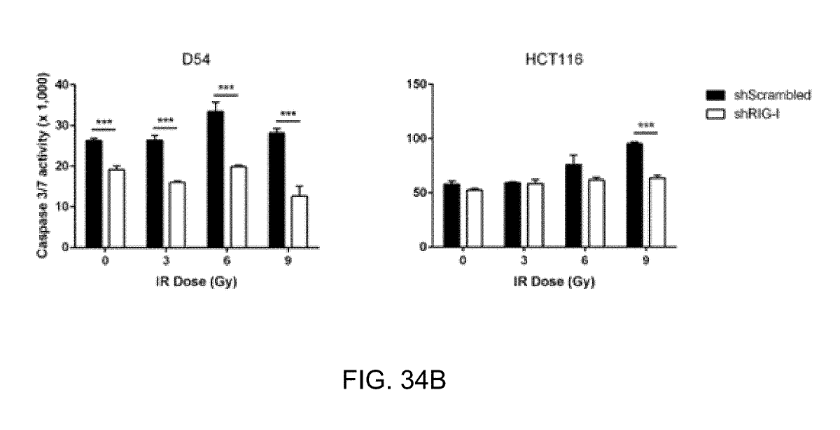

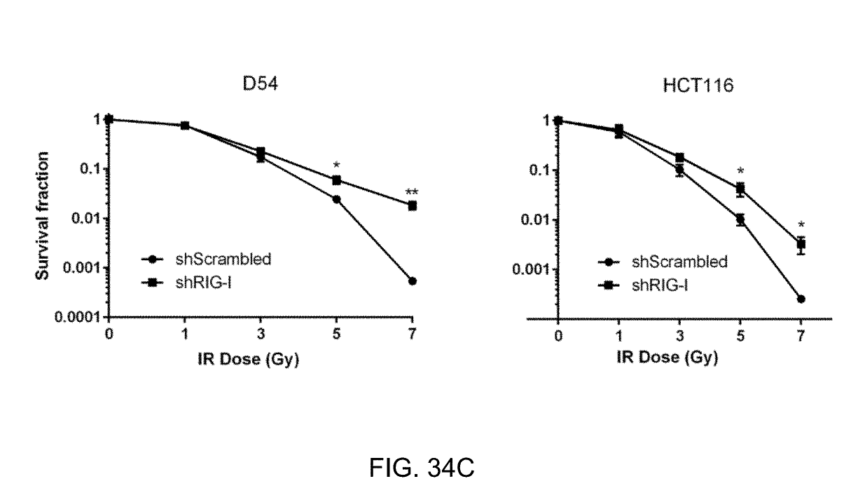

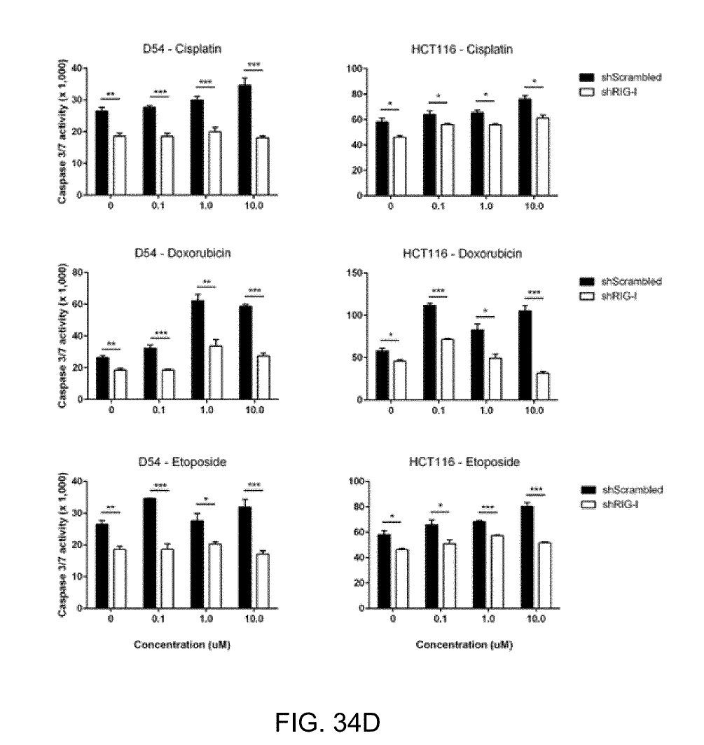

We further examined the relative contributions of RIG-I and MDA5 in IFN-beta induction after exposure to IR. Ectopic expression of MAVS or RIG-I activated the IFN-beta promoter in an IR-dependent manner (FIGS. 33A and 33B). In contrast, overexpression of MDA5 led to a modest activation of IFN-beta at the basal level, but not by IR (FIG. 33C). We therefore focused on the role of RIG-I in IR-induced cytotoxicity. We found that irradiated RIG-I^{-/-} MEFs were deficient in both the IFN-beta response and caspase 3/7 activity, and demonstrated increased survival as compared to wild-type MEFs (FIG. 27A). Reconstitution of MEFs by full-length RIG-I restored radiosensitivity (FIG. 33D). Similarly, D54 and HCT116 tumor cells depleted of RIG-I exhibited suppression of IFN-beta secretion and caspase 3/7 responses to IR as well as radioresistance in clonogenic assays (FIG. 27B and FIGS. 34A, 34B and 34C). To test the effects of tumor cell-derived IFN on in vivo growth and radioresistance, we established D54 human tumor xenografts with stable suppression of RIG-I in athymic nude mice (FIG. 27C). In the absence of radiation, depletion of RIG-I reduced tumor growth rate as compared to control cells. In contrast, tumor regrowth was greater in RIG-I knockdown tumors after IR treatment. Collectively, these data supported a critical role for RIG-I in mediating the RLR response of normal and tumor cells to IR.

Recently it was demonstrated that treatment of fibrosarcomas with anthracyclines, such as doxorubicin, led to a cell-autonomous induction of ISGs via Toll-like receptor 3 but not the cytosolic sensor MDA5 (Sistigu et al., 2014). We used three different classes of chemotherapy drugs (platinum—cisplatin, anthracycline—doxorubicin (Adriamycin) and topoisomerase II inhibitor—etoposide) to test the effects of RIG-I on the response to these drugs. Our results show that the absence or depletion of RIG-I reduced caspase 3/7 activity in the response to treatment when compared to control cells (FIG. 27D and FIG. 34D). Taken together, these data suggest that RIG-I is important for cell-intrinsic IFN production in the response to multiple classes of genotoxic anticancer therapies.

3. RIG-I is Activated by IR-Induced Endogenous Double-Stranded RNAs.

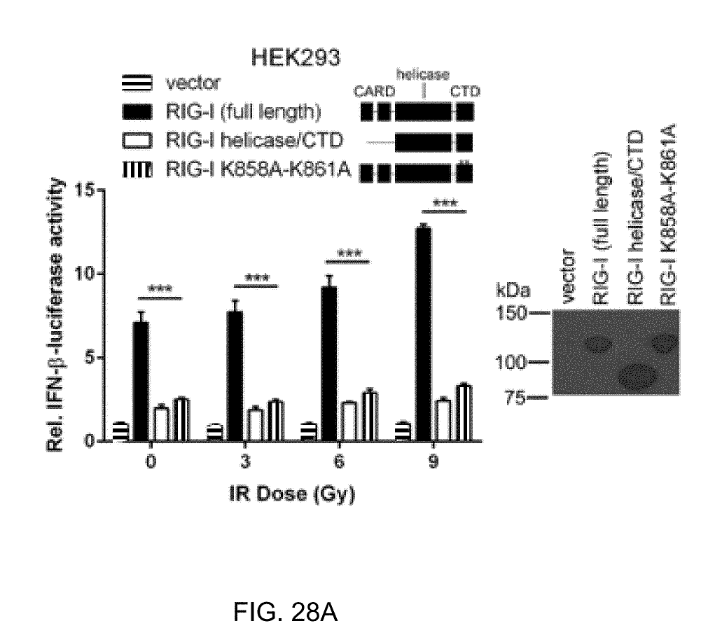

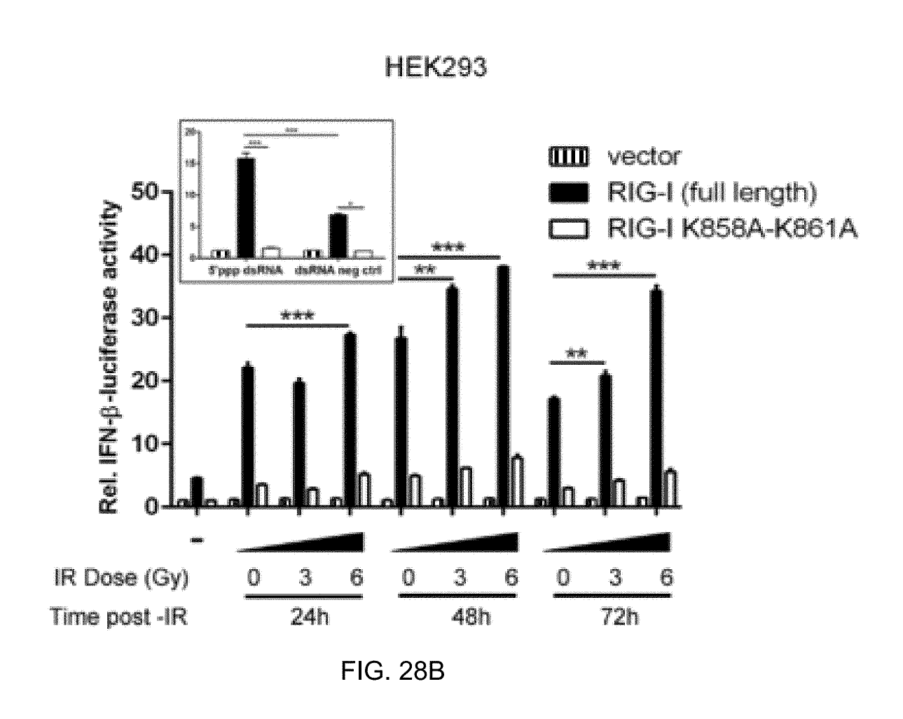

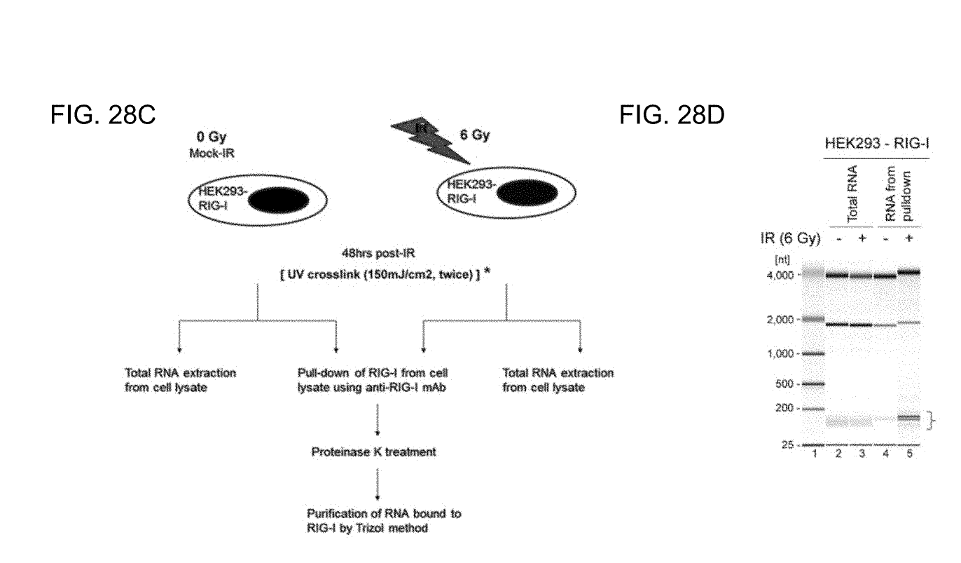

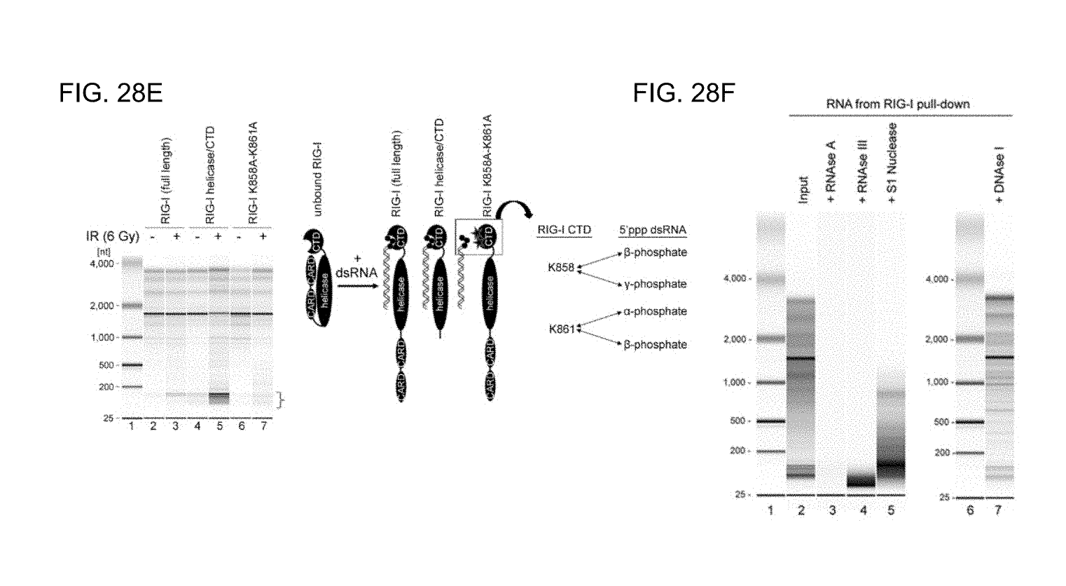

RIG-I is an RNA binding protein with two caspase recruitment domains (CARD) responsible for MAVS activation, an RNA helicase domain, and a C-terminal domain which determines the primary binding of 5′-phosphorylated dsRNA (Leung and Amarasinghe, 2012). Expression of the full-length RIG-I protein in HEK293 reporter cells led to an IR dose-dependent activation of the IFN-beta promoter (FIG. 28A). In contrast, deletion of both CARDs or mutations of C-terminal amino acids at positions K858 and K861, which are important for efficient RNA binding, abrogated IR-mediated IFN-beta expression (Wang et al., 2010; Lu et al., 2010). These findings supported a role for the RNA binding function of RIG-I in transduction of IR-dependent IFN signaling. We tested the hypothesis that IR induces the expression of RIG-I-activating RNAs. HEK293 IFN-beta luciferase reporter cells transfected with a full-length RIG-I, a K858A-K861A RNA binding deficient mutant, or an empty vector were stimulated with total RNA purified from control or irradiated donor HEK293 cells (FIG. 28B). HEK293 cells expressing full-length RIG-I, but not the RNA binding deficient K858A-K861A, demonstrated IFN-beta induction in a dose- and time-dependent manner (FIG. 28B). We therefore concluded that IR leads to the appearance of RNA species, able to activate RIG-I through its RNA binding pocket.

We further immunoprecipitated RNA bound to ectopically expressed RIG-I following IR (see scheme of the experiments in FIG. 28C). Non-irradiated and isotype control samples contained no detectable RNA, while, in contrast, we detected RNA in RIG-I complexes following IR (FIG. 28D). IR led to an enrichment of small RNA molecules (˜180 nucleotides) in RIG-I complexes (FIG. 28D lane 5 and FIG. 28E lane 3). As compared to full-length RIG-I, CARD deletion increased RNA binding, consistent with recent findings (Kowalinski et al., 2011) (FIG. 28E lane 5). In contrast, K858A-K861A RIG-I mutations diminished RNA binding (FIG. 28E lane 7). RIG-I-bound material was RNase A-sensitive, DNase I-resistant, partially resistant to single-stranded specific nuclease S1 but sensitive to double-stranded specific nuclease RNase III (FIG. 28F). These results indicated that RIG-I binds RNA molecules enriched with double-stranded regions, which is consistent with the known substrate specificity of the RIG-I protein (Schlee et al., 2009). Taken together, these findings suggested IR-induced activation of IFN signaling occurs through binding of endogenous RNA molecules which contain double-stranded regions with the C-terminal K858-K861 pocket of the RIG-I protein (see inset of FIG. 28E for the cartoon illustration).

4. Nuclear-Cytoplasmic Distribution of Small Non-Coding RNAs Leads to RIG-I-Mediated IFN-Beta Response

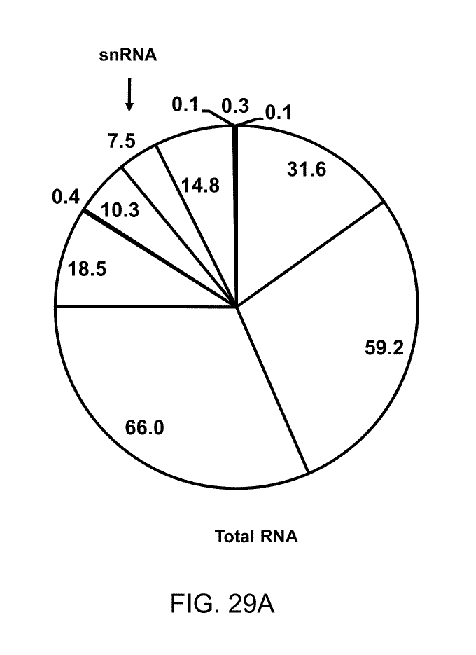

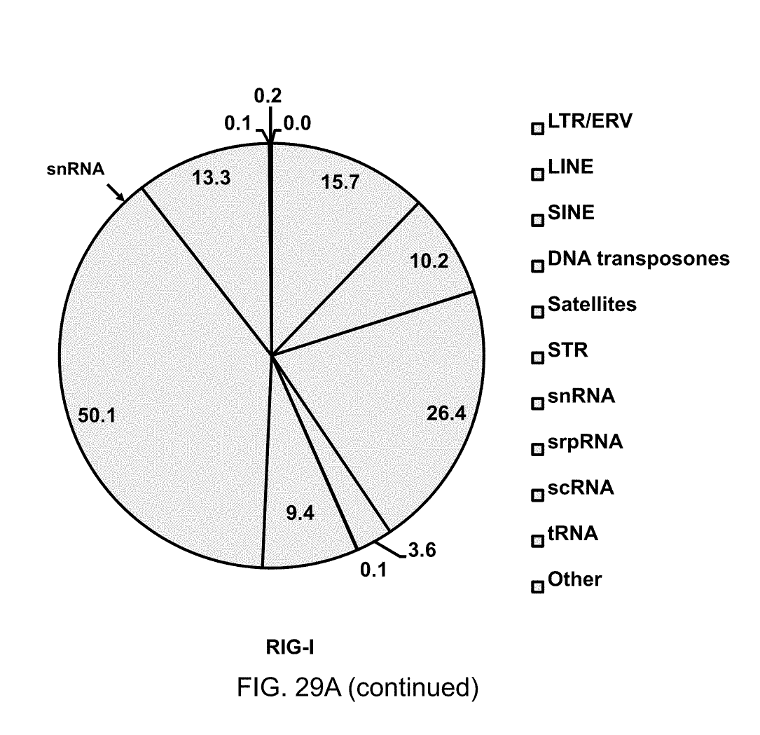

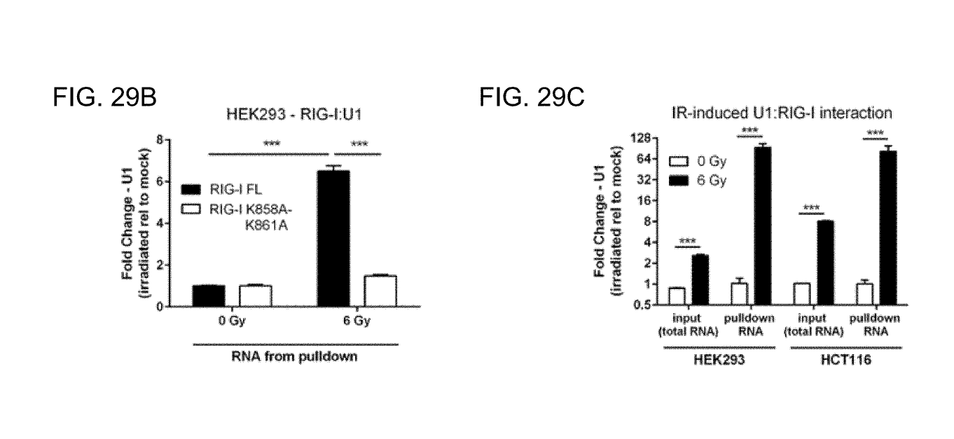

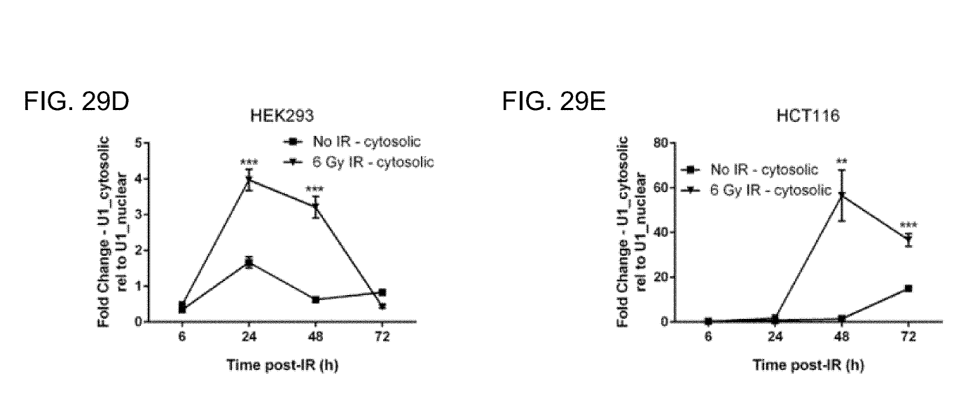

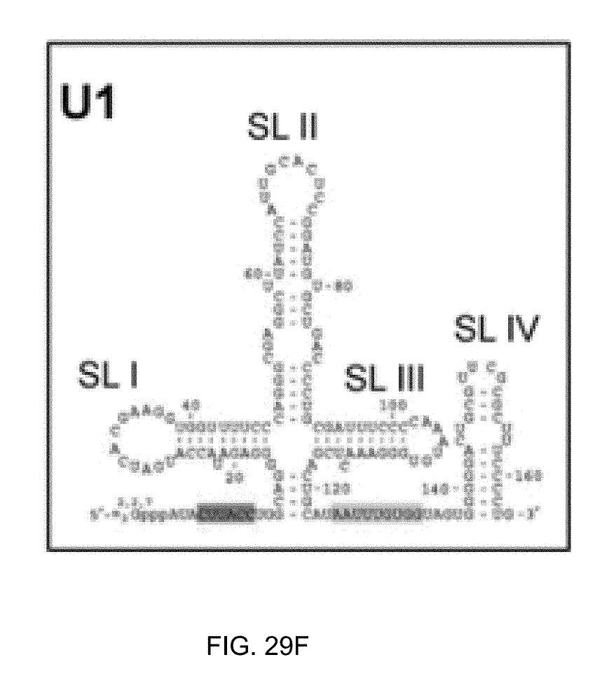

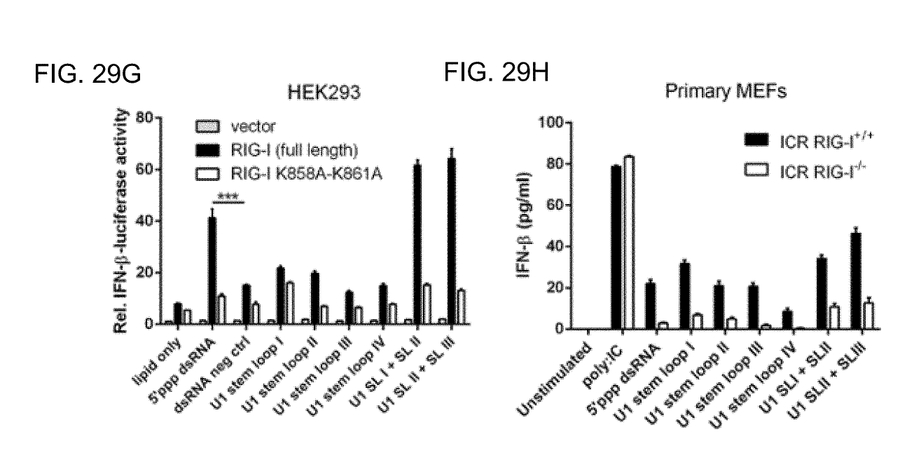

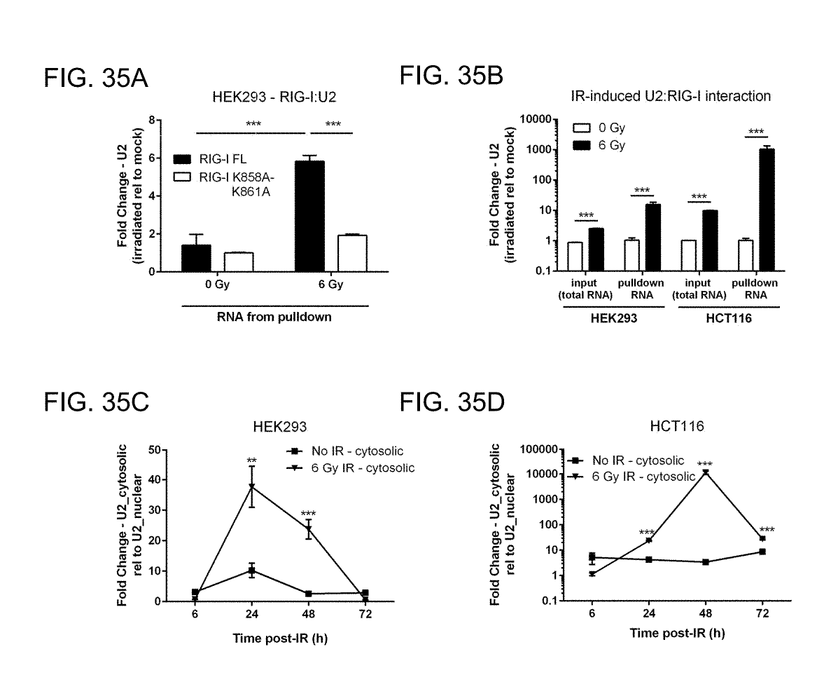

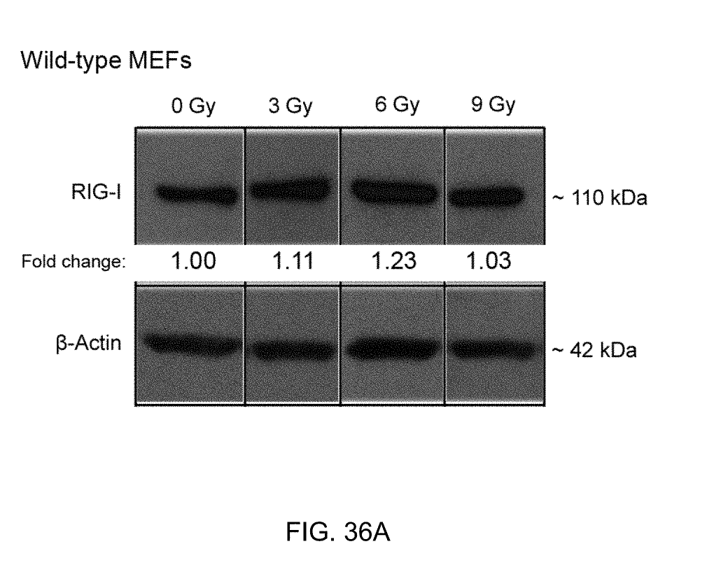

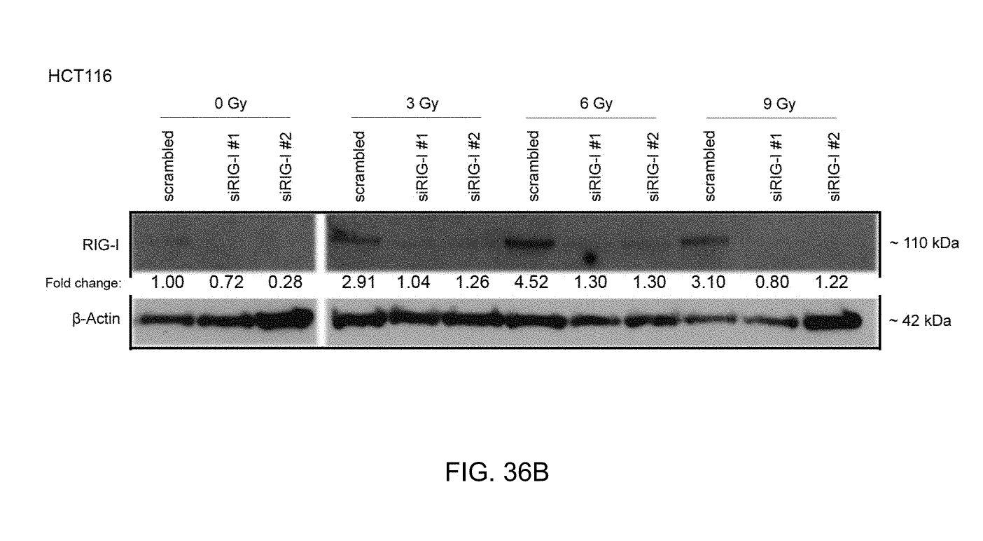

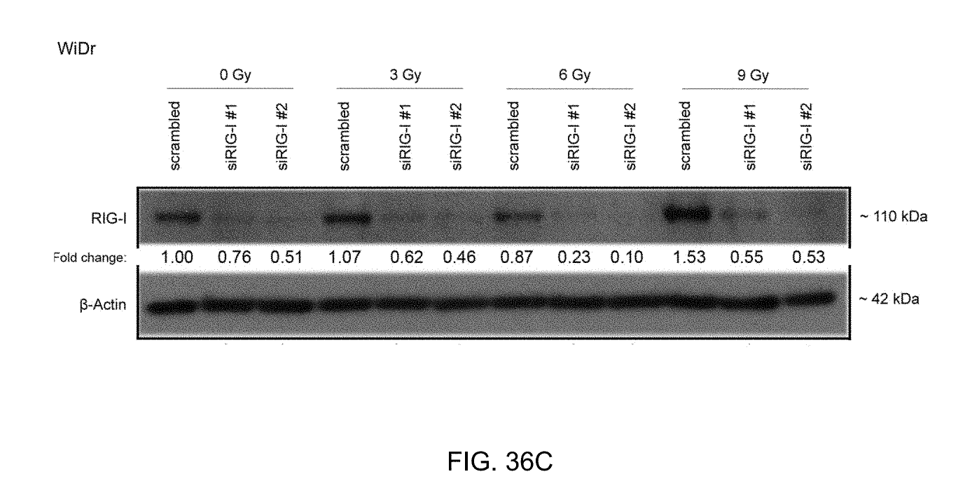

Previous reports indicate that genotoxic stress activates the transcription of repetitive and non-coding RNAs (Leonova et al., 2013; Rudin and Thompson, 2001; Tarallo et al., 2012). We used an RNA sequencing approach to preliminarily characterize RNAs bound to RIG-I post-IR. The most striking result of these experiments was an enrichment of RIG-I by small nuclear RNAs as U1 and U2 following IR (FIG. 29A and Table 5). To validate these pilot data, we used a combination of covalent UV-cross-linking with quantitative real-time PCR (CLIP-PCR). We found a 6-fold enrichment of U1 and U2 snRNA in purified RIG-I complexes from irradiated HEK293 cells as compared to non-irradiated controls (FIG. 29B and FIG. 35A). We did not detect increased levels of either U1 or U2 in HEK293 cells overexpressing the K858A-K861A RNA binding deficient mutant RIG-I. We should note that this is the most stringent negative control for these types of experiments, clearly demonstrating that IR induces specific binding of U1 and U2 to RIG-I. Importantly, pull-down of RIG-I: RNA complexes from HCT116 cells overexpressing RIG-I also demonstrated a significant enrichment by U1 and U2 in irradiated samples indicating a similar mechanism of RIG-I activation in tumor cells (FIG. 29C and FIG. 35B). Given that small nuclear RNAs predominantly reside in the nucleus, we hypothesized that following IR, U1 and U2 snRNAs translocate to the cytoplasm which permits interaction with RIG-I. Indeed, we observed a cytoplasmic redistribution of U1 and U2 RNAs following IR exposure in both HEK293 and HCT116 cells (FIGS. 29D and 29E and FIGS. 35C and 35D). In HEK293 cells, there was a 4-fold increase in the nuclear/cytoplasmic ratio of U1 RNA at 24 hours post-IR as compared to untreated cells (FIG. 29D). Similar dynamics were observed in HCT116 cells (FIG. 29E). Likewise, we observed cytoplasmic re-distribution of the U2 snRNA in both HEK293 and HCT116 cell lines starting at 24 hours post-IR (FIGS. 35C and 35D). Interestingly, higher cytoplasmic/nuclear ratios of U1 RNA levels in HCT116 cells as compared to HEK293 cells correlated with previous observations showing elevated levels of IFN-beta production in tumor cells relative to normal cells (FIGS. 25D, 25E and 25F). Importantly, IR also induced the cytoplasmic accumulation of RIG-I protein both in primary MEFs and in at least two different tumor cell lines (FIGS. 36A, 36B and 36C). Thus far, our data suggest that activation of RLR signaling by genotoxic stress is associated with nuclear to cytoplasmic redistribution of U1 (and U2) and the radio-inducibility of RIG-I.

To further confirm that RIG-I recognition of U1 induces IFN-beta signaling, we used in vitro transcribed (IVT) full length U1 RNA as agonist in our HEK293 dual luciferase reporter system. We demonstrated that U1 RNA has potent IFN-beta stimulatory activity in RIG-I overexpressing cells and is able to activate endogenous RIG-I in HEK293 cells (FIG. 37A). Digestion of U1 RNA by RNAse III markedly diminished RIG-I-dependent IFN-beta activation, indicating the importance of double-stranded regions of this molecule for induction of IFN response. Furthermore, treatment of U1 with calf intestinal alkaline phosphatase (CIAP) to remove the phosphate group at the 5′ end reduced IFN-beta reporter activity by two-fold (FIG. 37B). To assess this response in further detail, we chemically synthesized stem loop (SL) regions of U1 (FIG. 29F). We found that double-stranded regions of U1 (SL I+II or SL II+III) are potent inducers of IFN-beta response (FIGS. 29G and 29H). Interestingly, the same sequences of U1 have been reported to induce cytokine production in keratinocytes following exposure to ultraviolet radiation in a Toll-like receptor (TLR) 3-dependent manner (Bernard et al., 2012). These data support the notion that U1 is a potential endogenous activating ligand for RIG-I. Taken together, these data suggest that cell-intrinsic cytosolic accumulation of RIG-I: RNA complexes in irradiated cells activates MAVS-dependent IFN-signaling.

5. Enrichment of M5 and M8 in RIG-I Biding.

In further experiments, we examined the binding of M5 and M8 to RIG-I to activate the production of type I interferon upon addition of M5 or M8 to tumor cells such as HEK293 or HCT116 cells (Chiang et al., “Sequence-specific modifications enhance the broad spectrum antiviral response activated by RIG-I agonists”). To validate the binding, we used a combination of covalent UV-cross-linking with quantitative real-time PCR (CLIP-PCR). Similar to the binding behavior of U1 and U2, K858A-K861A RNA binding deficient mutant RIG-I did not bind M5 or M8 and was not able to produce type I interferons. In addition, RIG-I: RNA complexes isolated from HCT116 cells overexpressing RIG-I also demonstrated that RIG-I binds to M5 or M8.

To further confirm that RIG-I recognition of M5 or M8 induces IFN-beta signaling, we used in vitro transcribed (IVT) M5 or M8 RNA as agonist in our HEK293 dual luciferase reporter system. We demonstrated that either M5 or M8 has potent IFN-beta stimulatory activity in RIG-I overexpressing cells and is able to activate endogenous RIG-I in HEK293 cells similar to U1 or U2 activation of RIG-I described herein.

6. Rig-I Signaling Confers Response to DNA-Damaging Therapy

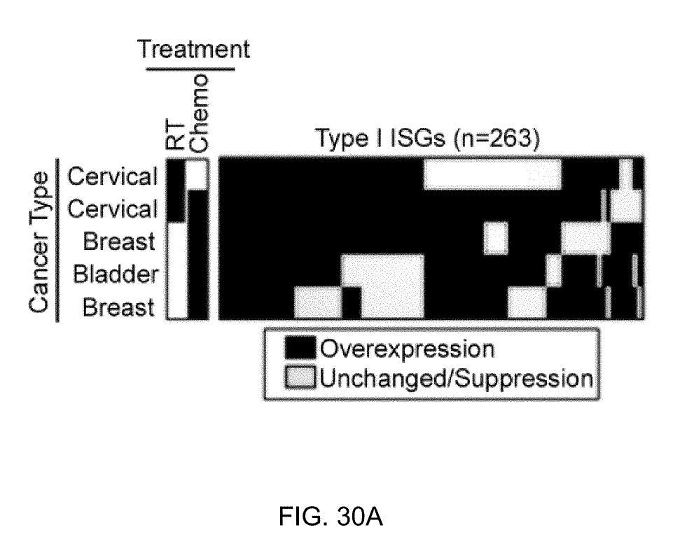

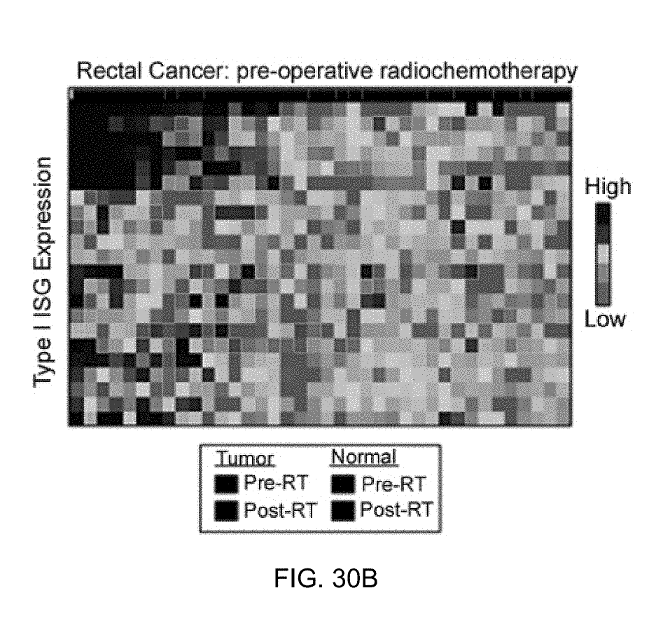

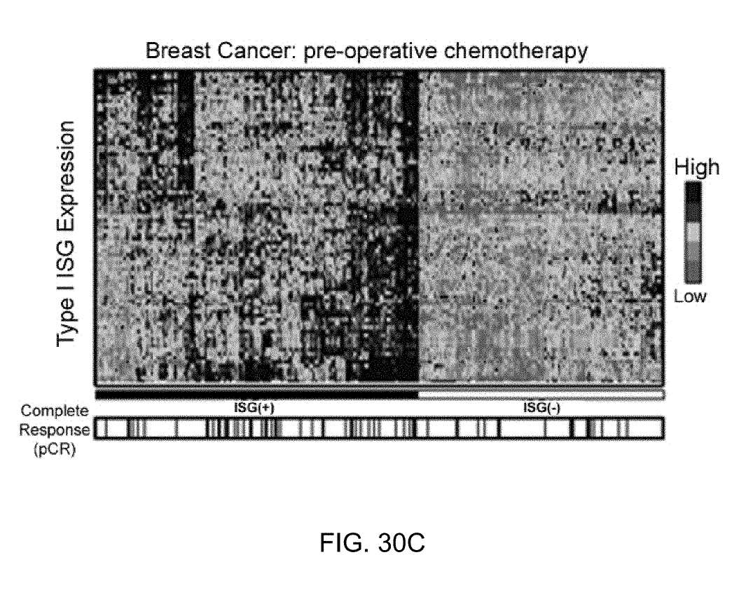

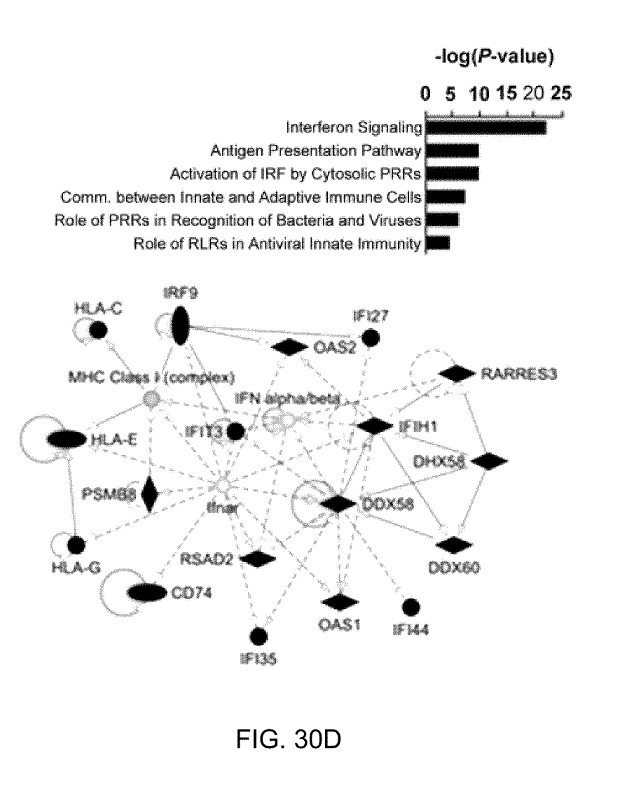

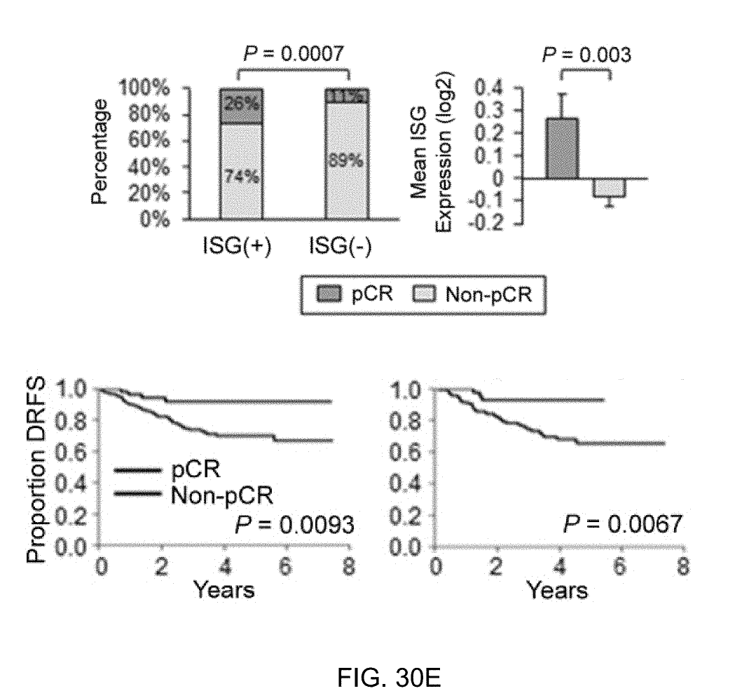

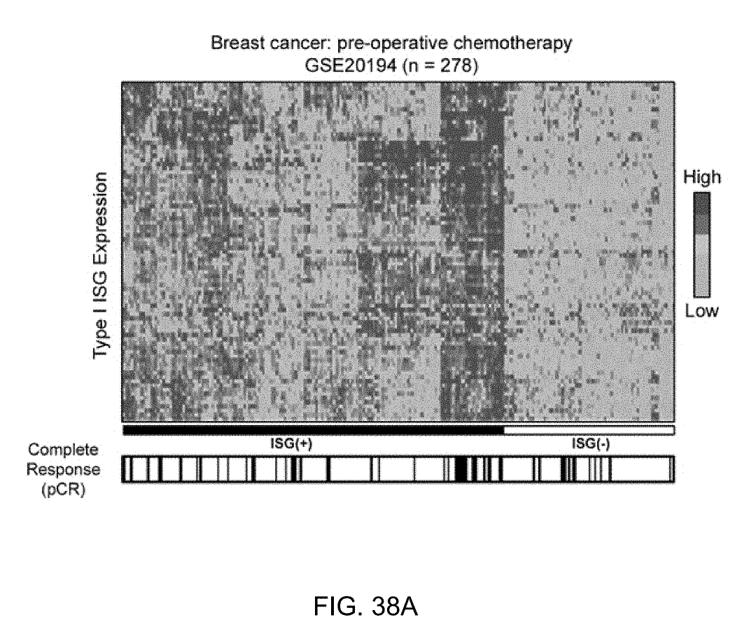

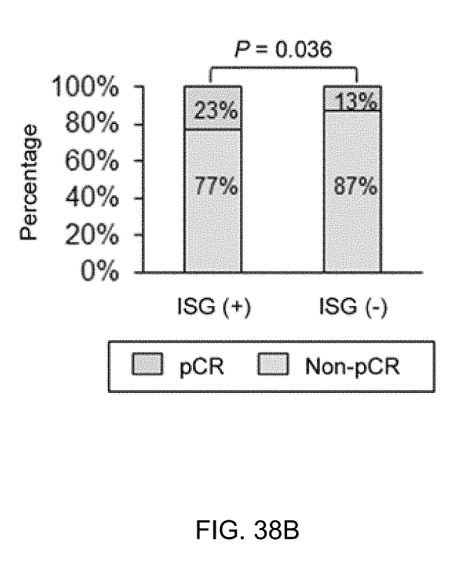

Based on our experimental data, we hypothesized that DNA damaging therapies induce Type I ISG expression in cancer patients. Of 371 Type I ISGs [39], 263 (71%) were induced in cervical, breast, and bladder cancers in the responses to genotoxic treatments (FIG. 30A). Tumors exhibited elevated ISG expression pre- and post-treatment in patients treated with radiotherapy and chemotherapy as compared to corresponding normal tissue (FIG. 30B). These findings are consistent with previous data demonstrating elevated levels of IFN signaling in tumor cells (FIGS. 25G and 25J). We identified an 81-gene subset of treatment-responsive ISGs that predicted a complete pathologic response (pCR) to pre-operative doxorubicin-based chemotherapy in a data set of 310 breast cancer patients (FIG. 30C). These findings were validated in an independent breast cancer data set of 278 patients (Extended Data FIG. 38A). Functional analysis of these ISGs highlighted functions mediating activation of IFN by cytosolic pattern recognition receptors and communication between innate and adaptive immune cells (FIG. 30D). Quantitatively, ISG(+) tumors were approximately 2.0-fold more likely to achieve a pCR as compared to ISG(−) tumors (FIG. 30E and Extended Data FIG. 38B). Importantly, the lack of pCR following pre-operative chemotherapy was associated with increased rates of distant relapse in two independent data sets totaling 588 patients (FIG. 30E). These findings demonstrate that DNA damaging therapies induce Type I interferon responses in multiple human tumors and support a link between Type I ISG expression and treatment efficacy for breast cancer patients.

7. Treatment of Tumors Using rbRNAs.

Resistance to adjuvant therapies such as ionizing radiation or chemotherapy impedes the ability to treat tumors, thus requiring additional treatments to the tumor in order to make adjuvant therapies more effective. The findings from these studies demonstrate that radiation increases the binding to oligonucleotides, such as rbRNAs (e.g., snRNAs) to a RIG-I and further sensitizes the tumor to adjuvant therapy. Therefore, to treat tumors in a patient and specifically adjuvant therapy resistant tumors, rbRNAs such as U1, U2, M5, or M8 can be administered to the patient prior to ionizing radiation treatment. A therapeutically effective dose of U1, U2, M5, M8 or a combination of one or more rbRNAs in a pharmaceutically acceptable carrier is administered directly to the tumor i.e., intratumorally to activate RIG-I. After administration, a therapeutically effective dose of ionizing radiation is administered to the tumor. As a result of the treatment combination, there is enhanced tumor cell killing by the body's immune response, effectively reducing tumor size and halting tumor growth.

Discussion

Recently, a growing body of evidence indicate a link between radio/chemotherapy of different types of tumors and Type I IFN signalling (Amundson et al., 2004; Khodarev et al., 2007; Tsai et al., 2007; Khodarev et al., 2004; Burnette et al., 2011; Lim et al., 2014; Boelens et al., 2014; Sistigu et al., 2014), reviewed in (Khodarev et al., 2012; Cheon et al., 2014; Minn, 2014; Burnette and Weichselbaum, 2013; Deng et al., 2016). Type I IFNs, induced by genotoxic stress in tumor cells may significantly modulate response of tumors to radio/chemotherapy. Through autocrine signaling they can sensitize tumor cells to genotoxic treatments and modulate the mode of the cell death, induced by IR (Widau et al., 2014; Khodarev et al., 2007; Khodarev et al., 2012). In paracrine signaling they are responsible for recruitment of immune cells in the tumor microenvironment (Burnette et al., 2011; Lim et al., 2014) thereby modulating immune response to anti-tumor therapy. Yet, molecular mechanisms of this link remained unclear. Our previous data with siRNA screen of Interferon-Stimulated Genes (ISGs) implicated LGP2 (DHX58), member of RLR pathway and suppressor of RIG-I/1VDA5 signaling, as the protein which negatively regulates IR-induced IFN response and thereby acts as powerful radioprotector in multiple types of cancer cells and tumors (Widau et al., 2014). Data presented in the current report indeed demonstrate that LGP2/RIG-I/MAVS pathway, traditionally associated with recognition of viral RNAs is necessary and sufficient for the ability of radio/chemotherapy to induce IFN signaling. We demonstrated that after treatment by IR/chemotherapy this signaling pathway is induced by small endogenous non-coding RNAs enriched with double-stranded structures, which binds to the cytoplasmic RNA sensor RIG-I. MDA5 seems to be redundant in the context of IR signaling (see FIGS. 26A, 26B, 26C and 26D and FIGS. 33A, 33B, 33C and 33D) and further investigations are necessary to evaluate its role in the response of tumor cells to genotoxic therapies. The relevance of these findings is confirmed by data that transgenic animals deficient in RIG-I are more radioresistant while animals depleted of the suppressor of RLR pathway—LGP2—are more radiosensitive (see FIGS. 26A, 26B, 26C and 26D). The role of LGP2/RIG-I/MAVS pathway in the IR-induced gastrointestinal injury (GI) is consistent with previous observations of TLR2/3/4 functions in the GI (Takemura et al., 2014) and can provide new targets for intestinal radioprotection. Furthermore, tumors with suppressed MAVS and RIG-I demonstrated clear radioresistance, while clinical data indicate that patients with proficient RIG-I/MAVS pathway are responsive to radio/chemotherapy (FIGS. 25M, 27C and 30). Taken together, these findings demonstrate that the RLR pathway is an essential component of tumor response to IR and drugs implicated in the anti-tumor therapy. These data pose intriguing questions about the origin of the dsRNA species as well as their role in mediating cytotoxic insult introduced by traditional DNA-damaging agents.

RNA response to genotoxic stress, associated with repetitive and transposable DNA elements in the human and mouse genome was reported previously. Rubin & Thompson demonstrated that exposure of apoptosis-resistant tumor cells to etoposide, cisplatin and IR led to the up-regulation of repetitive RNA transcripts from AluI and SINE elements (Rudin and Thompson, 2001). Importantly, IR also increased reverse transcriptase (RT) activity, associated with endogenous retrotransposons and the capability to transform RNA signals to DNA signals. The cytotoxicity of dsRNA enriched by repetitive AluI elements was further demonstrated in the retinal pigmentum epithelium (RPE) of patients with the age-related macular degeneration (AMD) and was associated with Dicer deficiency (Kaneko et al., 2011). Importantly, toxicity of AluI accumulation was conferred by activation of NLRP3 inflammasome and activation of IL18 (Tarallo et al., 2012), suggesting involvement of the innate immunity pathways in the recognition of endogenous dsRNA and activation of downstream cytokine response. More recently Leonova et al. (Leonova et al., 2013) described that DNA demethylation by 5-Aza-dC (inhibitor of DNA-methyltransferase I, DNMT1) leads to the induction of various types of repetitive non-coding dsRNAs, including SINES and microsatellite sequences and is associated with a cytotoxic IFN-beta production and accumulation of ISGs, which overlapped with the IRDS signature described by us previously (Weichselbaum et al., 2008). Authors demonstrated that wild-type p53 suppresses induction of these non-coding RNAs thereby acting as transcriptional repressor of such potentially toxic repetitive dsRNAs. p53-dependence of RNA signaling was also noted in TLR3/TRIF pathway (Takemura et al., 2014). Recent data from two independent groups confirmed these findings and indicated that DNA demethylation is associated with reactivation of small non-coding RNAs, enriched by endogenous retroviral sequences and associated with activation of TLR3 or/and MDA5/MAVS/IRF7 pathways (Chiappinelli et al., 2015; Li et al., 2014; Roulois et al., 2015). However, mechanisms of activation of these RNAs and their interaction with specific sensors were not clearly characterized in these publications.

One potential mechanism of the accumulation of toxic dsRNA can be presented by combination of sense- and anti-sense transcription (convergent transcription) of simple trinucleotide repeats (TNRs), usually found in genomic microsatellite sequences. Accumulation of the long (95 TNRs) tracks of such double stranded transcripts induced apoptosis and led to the death of the more than 50% of targeted cells (Lin et al., 2010; Lin et al., 2014). Convergent transcription can recruit ATR/CHK1/p53 pathway (consistent with data of Leonova et al. (Leonova et al., 2013) and Takemura et al. (Takemura et al., 2014) and alter cell cycle progression before induction of cell death (Lin et al., 2010). It is unknown whether the LGP2/RIG-I/MAVS pathway is implicated in recognition and signaling from these types of dsRNAs, but considering high levels of anti-sense transcription in genome and implication of satellite RNAs in induction of IFN-beta signaling, the mechanism of dsRNA generation through convergent transcription warrants further investigations in the context of radio/chemotherapy.

Our data indicate that IR and chemotherapy leads to transcriptional up-regulation of certain small non-coding RNAs and their nuclear to cytoplasmic translocation (see FIGS. 29D and 29E and FIGS. 35C and 35D), thus allowing them to bind to RIG-I. Interestingly, RIG-I is radioinducible protein (FIGS. 36A, 36B and 36C), which increases concentration of active cytoplasmic complexes between these RNA receptors and their ligand RNAs, thereby activating downstream signaling and IFN-beta production. We described this mechanism using mostly snRNAs U1 and U2, but further comprehensive RNA sequencing experiments are necessary to evaluate the pattern of different cellular RNAs interacting with individual members of RLR pathway in the context of radio- and chemotherapy and to estimate the role of transcriptional and post-transcriptional events in activation of this pathway. The importance of comprehensive characterization of such activating RNAs is emphasized by the recent data about differential expression in cancer cells of non-coding RNAs with motifs, specific for PRRs (Tanne et al., 2015). Potential immuno-stimulating properties of such activating RNAs and understanding of their “activating” modifications may essentially improve current empirical approaches to the design of RNA-based vaccines (Sahin et al., 2014).

Experiments, described in the current report represent cell-intrinsic RNA response to DNA damaging agents in tumor and normal cells. However, current literature indicate that RNA signaling can activate pattern recognition receptors using cell extrinsic, paracrine signaling. At least two pathways are described for such extrinsic signaling. One was demonstrated for U1 snRNA, which upon UV damage can leak in the extracellular space and bind to TLR3 receptors (Bernard et al., 2012). Interestingly, the regions of U1 that were reported sufficient for binding with TLR3 overlap with the stem loop regions we identified to be involved in interactions with RIG-I and subsequent induction of IFN-response (see FIGS. 29G and 29H and Bernard et al., 2012). Such ‘passive” leakage of dsRNAs from irradiated cells can be also essential for TLR3-dependent gastrointestinal injury, recently described by Takemura et al. (Takemura et al., 2014). Another extrinsic RNA-dependent pathway, described by Boelens et al., is presented by exosomes, which are secreted by stromal cells in the RAB27B-dependent manner (Boelens et al., 2014). These exosomes present various types of non-coding RNAs in the tumor cells, resulting in the activation of the RIG-I/MAVS pathway, which eventually induce the IRDS signature in tumor cells. Interestingly, these exosomes were found to contain non-coding snRNAs and are enriched in Alu/SINE and LINE elements as well as microsatellite RNA (Leonova et al., 2013; Rudin and Thompson, 2001; Lin et al., 2014).

Our data reveal the importance of RNA-dependent RLR-mediated IFN response to radio/chemotherapy of tumors. However, recently it was demonstrated that another cytoplasmic innate immunity pathway—DNA-dependent STING pathway (Pollpeter et al., 2011) is also implicated in the tumor response to IR (Deng et al., 2014). An intriguing difference is that the requirement for the STING pathway was demonstrated for host immune cells, primarily in myeloid and dendritic cells (DCs), and is activated through a cell-extrinsic mode by DNA molecules that are presumably released from irradiated tumor cells through a yet unidentified mechanism. Perhaps, the tumor and host immune cells may have alternative usage of RNA- and DNA dependent pathways of response to genotoxic stress. Recent findings indicate that indeed STING pathway may be deficient in certain types of tumor cells (Xia et al., 2016), which is consistent with sufficiency of RNA-dependent RLR response to radio/chemotherapy in tumor cells or/and cells of mesenchymal origin, described in this report.

In conclusion, our data provide the first comprehensive demonstration of the role of RIG-I/MAVS pathway in the Type I IFN induction in tumor cells exposed to IR and chemotherapy. Our study highlights the unusual role of small endogenous dsRNAs in DNA-damage response (DDR), previously associated almost exclusively with DNA repair/recombination machinery (Prise et al., 2005). Targeting the LGP2/RIG-I/MAVS/IFN-beta pathway may provide new strategies for radioprotection after exposure to total body or abdominal irradiation, as well as tumor sensitization to IR. We have also demonstrated that detection of structural elements of RNAs, which binds to RIG-I can be used for optimization of ligands with maximal capacity to induce type I IFNs and therefore activate adaptive immune response (see FIGS. 29G and 29H). Finally, these data suggest a co-evolution of cellular defences against pathogens and the response to IR, which warrants further investigations of RLR functions in tumor and normal cells.

### Example 5

Identification of RNAs

Technically, identification of RNAs was performed as follows: HEK293 cells were transiently transfected with 3×FLAG-tagged full-size RIG-I in pEF-BOS vector (Addgene; Cambridge, Mass.). Twenty-four hours post-transfection, cells were either mock-irradiated or exposed to IR (6 Gy). Forty-eight hours post-IR, cells were lysed with a modified lysis buffer (50 mM Tris-Cl pH 7.5, 0.15 M NaCl, 0.1% NP-40, 1.0% Triton X-100, 1 mM EDTA pH8.0, 1 mM EGTA pH8.0, 10% Glycerol, 2.5 mM MgCl₂, 1 mM DTT, 0.1 mM ATP, and 1× Halt Protease Inhibitor) and incubated on ice for 1 hour. Cell lysates were separated from cell debris by centrifugation at 12,000 rpm at 4° C. Protein concentration was measured by BCA kit. Anti-FLAG monoclonal antibody was added to the cell lysate at a 1:500 dilution, and incubated overnight at 4° C. Protein G sepharose beads were added to the lysates and incubated for at least 2 hours at 4° C. Beads containing the antibody-RIG-I complexes were washed five times in wash buffer (50 mM Tris-Cl pH 7.5, 0.15 M NaCl, 1 mM MgCl₂, 0.05% NP-40, 1 mM DTT) and proteins were eluted from the beads using a soft elution buffer (0.5% SDS, 0.1% Tween-20, 50 mM Tris pH 8.0) for 10 minutes at room temperature with vortexing every 2-3 minutes. Proteinase K was added to the eluates and incubated at 50° C. for 45 minutes. Trizol reagent was then added to the solution, and RNA bound to RIG-I was purified following manufacturer's protocol. RNA quality was analyzed using an Agilent Bioanalyzer 2100 with Pico Series II cartridges. RNA yield was measured using the Qubit RNA Broad Range kit. For qRT-PCR validation experiments of RIG-I pulled down RNA, UV cross-linking (2 doses at 150 mJ/cm²) was performed on HEK293 and HCT116 overexpressing RIG-I prior to cell lysis. The same protocol for pulldown and RNA purification experiments was performed as described above.

RNA Sequencing Analyses

We eluted total RNA and RNA bound to RIG-I from RIG-I over-expressing HEK293 cells 48 hours post IR (6 Gy). RNA purified from RIG-I pulldown as well as total RNA from HEK293 cells were used as templates to generate cDNA libraries for RNA sequencing using strand-specific NEBNext Ultra RNA Library Prep Kit for Illumina (New England Biolabs) following RiboZero (Epicentre) treatment for rRNA depletion. Libraries were sequenced on Illumina HiSeq2500 instrument to generate 50 bp pair-ended reads. Sequencing files in FastQ format were processed using AlienTrimmer (Criscuolo A and Brisse S, 2013) to remove adapter sequences and to trim low quality reads with Phred quality score <20. The preprocessed reads were aligned to the human reference genome (Ensembl GRCh38) using Spliced Transcripts Alignment to a Reference (STAR) software (Dobin et al., 2013). The featureCounts tool from Bioconductor package RSubread was used to summarize and quantify the abundances of genomic features of the mapped reads (Liao et al., 2013). Mapped reads were annotated using human GENCODE version 20 (Harrow et al., 2012) and were summarized to 35 gene/transcript and non-coding RNA biotypes annotated in GENCODE/Ensemble databases. RepEnrich program (Criscione et al., 2014) was used to identify and quantify the repetitive elements. The program uses Bowtie (Langmead et al., 2009) to align the reads to the human reference genome (Ensembl GRCh38) and human repetitive element pseudogenomes built upon RepeatMasker annotation library hg38.fa.out.gz (available at repeatmasker.org on the World Wide Web). The mapped reads were summarized by repetitive element subfamilies, families and classes (Tables 3, 4 and 5). To identify differentially expressed genomic features among RIG-I pulldown samples and total RNA (±IR treatment) samples, Bioconductor package DESeq2 (Love et al., 2014) and limma (Smyth G K, 2004; Law et al., 2014) were used.

RNAs for Stimulate IFN-Beta Production

We also used reporter HEK293 cells as simple technique to evaluate ability of the given RNA to stimulate IFN-beta production in preliminary in vitro experiments. Technically this was performed as follows: RNA stimulation of HEK293 IFN-beta reporter cells HEK293 cells were seeded in a 24-well plate overnight at a density of 1.5×10⁵ cells/ml (75,000 cells/well). Cells were co-transfected with 100 ng of plasmid construct (pCAGGS empty vector, pCAGGS-RIG-I full-length, pCAGGS-RIG-I helicase-RD mutant construct, and pCAGGS-RIG-I K858A-K861A mutant construct), together with 80 ng of a firefly luciferase reporter gene driven by an IFN-beta promoter and 20 ng of a Renilla luciferase (pRL-null) transfection control. Transfections were performed using a cationic lipid agent, Fugene HD (Promega), at a 3:1 lipid:DNA ratio. Twenty-four hours post-transfection, cells were then stimulated with 1 μg RNA* mixed with Fugene HD at 2:1 lipid: RNA ratio for 24 hours. 20 μl cell lysates were collected in opaque 96-well plates and analyzed for IFN-beta-luciferase and Renilla activity using a BioTek Synergy HT plate reader. The transfection efficiency across different wells was normalized by dividing the IFN-beta luciferase activity with the Renilla activity. All values were further normalized to the unstimulated controls in cells transfected with the empty vector. All pCAGGS RIG-I constructs used in this study were generous gifts from Dr. Jenish Patel and Dr. Adolfo Garcia-Sastre of The Icahn School of Medicine at Mount Sinai in New York City.

Total RNA Stimulation:

Total RNA from donor HEK293 cells was prepared from irradiated cells and harvested at different time points post-IR treatment (24, 48 and 72 hours post-IR). Trizol reagent was used to purify the total RNA. RNA yield was measured using Qubit RNA broad range kit.

Synthetic RNA Stimulation:

Synthetic RNA comprised of various stem loop regions of the human U1 snRNA were purchased from IDT Oligos as reported in (Bernard et al., 2012). U1 stem loop I sequence: 5′-GGGAGAACCAUGAUCACGAAGGUGGUUUUCCC-3′ (SEQ ID NO:15); U1 stem loop II sequence: 5′-GGGCGAGGCUUAUCCAUUGCACUCCGGAUGUGCUCCCC-3′ (SEQ ID NO:16); U1 stem loop III sequence: 5′-CGAUUUCCCCAAAUGUGGGAAACUCG-3′ (SEQ ID NO:17); U1 stem loop IV sequence: 5′-UAGUGGGGGACUGCGUUCGCGCUUUCCCCUG-3′ (SEQ ID NO:18); U1 stem loops I and II sequence: 5′-GGGAGATACCATGATCACGAAGGTGGTTTTCCCAGGGCGAGGCTTATCCATTGCACT CCGGATGTGCTGACCCC-3′ (SEQ ID NO:19); U1 stem loops II and III sequence: 5′-GGGCGAGGCTTATCCATTGCACTCCGGATGTGCTGACCCCTGCGATTTCCCCAAATG TGGGAAACTCGACTGC-3′ (SEQ ID NO:20).

U2 has the sequence of 5′ AUCGCUUCUCGGCCUUUUGGCUAAGAUCAAGUGUAGUAUCUGUUCUUAUCAGUU UAAUAUCUGAUACGUCCUCUAUCCGAGGACAAUAUAUUAAAUGGAUUUUUGGAG CAGGGAGAUGGAAUAGGAGCUUGCUCCGUCCACUCCACGCAUCGACCUGGUAUUG CAGUACCUCCAGGAACGGUGCACCC 3′ (SEQ ID NO:21).

A synthetic nucleotide of the present invention may comprise RNA-analogues or known modifications. For example, the synthetic oligonucleotides may comprise 2′-O-methyl-substituted RNA, locked nucleic acid or bridged nucleic acid, morpholino, or peptide nucleic acid. These modifications may improve the efficacy and stability of the rbRNAs. In some embodiments, the synthetic oligonucleotides may comprise unnatural base pair for example, d5SICS and dNaM (Malysehv et al., 2014). In other embodiments, the synthetic oligonucleotide may be modified with methyl groups such as the addition of a methyl group to the 2′-position of the ribose on the terminal nucleotide.

Double-stranded positive and negative RNA controls were purchased from InvivoGen (San Diego, Calif.).

Positive control (19-mer):
(SEQ ID NO: 22)
5′-pppGCAUGCGACCUCUGUUUGA-3′
(SEQ ID NO: 23)
3′-CGUACGCUGGAGACAAACU-5′;
Negative control (19-mer):
(SEQ ID NO: 24)
5′-GCAUGCGACCUCUGUUUGA-3′
(SEQ ID NO: 25)
3′-CGUACGCUGGAGACAAACU-5′

In vitro transcribed U1 RNA stimulation: Full length U1 (pT7U1) plasmid was generously provided by Dr. Joan Steitz (Yale School of Medicine, Yale University). In vitro transcription was performed using the HiScribe T7 Quick high yield RNA synthesis kit (New England Biolabs) following manufacturer's protocol. RNA was purified using the Trizol method.

Delivery Detected Small Endogenous RNAs in the Tumor Microenvironment and Effects of IR on their Persistence in the Tumor Bed

We further tested ability to deliver detected small endogenous RNAs in the tumor microenvironment and effects of IR on their persistence in the tumor bed. To this end MC-38 (1×10⁶ cells) were injected subcutaneously in C57BL/6 mice. Tumor growth was monitored until the volume reached 150-200 mm³ (9 days post-injection), at which point, the tumors from a subset of mice were locally irradiated at 20Gy. Four days after irradiation, Cy3-labeled U1 stem loops I+II RNA (10 μg) was intratumorally injected to mice with or without a cationic lipid carrier designed specifically for therapeutic delivery of small RNAs (10 μl of Polyplus in vivo-JetPEI, N/P ratio=6 (Polyplus-transfection SA, Illkirch France). The N/P ratio is the number of nitrogen residues of in vivo-jetPEI per nucleic acid phosphate. For in vivo nucleic acid delivery experiments, the recommended N/P ratio is 6 to 8 to maintain ionic balance within in vivo-jetPEI/nucleic acid complexes. About 2.5, 24, and 52 hours post-RNA injection, fluorescent intensities were quantified with IVIS 200 (Xenogen, MA, USA) imaging system at 535 nm excitation and 580 nm emission wavelength. As shown in FIG. 39, IR drastically increased stability of RNA in the tumor microenvironment (up to 52 hours). Pre-incubation of RNA with the jetPEI lipid further increased stability of RNA (see quantified fluorescent intensity table in FIG. 39). Together these data indicate that we designed the way to deliver selected RNAs into the tumor microenvironment in preclinical animal models.

To further test ability of delivered RNAs to affect tumor growth we irradiated MC38 tumors at 20Gy and injected irradiated tumors with stem-loop regions of U1 or U2 at 1, 7 and 14 dayspost-IR. Tumors were grown for 17 days post IR and each 3^{rd} day were measured as described in. As is shown in FIG. 40, injection of stem-loop structures of U1 in combination with jetPEI lipid and IR led to the 2-fold suppression of tumor growth as compared with IR only. These data show that U1 endogenous RNA detected in complexes with RIG-I, demonstrated to induce IFN-beta promoter in vitro, is a potent radiosensitizer of tumor in preclinical animal model.

To further test ability of delivered synthetic RNAs (M5 and M8) to affect tumor growth we irradiated MC38 tumors at 20Gy and injected irradiated tumors 1, 7 and 14 dayspost-IR. Tumors were grown for 17 days post IR and each 3rd day were measured as described in. Injection of synthetic RNAs, M5 and M8, in combination with jetPEI lipid and IR led to the 2-fold suppression of tumor growth as compared with IR only. These data show that the addition of rbRNAs are a potent radiosensitizer of tumor in preclinical animal model.

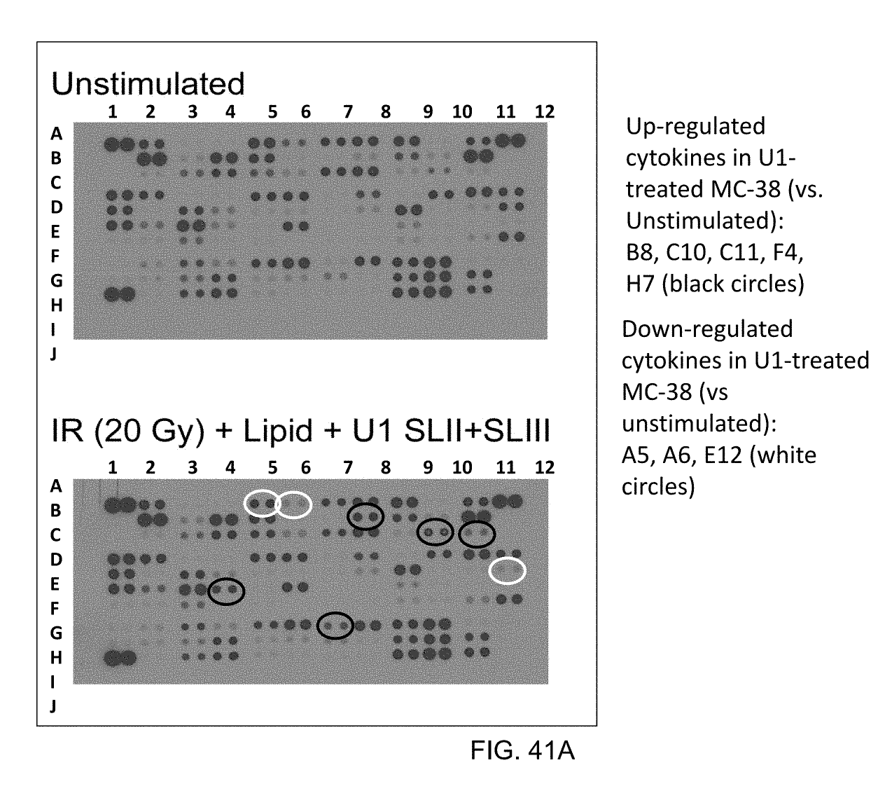

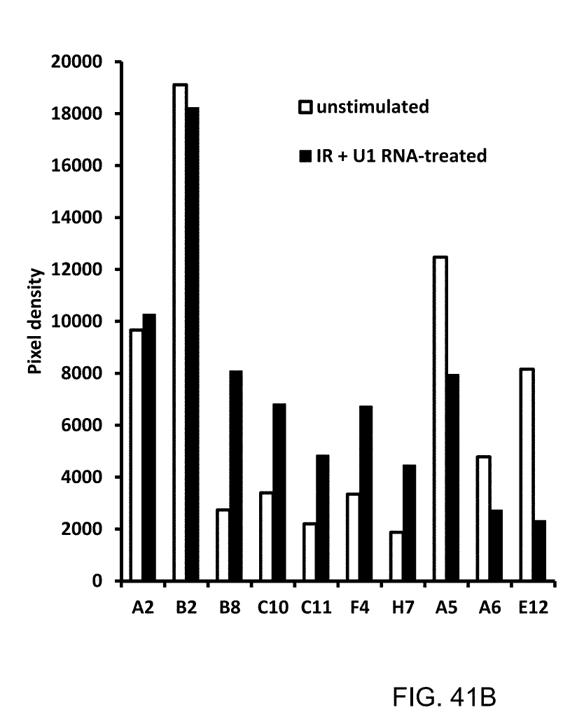

Finally we considered that there are two routes for exogenous RNA in the tumor microenvironment. First through intracellular delivery and activation of intracellular RIG-I, which can operate in tumor cells as described above. Second through extracellular binding with TLR3 receptors, which may involve host cells and lead to the alternative IL6/TNF-alpha/IL1 signaling, as described in Bernard et al. (Bernad, et al., 2012). Additionally, if different ligands can be activated by up-stream RLR and TLR receptors it is reasonable that for better radio/chemosensitization it might be useful to suppress ligands with potential pro-survival signaling. To test what ligands can be activated by RNA delivery we used protein arrays with loaded probes for multiple mouse cytokines and chemokines. As is shown in FIG. 41, injections of RNA-lipid complexes in tumors led to upregulation of several ligands with pro-survival properties. These experiments indicated that for improved suppressive effects of RNA ligands they may be combined with agents inhibiting pro-survival ligands induced by the given RNA. Overall this indicates that for further improvement of therapeutic potential of such RNA drug it is important to test pattern of cytokines induced by RNA injections.

### REFERENCES

Ablasser, A., Schmid-Burgk, J. L., Hemmerling, I., Horvath, G. L., Schmidt, T., Latz, E., and Hornung, V. (2013). Cell intrinsic immunity spreads to bystander cells via the intercellular transfer of cGAMP. Nature 503, 530-534.
Ahn, J., Gutman, D., Saijo, S., and Barber, G. N. (2012). STING manifests self DNA-dependent inflammatory disease. Proc Natl Acad Sci USA 109, 19386-19391. Apetoh, L., Ghiringhelli, F., Tesniere, A., Obeid, M., Ortiz, C., Criollo, A., Mignot,
G., Maiuri, M. C., Ullrich, E., Saulnier, P., et al. (2007). Toll-like receptor 4-dependent contribution of the immune system to anticancer chemotherapy and radiotherapy. Nat Med 13, 1050-1059.
Begg, A. C., Stewart, F. A., and Vens, C. (2011). Strategies to improve radiotherapy with targeted drugs. Nat Rev Cancer 11, 239-253.
Bernard, J. J., Cowing-Zitron, C., Nakatsuji, T., Muehleisen, B., Muto, J., Borkowski, A. W., Martinez, L., Greidinger, E. L., Yu, B. D., and Gallo, R. L. (2012). Ultraviolet radiation damages self noncoding RNA and is detected by TLR3. Nat Med 18, 1286-1290.
Burdette, D. L., and Vance, R. E. (2013). STING and the innate immune response to nucleic acids in the cytosol. Nat Immunol 14, 19-26.
Burnette, B. C., Liang, H., Lee, Y., Chlewicki, L., Khodarev, N. N., Weichselbaum, R. R., Fu, Y. X., and Auh, S. L. (2011). The efficacy of radiotherapy relies upon induction of type i interferon-dependent innate and adaptive immunity. Cancer Res 71, 2488-2496.
Chamilos, G., Gregorio, J., Meller, S., Lande, R., Kontoyiannis, D. P., Modlin, R. L., and Gilliet, M. (2012). Cytosolic sensing of extracellular self-DNA transported into monocytes by the antimicrobial peptide LL37. Blood 120, 3699-3707.
Chen, G. Y., and Nunez, G. (2010). Sterile inflammation: sensing and reacting to damage. Nat Rev Immunol 10, 826-837.
Chen, H., Sun, H., You, F., Sun, W., Zhou, X., Chen, L., Yang, J., Wang, Y., Tang, H., Guan, Y., et al. (2011). Activation of STATE by STING is critical for antiviral innate immunity. Cell 147, 436-446.
Deng, L., Liang, H., Burnette, B., Beckett, M., Darga, T., Weichselbaum, R. R., and Fu, Y. X. (2014). Irradiation and anti-PD-L1 treatment synergistically promote antitumor immunity in mice. J Clin Invest 124, 687-695.
Desmet, C. J., and Ishii, K. J. (2012). Nucleic acid sensing at the interface between innate and adaptive immunity in vaccination. Nat Rev Immunol 12, 479-491.
Diana, J., Simoni, Y., Furio, L., Beaudoin, L., Agerberth, B., Barrat, F., and Lehuen, A. (2013). Crosstalk between neutrophils, B-1a cells and plasmacytoid dendritic cells initiates autoimmune diabetes. Nat Med 19, 65-73.
Gall, A., Treuting, P., Elkon, K. B., Loo, Y. M., Gale, M., Jr., Barber, G. N., and Stetson, D. B. (2012). Autoimmunity initiates in nonhematopoietic cells and progresses via lymphocytes in an interferon-dependent autoimmune disease. Immunity 36, 120-131.
Gao, P., Ascano, M., Wu, Y., Barchet, W., Gaffney, B. L., Zillinger, T., Serganov, A. A., Liu, Y., Jones, R. A., Hartmann, G., et al. (2013a). Cyclic [G(2′,5′)pA(3′,5′)p] is the metazoan second messenger produced by DNA-activated cyclic GMP-AMP synthase. Cell 153, 1094-1107.
Gao, P., Ascano, M., Zillinger, T., Wang, W., Dai, P., Serganov, A. A., Gaffney, B. L., Shuman, S., Jones, R. A., Deng, L., et al. (2013b). Structure-function analysis of STING activation by c[G(2′,5′)pA(3′,5′)p] and targeting by antiviral DMXAA. Cell 154, 748-762.
Gehrke, N., Mertens, C., Zillinger, T., Wenzel, J., Bald, T., Zahn, S., Tuting, T., Hartmann, G., and Barchet, W. (2013). Oxidative damage of DNA confers resistance to cytosolic nuclease TREX1 degradation and potentiates STING-dependent immune sensing. Immunity 39, 482-495.
Holm, C. K., Jensen, S. B., Jakobsen, M. R., Cheshenko, N., Horan, K. A., Moeller, H. B., Gonzalez-Dosal, R., Rasmussen, S. B., Christensen, M. H., Yarovinsky, T. O., et al. (2012). Virus-cell fusion as a trigger of innate immunity dependent on the adaptor STING. Nat Immunol 13, 737-743.
Iida, N., Dzutsev, A., Stewart, C. A., Smith, L., Bouladoux, N., Weingarten, R. A., Molina, D. A., Salcedo, R., Back, T., Cramer, S., et al. (2013). Commensal bacteria control cancer response to therapy by modulating the tumor microenvironment. Science 342, 967-970.
Imanishi, T., Ishihara, C., Badr Mel, S., Hashimoto-Tane, A., Kimura, Y., Kawai, T., Takeuchi, O., Ishii, K. J., Taniguchi, S., Noda, T., et al. (2014). Nucleic acid sensing by T cells initiates Th2 cell differentiation. Nat Commun 5, 3566.
Ishii, K. J., Kawagoe, T., Koyama, S., Matsui, K., Kumar, H., Kawai, T., Uematsu, S., Takeuchi, O., Takeshita, F., Coban, C., and Akira, S. (2008). TANK-binding kinase-1 delineates innate and adaptive immune responses to DNA vaccines. Nature 451, 725-729.
Ishikawa, H., and Barber, G. N. (2008). STING is an endoplasmic reticulum adaptor that facilitates innate immune signalling. Nature 455, 674-678.
Ishikawa, H., Ma, Z., and Barber, G. N. (2009). STING regulates intracellular DNA-mediated, type I interferon-dependent innate immunity. Nature 461, 788-792.
Kono, H., and Rock, K. L. (2008). How dying cells alert the immune system to danger. Nat Rev Immunol 8, 279-289.
Lahaye, X., Satoh, T., Gentili, M., Cerboni, S., Conrad, C., Hurbain, I., El Marjou, A., Lacabaratz, C., Lelievre, J. D., and Manel, N. (2013). The capsids of HIV-1 and HIV-2 determine immune detection of the viral cDNA by the innate sensor cGAS in dendritic cells. Immunity 39, 1132-1142.
Lande, R., Gregorio, J., Facchinetti, V., Chatterjee, B., Wang, Y. H., Homey, B., Cao, W., Su, B., Nestle, F. O., Zal, T., et al. (2007). Plasmacytoid dendritic cells sense self-DNA coupled with antimicrobial peptide. Nature 449, 564-569.
Lee, Y., Auh, S. L., Wang, Y., Burnette, B., Meng, Y., Beckett, M., Sharma, R., Chin, R., Tu, T., Weichselbaum, R. R., and Fu, Y. X. (2009). Therapeutic effects of ablative radiation on local tumor require CD8+ T cells: changing strategies for cancer treatment. Blood 114, 589-595.
Li, X. D., Wu, J., Gao, D., Wang, H., Sun, L., and Chen, Z. J. (2013). Pivotal roles of cGAS-cGAMP signaling in antiviral defense and immune adjuvant effects. Science 341, 1390-1394.
Liang, H., Deng, L., Chmura, S., Burnette, B., Liadis, N., Darga, T., Beckett, M. A., Lingen, M. W., Witt, M., Weichselbaum, R. R., and Fu, Y. X. (2013). Radiation-induced equilibrium is a balance between tumor cell proliferation and T cell-mediated killing. J Immunol 190, 5874-5881.
Liauw, S. L., Connell, P. P., and Weichselbaum, R. R. (2013). New paradigms and future challenges in radiation oncology: an update of biological targets and technology. Sci Transl Med 5, 173sr172.
Lippmann, J., Muller, H. C., Naujoks, J., Tabeling, C., Shin, S., Witzenrath, M., Hellwig, K., Kirschning, C. J., Taylor, G. A., Barchet, W., et al. (2011). Dissection of a type I interferon pathway in controlling bacterial intracellular infection in mice. Cell Microbiol 13, 1668-1682.
Malyshev, Denis A.; Dhami, Kirandeep; Lavergne, Thomas; Chen, Tingjian; Dai, Nan; Foster, Jeremy M.; Corrëa, Ivan R.; Romesberg, Floyd E. (May 7, 2014). “A semi-synthetic organism with an expanded genetic alphabet”. Nature. 509: 385-388.
Marichal, T., Ohata, K., Bedoret, D., Mesnil, C., Sabatel, C., Kobiyama, K., Lekeux, P., Coban, C., Akira, S., Ishii, K. J., et al. (2011). DNA released from dying host cells mediates aluminum adjuvant activity. Nat Med 17, 996-1002.
Moeller, B. J., Cao, Y., Li, C. Y., and Dewhirst, M. W. (2004). Radiation activates HIF-1 to regulate vascular radiosensitivity in tumors: role of reoxygenation, free radicals, and stress granules. Cancer Cell 5, 429-441.
O'Neill, L. A., Golenbock, D., and Bowie, A. G. (2013). The history of Toll-like receptors—redefining innate immunity. Nat Rev Immunol 13, 453-460.
Paludan, S. R., and Bowie, A. G. (2013). Immune sensing of DNA. Immunity 38, 870-880.
Park, S., Jiang, Z., Mortenson, E. D., Deng, L., Radkevich-Brown, O., Yang, X., Sattar, H., Wang, Y., Brown, N. K., Greene, M., et al. (2010). The therapeutic effect of anti-HER2/neu antibody depends on both innate and adaptive immunity. Cancer Cell 18, 160-170.
Postow, M. A., Callahan, M. K., Barker, C. A., Yamada, Y., Yuan, J., Kitano, S., Mu, Z., Rasalan, T., Adamow, M., Ritter, E., et al. (2012). Immunologic correlates of the abscopal effect in a patient with melanoma. N Engl J Med 366, 925-931.
Sander, L. E., Davis, M. J., Boekschoten, M. V., Amsen, D., Dascher, C. C., Ryffel, B., Swanson, J. A., Muller, M., and Blander, J. M. (2011). Detection of prokaryotic mRNA signifies microbial viability and promotes immunity. Nature 474, 385-389.
Sharma, S., DeOliveira, R. B., Kalantari, P., Parroche, P., Goutagny, N., Jiang, Z., Chan, J., Bartholomeu, D. C., Lauw, F., Hall, J. P., et al. (2011). Innate immune recognition of an AT-rich stem-loop DNA motif in the Plasmodium falciparum genome. Immunity 35, 194-207.
Stagg, J., Loi, S., Divisekera, U., Ngiow, S. F., Duret, H., Yagita, H., Teng, M. W., and Smyth, M. J. (2011). Anti-ErbB-2 mAb therapy requires type I and II interferons and synergizes with anti-PD-1 or anti-CD137 mAb therapy. Proc Natl Acad Sci USA 108, 7142-7147.
Takeuchi, O., and Akira, S. (2010). Pattern recognition receptors and inflammation. Cell 140, 805-820.
Wu, J., and Chen, Z. J. (2014). Innate immune sensing and signaling of cytosolic nucleic acids. Annu Rev Immunol 32, 461-488.
Wu, J., Sun, L., Chen, X., Du, F., Shi, H., Chen, C., and Chen, Z. J. (2013). Cyclic GMP-AMP is an endogenous second messenger in innate immune signaling by cytosolic DNA. Science 339, 826-830.
Zhang, X., Shi, H., Wu, J., Sun, L., Chen, C., and Chen, Z. J. (2013). Cyclic GMP-AMP containing mixed phosphodiester linkages is an endogenous high-affinity ligand for STING. Mol Cell 51, 226-235.
Widau R C, Parekh A D, Ranck M C, Golden D W, Kumar K A, Sood R F, Pitroda S P, Liao Z, Huang X, Darga T E, Xu D, Huang L, Andrade J, Roizman B, Weichselbaum R R and Khodarev N N. RIG-I-like receptor LGP2 protects tumor cells from ionizing radiation. Proceedings of the National Academy of Sciences of the United States of America. 2014; 111(4):E484-491.
Amundson S A, Grace M B, McLeland C B, Epperly M W, Yeager A, Zhan Q, Greenberger J S and Fornace A J, Jr. Human in vivo radiation-induced biomarkers: gene expression changes in radiotherapy patients. Cancer research. 2004; 64(18):6368-6371.
Khodarev N N, Minn A J, Efimova E V, Darga T E, Labay E, Beckett M, Mauceri H J, Roizman B and Weichselbaum R R. Signal transducer and activator of transcription 1 regulates both cytotoxic and prosurvival functions in tumor cells. Cancer research. 2007; 67(19):9214-9220.
Tsai M H, Cook J A, Chandramouli G V, DeGraff W, Yan H, Zhao S, Coleman C N, Mitchell J B and Chuang E Y. Gene expression profiling of breast, prostate, and glioma cells following single versus fractionated doses of radiation. Cancer research. 2007; 67(8):3845-3852.
Khodarev N N, Beckett M, Labay E, Darga T, Roizman B and Weichselbaum R R. STAT1 is overexpressed in tumors selected for radioresistance and confers protection from radiation in transduced sensitive cells. Proceedings of the National Academy of Sciences of the United States of America. 2004; 101(6):1714-1719.
Lim J Y, Gerber S A, Murphy S P and Lord E M. Type I interferons induced by radiation therapy mediate recruitment and effector function of CD8(+) T cells. Cancer Immunol Immunother. 2014; 63(3):259-271.
Burnette B, Fu Y X and Weichselbaum R R. The confluence of radiotherapy and immunotherapy. Front Oncol. 2012; 2:143.
Khodarev N R, B, Weichselbaum, R. Molecular Pathways: Interferon/Stat1 pathway: role in the tumor resistance to genotoxic stress and aggressive growth Clinical Cancer Research. 2012; 18(11):1-7.
Bruns A M and Horvath C M. Antiviral RNA recognition and assembly by RLR family innate immune sensors. Cytokine & growth factor reviews. 2014.
Akira S, Uematsu S and Takeuchi O. Pathogen recognition and innate immunity. Cell. 2006; 124(4):783-801.
Loo Y M and Gale M, Jr. Immune signaling by RIG-I-like receptors. Immunity. 2011; 34(5):680-692.
Iwasaki A and Medzhitov R. Control of adaptive immunity by the innate immune system. Nature immunology. 2015; 16(4):343-353.
Medzhitov R and Janeway C, Jr. Innate immune recognition: mechanisms and pathways. Immunol Rev. 2000; 173:89-97.
Goubau D, Schlee M, Deddouche S, Pruijssers A J, Zillinger T, Goldeck M, Schuberth C, Van der Veen A G, Fujimura T, Rehwinkel J, Iskarpatyoti J A, Barchet W, Ludwig J, Dermody T S, Hartmann G and Reis E S C. Antiviral immunity via RIG-I-mediated recognition of RNA bearing 5′-diphosphates. Nature. 2014.
Cai X and Chen Z J. Prion-like polymerization as a signaling mechanism. Trends Immunol. 2014; 35(12):622-630.
Reikine S, Nguyen J B and Modis Y. Pattern Recognition and Signaling Mechanisms of RIG-I and MDA5. Front Immunol. 2014; 5:342.
Bruns A M, Leser G P, Lamb R A and Horvath C M. The Innate Immune Sensor LGP2 Activates Antiviral Signaling by Regulating MDA5-RNA Interaction and Filament Assembly. Molecular cell. 2014; 55(5):771-781.
Hagmann C A, Herzner A M, Abdullah Z, Zillinger T, Jakobs C, Schuberth C, Coch C, Higgins P G, Wisplinghoff H, Barchet W, Hornung V, Hartmann G and Schlee M. RIG-I detects triphosphorylated RNA of Listeria monocytogenes during infection in non-immune cells. PloS one. 2013; 8(4):e62872.
Devarkar S C, Wang C, Miller M T, Ramanathan A, Jiang F, Khan A G, Patel S S and Marcotrigiano J. Structural basis for m7G recognition and 2′-O-methyl discrimination in capped RNAs by the innate immune receptor RIG-I. Proceedings of the National Academy of Sciences of the United States of America. 2016; 113(3):596-601.
Boelens M C, Wu T J, Nabet B Y, Xu B, Qiu Y, Yoon T, Azzam D J, Twyman-Saint Victor C, Wiemann B Z, Ishwaran H, Ter Brugge P J, Jonkers J, Slingerland J and Minn A J. Exosome transfer from stromal to breast cancer cells regulates therapy resistance pathways. Cell. 2014; 159(3):499-513.
Weichselbaum R R, Ishwaran H, Yoon T, Nuyten D S, Baker S W, Khodarev N, Su A W, Shaikh A Y, Roach P, Kreike B, Roizman B, Bergh J, Pawitan Y, van de Vijver M J and Minn A J. An interferon-related gene signature for DNA damage resistance is a predictive marker for chemotherapy and radiation for breast cancer. Proceedings of the National Academy of Sciences of the United States of America. 2008; 105(47):18490-18495.
Duarte C W, Willey C D, Zhi D, Cui X, Harris J J, Vaughan L K, Mehta T, McCubrey R O, Khodarev N N, Weichselbaum R R and Gillespie G Y. Expression signature of IFN/STAT1 signaling genes predicts poor survival outcome in glioblastoma multiforme in a subtype-specific manner. PloS one. 7(1):e29653.
Yoneyama M, Kikuchi M, Matsumoto K, Imaizumi T, Miyagishi M, Taira K, Foy E, Loo Y M, Gale M, Jr., Akira S, Yonehara S, Kato A and Fujita T. Shared and unique functions of the DExD/H-box helicases RIG-I, MDA5, and LGP2 in antiviral innate immunity. Journal of immunology. 2005; 175(5):2851-2858.
Rothenfusser S, Goutagny N, DiPerna G, Gong M, Monks B G, Schoenemeyer A, Yamamoto M, Akira S and Fitzgerald K A. The RNA helicase Lgp2 inhibits TLR-independent sensing of viral replication by retinoic acid-inducible gene-I. Journal of immunology. 2005; 175(8):5260-5268.
Komuro A and Horvath C M. RNA- and virus-independent inhibition of antiviral signaling by RNA helicase LGP2. Journal of virology. 2006; 80(24):12332-12342.
Anno G H, Young R W, Bloom R M and Mercier J R. Dose response relationships for acute ionizing-radiation lethality. Health Phys. 2003; 84(5):565-575.
Hall E J and Giaccia A J. (2006). Radiobiology for the radiologist. (Philadelphia: Lippincott Williams & Wilkins).
Sistigu A, Yamazaki T, Vacchelli E, Chaba K, Enot D P, Adam J, Vitale I, Goubar A, Baracco E E, Remedios C, Fend L, Hannani D, Aymeric L, Ma Y, Niso-Santano M, Kepp O, et al. Cancer cell-autonomous contribution of type I interferon signaling to the efficacy of chemotherapy. Nat Med. 2014; 20(11):1301-1309.
Leung D W and Amarasinghe G K. Structural insights into RNA recognition and activation of RIG-I-like receptors. Curr Opin Struct Biol. 2012; 22(3):297-303.
Wang Y, Ludwig J, Schuberth C, Goldeck M, Schlee M, Li H, Juranek S, Sheng G, Micura R, Tuschl T, Hartmann G and Patel D J. Structural and functional insights into 5′-ppp RNA pattern recognition by the innate immune receptor RIG-I. Nature structural & molecular biology. 2010; 17(7):781-787.
Lu C, Xu H, Ranjith-Kumar C T, Brooks M T, Hou T Y, Hu F, Herr A B, Strong R K, Kao C C and Li P. The structural basis of 5′ triphosphate double-stranded RNA recognition by RIG-I C-terminal domain. Structure. 2010; 18(8):1032-1043.
Kowalinski E, Lunardi T, McCarthy A A, Louber J, Brunel J, Grigorov B, Gerlier D and Cusack S. Structural basis for the activation of innate immune pattern-recognition receptor RIG-I by viral RNA. Cell. 2011; 147(2):423-435.
Schlee M, Roth A, Hornung V, Hagmann C A, Wimmenauer V, Barchet W, Coch C, Janke M, Mihailovic A, Wardle G, Juranek S, Kato H, Kawai T, Poeck H, Fitzgerald K A, Takeuchi O, et al. Recognition of 5′ triphosphate by RIG-I helicase requires short blunt double-stranded RNA as contained in panhandle of negative-strand virus. Immunity. 2009; 31(1):25-34.
Leonova K I, Brodsky L, Lipchick B, Pal M, Novototskaya L, Chenchik A A, Sen G C, Komarova E A and Gudkov A V. p53 cooperates with DNA methylation and a suicidal interferon response to maintain epigenetic silencing of repeats and noncoding RNAs. Proceedings of the National Academy of Sciences of the United States of America. 2013; 110(1):E89-98.
Rudin C M and Thompson C B. Transcriptional activation of short interspersed elements by DNA-damaging agents. Genes, chromosomes & cancer. 2001; 30(1):64-71.
Tarallo V, Hirano Y, Gelfand B D, Dridi S, Kerur N, Kim Y, Cho W G, Kaneko H, Fowler B J, Bogdanovich S, Albuquerque R J, Hauswirth W W, Chiodo V A, Kugel J F, Goodrich J A, Ponicsan S L, et al. DICER1 loss and Alu RNA induce age-related macular degeneration via the NLRP3 inflammasome and MyD88. Cell. 2012; 149(4):847-859.
Schoggins J W, Wilson S J, Panis M, Murphy M Y, Jones C T, Bieniasz P and Rice C M. A diverse range of gene products are effectors of the type I interferon antiviral response. Nature. 2011; 472(7344):481-485.
Cheon H, Borden E C and Stark G R. Interferons and their stimulated genes in the tumor microenvironment. Seminars in oncology. 2014; 41(2):156-173.
Minn A J. Interferons and the Immunogenic Effects of Cancer Therapy. Trends Immunol. 2015.
Burnette B and Weichselbaum R R. Radiation as an immune modulator. Semin Radiat Oncol. 2013; 23(4):273-280.
Deng L, Liang H, Fu S, Weichselbaum R R and Fu Y X. From DNA Damage to Nucleic Acid Sensing: A Strategy to Enhance Radiation Therapy. Clin Cancer Res. 2016; 22(1):20-25.
Takemura N, Kawasaki T, Kunisawa J, Sato S, Lamichhane A, Kobiyama K, Aoshi T, Ito J, Mizuguchi K, Karuppuchamy T, Matsunaga K, Miyatake S, Mori N, Tsujimura T, Satoh T, Kumagai Y, et al. Blockade of TLR3 protects mice from lethal radiation-induced gastrointestinal syndrome. Nature communications. 2014; 5:3492.
Kaneko H, Dridi S, Tarallo V, Gelfand B D, Fowler B J, Cho W G, Kleinman M E, Ponicsan S L, Hauswirth W W, Chiodo V A, Kariko K, Yoo J W, Lee D K, Hadziahmetovic M, Song Y, Misra S, et al. DICER1 deficit induces Alu RNA toxicity in age-related macular degeneration. Nature. 2011; 471(7338):325-330.
Chiappinelli K B, Strissel P L, Desrichard A, Li H, Henke C, Akman B, Hein A, Rote N S, Cope L M, Snyder A, Makarov V, Buhu S, Slamon D J, Wolchok J D, Pardoll D M, Beckmann M W, et al. Inhibiting DNA Methylation Causes an Interferon Response in Cancer via dsRNA Including Endogenous Retroviruses. Cell. 2015; 162(5):974-986.
Li H, Chiappinelli K B, Guzzetta A A, Easwaran H, Yen R W, Vatapalli R, Topper M J, Luo J, Connolly R M, Azad N S, Stearns V, Pardoll D M, Davidson N, Jones P A, Slamon D J, Baylin S B, et al. Immune regulation by low doses of the DNA methyltransferase inhibitor 5-azacitidine in common human epithelial cancers. Oncotarget. 2014; 5(3):587-598.
Roulois D, Loo Yau H, Singhania R, Wang Y, Danesh A, Shen S Y, Han H, Liang G, Jones P A, Pugh T J, O'Brien C and De Carvalho D D. DNA-Demethylating Agents Target Colorectal Cancer Cells by Inducing Viral Mimicry by Endogenous Transcripts. Cell. 2015; 162(5): 961-973.
Lin Y, Leng M, Wan M and Wilson J H. Convergent transcription through a long CAG tract destabilizes repeats and induces apoptosis. Molecular and cellular biology. 2010; 30(18):4435-4451.
Lin W Y, Lin Y and Wilson J H. Convergent transcription through microsatellite repeat tracts induces cell death. Molecular biology reports. 2014; 41(9):5627-5634.
Tanne A, Muniz L R, Puzio-Kuter A, Leonova K I, Gudkov A V, Ting D T, Monasson R, Cocco S, Levine A J, Bhardwaj N and Greenbaum B D. Distinguishing the immunostimulatory properties of noncoding RNAs expressed in cancer cells. Proceedings of the National Academy of Sciences of the United States of America. 2015; 112(49):15154-15159.
Sahin U, Kariko K and Tureci O. mRNA-based therapeutics—developing a new class of drugs. Nat Rev Drug Discov. 2014; 13(10):759-780.
Pollpeter D, Komuro A, Barber G N and Horvath C M. Impaired cellular responses to cytosolic DNA or infection with Listeria monocytogenes and vaccinia virus in the absence of the murine LGP2 protein. PloS one. 2011; 6(4):e18842.
Deng L, Liang H, Xu M, Yang X, Burnette B, Arina A, Li X D, Mauceri H, Beckett M, Darga T, Huang X, Gajewski T F, Chen Z J, Fu Y X and Weichselbaum R R. STING-Dependent Cytosolic DNA Sensing Promotes Radiation-Induced Type I Interferon-Dependent Antitumor Immunity in Immunogenic Tumors. Immunity. 2014; 41(5):843-852.
Xia T, Konno H, Ahn J and Barber G N. Deregulation of STING Signaling in Colorectal Carcinoma Constrains DNA Damage Responses and Correlates With Tumorigenesis. Cell Rep. 2016; 14(2):282-297.
Prise K M, Schettino G, Folkard M and Held K D. New insights on cell death from radiation exposure. Lancet Oncol. 2005; 6(7):520-528.
Zheng C and Wu H. RIG-I “sees” the 5′-triphosphate. Structure. 2010; 18(8):894-896.
Abe Y, Fujii K, Nagata N, Takeuchi O, Akira S, Oshiumi H, Matsumoto M, Seya T and Koike S. The toll-like receptor 3-mediated antiviral response is important for protection against poliovirus infection in poliovirus receptor transgenic mice. Journal of virology. 2012; 86(1):185-194.
Tusher V G, Tibshirani R and Chu G. Significance analysis of microarrays applied to the ionizing radiation response. Proceedings of the National Academy of Sciences of the United States of America. 2001; 98(9):5116-5121.
Liu X and Fagotto F. A method to separate nuclear, cytosolic, and membrane-associated signaling molecules in cultured cells. Science signaling. 2011; 4(203):pl2.
Criscuolo A and Brisse S. AlienTrimmer: a tool to quickly and accurately trim off multiple short contaminant sequences from high-throughput sequencing reads. Genomics. 2013; 102(5-6):500-506.
Dobin A, Davis C A, Schlesinger F, Drenkow J, Zaleski C, Jha S, Batut P, Chaisson M and Gingeras T R. STAR: ultrafast universal RNA-seq aligner. Bioinformatics. 2013; 29(1):15-21.
Liao Y, Smyth G K and Shi W. The Subread aligner: fast, accurate and scalable read mapping by seed-and-vote. Nucleic acids research. 2013; 41(10):e108.
Harrow J, Frankish A, Gonzalez J M, Tapanari E, Diekhans M, Kokocinski F, Aken B L, Barrell D, Zadissa A, Searle S, Barnes I, Bignell A, Boychenko V, Hunt T, Kay M, Mukherjee G, et al. GENCODE: the reference human genome annotation for The ENCODE Project. Genome research. 2012; 22(9):1760-1774.
Criscione S W, Zhang Y, Thompson W, Sedivy J M and Neretti N. Transcriptional landscape of repetitive elements in normal and cancer human cells. BMC genomics. 2014; 15:583.
Langmead B, Trapnell C, Pop M and Salzberg S L. Ultrafast and memory-efficient alignment of short DNA sequences to the human genome. Genome biology. 2009; 10(3):R25.
Love M I, Huber W and Anders S. Moderated estimation of fold change and dispersion for RNA-seq data with DESeq2. Genome biology. 2014; 15(12):550.
Smyth G K. Linear models and empirical bayes methods for assessing differential expression in microarray experiments. Stat Appl Genet Mol Biol. 2004; 3:Article3.
Law C W, Chen Y, Shi W and Smyth G K. voom: Precision weights unlock linear model analysis tools for RNA-seq read counts. Genome biology. 2014; 15(2):R29.

The invention has been described in an illustrative manner and it is to be understood the terminology used is intended to be in the nature of description rather than of limitation. All patents and other references cited herein are incorporated herein by reference in their entirety. It is also understood that many modifications, equivalents, and variations of the present invention are possible in light of the above teachings. Therefore, it is to be understood that within the scope of the appended claims, the invention may be practiced other than as specifically described.

We claim:

1. A composition for treating cancer in a subject in need thereof, comprising:
a therapeutically effective amount of at least one rbRNA (e.g., snRNA) or its functionally equivalent fragment, and
a pharmaceutically acceptable carrier,
wherein the at least one rbRNA (e.g., snRNA) or its functionally equivalent fragment activates primary RNA or DNA sensors and
wherein the composition is administered to the subject before a dose of ionized radiation is administered to the subject.

2. A composition for treating cancer in a subject in need thereof, comprising:
a therapeutically effective amount of at least two rbRNAs (e.g., snRNAs) or their functionally equivalent fragments, and
a pharmaceutically acceptable carrier,
wherein the at least two rbRNAs (e.g., snRNAs) or their functionally equivalent fragment activates primary RNA or DNA sensors and
wherein the composition is administered to the subject before a dose of ionized radiation is administered to the subject.

3. The composition of claim 1, wherein the at least one rbRNA (e.g., snRNA) is selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

4. The composition of claim 1, wherein the at least one rbRNA (e.g., snRNA) is U2.

5. The composition of claim 1, wherein the at least one rbRNA (e.g., snRNA) is selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

6. The composition of claim 1, wherein the primary RNA or DNA sensor comprises at least one of RIG1, MDA5, DAI, IFI16, Aim2, and cGAS.

7. The composition of claim 2, wherein the at least two rbRNAs (e.g., snRNAs) are selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

8. The composition of claim 2, wherein the at least two rbRNAs (e.g., snRNAs) comprise U2.

9. The composition of claim 2, wherein the at least two rbRNAs (e.g., snRNAs) comprise U1.

10. The composition of claim 2, wherein the at least two rbRNAs (e.g., snRNAs) are selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

11. The composition of claim 2, wherein the primary RNA or DNA sensors comprise at least one of RIG1, MDA5, DAI, IFI16, Aim2, and cGAS.

12. The composition of claim 1, wherein the composition further comprises another therapeutic agent.

13. The composition of claim 12, wherein the other therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

14. The composition of claim 2, wherein the composition further comprises another therapeutic agent.

15. The composition of claim 14, wherein the other therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

16. A method of treating cancer in a subject in need thereof, comprising:
(a) administering to the subject a pharmaceutical composition comprising:
a therapeutically effective amount of at least one rbRNA (e.g., snRNA) or its functionally equivalent fragment, and
a pharmaceutically acceptable carrier,
wherein the at least one rbRNA (e.g., snRNA) or its functionally equivalent fragment activates a primary RNA or DNA sensor, and
wherein the endogenous IFNbeta (IFNβ production of the subject is regulated, and
(b) administering to the subject a therapeutic amount of ionizing radiation.

17. The method of claim 16, wherein the least one rbRNA (e.g., snRNA) or its functionally equivalent fragment is a double-stranded RNA.

18. The method of claim 16, wherein the at least one rbRNA (e.g., snRNA) is selected from the group consisting of EEF1A1P12, EEF1A1P22, RPL31P63, RP11-472I20.1, RNA28S5, RP11-506M13.3, MTND4P12, RPL7P19, MCTS2P, RP11-386I14.4, RP11-506B6.3, RPS4XP13, RP11-332M2.1, RP11-380B4.3, EEF1A1P25, RPS4XP2, RBBP4P1, RP11-304F15.3, RP4-604A21.1, RPL7P16, RP11-165H4.2, CTB-36O1.7, CTD-2006C1.6, RP11-563H6.1, RP5-890O3.9, RPL23P8, CTA-392E5.1, RP5-857K21.11, AC139452.2, RP11-393N4.2, RP11-133K1.1, RP11-378J18.8, RPL5P34, RPS4XP3, RAD21-AS1, EEF1A1P4, MT-TL1, HNRNPA3P3, RP13-216E22.4, RPL5P23, SLIT2-IT1, RP11-785H5.1, RP11-627K11.1, RP11-750B16.1, EEF1B2P3, RP11-17A4.1, CTD-2161E19.1, AC022210.2, and HNRNPA1P35.

19. The method of claim 16, wherein the at least one rbRNA (e.g., snRNA) is selected from the group consisting of U1, U2, M5, M8, LTR25-int, tRNA-Leu-TTA, LTR6A, MamGypsy2-LTR, L1MA2, SSU-rRNA_Hsa, tRNA-Ile-ATT, tRNA-Ser-TCG, G-rich, tRNA-Ser-TCA, LTR103_Mam, MER76, tRNA-Ala-GCG, MER21A, tRNA-Pro-CCG, tRNA-Leu-CTG, tRNA-Val-GTG, LTR21A, GA-rich, tRNA-Pro-CCA, tRNA-Pro-CCY, tRNA-Gln-CAG, tRNA-Gly-GGA, LTR06, tRNA-Val-GTA, LTR78, AmnSINE2, Charlie17, tRNA-Gly-GGY, LTR16E1, AluYk2, LTR46-int, Eulor2B, MER70B, MARE6, tRNA-Thr-ACA, Charlie9, LTR2B, X9_LINE, tRNA-Arg-CGA, LTR30, LTR58, MSR1, AluJo, FRAM, MamGyp-int, tRNA-Arg-AGA, and HY3.

20. The method of claim 16, wherein the at least one rbRNA (e.g., snRNA) is U2.

21. The method of claim 16, wherein the composition further comprises another therapeutic agent.

22. The method of claim 21, wherein the other therapeutic agent is selected from the group consisting of anthracyclines, DNA-topoisomerases inhibitors and cis-platinum preparations or platinum derivatives, such as Cisplatin, camptothecin, the MEK inhibitor: UO 126, a KSP (kinesin spindle protein) inhibitor, adriamycin and interferons.

23. The method of claim 16, wherein at least one rbRNA (e.g., snRNA) or its functionally equivalent fragment is further covalently attached to a reporter group.

24. The method of claim 16, wherein the pharmaceutically acceptable carrier comprises at least one of a nanocarrier, a conjugate, a nucleic-acid-lipid particle, a vesicle, an exosome, a protein capsid, a liposome, a dendrimer, a lipoplex, a micelle, a virosome, a virus like particle, and a nucleic acid complexes.

25. The method of claim 16, wherein the primary RNA or DNA sensor comprises at least one of RIG1, MDA5, DAI, IFI16, Aim2, and cGAS.

26. The method of claim 16, wherein the ionizing radiation comprises at least one of brachytherapy, external beam radiation therapy, and radiation from cesium, iridium, iodine, and cobalt.

27. The method of claim 16, where the subject is a human being.
